SEQUENCE LISTING (311 sequences)

SEQ ID NO: 1 - protein, 28 aa, synthetic construct
ADDQNPWRAYLDLLFPTDTLLLDLLWCG

SEQ ID NO: 2 - protein, 35 aa, synthetic construct
AEQNPIYWARYADWLFTTPLLLLDLALLVDADECG

SEQ ID NO: 3 - protein, 32 aa, synthetic construct
ADDQNPWRAYLDLLFPTDTLLLDLLWDADECG

SEQ ID NO: 4 - protein, 39 aa, synthetic construct
  MOD_RES 1: note = acetylation
AAEQNPIYWARYADWLFTTPLLLLDLALLVDADEGTKCG

SEQ ID NO: 5 - protein, 37 aa, synthetic construct
AAEQNPIYWARYADWLFTTPLLLLDLALLVDADEGTC

SEQ ID NO: 6 - protein, 39 aa, unidentified
AAEQNPIYWWARYADWLFTTPLLLLDLALLVDADEGTCG

SEQ ID NO: 7 - protein, 35 aa, unidentified
AEQNPIYWARYADWLFTTPLLLLDLALLVDADEGT

SEQ ID NO: 8 - protein, 36 aa, unidentified
GGEQNPIYWARYADWLFTTPLLLLDLALLVDADEGT

SEQ ID NO: 9 - protein, 35 aa, unidentified; identical to SEQ ID NO: 7

SEQ ID NO: 10 - protein, 38 aa, synthetic construct
AAEQNPIYWARYADWLFTTPLLLLDLALLVDADEGTCG

SEQ ID NO: 11 - protein, 38 aa, synthetic construct
GGEQNPIYWARYADWLFTTPLLLLDLALLVDADEGTCG

SEQ ID NO: 12 - protein, 37 aa, synthetic construct
ACEQNPIYWARYADWLFTTPLLLLDLALLVDADEGTG

SEQ ID NO: 13 - protein, 36 aa, synthetic construct
ACEQNPIYWARYADWLFTTPLLLLDLALLVDADEGT

SEQ ID NO: 14 - protein, 36 aa, synthetic construct
AKEQNPIYWARYADWLFTTPLLLLDLALLVDADEGT

SEQ ID NO: 15 - protein, 39 aa, synthetic construct; identical to SEQ ID NO: 4

SEQ ID NO: 16 - protein, 36 aa, synthetic construct
AKEQNPIYWARYADWLFTTPLLLLDLALLVDADECT

SEQ ID NO: 17 - protein, 37 aa, synthetic construct
ACEQNPIYWARYANWLFTTPLLLLNLALLVDADEGTG

SEQ ID NO: 18 - protein, 37 aa, synthetic construct
ACEQNPIYWARYAKWLFTTPLLLLKLALLVDADEGTG

SEQ ID NO: 19 - protein, 36 aa, synthetic construct
GGEQNPIYWARYADWLFTTPLLLLDLALLVNANQGT

SEQ ID NO: 20 - protein, 36 aa, synthetic construct
AAEQNPIYWARYADWLFTTPLLLLALALLVDADEGT

SEQ ID NO: 21 - protein, 36 aa, synthetic construct
AAEQNPIYWARYAAWLFTTPLLLLDLALLVDADEGT

SEQ ID NO: 22 - protein, 36 aa, synthetic construct
AAEQNPIYWARYADWLFTTALLLLDLALLVDADEGT

SEQ ID NO: 23 - protein, 36 aa, synthetic construct
AAEQNPIYWARYADWLFTTPLLLLELALLVDADEGT

SEQ ID NO: 24 - protein, 36 aa, synthetic construct
AAEQNPIYWARYAEWLFTTPLLLLDLALLVDADEGT

SEQ ID NO: 25 - protein, 39 aa, synthetic construct
AAEQNPIIYWARYADWLFTDLPLLLLDLLALLVDADEGT

SEQ ID NO: 26 - protein, 37 aa, synthetic construct
GEQNPIYWAQYADWLFTTPLLLLDLALLVDADEGTCG

SEQ ID NO: 27 - protein, 38 aa, synthetic construct
GGEQNPIYWARYADWLFTTPLLLDLLALLVDADEGTCG

SEQ ID NO: 28 - protein, 38 aa, synthetic construct
GGEQNPIYWARYADWLFTTPLLLLLDALLVDADEGTCG

SEQ ID NO: 29 - protein, 38 aa, synthetic construct
GGEQNPIYWARYDAWLFTTPLLLLDLALLVDADEGTCG

SEQ ID NO: 30 - protein, 38 aa, synthetic construct
GGEQNPIYWARYAWDLFTTPLLLLDLALLVDADEGTCG

SEQ ID NO: 31 - protein, 36 aa, synthetic construct
AAEQNPIYWARYADWLFTTGLLLLDLALLVDADEGT

SEQ ID NO: 32 - protein, 37 aa, synthetic construct
DDDEDNPIYWARYADWLFTTPLLLLHGALLVDADECT

SEQ ID NO: 33 - protein, 38 aa, synthetic construct
DDDEDNPIYWARYAHWLFTTPLLLLHGALLVDADEGCT

SEQ ID NO: 34 - protein, 37 aa, synthetic construct
DDDEDNPIYWARYAHWLFTTPLLLLHGALLVNADECT

SEQ ID NO: 35 - protein, 37 aa, synthetic construct
DDDEDNPIYWARYAHWLFTTPLLLLHGALLVNANECT

SEQ ID NO: 36 - protein, 34 aa, synthetic construct
AEQNPIYWARYADFLFTTPLLLLDLALLVDADET

SEQ ID NO: 37 - protein, 35 aa, synthetic construct
AEQNPIYFARYADWLFTTPLLLLDLALLVDADEGT

SEQ ID NO: 38 - protein, 34 aa, synthetic construct
AEQNPIYFARYADFLFTTPLLLLDLALLWDADET

SEQ ID NO: 39 - protein, 32 aa, synthetic construct
AKEDQNPYWARYADWLFTTPLLLLDLALLVDG

SEQ ID NO: 40 - protein, 32 aa, synthetic construct
ACEDQNPYWARYADWLFTTPLLLLDLALLVDG

SEQ ID NO: 41 - protein, 32 aa, synthetic construct
AEDQNPYWARYADWLFTTPLLLLDLALLVDCG

SEQ ID NO: 42 - protein, 32 aa, synthetic construct
AEDQNPYWARYADWLFTTPLLLLELALLVECG

SEQ ID NO: 43 - protein, 30 aa, synthetic construct
AKEDQNPYWRAYADLFTPLTLLDLLALWDG

SEQ ID NO: 44 - protein, 30 aa, synthetic construct
ACEDQNPYWRAYADLFTPLTLLDLLALWDG

SEQ ID NO: 45 - protein, 27 aa, synthetic construct
ACDDQNPWRAYLDLLFPTDTLLLDLLW

SEQ ID NO: 46 - protein, 34 aa, synthetic construct
TEDADVLLALDLLLLPTTFLWDAYRAWYPNQECA

SEQ ID NO: 47 - protein, 20 aa, synthetic construct
AEQNPIYWARYADWLFTTPL

SEQ ID NO: 48 - protein, 21 aa, synthetic construct
AEQNPIYWARYADWLFTTPCL

SEQ ID NO: 49 - protein, 21 aa, synthetic construct
ACEQNPIYWARYADWLFTTPL

SEQ ID NO: 50 - protein, 20 aa, synthetic construct
AEQNPIYFARYADWLFTTPL

SEQ ID NO: 51 - protein, 21 aa, synthetic construct
KEDQNPWARYADLLFPTTLAW

SEQ ID NO: 52 - protein, 22 aa, synthetic construct
ACEDQNPWARYADLLFPTTLAW

SEQ ID NO: 53 - protein, 24 aa, synthetic construct
ACEDQNPWARYADWLFPTTLLLLD

SEQ ID NO: 54 - protein, 22 aa, synthetic construct
ACEEQNPWARYAELLFPTTLAW

SEQ ID NO: 55 - protein, 24 aa, synthetic construct
ACEEQNPWARYAEWLFPTTLLLLE

SEQ ID NO: 56 - protein, 25 aa, synthetic construct
ACEEQNPWARYLEWLFPTETLLLEL

SEQ ID NO: 57 - protein, 36 aa, synthetic construct; identical to SEQ ID NO: 8

SEQ ID NO: 58 - protein, 35 aa, synthetic construct
ACEQNPIYWARYADWLFTTPLLLLDLALLVDADET

SEQ ID NO: 59 - protein, 38 aa, synthetic construct; identical to SEQ ID NO: 10

SEQ ID NO: 60 - protein, 36 aa, synthetic construct
AEQNPIYWARYADWLFTTPLLLLDLALLVDADEGCT

SEQ ID NO: 61 - protein, 38 aa, synthetic construct; identical to SEQ ID NO: 11

SEQ ID NO: 62 - protein, 36 aa, synthetic construct; identical to SEQ ID NO: 13

SEQ ID NO: 63 - protein, 36 aa, synthetic construct; identical to SEQ ID NO: 14

SEQ ID NO: 64 - protein, 36 aa, synthetic construct; identical to SEQ ID NO: 16

SEQ ID NO: 65 - protein, 36 aa, synthetic construct; identical to SEQ ID NO: 22

SEQ ID NO: 66 - protein, 37 aa, synthetic construct
ACAEQNPIYWARYADWLFTTGLLLLDLALLVDADEGT

SEQ ID NO: 67 - protein, 34 aa, synthetic construct; identical to SEQ ID NO: 36

SEQ ID NO: 68 - protein, 35 aa, synthetic construct; identical to SEQ ID NO: 37

SEQ ID NO: 69 - protein, 34 aa, synthetic construct; identical to SEQ ID NO: 38

SEQ ID NO: 70 - protein, 32 aa, synthetic construct; identical to SEQ ID NO: 39

SEQ ID NO: 71 - protein, 32 aa, synthetic construct; identical to SEQ ID NO: 40

SEQ ID NO: 72 - protein, 31 aa, synthetic construct
AEDQNPYWARYADWLFTTPLLLLDLALLVDG

SEQ ID NO: 73 - protein, 32 aa, synthetic construct; identical to SEQ ID NO: 42

SEQ ID NO: 74 - protein, 30 aa, synthetic construct; identical to SEQ ID NO: 43

SEQ ID NO: 75 - protein, 30 aa, synthetic construct; identical to SEQ ID NO: 44

SEQ ID NO: 76 - protein, 32 aa, synthetic construct
AKEDQNDPYWARYADWLFTTPLLLLDLALLVG

SEQ ID NO: 77 - protein, 34 aa, synthetic construct; identical to SEQ ID NO: 46

SEQ ID NO: 78 - protein, 36 aa, unidentified; identical to SEQ ID NO: 8

SEQ ID NO: 79 - protein, 20 aa, synthetic construct
AEQNPIYWARYADWLFTTPL

SEQ ID NO: 80 - protein, 21 aa, synthetic construct
AEQNPIYWARYADWLFTTPCL

SEQ ID NO: 81 - protein, 21 aa, synthetic construct
ACEQNPIYWARYADWLFTTPL

SEQ ID NO: 82 - protein, 21 aa, synthetic construct
ACEQNPIYFARYADWLFTTPL

SEQ ID NO: 83 - protein, 27 aa, synthetic construct
ACDDQNPWRAYLDLLFPTDTLLLDLLW

SEQ ID NO: 84 - protein, 27 aa, synthetic construct
ACEEQNPWRAYLELLFPTETLLLELLW

SEQ ID NO: 85 - protein, 25 aa, synthetic construct
ACDDQNPWARYLDWLFPTDTLLLDL

SEQ ID NO: 86 - protein, 24 aa, synthetic construct
CDNNNPWRAYLDLLFPTDTLLLDW

SEQ ID NO: 87 - protein, 25 aa, synthetic construct
ACEEQNPWARYLEWLFPTETLLLEL

SEQ ID NO: 88 - protein, 24 aa, synthetic construct
ACEDQNPWARYADWLFPTTLLLLD

SEQ ID NO: 89 - protein, 24 aa, synthetic construct
ACEEQNPWARYAEWLFPTTLLLLE

SEQ ID NO: 90 - protein, 22 aa, synthetic construct
ACEDQNPWARYADLLFPTTLAW

SEQ ID NO: 91 - protein, 21 aa, synthetic construct
ACEDQNPWARYAELLFPTTLW

SEQ ID NO: 92 - protein, 20 aa, synthetic construct
KEDQNPWARYADLLFPTTLW

SEQ ID NO: 93 - protein, 37 aa, synthetic construct
DDDEDNPIYWARYAHWLFTTPLLLLHGALLVDADECT

SEQ ID NO: 94 - protein, 37 aa, synthetic construct
DDDEDNPIYWARYAHWLFTTPLLLLDGALLVDADECT

SEQ ID NO: 95 - protein, 37 aa, synthetic construct; identical to SEQ ID NO: 34

SEQ ID NO: 96 - protein, 37 aa, synthetic construct; identical to SEQ ID NO: 35

SEQ ID NO: 97 - protein, 37 aa, synthetic construct; identical to SEQ ID NO: 32

SEQ ID NO: 98 - protein, 37 aa, synthetic construct; identical to SEQ ID NO: 12

SEQ ID NO: 99 - protein, 35 aa, synthetic construct; identical to SEQ ID NO: 58

SEQ ID NO: 100 - protein, 36 aa, synthetic construct; identical to SEQ ID NO: 13

SEQ ID NO: 101 - protein, 38 aa, synthetic construct; identical to SEQ ID NO: 27

SEQ ID NO: 102 - protein, 38 aa, synthetic construct; identical to SEQ ID NO: 28

SEQ ID NO: 103 - protein, 38 aa, synthetic construct; identical to SEQ ID NO: 30

SEQ ID NO: 104 - protein, 38 aa, synthetic construct
AAEQNPIYWARYAEWLFTTPLLLLDLALLVDADEGTCG

SEQ ID NO: 105 - protein, 38 aa, synthetic construct
AAEQNPIYWARYAEWLFTTPLLLLELALLVDADEGTCG

SEQ ID NO: 106 - protein, 38 aa, synthetic construct; identical to SEQ ID NO: 29

SEQ ID NO: 107 - protein, 38 aa, synthetic construct
GGEQNPIYWAQYDAWLFTTPLLLLDLALLVDADEGTCG

SEQ ID NO: 108 - protein, 38 aa, synthetic construct
GGEQNPIYWAQDYAWLFTTPLLLLDLALLVDADEGTCG

SEQ ID NO: 109 - protein, 38 aa, synthetic construct
AAEQNPIYWARYAAWLFTTPLLLLDLALLVDADEGTCG

SEQ ID NO: 110 - protein, 37 aa, synthetic construct; identical to SEQ ID NO: 17

SEQ ID NO: 111 - protein, 37 aa, synthetic construct; identical to SEQ ID NO: 35

SEQ ID NO: 112 - protein, 37 aa, synthetic construct; identical to SEQ ID NO: 34

SEQ ID NO: 113 - protein, 37 aa, synthetic construct; identical to SEQ ID NO: 32

SEQ ID NO: 114 - protein, 37 aa, synthetic construct; identical to SEQ ID NO: 93

SEQ ID NO: 115 - protein, 37 aa, synthetic construct; identical to SEQ ID NO: 94

SEQ ID NO: 116 - protein, 36 aa, synthetic construct; identical to SEQ ID NO: 19

SEQ ID NO: 117 - protein, 38 aa, synthetic construct
AAEQNPIYWARYADWLFTTPLLLLELALLVDADEGTCG

SEQ ID NO: 118 - protein, 38 aa, synthetic construct; identical to SEQ ID NO: 105

SEQ ID NO: 119 - protein, 39 aa, synthetic construct
AAEQNPIYWARYADWLFTTPLLLLELALLVDADEGTKCG

SEQ ID NO: 120 - protein, 38 aa, synthetic construct
GGEQNPIYWAQYADWLFTTPLLLLDLALLVDADEGTCG

SEQ ID NO: 121 - protein, 38 aa, synthetic construct; identical to SEQ ID NO: 107

SEQ ID NO: 122 - protein, 38 aa, synthetic construct; identical to SEQ ID NO: 108

SEQ ID NO: 123 - protein, 36 aa, synthetic construct; identical to SEQ ID NO: 19

SEQ ID NO: 124 - protein, 37 aa, synthetic construct; identical to SEQ ID NO: 34

SEQ ID NO: 125 - protein, 37 aa, synthetic construct; identical to SEQ ID NO: 35

SEQ ID NO: 126 - protein, 37 aa, synthetic construct; identical to SEQ ID NO: 18

SEQ ID NO: 127 - protein, 38 aa, synthetic construct; identical to SEQ ID NO: 108

SEQ ID NO: 128 - protein, 38 aa, synthetic construct; identical to SEQ ID NO: 107

SEQ ID NO: 129 - protein, 38 aa, synthetic construct; identical to SEQ ID NO: 120

SEQ ID NO: 130 - protein, 38 aa, synthetic construct; identical to SEQ ID NO: 109

SEQ ID NO: 131 - protein, 38 aa, synthetic construct
AAEQNPIYWARYADWLFTDLPLLLLDLLALLVDADEGT

SEQ ID NO: 132 - protein, 38 aa, synthetic construct; identical to SEQ ID NO: 28

SEQ ID NO: 133 - protein, 38 aa, synthetic construct; identical to SEQ ID NO: 27

SEQ ID NO: 134 - protein, 36 aa, synthetic construct; identical to SEQ ID NO: 31

SEQ ID NO: 135 - protein, 38 aa, synthetic construct; identical to SEQ ID NO: 109

SEQ ID NO: 136 - protein, 38 aa, synthetic construct; identical to SEQ ID NO: 107

SEQ ID NO: 137 - protein, 38 aa, synthetic construct; identical to SEQ ID NO: 108

SEQ ID NO: 138 - protein, 38 aa, synthetic construct; identical to SEQ ID NO: 29

SEQ ID NO: 139 - protein, 38 aa, synthetic construct
AAEQNPIYWARYADWLFTTPLLLLALALLVDADEGTCG

SEQ ID NO: 140 - protein, 39 aa, synthetic construct; identical to SEQ ID NO: 4
  MOD_RES 37: note = Lys(rhodamine)
  MOD_RES 38: note = Cys(phalloidin)

SEQ ID NO: 141 - protein, 39 aa, synthetic construct; identical to SEQ ID NO: 119
  MOD_RES 37: note = Lys(rhodamine)
  MOD_RES 38: note = Cys(phalloidin)

SEQ ID NO: 142 - protein, 38 aa, synthetic construct; identical to SEQ ID NO: 10
  MOD_RES 37: note = Cys(phalloidin)

SEQ ID NO: 143 - protein, 38 aa, synthetic construct; identical to SEQ ID NO: 10
  MOD_RES 37: note = Cys(phalloidin)

SEQ ID NO: 144 - protein, 38 aa, synthetic construct; identical to SEQ ID NO: 11

SEQ ID NO: 145 - protein, 35 aa, synthetic construct; identical to SEQ ID NO: 58

SEQ ID NO: 146 - protein, 37 aa, synthetic construct; identical to SEQ ID NO: 12

SEQ ID NO: 147 - protein, 36 aa, synthetic construct; identical to SEQ ID NO: 13

SEQ ID NO: 148 - protein, 36 aa, synthetic construct; identical to SEQ ID NO: 19

SEQ ID NO: 149 - protein, 37 aa, synthetic construct; identical to SEQ ID NO: 34

SEQ ID NO: 150 - protein, 37 aa, synthetic construct; identical to SEQ ID NO: 35

SEQ ID NO: 151 - protein, 38 aa, synthetic construct; identical to SEQ ID NO: 11

SEQ ID NO: 152 - protein, 38 aa, synthetic construct; identical to SEQ ID NO: 10
  MOD_RES 37: note = Cys(phalloidin)

SEQ ID NO: 153 - protein, 39 aa, synthetic construct; identical to SEQ ID NO: 119
  MOD_RES 37: note = Lys(rhodamine)
  MOD_RES 38: note = Cys(phalloidin)

SEQ ID NO: 154 - protein, 39 aa, synthetic construct; identical to SEQ ID NO: 4
  MOD_RES 37: note = Lys(rhodamine)
  MOD_RES 38: note = Cys(phalloidin)

SEQ ID NO: 155 - protein, 37 aa, synthetic construct; identical to SEQ ID NO: 93

SEQ ID NO: 156 - protein, 37 aa, synthetic construct; identical to SEQ ID NO: 94

SEQ ID NO: 157 - protein, 37 aa, synthetic construct; identical to SEQ ID NO: 34

SEQ ID NO: 158 - protein, 37 aa, synthetic construct; identical to SEQ ID NO: 35

SEQ ID NO: 159 - protein, 37 aa, synthetic construct; identical to SEQ ID NO: 32

SEQ ID NO: 160 - protein, 37 aa, synthetic construct; identical to SEQ ID NO: 32

SEQ ID NO: 161 - protein, 37 aa, synthetic construct; identical to SEQ ID NO: 94

SEQ ID NO: 162 - protein, 37 aa, synthetic construct; identical to SEQ ID NO: 93

SEQ ID NO: 163 - protein, 37 aa, synthetic construct; identical to SEQ ID NO: 34

SEQ ID NO: 164 - protein, 37 aa, synthetic construct; identical to SEQ ID NO: 35

SEQ ID NO: 165 - protein, 37 aa, synthetic construct; identical to SEQ ID NO: 35

SEQ ID NO: 166 - protein, 37 aa, synthetic construct; identical to SEQ ID NO: 34

SEQ ID NO: 167 - protein, 37 aa, synthetic construct; identical to SEQ ID NO: 32

SEQ ID NO: 168 - protein, 37 aa, synthetic construct; identical to SEQ ID NO: 93

SEQ ID NO: 169 - protein, 37 aa, synthetic construct; identical to SEQ ID NO: 94

SEQ ID NO: 170 - protein, 36 aa, synthetic construct; identical to SEQ ID NO: 19

SEQ ID NO: 171 - protein, 37 aa, synthetic construct; identical to SEQ ID NO: 34

SEQ ID NO: 172 - protein, 37 aa, synthetic construct; identical to SEQ ID NO: 32

SEQ ID NO: 173 - protein, 37 aa, synthetic construct; identical to SEQ ID NO: 93

SEQ ID NO: 174 - protein, 37 aa, synthetic construct; identical to SEQ ID NO: 94

SEQ ID NO: 175 - protein, 37 aa, synthetic construct; identical to SEQ ID NO: 35

SEQ ID NO: 176 - protein, 37 aa, synthetic construct; identical to SEQ ID NO: 94

SEQ ID NO: 177 - protein, 37 aa, synthetic construct; identical to SEQ ID NO: 32

SEQ ID NO: 178 - protein, 37 aa, synthetic construct; identical to SEQ ID NO: 93

SEQ ID NO: 179 - protein, 37 aa, synthetic construct; identical to SEQ ID NO: 34

SEQ ID NO: 180 - protein, 37 aa, synthetic construct; identical to SEQ ID NO: 35

SEQ ID NO: 181 - protein, 36 aa, synthetic construct; identical to SEQ ID NO: 31

SEQ ID NO: 182 - protein, 38 aa, synthetic construct; identical to SEQ ID NO: 30

SEQ ID NO: 183 - protein, 38 aa, synthetic construct; identical to SEQ ID NO: 29

SEQ ID NO: 184 - protein, 38 aa, synthetic construct; identical to SEQ ID NO: 107

SEQ ID NO: 185 - protein, 38 aa, synthetic construct; identical to SEQ ID NO: 108

SEQ ID NO: 186 - protein, 38 aa, synthetic construct; identical to SEQ ID NO: 109

SEQ ID NO: 187 - protein, 38 aa, synthetic construct; identical to SEQ ID NO: 28

SEQ ID NO: 188 - protein, 38 aa, synthetic construct; identical to SEQ ID NO: 27

SEQ ID NO: 189 - protein, 38 aa, synthetic construct; identical to SEQ ID NO: 27

SEQ ID NO: 190 - protein, 38 aa, synthetic construct; identical to SEQ ID NO: 28

SEQ ID NO: 191 - protein, 38 aa, synthetic construct; identical to SEQ ID NO: 120

SEQ ID NO: 192 - protein, 38 aa, synthetic construct; identical to SEQ ID NO: 107

SEQ ID NO: 193 - protein, 38 aa, synthetic construct; identical to SEQ ID NO: 108

SEQ ID NO: 194 - protein, 38 aa, synthetic construct; identical to SEQ ID NO: 107

SEQ ID NO: 195 - protein, 38 aa, synthetic construct; identical to SEQ ID NO: 108

SEQ ID NO: 196 - protein, 38 aa, synthetic construct; identical to SEQ ID NO: 120

SEQ ID NO: 197 - protein, 38 aa, synthetic construct; identical to SEQ ID NO: 109

SEQ ID NO: 198 - protein, 38 aa, synthetic construct; identical to SEQ ID NO: 108

SEQ ID NO: 199 - protein, 38 aa, synthetic construct; identical to SEQ ID NO: 107

SEQ ID NO: 200 - protein, 38 aa, synthetic construct; identical to SEQ ID NO: 120

SEQ ID NO: 201 - protein, 38 aa, synthetic construct; identical to SEQ ID NO: 109

SEQ ID NO: 202 - protein, 39 aa, synthetic construct; identical to SEQ ID NO: 119

SEQ ID NO: 203 - protein, 6 aa, synthetic construct
  MOD_RES 4: note = Lys(rhodamine)
  MOD_RES 5: note = Cys(phalloidin)
EGTKCG

SEQ ID NO: 204 - protein, 39 aa, synthetic construct; identical to SEQ ID NO: 4
  MOD_RES 37: note = Lys(rhodamine)
  MOD_RES 38: note = Cys(phalloidin)

SEQ ID NO: 205 - protein, 37 aa, synthetic construct; identical to SEQ ID NO: 12

SEQ ID NO: 206 - protein, 38 aa, synthetic construct; identical to SEQ ID NO: 10
  MOD_RES 37: note = Cys(phalloidin)

SEQ ID NO: 207 - protein, 39 aa, synthetic construct; identical to SEQ ID NO: 4
  MOD_RES 37: note = Lys(rhodamine)
  MOD_RES 38: note = Cys(phalloidin)

SEQ ID NO: 208 - protein, 39 aa, synthetic construct; identical to SEQ ID NO: 119
  MOD_RES 37: note = Lys(rhodamine)
  MOD_RES 38: note = Cys(phalloidin)

SEQ ID NO: 209 - protein, 38 aa, synthetic construct; identical to SEQ ID NO: 131

SEQ ID NO: 210 - protein, 38 aa, synthetic construct; identical to SEQ ID NO: 109

SEQ ID NO: 211 - protein, 38 aa, synthetic construct; identical to SEQ ID NO: 107

SEQ ID NO: 212 - protein, 38 aa, synthetic construct; identical to SEQ ID NO: 108

SEQ ID NO: 213 - protein, 38 aa, synthetic construct; identical to SEQ ID NO: 29

SEQ ID NO: 214 - protein, 38 aa, synthetic construct; identical to SEQ ID NO: 104

SEQ ID NO: 215 - protein, 38 aa, synthetic construct; identical to SEQ ID NO: 105

SEQ ID NO: 216 - protein, 38 aa, synthetic construct; identical to SEQ ID NO: 139

SEQ ID NO: 217 - protein, 38 aa, synthetic construct; identical to SEQ ID NO: 117

SEQ ID NO: 218 - protein, 38 aa, synthetic construct; identical to SEQ ID NO: 105

SEQ ID NO: 219 - protein, 39 aa, synthetic construct; identical to SEQ ID NO: 119
  MOD_RES 37: note = Lys(rhodamine)
  MOD_RES 38: note = Cys(phalloidin)

SEQ ID NO: 220 - protein, 37 aa, synthetic construct; identical to SEQ ID NO: 18

SEQ ID NO: 221 - protein, 37 aa, synthetic construct; identical to SEQ ID NO: 17

SEQ ID NO: 222 - protein, 36 aa, synthetic construct; identical to SEQ ID NO: 22

SEQ ID NO: 223 - protein, 22 aa, synthetic construct
AEQNPIYFARYADLLFPTTLAW

SEQ ID NO: 224 - protein, 22 aa, synthetic construct
AEQNPIYWARYADLLFPTTLAF

SEQ ID NO: 225 - protein, 22 aa, synthetic construct
AEQNPIYWARYADLLFPTTLAW

SEQ ID NO: 226 - protein, 35 aa, synthetic construct; identical to SEQ ID NO: 58

SEQ ID NO: 227 - protein, 36 aa, synthetic construct; identical to SEQ ID NO: 8

SEQ ID NO: 228 - protein, 38 aa, synthetic construct; identical to SEQ ID NO: 10

SEQ ID NO: 229 - protein, 39 aa, synthetic construct; identical to SEQ ID NO: 4

SEQ ID NO: 230 - protein, 36 aa, synthetic construct; identical to SEQ ID NO: 16

SEQ ID NO: 231 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 232 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 233 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 234 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 235 - DNA, 18 bp, synthetic construct
  source 1..18: mol_type = other DNA
  modified_base 1..18: mod_base = OTHER; note = N-(2-aminoethyl) glycine nucleosides
cctctgacctcatttaca

SEQ ID NO: 236 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 237 - DNA, 18 bp, synthetic construct
  source 1..18: mol_type = other DNA
  modified_base 1..18: mod_base = OTHER; note = N-(2-aminoethyl) glycine nucleosides
cctctgacctcatttaca

SEQ ID NO: 238 - DNA, 18 bp, synthetic construct
  source 1..18: mol_type = other DNA
  modified_base 1..18: mod_base = OTHER; note = N-(2-aminoethyl) glycine nucleosides
cctcttacctcagttaca

SEQ ID NO: 239 - protein, 38 aa, unidentified; identical to SEQ ID NO: 10

SEQ ID NO: 240 - protein, 31 aa, synthetic construct
AEDQNPYWARYDWLFTTPLLLLDLALLVDCG

SEQ ID NO: 241 - protein, 32 aa, synthetic construct; identical to SEQ ID NO: 42

SEQ ID NO: 242 - protein, 36 aa, synthetic construct; identical to SEQ ID NO: 60

SEQ ID NO: 243 - protein, 35 aa, synthetic construct; identical to SEQ ID NO: 58

SEQ ID NO: 244 - protein, 35 aa, synthetic construct; identical to SEQ ID NO: 7

SEQ ID NO: 245 - protein, 31 aa, synthetic construct; identical to SEQ ID NO: 72

SEQ ID NO: 246 - protein, 31 aa, synthetic construct
AEDQNDPYWARYADWLFTTPLLLLDLALLVG

SEQ ID NO: 247 - protein, 34 aa, synthetic construct; identical to SEQ ID NO: 36

SEQ ID NO: 248 - protein, 34 aa, synthetic construct
AEQNPIYFARYADWLFTTPLLLLDLALLVDADET

SEQ ID NO: 249 - protein, 34 aa, synthetic construct; identical to SEQ ID NO: 38

SEQ ID NO: 250 - protein, 36 aa, synthetic construct; identical to SEQ ID NO: 60

SEQ ID NO: 251 - protein, 34 aa, synthetic construct
AEDQNPIYWARYADWLFTTPLLLLDLALLVDCGT

SEQ ID NO: 252 - protein, 35 aa, synthetic construct
AEDQNDPIYWARYADWLFTTPLLLLELALLVECGT

SEQ ID NO: 253 - protein, 25 aa, synthetic construct
ACEEQNPWARYLEWLFPTETLLLEL

SEQ ID NO: 254 - protein, 35 aa, synthetic construct; identical to SEQ ID NO: 7

SEQ ID NO: 255 - protein, 32 aa, synthetic construct; identical to SEQ ID NO: 39

SEQ ID NO: 256 - protein, 32 aa, synthetic construct; identical to SEQ ID NO: 76

SEQ ID NO: 257 - protein, 35 aa, synthetic construct
AEQNPIYWARYADWLFTTPLLLLDLALLVDADEGC

SEQ ID NO: 258 - protein, 31 aa, synthetic construct
AEDQNPYWARYADWLFTTPLLLLDLALLVDC

SEQ ID NO: 259 - protein, 31 aa, synthetic construct
AEDQNPYWARYADWLFTTPLLLLELALLVEC

SEQ ID NO: 260 - protein, 36 aa, synthetic construct; identical to SEQ ID NO: 13

SEQ ID NO: 261 - protein, 32 aa, synthetic construct; identical to SEQ ID NO: 40

SEQ ID NO: 262 - protein, 30 aa, synthetic construct; identical to SEQ ID NO: 44

SEQ ID NO: 263 - protein, 27 aa, synthetic construct
ACDDQNPWRAYLDLLFPTDTLLLDLLW

SEQ ID NO: 264 - protein, 27 aa, synthetic construct
ACEEQNPWRAYLELLFPTETLLLELLW

SEQ ID NO: 265 - protein, 25 aa, synthetic construct
ACDDQNPWARYLDWLFPTDTLLLDL

SEQ ID NO: 266 - protein, 24 aa, synthetic construct
CDNNNPWRAYLDLLFPTDTLLLDW

SEQ ID NO: 267 - protein, 25 aa, synthetic construct
ACEEQNPWARYLEWLFPTETLLLEL

SEQ ID NO: 268 - protein, 24 aa, synthetic construct
CEEQQPWAQYLELLFPTETLLLEW

SEQ ID NO: 269 - protein, 24 aa, synthetic construct
CEEQQPWRAYLELLFPTETLLLEW

SEQ ID NO: 270 - protein, 24 aa, synthetic construct
ACEDQNPWARYADWLFPTTLLLLD

SEQ ID NO: 271 - protein, 24 aa, synthetic construct
ACEEQNPWARYAEWLFPTTLLLLE

SEQ ID NO: 272 - protein, 22 aa, synthetic construct
ACEDQNPWARYADLLFPTTLAW

SEQ ID NO: 273 - protein, 22 aa, synthetic construct
ACEEQNPWARYAELLFPTTLAW

SEQ ID NO: 274 - protein, 34 aa, synthetic construct; identical to SEQ ID NO: 46

SEQ ID NO: 275 - protein, 37 aa, synthetic construct
CDDDDDNPNYWARYANWLFTTPLLLLNGALLVEAEET

SEQ ID NO: 276 - protein, 37 aa, synthetic construct
CDDDDDNPNYWARYAPWLFTTPLLLLPGALLVEAEET

SEQ ID NO: 277 - protein, 36 aa, synthetic construct; identical to SEQ ID NO: 60

SEQ ID NO: 278 - protein, 37 aa, synthetic construct
AKEQNPIYWARYADWLFTTPLLLLDLALLVDADEGTG

SEQ ID NO: 279 - protein, 36 aa, synthetic construct
ACEQNPIYWARYANWLFTTPLLLLNLALLVDADEGT

SEQ ID NO: 280 - protein, 36 aa, synthetic construct
DDDEDNPIYWARYADWLFTTPLLLLHGALLVDADET

SEQ ID NO: 281 - protein, 36 aa, synthetic construct; identical to SEQ ID NO: 280

SEQ ID NO: 282 - protein, 37 aa, synthetic construct
CDDDEDNPIYWARYAHWLFTTPLLLLHGALLVDADET

SEQ ID NO: 283 - protein, 37 aa, synthetic construct
DDDEDNPIYWARYAHWLFTTPLLLLHGALLVDADEGT

SEQ ID NO: 284 - protein, 37 aa, synthetic construct
DDDEDNPIYWARYAHWLFTTPLLLLHGALLVNADEGT

SEQ ID NO: 285 - protein, 37 aa, synthetic construct
DDDEDNPIYWARYAHWLFTTPLLLLHGALLVNANEGT

SEQ ID NO: 286 - protein, 32 aa, synthetic construct; identical to SEQ ID NO: 76

SEQ ID NO: 287 - protein, 31 aa, synthetic construct
AEDQNPYWARYADWLFTTPLLLLELALLVCG

SEQ ID NO: 288 - protein, 28 aa, synthetic construct
AKDDQNPWRAYLDLLFPTDTLLLDLLWC

SEQ ID NO: 289 - protein, 27 aa, synthetic construct
ACEEQNPWRAYLELLFPTETLLLELLW

SEQ ID NO: 290 - protein, 25 aa, synthetic construct
ACDDQNPWARYLDWLFPTDTLLLDL

SEQ ID NO: 291 - protein, 24 aa, synthetic construct
CDNNNPWRAYLDLLFPTDTLLLDW

SEQ ID NO: 292 - protein, 24 aa, synthetic construct
CEEQQPWAQYLELLFPTETLLLEW

SEQ ID NO: 293 - protein, 24 aa, synthetic construct
CEEQQPWRAYLELLFPTETLLLEW

SEQ ID NO: 294 - protein, 37 aa, synthetic construct; identical to SEQ ID NO: 275

SEQ ID NO: 295 - protein, 36 aa, synthetic construct
CDDDDDNPNYWARYAPWLFTTPLLLLPGALLVEAEE

SEQ ID NO: 296 - protein, 22 aa, synthetic construct
AEQNPIYFARYADLLFPTTLAW

SEQ ID NO: 297 - protein, 22 aa, synthetic construct
AEQNPIYWARYADLLFPTTLAF

SEQ ID NO: 298 - protein, 22 aa, synthetic construct
AEQNPIYWARYADLLFPTTLAW

SEQ ID NO: 299 - protein, 20 aa, synthetic construct
KEDQNPWARYADLLFPTTLW

SEQ ID NO: 300 - protein, 24 aa, synthetic construct
ACEEQNPQAEYAEWLFPTTLLLLE

SEQ ID NO: 301 - protein, 25 aa, synthetic construct
AAEEQNPWARYLEWLFPTETLLLEL

SEQ ID NO: 302 - protein, 25 aa, synthetic construct
AKEEQNPWARYLEWLFPTETLLLEL

SEQ ID NO: 303 - protein, 38 aa, unidentified
AAEQNPIYWARYADWLFTTPLLLLDLALLVDADEGTGG

SEQ ID NO: 304 - protein, 38 aa, synthetic construct
  VARIANT 1: note = Ala or Asp
  VARIANT 2: note = Ala, Asp or Cys
  VARIANT 4: note = Gln or Asp
  VARIANT 11: note = Arg or Gln
  VARIANT 12: note = Tyr or Asp
  VARIANT 13: note = Ala, Asp or Tyr
  VARIANT 14: note = Asp, Asn, Glu, His, Lys, Ala or Trp
  VARIANT 15: note = Trp or Asp
  VARIANT 19: note = Thr or Asp
  VARIANT 20: note = Pro, Gly or Ala
  VARIANT 24: note = Leu or Asp
  VARIANT 26: note = Leu, Asp or Gly
  VARIANT 31: note = Asp or Asn
  VARIANT 33: note = Asp or Asn
  VARIANT 34: note = Glu or Gln
  VARIANT 35: note = Gly or Cys
  VARIANT 37: note = Gly or Cys
  VARIANT 25: note = Asp, Leu, Asn, Glu, His, Lys or Ala
XXEXNPIYWAXXXXXLFTXXLLLXXXALLVXAXXXTXG

SEQ ID NO: 305 - protein, 42 aa, synthetic construct
  SITE 1: note = may or may not be present
  SITE 29: note = may or may not be present
  SITE 40: note = may or may not be present
  SITE 21: note = may or may not be present
DAAEQNPIYWARYADWLFTTLPLLLLDLLALLVDADEGTKGG

SEQ ID NO: 306 - protein, 38 aa, unidentified
GGEQNPIYWARYADWLFTTPLLLLDLALLVDADEGTGG

SEQ ID NO: 307 - protein, 38 aa, synthetic construct
  VARIANT 1: note = Gly, Asp or Ala
  VARIANT 2: note = Gly, Asp or Cys
  VARIANT 4: note = Gln or Asp
  VARIANT 11: note = Arg or Gln
  VARIANT 12: note = Tyr or Asp
  VARIANT 13: note = Ala, Asp or Tyr
  VARIANT 14: note = Asp, Asn, Glu, His, Lys, Ala or Trp
  VARIANT 15: note = Trp or Asp
  VARIANT 19: note = Thr or Asp
  VARIANT 20: note = Pro, Gly or Ala
  VARIANT 24: note = Leu or Asp
  VARIANT 25: note = Asp, Leu, Asn, Glu, His, Lys or Ala
  VARIANT 33: note = Asp or Asn
  VARIANT 35: note = Gly or Cys
  VARIANT 26: note = Leu, Asp or Gly
  VARIANT 31: note = Ala or Aln
  VARIANT 34: note = Glu or Gln
XXEXNPIYWAXXXXXLFTXXLLLXXXALLVXAXXXTGG

SEQ ID NO: 308 - protein, 44 aa, synthetic construct
  VARIANT 1: note = May or may not be present
  VARIANT 7: note = May or may not be present
  VARIANT 22: note = May or may not be present
  VARIANT 30: note = May or may not be present
  VARIANT 40: note = May or may not be present
  VARIANT 42: note = Lys, Cys or absent
DGGEQNDPIYWARYADWLFTTLPLLLLDLLALLVDADEGCTXGG

SEQ ID NO: 309 - protein, 38 aa, synthetic construct; identical to SEQ ID NO: 10
  MOD_RES 37: note = S-S linker attached to the nitrogen of amino-phalloidin

SEQ ID NO: 310 - protein, 31 aa, synthetic construct; identical to SEQ ID NO: 240
  MOD_RES 30: note = S-S linker attached to 2-amino phalloidin
  MOD_RES 29: note = alkyl linker attached to rhodamine
  MOD_RES 1: note = acetylated

SEQ ID NO: 311 - protein, 34 aa, synthetic construct
GLAGLAGLLGLEGLLGLPLGLLEGLWLGLELEGN

(19) United States
(12) Patent Application Publication
(10) Pub. No.: US 2023/0416331 A1
(43) Pub. Date: Dec. 28, 2023

(54) PEPTIDE CONJUGATES OF PEPTIDIC TUBULIN INHIBITORS AS THERAPEUTICS

(71) Applicant: Cybrexa 4, Inc., New Haven, CT (US)

(72) Inventors: Robert John Maguire, Madison, CT (US); Johanna Marie Csengery, New Haven, CT (US)

(21) Appl. No.: 18/056,117

(22) Filed: Nov. 16, 2022

Related U.S. Application Data
(60) Provisional application No. 63/280,409, filed on Nov. 17, 2021.

(51) Int. Cl. C07K 14/655; C07K 14/00; A61P 35/00

(52) U.S. Cl. CPC C07K 14/655; A61P 35/00; C07K 14/001

(57) ABSTRACT

The present invention relates to peptide conjugates of peptidic tubulin inhibitors (e.g., monomethyl auristatins) which are useful for the treatment of diseases such as cancer.

### CROSS-REFERENCE TO RELATED APPLICATIONS

This application claims the benefit of U.S. Provisional Application Ser. No. 63/280,409 filed Nov. 17, 2021, which is incorporated herein by reference in its entirety.

### FIELD OF THE INVENTION

The present invention relates to peptide conjugates of peptidic tubulin inhibitors, such as monomethyl auristatins, which are useful for the treatment of diseases such as cancer.

### SEQUENCE LISTING

This application contains a Sequence Listing that has been submitted electronically as an XML file named “43236-0020001_SL_ST26.XML”. The XML file, created on Jun. 14, 2023, is 309,653 bytes in size. The material in the XML file is hereby incorporated by reference in its entirety.

### BACKGROUND OF THE INVENTION

Cancer is a group of diseases characterized by aberrant control of cell growth. The annual incidence of cancer is estimated to be in excess of 1.6 million in the United States alone. While surgery, radiation, chemotherapy, and hormones are used to treat cancer, it remains the second leading cause of death in the U.S. It is estimated that about 600,000 Americans will die from cancer each year.

Treatment of cancer in humans by systemic administration of pharmaceutical agents often functions by slowing or terminating the uncontrolled replication that is a characteristic of cancer cells. Peptidic tubulin inhibitors such as dolastatins, the dolastatin-derived auristatins, monomethyl auristatins (e.g., monomethyl auristatin E and monomethyl auristatin F), and tubulysins are a class of antimitotic agents that inhibit tubulin polymerization and can display high potency on a broad array of cancer cells. Due to their often high cytotoxicity, peptidic tubulin inhibitors, such as the monomethyl auristatins, have been conjugated to tumor targeting agents such as antibodies in order to reduce off-target effects. Even so, antibody drug conjugates of peptidic tubulin inhibitors (e.g., monomethyl auristatins) can exhibit several severe side-effects, including neutropenia, neuropathy, thrombocytopenia, and ocular toxicities. Thus, there is a need for more selective delivery of peptidic tubulin inhibitor compounds to diseased tissue.

### SUMMARY

The present disclosure provides, inter alia, a compound of Formula (I):

R²-L-R¹ (I)

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure also provides methods of treating a disease or condition (e.g., cancer) by administering to a human or other mammal in need of such treatment a therapeutically effective amount of a compound of the disclosure. In some embodiments, the disease or condition is characterized by acidic or hypoxic diseased tissues.

The present disclosure also provides use of a compound described herein in the manufacture of a medicament for use in therapy. The present disclosure also provides the compounds described herein for use in therapy.

The present disclosure also provides methods for synthesizing the compounds of the disclosure and intermediates useful in these methods.

### BRIEF DESCRIPTION OF THE DRAWINGS

FIG. 1A shows a plot of the growth delay of HCT116 colorectal cells in vitro after four day incubation with the indicated concentrations of Compound 2 or unconjugated MMAE.

FIG. 1B shows a plot of the growth delay of PC3 prostate cells in vitro after four day incubation with the indicated concentrations of Compound 2 or unconjugated MMAE.

FIG. 1C shows a plot of the growth delay of NCI-H1975 NSCLC cells in vitro after four day incubation with the indicated concentrations of Compound 2 or unconjugated MMAE.

FIG. 1D shows a plot of the growth delay of NCI-H292 NSCLC cells in vitro after four day incubation with the indicated concentrations of Compound 2 or unconjugated MMAE.

FIG. 2A shows a cell cycle analysis of HCT116 colorectal cells in vitro after 24 h incubation with the indicated doses of unconjugated MMAE.

FIG. 2B shows cell cycle analysis of HCT116 colorectal cells in vitro after 24 h incubation with the indicated doses of Compound 2.

FIG. 3 shows a plot of the plasma concentration of Compound 2 and released MMAE after a single IV dose of 10 mg/kg of Compound 2 in the rat (data are expressed as means±SD).

FIG. 4A shows a plot of the levels of unconjugated MMAE in mouse tumor determined by LCMS after a single intraperitoneal injection of either 0.5 mg/kg MMAE or 3 mg/kg Compound 2 in HCT116 colorectal tumor bearing female nude mice.

FIG. 4B shows a plot of the levels of unconjugated MMAE in mouse muscle determined by LCMS after a single intraperitoneal injection of either 0.5 mg/kg MMAE or 3 mg/kg Compound 2 in HCT116 colorectal tumor bearing female nude mice.

FIG. 4C shows a plot of the levels of unconjugated MMAE in mouse bone marrow determined by LCMS after a single intraperitoneal injection of either 0.5 mg/kg MMAE or 3 mg/kg Compound 2 in HCT116 colorectal tumor bearing female nude mice.

FIG. 5A shows a plot of the mean tumor volume resulting from dosing either 0.25 mg/kg MMAE or 40 mg/kg Compound 1 (7 mg/kg MMAE equivalent) in nude mice bearing HCT116 HER2 negative colorectal flank tumors. Animals were dosed once daily intraperitoneally for a total of two days.

FIG. 5B shows a plot of the percent change in body weight of nude mice bearing HCT116 HER2 negative colorectal flank tumors, dosed with either 0.25 mg/kg MMAE or 40 mg/kg Compound 1 (7 mg/kg MMAE equivalent).

FIG. 6A shows a plot of the mean tumor volume resulting from dosing 20 mg/kg Compound 2 in nude mice bearing PC3 prostate adenocarcinoma flank tumors. Animals were dosed once daily two times per week intraperitoneally for three weeks.

FIG. 6B displays percent change in body weight of animals in the study of Example F. Data are expressed as means±SEM.

FIG. 7A shows a plot of the mean tumor volume resulting from dosing 10 or 20 mg/kg Compound 2 in nude mice bearing NCI-H1975 non-small cell lung cancer flank tumors. Animals were dosed once daily two times per week intraperitoneally for three weeks.

FIG. 7B displays percent change in body weight of animals in the study of Example G. Data are expressed as means±SEM.

FIG. 8 shows a plot of body weights of nude mice dosed with 10 mg/kg Compound 1 and Compound 2 once daily for four consecutive days.

FIG. 9A shows a plot of the peptide concentrations in tumor after a single 10 mg/kg IP dose of either Compound 7 or Compound 13 in HCT116 colorectal tumor bearing female nude mice (data are expressed as means±SD).

FIG. 9B shows a plot of the MMAE concentrations in tumor after a single 10 mg/kg IP dose of either Compound 7 or Compound 13 in HCT116 colorectal tumor bearing female nude mice (data are expressed as means±SD).

FIG. 10A shows a plot of the mean tumor volume resulting from dosing 5 mg/kg Compound 13 in nude mice bearing HT-29 colorectal cancer flank tumors. Animals were dosed once daily intraperitoneally on days 0-3, 5 and 16-19.

FIG. 10B displays percent change in body weight of animals in the study of Example J. Data are expressed as means±SEM.

FIG. 11A shows a plot of the mean tumor volume resulting from dosing 40 and 80 mg/kg Compound 7 in nude mice bearing HT-29 colorectal cancer flank tumors. Animals were dosed once daily intraparenterally for four consecutive days a week for two weeks.

FIG. 11B displays percent change in body weight of animals in the study of Example K. Data are expressed as means±SEM.

FIG. 12A shows a plot of the peptide concentrations in tumor after a single 10 mg/kg intraperitoneal dose of Compound 13, Compound 1, or Compound 2 in HCT116 colorectal tumor bearing female nude mice (data are expressed as means±SD).

FIG. 12B shows a plot of the MMAE concentrations in tumor after a single 10 mg/kg intraperitoneal dose of Compound 13, Compound 1, or Compound 2 in HCT116 colorectal tumor bearing female nude mice (data are expressed as means±SD).

FIG. 13A shows a plot of the levels of peptide in mouse tumor determined by ELISA and LCMS after a single 10 mg/kg intraperitoneal injection of Compound 13, Compound 7, Compound 5, or Compound 6 in HCT116 colorectal tumor bearing female nude mice (data are expressed as means±SD).

FIG. 13B shows a plot of the levels of unconjugated MMAE in mouse tumor determined by ELISA and LCMS after a single 10 mg/kg intraperitoneal injection Compound 13, Compound 7, Compound 5, or Compound 6 in HCT116 colorectal tumor bearing female nude mice (data are expressed as means±SD).

FIG. 14A shows a plot of the mean tumor volume resulting from dosing 1, 5 and 10 mg/kg Compound 5 in nude mice bearing HCT116 colorectal cancer flank tumors. Animals were dosed once daily intraparenterally for four consecutive days.

FIG. 14B displays percent change in body weight of animals in the study of Example N. Data are expressed as means±SEM.

### DETAILED DESCRIPTION

Provided herein is a compound of Formula (I):

R²-L-R¹ (I)

or a pharmaceutically acceptable salt thereof, wherein:
R¹ is a peptide;
R² is a radical of a peptidic tubulin inhibitor; and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide having 5 to 50 amino acids;
R² is a radical of a peptidic tubulin inhibitor; and
L is a linker, which is covalently linked to moiety R¹ and R².

Also provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;

R² is a radical of a peptidic tubulin inhibitor; and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide;
R² is a radical of an auristatin compound; and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide having 5 to 50 amino acids;
R² is a radical of an auristatin compound; and
L is a linker, which is covalently linked to moiety R¹ and R².

Also provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is a radical of an auristatin compound; and
L is a linker, which is covalently linked to moiety R¹ and R².

In some embodiments, the auristatin compound is a monomethyl auristatin compound.

In some embodiments, L is a linker having the structure:

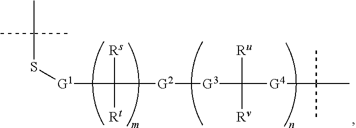

wherein the S atom of the linker is bonded with a cysteine residue of the peptide to form a disulfide bond; and wherein:
G¹ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G¹ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each R^{s} and R^{t} are independently selected from H, halo, C₁₋₆ alkyl, and C₁₋₆ haloalkyl;
G² is selected from —NR^{G}C(O)—, —NR^{G}—O—, —S—, —C(O)O—, —OC(O)—, —NR^{G}C(O)—, —OC(O)NR^{G}—, and —S(O₂)—;
G³ is selected from C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, and C₂₋₆ alkynyl substituent of G³ are optionally substituted with 1, 2, or 3 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{u} and R^{v} are independently selected from H, halo, C₁₋₆ alkyl, and C₁₋₆ haloalkyl;
G⁴ is selected from —C(O)—, —NR^{G}C(O)—, —NR^{G}—O—, —S—, —C(O)—, —OC(O)—, —NR^{G}C(O)—, and —S(O₂)—;
each R^{G} is independently selected from H and C₁₋₄ alkyl;
each R^{a}, R^{b}, R^{c}, R^{d}, R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl of R^{a}, R^{b}, R^{c}, R^{d}, R^{a1}, R^{b1}, R^{c1} and R^{d1} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)NR^{c2}R^{d2}, NR^{c2}C(O)OR^{a2}, C(═NR^{e2})NR^{c2}R^{d2}, NR^{c2}C(═NR^{e2})NR^{c2}R^{d2} S(O)R^{b2}, S(O)NR^{c2}R^{d2}, S(O)₂R^{b2}, NR^{c2}S(O)₂R^{b2}, NR²S(O)₂NR^{c2}R^{d2}, and S(O)₂NR^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, and C₂₋₆ alkynyl, wherein said C₁₋₆ alkyl, C₁₋₆ haloalkyl, C₂₋₆ alkenyl, and C₂₋₆ alkynyl of R^{a2}, R^{b2}, R^{c2}, and R^{d2} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, and R^{e2} is independently selected from H and C₁₋₄ alkyl;
m is 0, 1, 2, 3, or 4; and
n is 0 or 1.

Provided herein is a compound of Formula (I):

R²-L-R¹ (I)

or a pharmaceutically acceptable salt thereof, wherein:
R¹ is a peptide;
R² is a radical of an auristatin compound; and
L is a linker having a structure selected from:

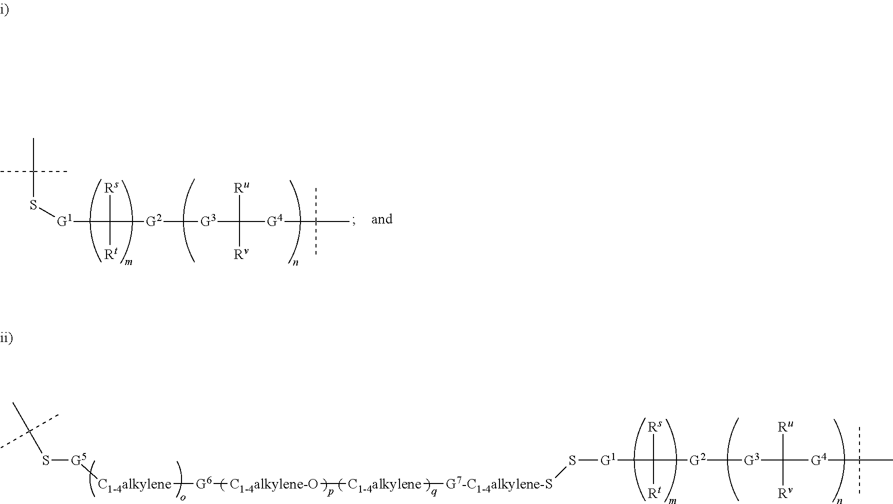

wherein the terminal S atom of the linker is bonded with a cysteine residue of the peptide to form a disulfide bond; and wherein:
G¹ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NRS(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G¹ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR)NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
G² is selected from —NR^{G}C(O)—, —NR^{G}—O—, —S—, —C(O)O—, —OC(O)—, —NR^{G}C(O)—, —OC(O)NR^{G}—, and —S(O₂)—;
G³ is selected from C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, and C₂₋₆ alkynyl substituent of G³ are optionally substituted with 1, 2, or 3 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
G⁴ is selected from —C(O)—, —NR^{G}C(O)—, —NR^{G}—O—, —S—, —OC(O)—, —NR^{G}C(O)—, and —S(O₂)—;
G⁵ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G⁵ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G⁵ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR)NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
G⁶ is selected from —NR^{G}C(O)—, —NR^{G}, —S—, —C(O)O—, —OC(O)—, —NR^{G}C(O)—, —OC(O)NR^{G}—, and —S(O₂)—;
G⁷ is selected from —NR^{G}C(O)—, —NR^{G}—S—, —C(O)—, —OC(O)—, —NR^{G}C(O)—, —OC(O)NR^{G}—, and —S(O₂)—;
each R^{s} and R^{t} are independently selected from H, halo, C₁₋₆ alkyl, and C₁₋₆ haloalkyl; or each R^{s} and R^{t}, together with the C atom to which they are attached, form a C₃₋₆ cycloalkyl ring;
R^{u} and R^{v} are independently selected from H, halo, C₁₋₆ alkyl, and C₁₋₆ haloalkyl;
each R^{G} is independently selected from H and C₁₋₄ alkyl;
each R^{a}, R^{b}, R^{c}, R^{d}, R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR²C(O)R^{b2} NR^{c2}C(O)NR^{c2}R^{d2}, NR^{c2}C(O)OR^{a2}, C(═NR^{e2})NR^{c2}R^{d2}, NR^{c2}C(═NR^{e2})NR^{c2}R^{d2} S(O)R^{b2}, S(O)NR^{c2}R^{d2}, S(O)₂R^{b2}, NR^{c2}S(O)₂R^{b2}, NR^{c2}S(O)₂NR^{c2}R^{d2}, and S(O)₂NR^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, and C₂₋₆ alkynyl, wherein said C₁₋₆ alkyl, C₁₋₆ haloalkyl, C₂₋₆ alkenyl, and C₂₋₆ alkynyl of R^{a2}, R^{b2}, R^{c2}, and R^{d2} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, and R^{e2} is independently selected from H and C₁₋₄ alkyl;
m is 0, 1, 2, 3, or 4;
n is 0 or 1;
is 0 or 1;
p is 1, 2, 3, 4, 5, or 6; and
q is 0 or 1.

Provided herein is a compound of Formula (I):

R²-L-R¹ (I)

or a pharmaceutically acceptable salt thereof, wherein:
R¹ is a peptide;
R² is a radical of an auristatin compound; and
L is a linker having the structure:

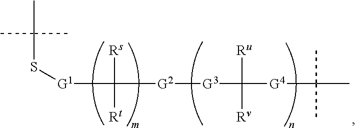

wherein the S atom of the linker is bonded with a cysteine residue of the peptide to form a disulfide bond; and wherein:
G¹ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G¹ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each R^{s} and R^{t} are independently selected from H, halo, C₁₋₆ alkyl, and C₁₋₆ haloalkyl;
G² is selected from —NR^{G}C(O)—, —NR^{G}—O—, —S—, —C(O)—, —OC(O)—, —NR^{G}C(O)—, —OC(O)NR^{G}—, and —S(O₂)—;
G³ is selected from C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, and C₂₋₆ alkynyl substituent of G³ are optionally substituted with 1, 2, or 3 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{b1}, S(O)₂R^{b1}, and S(O)₂NR^{c1}R^{d1};
R^{u} and R^{v} are independently selected from H, halo, C₁₋₆ alkyl, and C₁₋₆ haloalkyl;
G⁴ is selected from —C(O)—, —NR^{G}C(O)—, —NR^{G}—O—, —S—, —C(O)O—, —OC(O)—, —NR^{G}C(O)—, and —S(O₂)—;
each R^{G} is independently selected from H and C₁₋₄ alkyl;
each R^{a}, R^{b}, R^{c}, R^{d}, R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a2}, SR^{a2}, C(O)R^{b2}, C(O)NR^{c2}R^{d2}, C(O)OR^{a}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, C(═NR^{e2})NR^{e2}R^{d2}, NR^{c2}C(═NR^{e2})NR^{c2}R^{d2}, S(O)R^{b2}, S(O)NR^{c2}R^{d2}, S(O)₂R^{b2}, NR^{c2}S(O)₂R^{b2}, NR²S(O)₂NR^{c2}R^{d2}, and S(O)₂NR^{c2}R^{d2};
each R^{a2}, R^{b2}, R^{c2}, and R^{d2} is independently selected from H, C₁₋₆ alkyl, C₁₋₆ haloalkyl, C₂₋₆ alkenyl, and C₂₋₆ alkynyl, wherein said C₁₋₆ alkyl, C₁₋₆ haloalkyl, C₂₋₆ alkenyl, and C₂₋₆ alkynyl of R^{a2}, R^{b2}, R^{c2}, and R^{d2} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, and R^{e2} is independently selected from H and C₁₋₄ alkyl;
m is 0, 1, 2, 3, or 4; and
n is 0 or 1.

In some embodiments, the lefthand side of L attaches to R¹ and the righthand side of L attaches to R².

As used herein, “peptide” refers to a targeting moiety comprising a 10-50 amino acid sequence, made up of naturally-occurring amino acid residues and optionally one or more non-naturally-occurring amino acids. In some embodiments, the peptide of R¹ is a peptide of 20 to 40, 20 to 30 amino acids, or 30 to 40 residues. Peptides suitable for use in the compounds of the invention are those that can insert across a cell membrane via a conformational change or a change in secondary structure in response to environmental pH changes. In this way, the peptide can target acidic tissue and selectively translocate polar, cell-impermeable molecules across cell membranes in response to low extracellular pH. In some embodiments, the peptide is capable of selectively delivering a conjugated moiety (e.g., R²L-) across a cell membrane having an acidic or hypoxic mantle having a pH less than about 6.0. In some embodiments, the peptide is capable of selectively delivering a conjugated moiety (e.g., R²L-) across a cell membrane having an acidic or hypoxic mantle having a pH less than about 6.5. In some embodiments, the peptide is capable of selectively delivering a conjugated moiety (e.g., R²L-) across a cell membrane having an acidic or hypoxic mantle having a pH less than about 5.5. In some embodiments, the peptide is capable of selectively delivering a conjugated moiety (e.g., R²L-) across a cell membrane having an acidic or hypoxic mantle having a pH between about 5.0 and about 6.0.

In certain embodiments, the peptide of R¹ includes a cysteine residue which can form the site of attachment to a payload moiety (e.g., R²L-) to be delivered across a cell membrane. In some embodiments, R¹ is attached to L through a cysteine residue of R¹. In some embodiments, the sulfur atom of the cysteine residue can form part of the disulfide bond of the disulfide bond-containing compound.

Suitable peptides, that can conformationally change based on pH and insert across a cell membrane, are described, for example, in U.S. Pat. Nos. 8,076,451, 9,289,508, 10,933,069, and U.S. Application Publication Nos. 2021/0009536 and 2021/0009719 (each of which is incorporated herein in its entirety). Other suitable peptides are described, for example, in Weerakkody, et al., PNAS 110 (15), 5834-5839 (Apr. 9, 2013), which is also incorporated herein by reference in its entirety.

In some embodiments, R¹ is a peptide comprising at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG;
(SEQ ID NO. 4; Pv4)
Ac-AAEQNPIYWARYADWLFTTPLLLLDLALLVDADEGTKCG;
(SEQ ID No. 5; Pv5)
AAEQNPIYWARYADWLFTTPLLLLDLALLVDADEGTC;
and
(SEQ ID No. 6; Pv6)
AAEQNPIYWWARYADWLFTTPLLLLDLALLVDADEGTCG;

wherein R¹ is attached to L through a cysteine residue of R¹.

In some embodiments, R¹ is a peptide comprising at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG;
and
(SEQ ID No. 6; Pv6)
AAEQNPIYWWARYADWLFTTPLLLLDLALLVDADEGTCG;

wherein R¹ is attached to L through a cysteine residue of R¹.

In some embodiments, R¹ is a peptide comprising the sequence

(SEQ ID NO. 1; Pv1)
ADDQNPWRAYLDLLFPTDTLLLDLLWCG.

In some embodiments, R¹ is a peptide comprising the sequence

(SEQ ID NO. 2; Pv2)
AEQNPIYWARYADWLFTTPLLLLDLALLVDADECG.

In some embodiments, R¹ is a peptide comprising the sequence

(SEQ ID NO. 3; Pv3)
ADDQNPWRAYLDLLFPTDTLLLDLLWDADECG.

In some embodiments, R¹ is a peptide comprising the sequence

(SEQ ID NO. 4; Pv4)
Ac-AAEQNPIYWARYADWLFTTPLLLLDLALLVDADEGTKCG.

In some embodiments, R¹ is a peptide comprising the sequence

(SEQ ID NO. 5; Pv5)
AAEQNPIYWARYADWLFTTPLLLLDLALLVDADEGTC.

In some embodiments, R¹ is a peptide comprising the sequence

(SEQ ID NO. 6; Pv6)
AAEQNPIYWWARYADWLFTTPLLLLDLALLVDADEGTCG.

In some embodiments, R¹ is a peptide consisting of the sequence

(SEQ ID NO. 1; Pv1)
ADDQNPWRAYLDLLFPTDTLLLDLLWCG.

In some embodiments, R¹ is a peptide consisting of the sequence

(SEQ ID NO. 2; Pv2)
AEQNPIYWARYADWLFTTPLLLLDLALLVDADECG.

In some embodiments, R¹ is a peptide consisting of the sequence

(SEQ ID NO. 3; Pv3)
ADDQNPWRAYLDLLFPTDTLLLDLLWDADECG.

In some embodiments, R¹ is a peptide consisting of the sequence Ac—

(SEQ ID NO. 4; Pv4)
AAEQNPIYWARYADWLFTTPLLLLDLALLVDADEGTKCG.

In some embodiments, R¹ is a peptide consisting of the sequence

(SEQ ID NO. 5; Pv5)
AAEQNPIYWARYADWLFTTPLLLLDLALLVDADEGTC.

In some embodiments, R¹ is a peptide consisting of the sequence

(SEQ ID NO. 6; Pv6)
AAEQNPIYWWARYADWLFTTPLLLLDLALLVDADEGTCG.

In some embodiments, R¹ is a peptide comprising at least one sequence selected from SEQ ID NO: 7 to SEQ ID NO: 311 as shown in Table 1.

In some embodiments, R¹ is a peptide consisting of a sequence selected from SEQ ID NO: 7 to SEQ ID NO: 311 as shown in Table 1.

TABLE 1
Additional R¹ Sequences

| SEQ | |
|---|---|
| ID | |
| NO. | Sequence |
| 7 | AEQNPIYWARYADWLFTTPLLLLDLALLVDADEGT |
| 8 | GGEQNPIYWARYADWLFTTPLLLLDLALLVDADEGT |
| 9 | AEQNPIYWARYADWLFTTPLLLLDLALLVDADEGT |
| 10 | AAEQNPIYWARYADWLFTTPLLLLDLALLVDADEGTCG |
| 11 | GGEQNPIYWARYADWLFTTPLLLLDLALLVDADEGTCG |
| 12 | ACEQNPIYWARYADWLFTTPLLLLDLALLVDADEGTG |
| 13 | ACEQNPIYWARYADWLFTTPLLLLDLALLVDADEGT |
| 14 | AKEQNPIYWARYADWLFTTPLLLLDLALLVDADEGT |
| 15 | AAEQNPIYWARYADWLFTTPLLLLDLALLVDADEGTKCG |
| 16 | AKEQNPIYWARYADWLFTTPLLLLDLALLVDADECT |
| 17 | ACEQNPIYWARYANWLFTTPLLLLNLALLVDADEGTG |
| 18 | ACEQNPIYWARYAKWLFTTPLLLLKLALLVDADEGTG |
| 19 | GGEQNPIYWARYADWLFTTPLLLLDLALLVNANQGT |
| 20 | AAEQNPIYWARYADWLFTTPLLLLALALLVDADEGT |
| 21 | AAEQNPIYWARYAAWLFTTPLLLLDLALLVDADEGT |
| 22 | AAEQNPIYWARYADWLFTTALLLLDLALLVDADEGT |
| 23 | AAEQNPIYWARYADWLFTTPLLLLELALLVDADEGT |
| 24 | AAEQNPIYWARYAEWLFTTPLLLLDLALLVDADEGT |
| 25 | AAEQNPIIYWARYADWLFTDLPLLLLDLLALLVDADEGT |
| 26 | GEQNPIYWAQYADWLFTTPLLLLDLALLVDADEGTCG |
| 27 | GGEQNPIYWARYADWLFTTPLLLDLLALLVDADEGTCG |
| 28 | GGEQNPIYWARYADWLFTTPLLLLLDALLVDADEGTCG |
| 29 | GGEQNPIYWARYDAWLFTTPLLLLDLALLVDADEGTCG |
| 30 | GGEQNPIYWARYAWDLFTTPLLLLDLALLVDADEGTCG |
| 31 | AAEQNPIYWARYADWLFTTGLLLLDLALLVDADEGT |
| 32 | DDDEDNPIYWARYADWLFTTPLLLLHGALLVDADECT |
| 33 | DDDEDNPIYWARYAHWLFTTPLLLLHGALLVDADEGCT |
| 34 | DDDEDNPIYWARYAHWLFTTPLLLLHGALLVNADECT |
| 35 | DDDEDNPIYWARYAHWLFTTPLLLLHGALLVNANECT |
| 36 | AEQNPIYWARYADFLFTTPLLLLDLALLVDADET |
| 37 | AEQNPIYFARYADWLFTTPLLLLDLALLVDADEGT |
| 38 | AEQNPIYFARYADFLFTTPLLLLDLALLWDADET |
| 39 | AKEDQNPYWARYADWLFTTPLLLLDLALLVDG |
| 40 | ACEDQNPYWARYADWLFTTPLLLLDLALLVDG |
| 41 | AEDQNPYWARYADWLFTTPLLLLDLALLVDCG |
| 42 | AEDQNPYWARYADWLFTTPLLLLELALLVECG |
| 43 | AKEDQNPYWRAYADLFTPLTLLDLLALWDG |
| 44 | ACEDQNPYWRAYADLFTPLTLLDLLALWDG |
| 45 | ACDDQNPWRAYLDLLFPTDTLLLDLLW |
| 46 | TEDADVLLALDLLLLPTTFLWD |
| 47 | AEQNPIYWARYADWLFTTPL |
| 48 | AEQNPIYWARYADWLFTTPCL |
| 49 | ACEQNPIYWARYADWLFTTPL |
| 50 | AEQNPIYFARYADWLFTTPL |
| 51 | KEDQNPWARYADLLFPTTLAW |
| 52 | ACEDQNPWARYADLLFPTTLAW |
| 53 | ACEDQNPWARYADWLFPTTLLLLD |
| 54 | ACEEQNPWARYAELLFPTTLAW |
| 55 | ACEEQNPWARYAEWLFPTTLLLLE |
| 56 | ACEEQNPWARYLEWLFPTETLLLEL |
| 57 | GGEQNPIY WARYADWLFTTPLLLLDLALLV DADEGT |
| 58 | ACEQNPIY WARYADWLFTTPLLLLDLALLV |
| 59 | WARYADWLFTTPLLLLDLALLV DADEGTCG |
| 60 | WARYADWLFTTPLLLLDLALLV DADEGCT |
| 61 | GGEQNPIY WARYADWLFTTPLLLLDLALLV DADEGTCG |
| 62 | ACEQNPIY WARYADWLFTTPLLLLDLALLV DADEGT |
| 63 | AKEQNPIY WARYADWLFTTPLLLLDLALLV DADEGT |
| 64 | AKEQNPIY WARYADWLFTTPLLLLDLALLV DADECT |
| 65 | AAEQNPIY WARYADWLFTTALLLLDLALLV DADEGT |
| 66 | ACAEQNPIY WARYADWLFTTGLLLLDLALLV DADEGT |
| 67 | AEQNPIY WARYADFLFTTALLLLDLALLV DADE_T |
| 68 | AEQNPIY FARYADWLFTTPLLLLDLALLV DADEGT |
| 69 | AEQNPIY FARYADFLFTTPLLLLDLALLW DADE_T |
| 70 | AKEDQNP_Y WARYADWLFTTPLLLLDLALLV DG__ |
| 71 | ACEDQNP_Y WARYADWLFTTPLLLLDLALLV DG__ |
| 72 | AEDQNP_Y WARYADWLFTTPLLLLDLALLV DG___ |
| 73 | AEDQNP_Y WARYADWLFTTPLLLLELALLV ECG__ |
| 74 | AKEDQNP_Y WRAYAD_LFT_PLTLLDLLALW DG__ |
| 75 | ACEDQNP_Y WRAYAD_LFT_PLTLLDLLALW DG__ |
| 76 | AKEDQNDP_Y WARYADWLFTTPLLLLDLALLV G__ |
| 77 | TEDADVLLALDLLLLPTTFLWDAYRAWYPNQECA |
| 78 | GGEQNPIY WARYADWLFTTPLLLLDLALLV DADEGT |
| 79 | AEQNPIY WARYADWLFTTPL |
| 80 | AEQNPIY WARYADWLFTTPCL |
| 81 | ACEQNPIY WARYADWLFTTPL |
| 82 | ACEQNPIY FARYADWLFTTPL |
| 83 | ACDDQNP WRAYLDLLFPTDTLLLDLLW |
| 84 | ACEEQNP WRAYLELLFPTETLLLELLW |
| 85 | ACDDQNP WARYLDWLFPTDTLLLDL |
| 86 | CDNNNP WRAYLDLLFPTDTLLLDW |
| 87 | ACEEQNP WARYLEWLFPTETLLLEL |
| 88 | ACEDQNP WARYADWLFPTTLLLLD |
| 89 | ACEEQNP WARYAEWLFPTTLLLLE |
| 90 | ACEDQNP WARYADLLFPTTLAW |
| 91 | ACEDQNP WARYAELLFPTTLW |
| 92 | KEDQNP WARYADLLFPTTLW |
| 93 | DDDEDNP IYWARYAHWLFTTPLLLLHGALLVDADECT |
| 94 | DDDEDNPIYWARYAHWLFTTPLLLLDGALLVDADECT |
| 95 | DDDEDNPIYWARYAHWLFTTPLLLLHGALLVNADECT |
| 96 | DDDEDNPIYWARYAHWLFTTPLLLLHGALLVNANECT |
| 97 | DDDEDNPIYWARYADWLFTTPLLLLHGALLVDADECT |
| 98 | ACEQNPIYWARYADWLFTTPLLLLDLALLVDADEGIG |
| 99 | ACEQNPIYWARYADWLFTTPLLLLDLALLVDADET |
| 100 | ACEQNPIYWARYADWLFTTPLLLLDLALLVDADEGT |
| 101 | GGEQNPIYWARYADWLFTTPLLLDLLALLVDADEGTCG |
| 102 | GGEQNPIYWARYADWLFTTPLLLLLDALLVDADEGTCG |
| 103 | GGEQNPIYWARYAWDLFTTPLLLLDLALLVDADEGTCG |
| 104 | AAEQNPIYWARYAEWLFTTPLLLLDLALLVDADEGTCG |
| 105 | AAEQNPIYWARYAEWLFTTPLLLLELALLVDADEGTCG |
| 106 | GGEQNPIYWARYDAWLFTTPLLLLDLALLVDADEGTCG |
| 107 | GGEQNPIYWAQYDAWLFTTPLLLLDLALLVDADEGTCG |
| 108 | GGEQNPIYWAQDYAWLFTTPLLLLDLALLVDADEGTCG |
| 109 | AAEQNPIYWARYAAWLFTTPLLLLDLALLVDADEGTCG |
| 110 | ACEQNPIYWARYANWLFTTPLLLLNLALLVDADEGTG |
| 111 | DDDEDNPIYWARYAHWLFTTPLLLLHGALLVNANECT |
| 112 | DDDEDNPIYWARYAHWLFTTPLLLLHGALLVNADECT |
| 113 | DDDEDNPIYWARYADWLFTTPLLLLHGALLVDADECT |
| 114 | DDDEDNPIYWARYAHWLFTTPLLLLHGALLVDADECT |
| 115 | DDDEDNPIYWARYAHWLFTTPLLLLDGALLVDADECT |
| 116 | GGEQNPIYWARYADWLFTTPLLLLDLALLVNANQGT |
| 117 | AAEQNPIYWARYADWLFTTPLLLLELALLVDADEGTCG |
| 118 | AAEQNPIYWARYAEWLFTTPLLLLELALLVDADEGTCG |
| 119 | AAEQNPIYWARYADWLFTTPLLLLELALLVDADEGTKCG |
| 120 | GGEQNPIYWAQYADWLFTTPLLLLDLALLVDADEGTCG |
| 121 | GGEQNPIYWAQYDAWLFTTPLLLLDLALLVDADEGTCG |
| 122 | GGEQNPIYWAQDYAWLFTTPLLLLDLALLVDADEGTCG |
| 123 | GGEQNPIYWARYADWLFTTPLLLLDALLVNANQGT |
| 124 | DDDEDNPIYWARYAHWLFTTPLLLLHGALL VNADECT |
| 125 | DDDEDNPIYWARYAHWLFTTPLLLLHGALL VNANECT |
| 126 | ACEQNPIYWARYAKWLFTTPLLLLKLALLVDADEGTG |
| 127 | GGEQNPIYWAQDYAWLFTTPLLLLDLALLVDADEGTCG |
| 128 | GGEQNPIYWAQYDAWLFTTPLLLLDLALLVDADEGTCG |
| 129 | GGEQNPIYWAQYADWLFTTPLLLLDLALLVDADEGTCG |
| 130 | AAEQNPIYWARYAAWLFTTPLLLLDLALLVDADEGTCG |
| 131 | AAEQNPIYWARYADWLFTDLPLLLLDLLALLVDADEGT |
| 132 | GGEQNPIYWARYADWLFTTPLLLLLDALLVDADEGTCG |
| 133 | GGEQNPIYWARYADWLFTTPLLLDLLALLVDADEGTCG |
| 134 | AAEQNPIYWARYADWLFTTGLLLLDLALL VDADEGT |
| 135 | AEQNPIYWARYAAWLFTTPLLLLDLALL VDADEGTCG |
| 136 | GGEQNPIYWAQYDAWLFTTPLLLLDLALLVDADEGTCG |
| 137 | GGEQNPIYWAQDYAWLFTTPLLLLDLALLDADEGTCG |
| 138 | GGEQNPIYWARYDAWLFTTPLLLLDLALLVDADEGTCG |
| 139 | AAEQNPIYWARYADWLFTTPLLLLALALL VDADEGTCG |
| 140 | AAEQNPIYWARYADWLFTTPLLLLDLALLVDADEGTKCG |
| | .........EGTK(rhodamine)C(phalloidin)G |
| 141 | AAEQNPIYWARYADWLFTTPLLLLELALLDADEGTKCG |
| 142 | AAEQNPIYWARYADWLFTTPLLLLDLALLVDADEGTCG |
| 143 | AAEQNPIYWARYADWLFTTPLLLLDLALLVDADEGTC |
| | (phalloidin)G |
| 144 | GGEQNPIYWARYADWLFTTPLLLLDLALLVDADEGTCG |
| 145 | ACEQNPIYWARYADWLFTTPLLLLDLALL VDADET |
| 146 | ACEQNPIYWARYADWLFTTPLLLLDLALL VDADEGTG |
| 147 | ACEQNPIYWARYADWLFTTPLLLLDLALL VDADEGT |
| 148 | GGEQNPIYWARYADWLFTTPLLLLDLALLVNANQGT |
| 149 | DDDEDNPIYWARYAHWLFTTPLLLLHGALLVNADECT |
| 150 | DDDEDNPIYWARYAHWLFTTPLLLLHGALL VNANECT |
| 151 | GGEQNPIYWARYADWLFTTPLLLLDLALLVDADEGTCG |
| 152 | AAEQNPIYWARYADWLFTTPLLLLDLALLVDADEGTC |
| | (phalloidin)G |
| 153 | AAEQNPIYWARYADWLFTTPLLLLELALLVDADEGTKCG |
| 154 | AAEQNPIYWARYADWLFTTPLLLLDLALLVDADEGTKCG |
| 155 | DDDEDNPIYWARYAHWLFTTPLLLLBGALL VDADECT |
| 156 | DDDEDNPIYWARYAHWLFTTPLLLLDGALL VDADECT |
| 157 | DDDEDNPIYWARYAHWLFTTPLLLLBGALLVNADECT |
| 158 | DDDEDNPIYWARYAHWLFTTPLLLLBGALL VNANECT |
| 159 | DDDEDNPIYWARYADWLFTTPLLLLIBGALLVDADECT |
| 160 | DDDEDNPIYWARYADWTFTTPLLLLHGALLVDADECT |
| 16 | DDDEDNPIYWARYAHWLFTTPLLLLDGALL VDADECT |
| 162 | DDDEDNPIYWARYAHWLFTTPLLLLHGALL VDADECT |
| 163 | DDDEDNPIYWARYAHWLFTTPLLLLHGALL VNADECT |
| 164 | DDDEDNPIYWARYHWLFTTPLLLLHGALLVNANECT |
| 165 | DDDEDNPIYWARYAHWLFTTPLLLLHGALLVNANECT |
| 166 | DDDEDNPIYWARYAHWLFTTPLLLLHGALL VNADECT |
| 167 | DDDEDNPIYWARYADWLFTTPLLLLHGALL VDADECT |
| 168 | DDDEDNPIYWARYAHWLFTTPLLLLHGALL VDADECT |
| 169 | DDDEDNPIYWARYAHWLFTTPLLLLDGALLVDADECT |
| 170 | GGEQNPIYWARYADWLFTTPLLLLDLALLVNANQGT |
| 171 | DDDEDNPIYWARYAHWLFTTPLLLLHGALLVNADECT |
| 172 | DDDEDNPIYWARYADWLFTTPLLLLHGALL VDADECT |
| 173 | DDDEDNPIYWARYAHWLFTTPLLLLHGALL VDADECT |
| 174 | DDDEDNPIYWARYAHMLFTTPLLLLDGALLVDADECT |
| 175 | DDDEDNPIYWARYAHWLFTTPLLLLHGALLVNANECT |
| 176 | DDDEDNPIYWARYAHWLFTTPLLLLDGALLVDADECT |
| 177 | DDDEDNPIYWARYADWLFTTPLLLLHGALL VDADECT |
| 178 | DDDEDNPIYWARYAHWLFTTPLLLLHGALLVDADECT |
| 179 | DDDEDNPIYWARYAHWLFTTPLLLLHGALL VNADECT |
| 180 | DDDEDNPIYWARYAHWLFTTPLLLLHGALL VNANECT |
| 181 | AAEQNPIYWARYADWLFTTGLLLLDLALLVDADEGT |
| 182 | GGEQNPIYWARYAWDLFTTPLLLLDLALLVDADEGTCG |
| 183 | GGEQNPIYWARYDAWLFTTPLLLLDLALLVDADEGTCG |
| 184 | GGEQNPIYWAQYDAWLFTTPLLLLDLALLVDADEGTCG |
| 185 | GGEQNPIYWAQDYAWLFTTPLLLLDLALLVDADEGTCG |
| 186 | AAEQNPIYWARYAAWLFTTPLLLLDLALL VDADEGTCG |
| 187 | GGEQNPIYWARYADWLFTTPLLLLDALLVDADEGTCG |
| 188 | GGEQNPIYWARYADWLFTTPLLLDLLALL VDADEGTCG |
| 189 | GGEQNPIYWARYADWLFTTPLLLDLLALLVDADEGTCG |
| 190 | GGEQNPIYWARYADWLFTTPLLLLLDALLVDADEGTCG |
| 191 | GGEQNPIYWAQYADWLFTTPLLLLDLALLVDADEGTCG |
| 192 | GGEQNPIYWAQYDAWLFTTPLLLLDLALLVDADEGTCG |
| 193 | GGEQNPIYWAQDYAWLFTTPLLLLDLALLVDADEGTCG |
| 194 | GGEQNPIYWAQYDAWLFTTPLLLLDLALLVDADEGTCG |
| 195 | GGEQNPIYWAQDYAWLFTTPLLLLDLALLVDADEGTCG |
| 196 | GGEQNPIYWAQYADWLFTTPLLLLDLALLVDADEGTCG |
| 197 | AAEQNPIYWARYAAWLFTTPLLLLDLALLVDADEGTCG |
| 198 | GGEQNPIYWAQDYAWLFTTPLLLLDLALLVDADEGTCG |
| 199 | GGEQNPIYWAQYDAWLFTTPLLLLDLALLVDADEGTCG |
| 200 | GGEQNPIYWAQYADWLFTTPLLLLDLALLVDADEGTCG |
| 201 | AAEQNPIYWARYAAWLFTTPLLLLDLALLVDADEGTCG |
| 202 | AAEQNPIYWARYADWLFTTPLLLLELALLVDADEGTKCG |
| 203 | ................EGTK(rhidamine)C |
| | (phalloidin)G |
| 204 | AAEQNPIYWARYADWLFTTPLLLLDLALLVDADEGTKCG |
| 205 | ACEQNPIYWARYADWLFTTPLLLLDLALLVDADEGTG |
| 206 | AAEQNPIYWARYADWLFTTPLLLLDLALLVDADEGTC |
| | (phalloidin)G |
| 207 | AAEQNPIYWARYADWLFTTPLLLLDLALLVDADEGTKCG |
| 208 | AAEQNPIYWARYADWLFTTPLLLLELALLVDADEGTKCG |
| 209 | AAEQNPIYWARYADWLFTDLPLLLLDLLALLVDADEGT |
| 210 | AAEQNPIYWARYAAWLFTTPLLLLDLALLVDADEGTCG |
| 211 | GGEQNPIYWAQYDAWLFTTPLLLLDLALLVDADEGTCG |
| 212 | GGEQNPIYWAQDYAWLFTTPLLLLDLALLVDADEGTCG |
| 213 | GGEQNPIYWARYDAWLFTTPLLLLDLALLVDADEGTCG |
| 214 | AAEQNPIYWARYAEWLFTTPLLLLDLALLVDADEGTCG |
| 215 | AAEQNPIYWARYAEWLFTTPLLLLELALLVDADEGTCG |
| 216 | AAEQNPIYWARYADWLFTTPLLLLALALLVDADEGTCG |
| 217 | AAEQNPIYWARYADWLFTTPLLLLELALLVDADEGTCG |
| 218 | AAEQNPIYWARYAEWLFTTPLLLLELALLVDADEGTCG |
| 219 | AAEQNPIYWARYADWLFTTPLLLLELALLVDADEGTKCG |
| 220 | ACEQNPIYWARYAKWLFTTPLLLLKLALLVDADEGTG |
| 221 | ACEQNPIYWARYANWLFTTPLLLLNLALLVDADEGTG |
| 222 | AAEQNPIYWARYADWLFTTALLLLDLALLVDADEGT |
| 223 | AEQNPIYFARYADLLFPTTLAW |
| 224 | AEQNPIYWARYADLLFPTTLAF |
| 225 | AEQNPIYWARYADLLFPTTLAW |
| 226 | ACEQNPIYWARYADWLFTTPLLLLDLALLVDADET |
| 227 | GGEQNPIYWARYADWLFTTPLLLLDLALLVDADEGT |
| 228 | AAEQNPIYWARYADWLFTTPLLLLDLALLVDADEGTCG |
| 229 | AAEQNPIYWARYADWLFTTPLLLLDLALLVDADEGTKCG |
| 230 | AKEQNPIYWARYADWLFTTPLLLLDLALLVDADECT |
| 231 | CCTCTTACCTCAGTTACA |
| 232 | D-Arg8D-Arg8-CCTCTTACCTCAGTTACA |
| 233 | D-Lys4D-Lys4-CCTCTTACCTCAGTTACA |
| 234 | S-S-CCTCTTACCTCAGTTACA |
| 235 | S-S-CCTCTGACCTCATTTACA |
| 236 | D-Arg8-Deca D-Arg8-Deca-CCTCTTACCTCAG |
| | TTACA |
| 237 | D-Arg8-Deca-mismatch D-Arg8-Deca- |
| | CCTCTGACCTCATTTACA |
| 238 | S-S-CCTCTTACCTCAGTTACA |
| 239 | AAEQNPIYWARYADWLFTTPLLLLDLALLVDADEGTCG |
| 240 | AEDQNPYWARYDWLFTTPLLLLDLALL VDCG |
| 241 | AEDQNPYWARYADWLFTTPLLLLELALLVECG |
| 242 | AEQNPIYWARYADWLFTTPLLLLDLALL VDADEGCT |
| 243 | ACEQNPIYWARYADWLFTTPLLLLDLALL VDADET |
| 244 | AE-QN-PI YWARYADWLFTTPLLLLDLALLV DADEGT- |
| | COOH |
| 245 | AEDQN-P- YWARYADWLFTTPLLLLDLALLV D---G-- |
| | COOH |
| 246 | AEDQNDP-YWARYADWLFTTPLLLLDLALLV----G-- |
| | COOH |
| 247 | AEQNPI YWARYADFLFTTPLLLLDLALLV DADET-COOH |
| 248 | AEQNPI YFARYADWLFTTPLLLLDLALLV DADET-COOH |
| 249 | AEQNPI YFARYADFLFTTPLLLLDLALLW DADET-COOH |
| 250 | AE-QN-PI YWARYADWLFTTPLLLLDLALLV DADEGCT- |
| | COOH |
| 251 | AEDQN-PI YWARYADWLFTTPLLLLDLALLV DC--G-T- |
| | COOH |
| 252 | AEDQNDPI YWARYADWLFTTPLLLLELALLVEC---G-T- |
| | COOH |
| 253 | Chelate-ACEEQNPWARYLEWLFPTETLLLEL |
| 254 | AEQNPIY WARYADWLFTTPLLLLDLALLV DADEGT- |
| | COOH |
| 255 | AKEDQNPY WARYADWLFTTPLLLLDLALLV DG-COOH |
| 256 | AKEDQNDPY WARYADWLFTTPLLLLDLALLV G-COOH |
| 257 | AEQNPI YWARYADWLFTTPLLLLDLALLV DADEGC- |
| | Biotin-T-COOH |
| 258 | AEDQNP YWARYADWLFTTPLLLLDLALLV DC- |
| | Biotin-G-COOH |
| 259 | AEDQNP YWARYADWLFTTPLLLLELALLV EC- |
| | Biotin-G-COOH |
| 260 | ACEQNPIY WARYADWLFTTPLLLLDLALLV DADEGT |
| 261 | ACEDQNPY WARYADWLFTTPLLLLDLALL V DG |
| 262 | ACEDQNPY WRAYADLFTPLTLLDLLALW DG |
| 263 | ACDDQNP WRAYLDLLFPTDTLLLDLLW |
| 264 | WRAYLELLFPTETLLLELLW |
| 265 | WARYLDWLFPTDTLLLDL |
| 266 | WRAYLDLLFPTDTLLLDW |
| 267 | WARYLEWLFPTETLLLEL |
| 268 | WAQYLELLFPTETLLLEW |
| 269 | WRAYLELLFPTETLLLEW |
| 270 | WARYADWLFPTTLLLLD |
| 271 | WARYAEWLFPTTLLLLE |
| 272 | ACEDQNP WARYADLLFPTTLAW |
| 273 | ACEEQNP WARYAELLFPTTLAW |
| 274 | Ac-TEDAD VLLALDLLLLPTTFLWDAYRAW YPNQECA-Am |
| 275 | CDDDDDNPNY WARYANWLFTTPLLLLNGALLV EAEET |
| 276 | CDDDDDNPNY WARYAPWLFTTPLLLLPGALLV EAEET |
| 277 | Ac-AEQNPIYWARYADWLFTTPLLLLDLALLVDADEGCT |
| 278 | Ac-AKEQNPIYWARYADWLFTTPLLLLDLALLVDADEGTG |
| 279 | ACEQNPIYWARYANWLFTTPLLLLNLALL VDADEGT |
| 280 | Ac-AAEQNPIYWARYADWLFTTPLLLLELALLVDADEGTKCG |
| 281 | DDDEDNPIYWARYADWLFTTPLLLLHGALLVDADET |
| 282 | CDDDEDNPIYWARYAHWLFTTPLLLLHGALLVDADET |
| 283 | DDDEDNPIYWARYAHWLFTTPLLLLHGALL VDADEGT |
| 284 | DDDEDNPIYWARYAHWLFTTPLLLLHGALL VNADEGT |
| 285 | DDDEDNPIYWARYAHWLFTTPLLLLHGALLVNANEGT |
| 286 | AKEDQNDPYWARYADWLFTTPLLLLDLALLVG |
| 287 | AEDQNPYWARYADWLFTTPLLLLELALLVCG |
| 288 | AKDDQNPWRAYLDLLFPTDTLLLDLLWC |
| 289 | ACEEQNPWRAYLELLFPTETLLLELLW |
| 290 | ACDDQNPWARYLDWLFPTDTLLLDL |
| 291 | CDNNNPWRAYLDLLFPTDTLLLDW |
| 292 | CEEQQPWAQYLELLFPTETLLLEW |
| 293 | EEQQPWRAYLELLFPTETLLLEW |
| 294 | CDDDDDNPNYWARYANWLFTTPLLLLNGALLVEAEET |
| 295 | CDDDDDNPNYWARYAPWLFTTPLLLLPGALLVEAEE |
| 296 | AEQNPIYFARYADLLFPTTLAW |
| 297 | AEQNPIYWARYADLLFPTTLAF |
| 298 | AEQNPIYWARYADLLFPTTLAW |
| 299 | KEDQNPWARYADLLFPTTLW |
| 300 | ACEEQNPQAEYAEWLFPTTLLLLE |
| 301 | AAEEQNPWARYLEWLFPTETLLLEL |
| 302 | AKEEQNPWARYLEWLFPTETLLLEL |
| 303 | AAEQNPIYWARYADWLFTTPLLLLDLALL VDADEGTGG |
| 304 | XXEXNPIYWAXXXXXLFTXXLLLXXXALLVXAXXXTXG |
| 305 | DAAEQNPIYWARYADWLFTTLPLLLLDLLALLVDADEG |
| | TKGG |
| 306 | GGEQNPIYWARYADWLFTTPLLLLDLALLVDADEGTGG |
| 307 | XXEXNPIYWAXXXXXLFTXXLLLXXXALLVXAXXXTGG |
| 308 | DGGEQNDPIYWARYADWLFTTLPLLLLDLLALLVDADE |
| | GCTXGG |
| 309 | AAEQNPIYWARYADWLFTTPLLLLDLALLVDADEGTCG |
| 310 | AEDQNPYWARYDWLFTTPLLLLDLALLVDCG |
| 311 | GLAGLAGLLGLEGLLGLPLGLLEGLWLGLELEGN |

Any of the recited peptides useful in the present invention can be modified to include a cysteine residue by replacing a non-cysteine residue with cysteine, or appending a cysteine residue to either the N-terminus or C-terminus.

In some embodiments, the peptide of R¹ is a conformationally restricted peptide. A conformationally restricted peptide can include, for example, macrocyclic peptides and stapled peptides. A stapled peptide is a peptide constrained by a covalent linkage between two amino acid side-chains, forming a peptide macrocycle. Conformationally restricted peptides are described, for example, in Guerlavais et al., Annual Reports in Medicinal Chemistry 2014, 49, 331-345; Chang et al., Proceedings of the National Academy of Sciences of the United States of America (2013), 110(36), E3445-E3454; Tesauro et al., Molecules 2019, 24, 351-377; Dougherty et al., Journal of Medicinal Chemistry (2019), 62(22), 10098-10107; and Dougherty et al., Chemical Reviews (2019), 119(17), 10241-10287, each of which is incorporated herein by reference in its entirety.

In some embodiments, R¹ is a peptide having 10 to 50 amino acids. In some embodiments, R¹ is a peptide having 20 to 40 amino acids. In some embodiments, R¹ is a peptide having 20 to 40 amino acids. In some embodiments, R¹ is a peptide having 10 to 20 amino acids. In some embodiments, R¹ is a peptide having 20 to 30 amino acids. In some embodiments, R¹ is a peptide having 30 to 40 amino acids.

The term “peptidic tubulin inhibitors” (e.g., R²) refers to compounds that comprise at least two amino acids and are inhibitors of tubulin polymerization. In some embodiments, the peptidic tubulin inhibitor is a small molecule peptidic tubulin inhibitor. In some embodiments, the peptidic tubulin inhibitor is less than 1500 Da. In some embodiments, the peptidic tubulin inhibitor is an auristatin compound, dolastatin, or tubulysin, or derivatives thereof.

Suitable auristatin compounds (e.g., R²) include auristatin derivatives that demonstrate anti-tubulin activity (e.g., the inhibition of tubulin polymerization). Auristatin compounds are known in the art and have been used as part of antibody-drug conjugates. See, for example, S. O. Doronina and P. D. Senter in Cytotoxic Payloads for Antibody-Drug Conjugates (Royal Society for Chemistry, 2019), Chapter 4: Auristatin Payloads for Antibody-Drug Conjugates, p 73-99; N. Joubert, A. Beck, C. Dumontet, C. Denevault-Sabourin, Pharmaceuticals, 2020, 13, 245; J. D. Bargh, A. Isidrio-Llobet, J. S. Parker, D. R. Spring, Chem. Soc. Rev., 2019, 48, 4361-4374; and Kostova, V., Desos, P., Starck, J.-B., Kotschy, A, The Chemistry Behind ADCs, Pharmaceuticals 2021, 14, 442; Mckertish et al., Biomedicines, 2021, 9(8):872, pp. 1-25, each of which is incorporated by reference in its entirety.

In some embodiments, the auristatin is a monomethyl auristatin. There are two major classes of auristatins: monomethyl auristatin E-type molecules and monomethyl auristatin F compounds. The structure of monomethyl auristatin E is shown below:

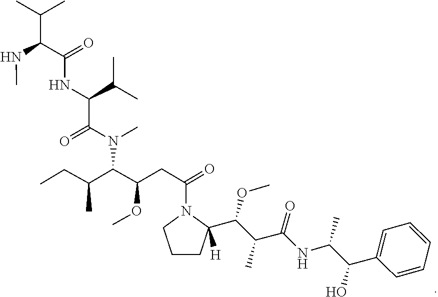 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Monomethyl auristatin E can also be referred to as “MMAE.”

The structure of monomethyl auristatin F is shown below:

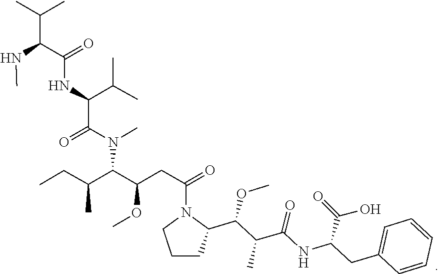 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

Monomethyl auristatin F can also be referred to as “MMAF.”

In some embodiments, R² is a radical of a monomethyl auristatin compound.

In some embodiments, R² is a radical of monomethyl auristatin E.

In some embodiments, R² is a radical of monomethyl auristatin F.

In some embodiments, R² has the structure:

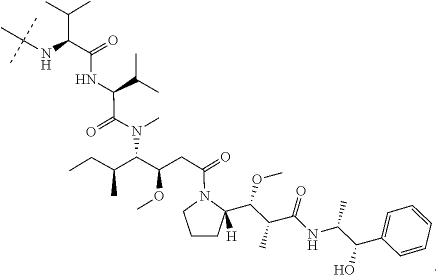

In some embodiments, R² has the structure:

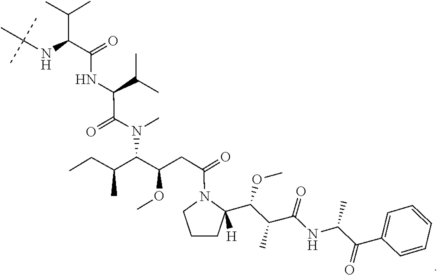

In some embodiments, R² has the structure:

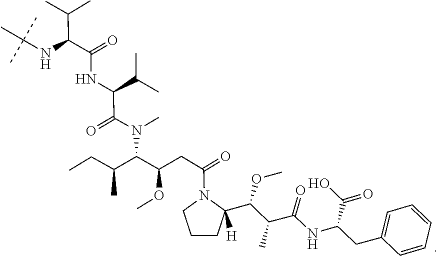

In some embodiments, L is a linking moiety that covalently connects R¹ and R², and functions to release a moiety containing R² in the vicinity of acidic or hypoxic tissue, such as inside a cell of diseased tissue.

In some embodiments, L is a linker having the structure:

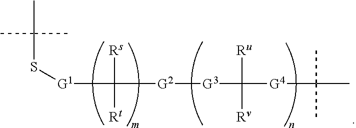

In some embodiments, L is a linker having the structure:

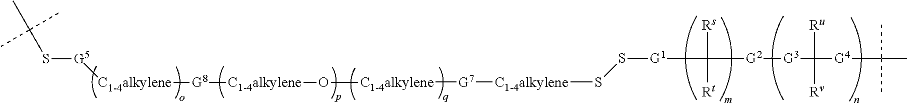

In some embodiments, G¹ is selected from a bond, C₆₋₁₀ aryl, C₃₋₁₄ cycloalkyl, 5-14 membered heteroaryl, and 4-14 membered heterocycloalkyl. In some embodiments, G¹ is selected from a bond, C₆₋₁₀ aryl, and C₃₋₁₄ cycloalkyl. In some embodiments, G¹ is selected from C₆₋₁₀ aryl and C₃₋₁₄ cycloalkyl.

In some embodiments, G¹ is selected from a bond and C₃₋₁₄ cycloalkyl.

In some embodiments, G¹ is a bond.

In some embodiments, G¹ is selected from a bond, phenyl, and C₄₋₆ cycloalkyl. In some embodiments, G¹ is selected from phenyl and C₄₋₆ cycloalkyl.

In some embodiments, G¹ is C₃₋₁₄ cycloalkyl.

In some embodiments, G¹ is cyclopentyl or cyclohexyl, wherein said cyclopentyl and cyclohexyl are each optionally fused with a phenyl group.

In some embodiments, G¹ is phenyl.

In some embodiments, G¹ is cyclopentyl, cyclohexyl, or phenyl, wherein said cyclopentyl and cyclohexyl are each optionally fused with a phenyl group.

In some embodiments, each R^{s} and R^{t} are independently selected from H and C₁₋₆ alkyl.

In some embodiments, each R^{s} and R^{t} are independently selected from H and isopropyl.

In some embodiments, each R^{s} and R^{t} are independently selected from H, methyl, and isopropyl.

In some embodiments, R^{s} and R^{t} together with the C atom to which they are attached form a C₄₋₆ cycloalkyl group.

In some embodiments, R^{s} and R^{t} together with the C atom to which they are attached form a cyclobutyl ring.

In some embodiments, m is 0, 1, or 2. In some embodiments, m is 0. In some embodiments, m is 1. In some embodiments, m is 2.

In some embodiments, G² is selected from —OC(O)— and —OC(O)NR^{G}—.

In some embodiments, G² is —OC(O)—.

In some embodiments, G³ is selected from C₆₋₁₀ aryl and 5-14 membered heteroaryl.

In some embodiments, G³ is C₆₋₁₀ aryl.

In some embodiments, G³ is phenyl.

In some embodiments, R^{u} and R^{v} are each H.

In some embodiments, G⁴ is —OC(O)—.

In some embodiments, G⁵ is 4-14 membered heterocycloalkyl, wherein said 4-14 membered heterocycloalkyl of G⁵ is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G⁵ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NWS(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}.

In some embodiments, G⁵ is the following group:

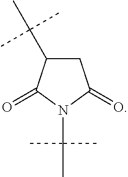

In some embodiments, G⁶ is —NR^{G}C(O)—.

In some embodiments, G⁷ is —NR^{G}C(O)—.

In some embodiments, n is 0. In some embodiments, n is 1.

In some embodiments, o is 0. In some embodiments, o is 1.

In some embodiments, p is 2, 3, 4, or 5. In some embodiments, p is 3, 4, or 5. In some embodiments, p is 3. In some embodiments, p is 4. In some embodiments, p is 5.

In some embodiments, q is 0. In some embodiments, q is 1.

In some embodiments, each R^{G} is independently selected from H and methyl. In some embodiments, each R^{G} is H. In some embodiments, each R^{G} is methyl.

In some embodiments, L has the following structure:

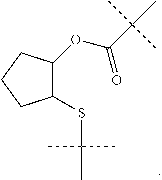

In some embodiments, L has the following structure:

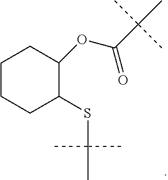

In some embodiments, L has the following structure:

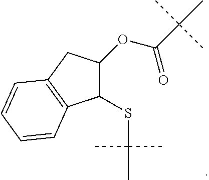

In some embodiments, L has the following structure:

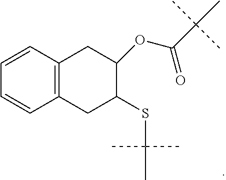

In some embodiments, L has the following structure:

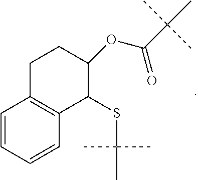

In some embodiments, L has the following structure:

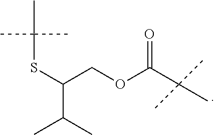

In some embodiments, L has the following structure:

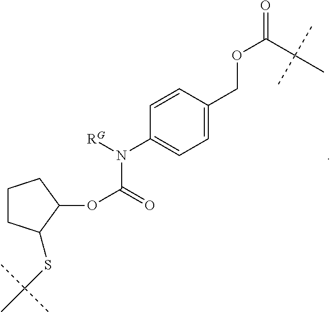

In some embodiments, L has the following structure:

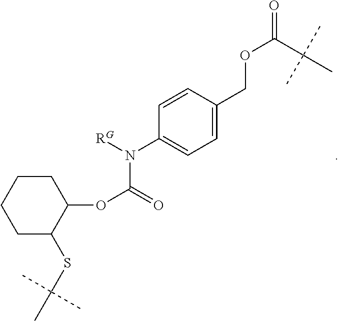

In some embodiments, L has the following structure:

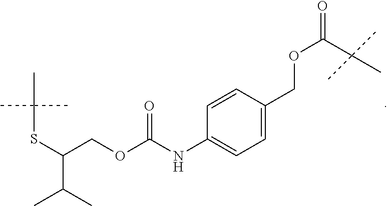

In some embodiments, L has the following structure:

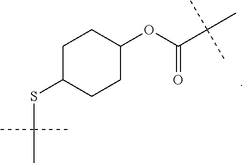

In some embodiments, L has the following structure:

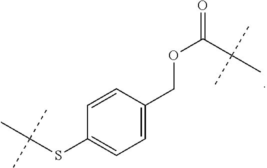

In some embodiments, L has the following structure:

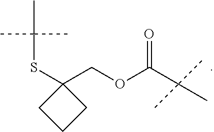

In some embodiments, L has the following structure:

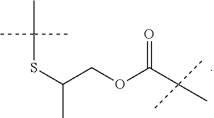

In some embodiments, L has the following structure:

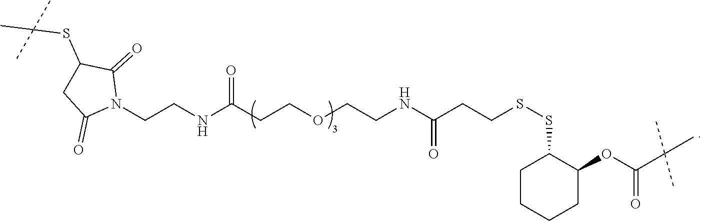

In some embodiments, L has the following structure:

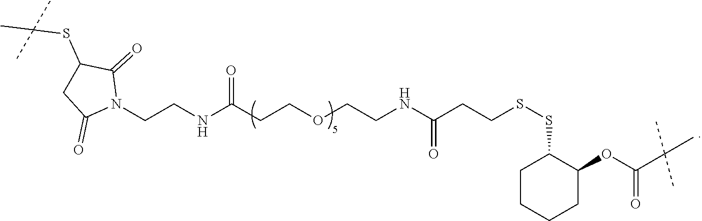

In some embodiments, the compound of the invention is a compound of Formula (II):

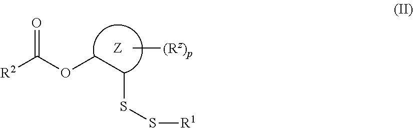

or a pharmaceutically acceptable salt thereof, wherein:
R¹ is a peptide;
R² is a radical of a peptidic tubulin inhibitor;
Ring Z is a monocyclic C₅₋₇ cycloalkyl ring or a monocyclic 5-7 membered heterocycloalkyl ring;
each R^{Z} is independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d} NR^{c}R^{d}NR^{c}C(O)R^{b}, NR^{c}C(O)OR^{a}, and NR^{c}C(O)NR^{c}R^{d};
or two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₆ alkyl, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{d}, and NR^{c}C(O)NR^{c}R^{d}; R^{a}, R^{b}, R^{c}, and R^{d} are each independently selected from H, C₁₋₄ alkyl, C₂₋₄ alkenyl, C₂₋₄ alkynyl, each optionally substituted with 1, 2, or 3 substituents independently selected from halo, OH, CN, and NO₂; and
p is 0, 1, 2, or 3.

In some embodiments, the compound of the invention is a compound of Formula (II):

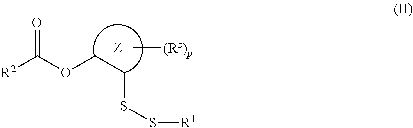

or a pharmaceutically acceptable salt thereof, wherein:
R¹ is a peptide;
R² is a radical of an auristatin compound;
Ring Z is a monocyclic C₅₋₇ cycloalkyl ring or a monocyclic 5-7 membered heterocycloalkyl ring;
each R^{Z} is independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d} NR^{c}R^{d}NR^{c}C(O)R^{b}, NR^{c}C(O)OR^{a}, and NR^{c}C(O)NR^{c}R^{d};
or two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₆ alkyl, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{d}, and NR^{c}C(O)NR^{c}R^{d};
R^{a}, R^{b}, R^{c}, and R^{d} are each independently selected from H, C₁₋₄ alkyl, C₂₋₄ alkenyl, C₂₋₄ alkynyl, each optionally substituted with 1, 2, or 3 substituents independently selected from halo, OH, CN, and NO₂; and
p is 0, 1, 2, or 3.

In some embodiments of compounds of Formula (II), R¹ is a peptide comprising the sequence of SEQ ID NO: 1, SEQ ID NO:2, or SEQ ID NO:3.

In some embodiments of compounds of Formula (II), R¹ is Pv1, Pv2, or Pv3.

In some embodiments of compounds of Formula (II), R¹ is attached to the core via a cysteine residue of R¹ wherein one of the sulfur atoms of the disulfide moiety in Formula II is derived from the cysteine residue.

In some embodiments of compounds of Formula (II), R² is a radical of a monomethyl auristatin compound.

In some embodiments of compounds of Formula (II), R² is a radical of monomethyl auristatin E.

In some embodiments of compounds of Formula (II), R² is a radical of monomethyl auristatin F.

In some embodiments of compounds of Formula (II), R² has the structure:

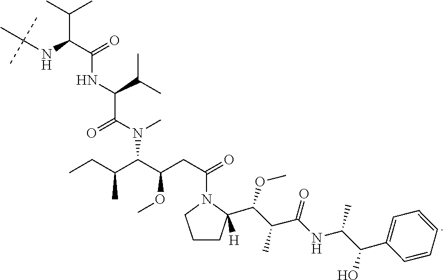

In some embodiments of compounds of Formula (II), R has the structure:

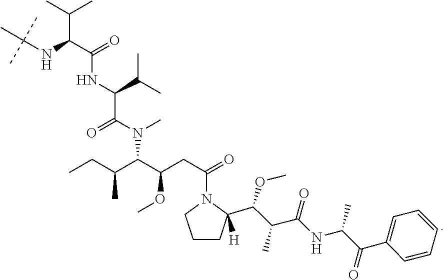

In some embodiments of compounds of Formula (II), Ring Z is a monocyclic C₅₋₇ cycloalkyl ring.

In some embodiments of compounds of Formula (II), Ring Z is a cyclopentyl ring.

In some embodiments of compounds of Formula (II), Ring Z is a cyclohexyl ring.

In some embodiments of compounds of Formula (II), two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{d}, and NR^{c}C(O)NR^{c}R^{d}.

In some embodiments of compounds of Formula (II), p is 0.

In some embodiments of compounds of Formula (II), p is 1.

In some embodiments of compounds of Formula (II), p is 2.

In some embodiments of compounds of Formula (II), p is 3.

In some embodiments, the compound of the invention is a compound of Formula (III) or Formula (IV):

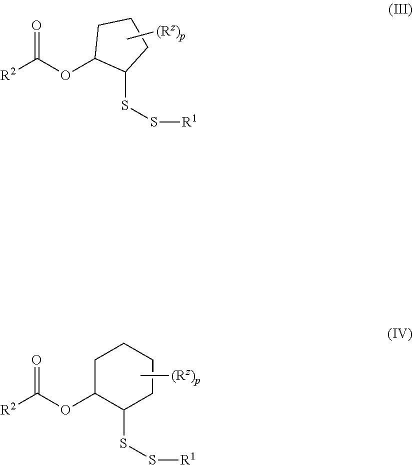

or a pharmaceutically acceptable salt thereof, wherein R¹, R², R^{Z}, and p are as defined herein.

In some embodiments of the compounds of Formulas (III) and (IV), R¹ is a peptide comprising the sequence of SEQ ID NO: 1, SEQ ID NO:2, or SEQ ID NO:3.

In some embodiments of compounds of Formulas (III) and (IV), R¹ is Pv1, Pv2, or Pv3.

In some embodiments of compounds of Formulas (III) and (IV), R¹ is attached to the core via a cysteine residue of R¹ wherein one of the sulfur atoms of the disulfide moiety in Formulas (III) and (IV) is derived from the cysteine residue.

In some embodiments of compounds of Formulas (III) and (IV), R² is a radical of a monomethyl auristatin compound.

In some embodiments of compounds of Formulas (III) and (IV), R² is a radical of monomethyl auristatin E.

In some embodiments of compounds of Formulas (III) and (IV), R² is a radical of monomethyl auristatin F.

In some embodiments, the compound of formula (I) is selected from:

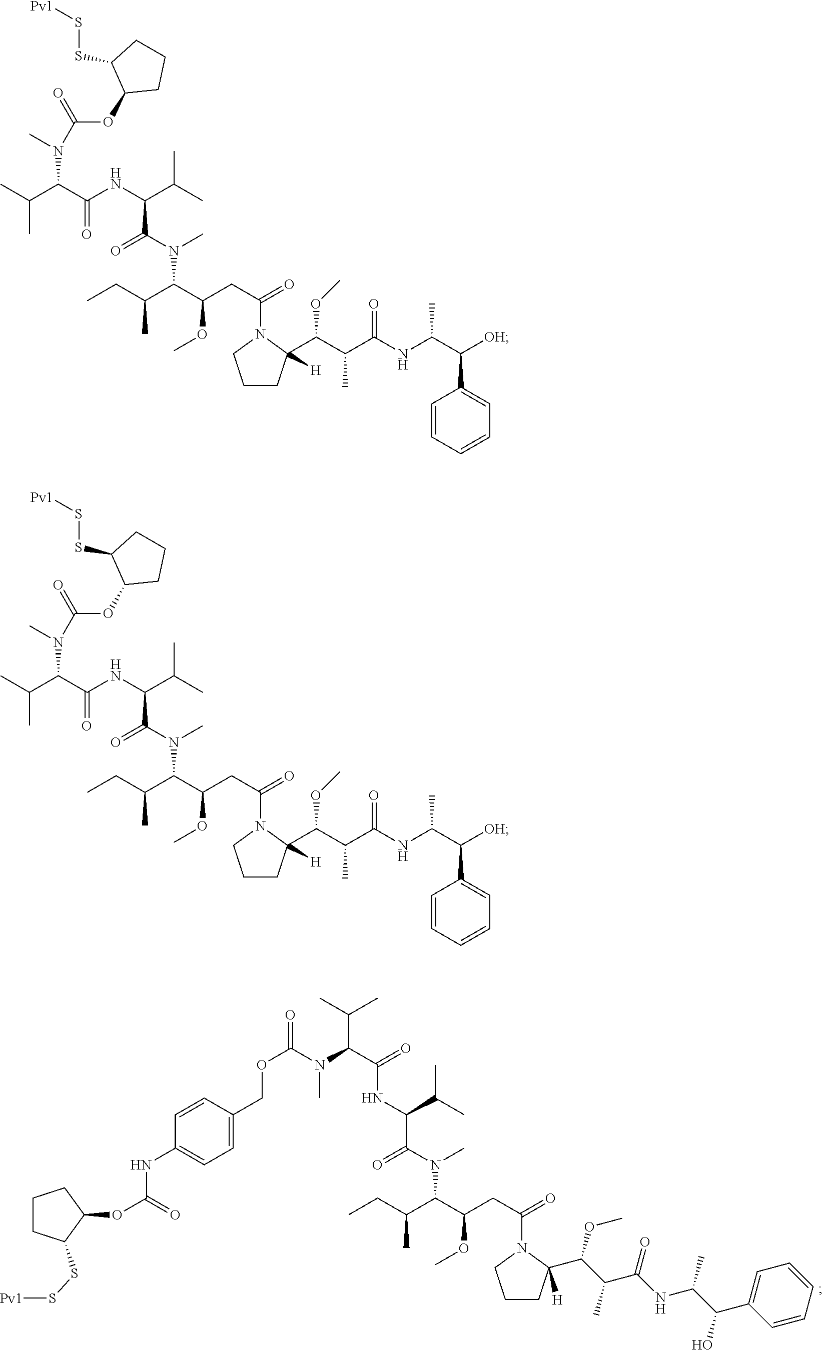

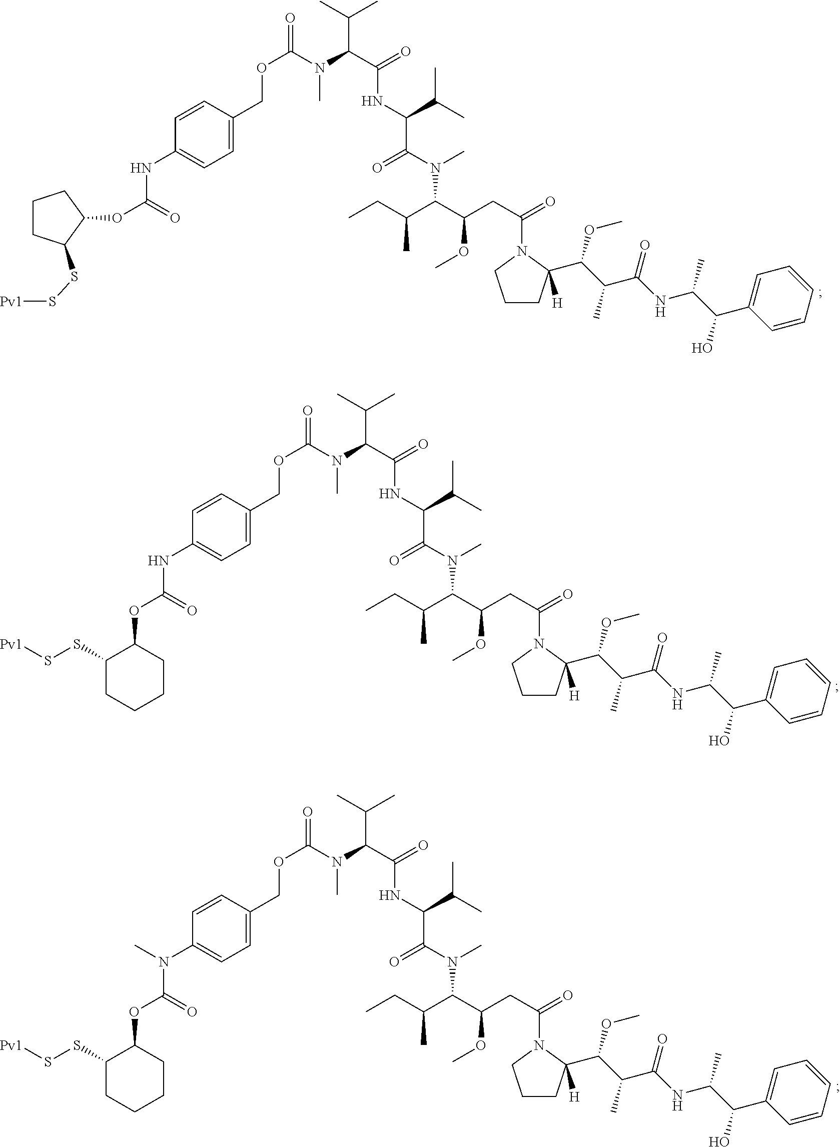

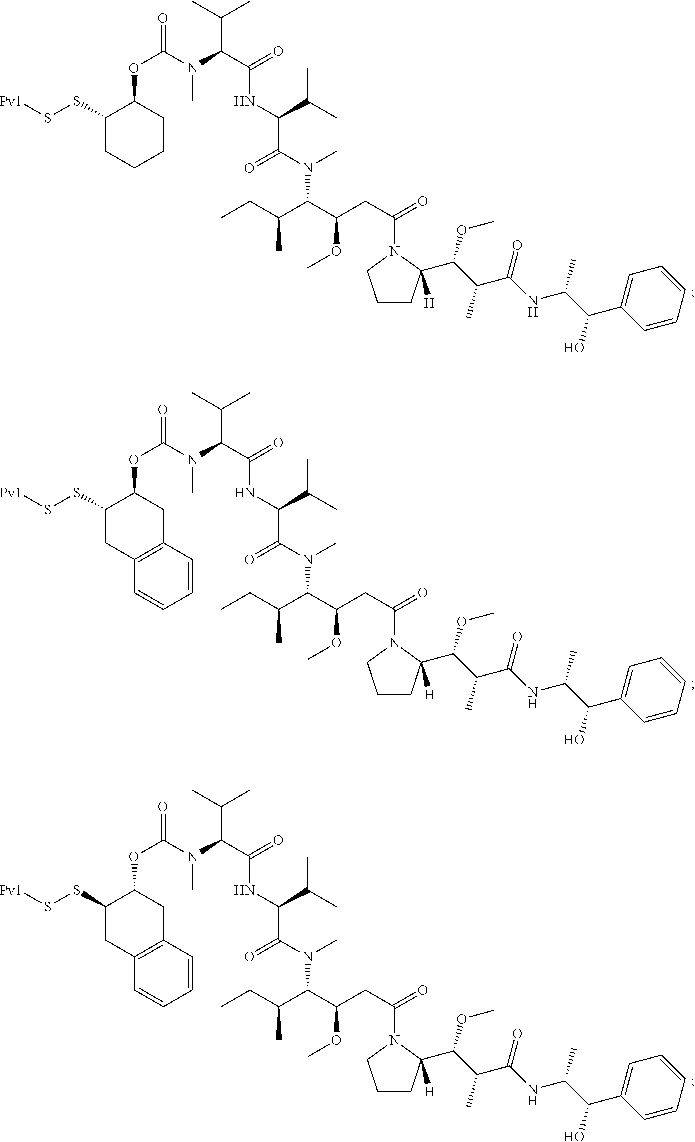

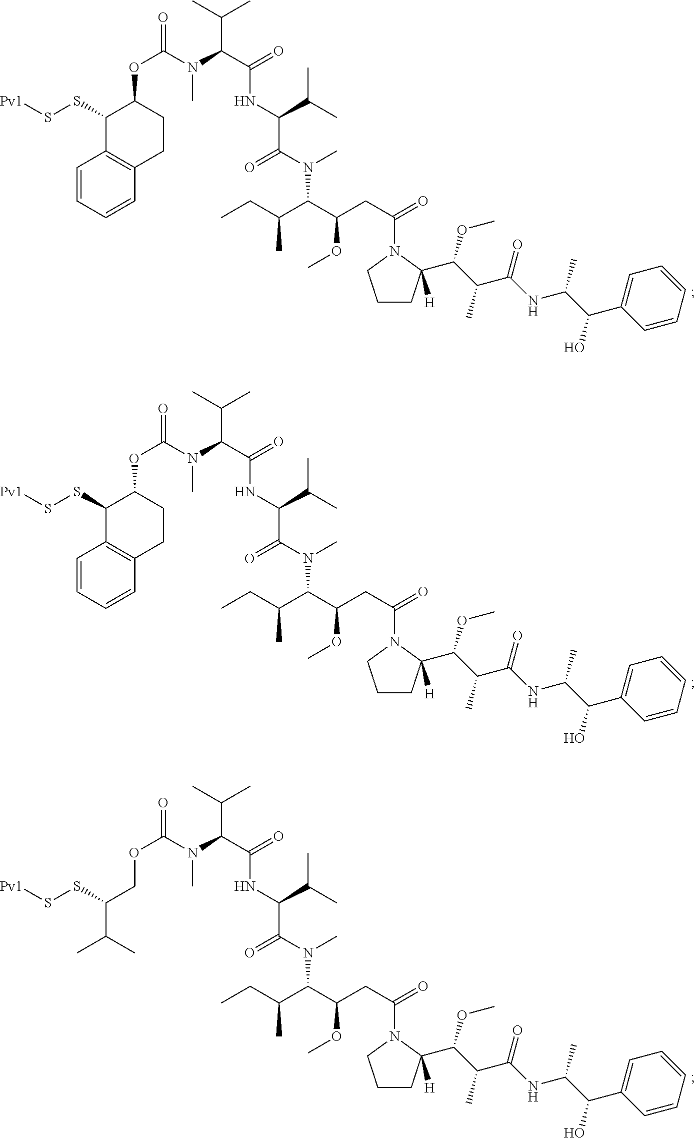

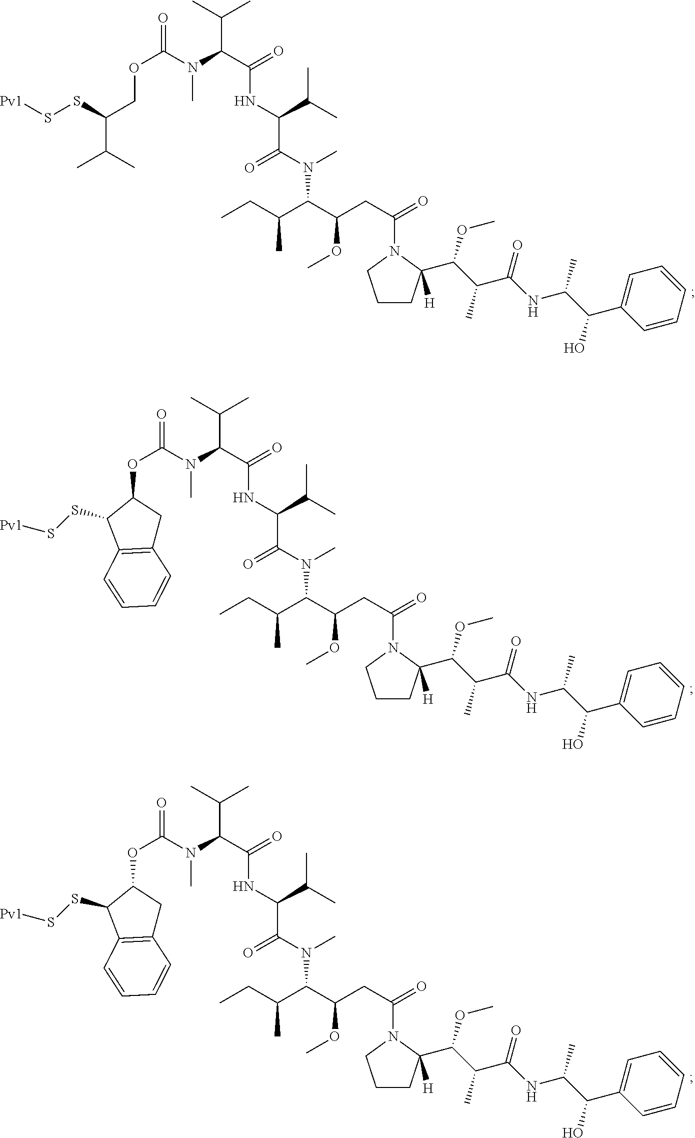

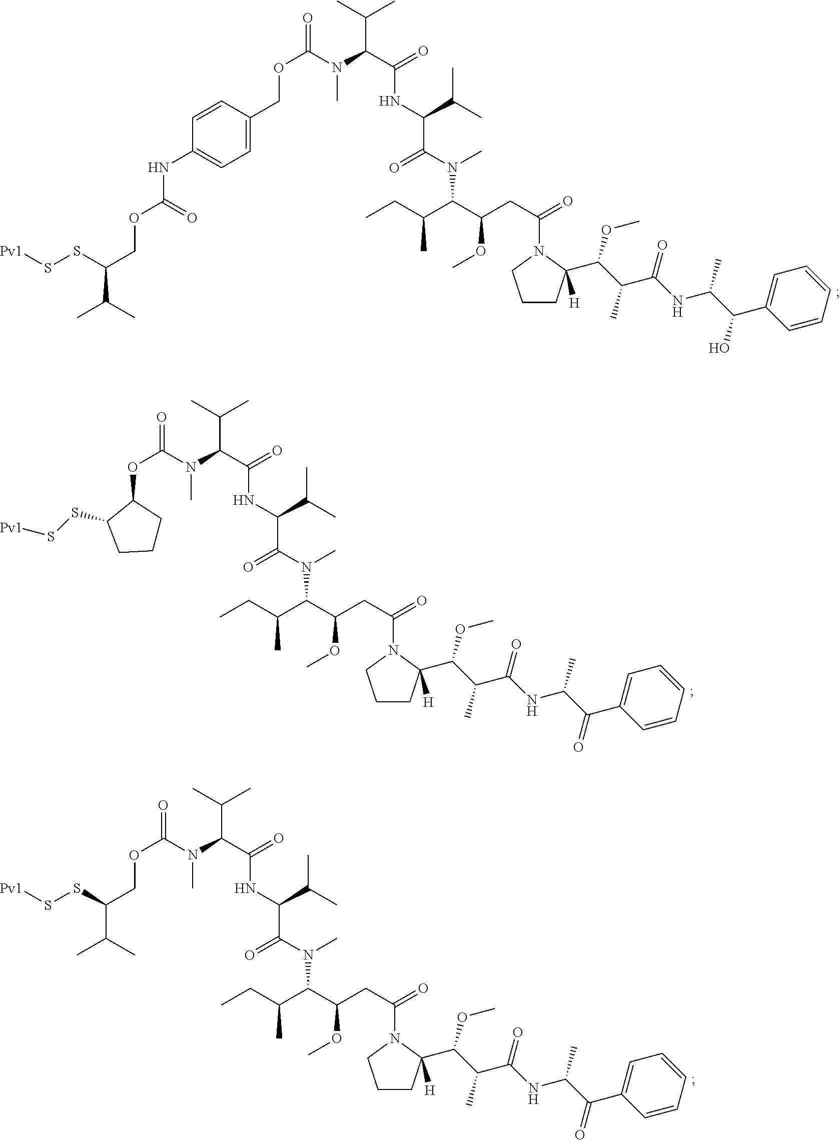

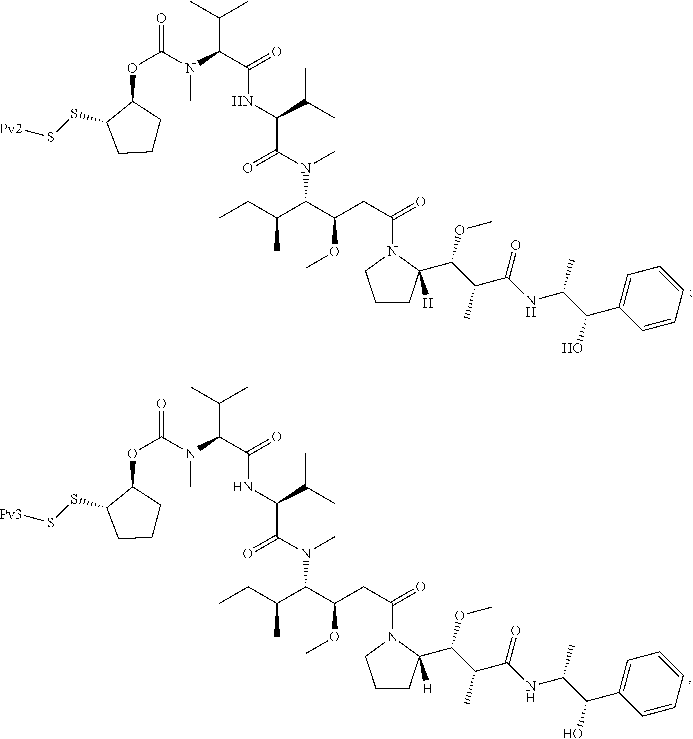

or a pharmaceutically acceptable salt of any of the aforementioned, wherein:
Pv1 is a peptide comprising the sequence:

(SEQ ID NO: 1)
ADDQNPWRAYLDLLFPTDTLLLDLLWCG;

Pv2 is a peptide comprising the sequence:

(SEQ ID NO: 2)
AEQNPIYWARYADWLFTTPLLLLDLALLVDADECG;

and
Pv3 is a peptide comprising the sequence:

(SEQ ID NO: 3)
ADDQNPWRAYLDLLFPTDTLLLDLLWDADECG.

In some embodiments, the compound of Formula (I) is selected from:

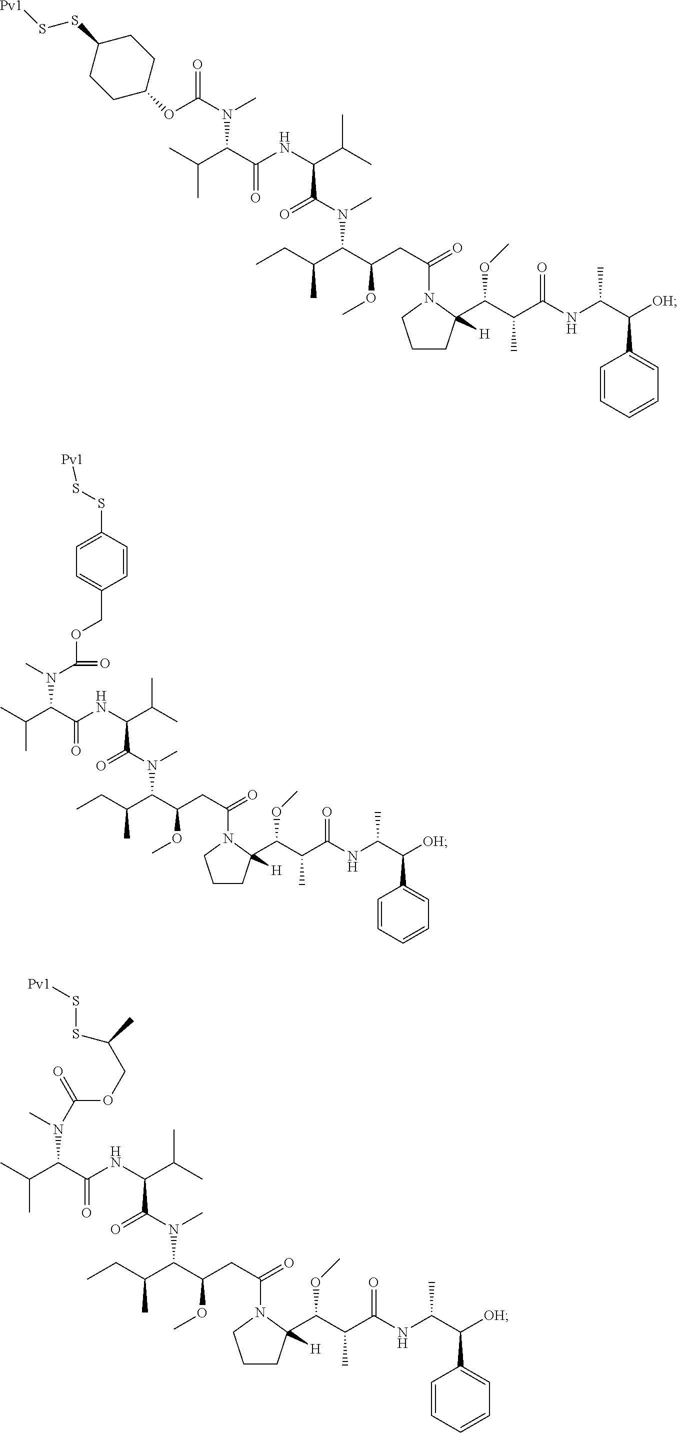

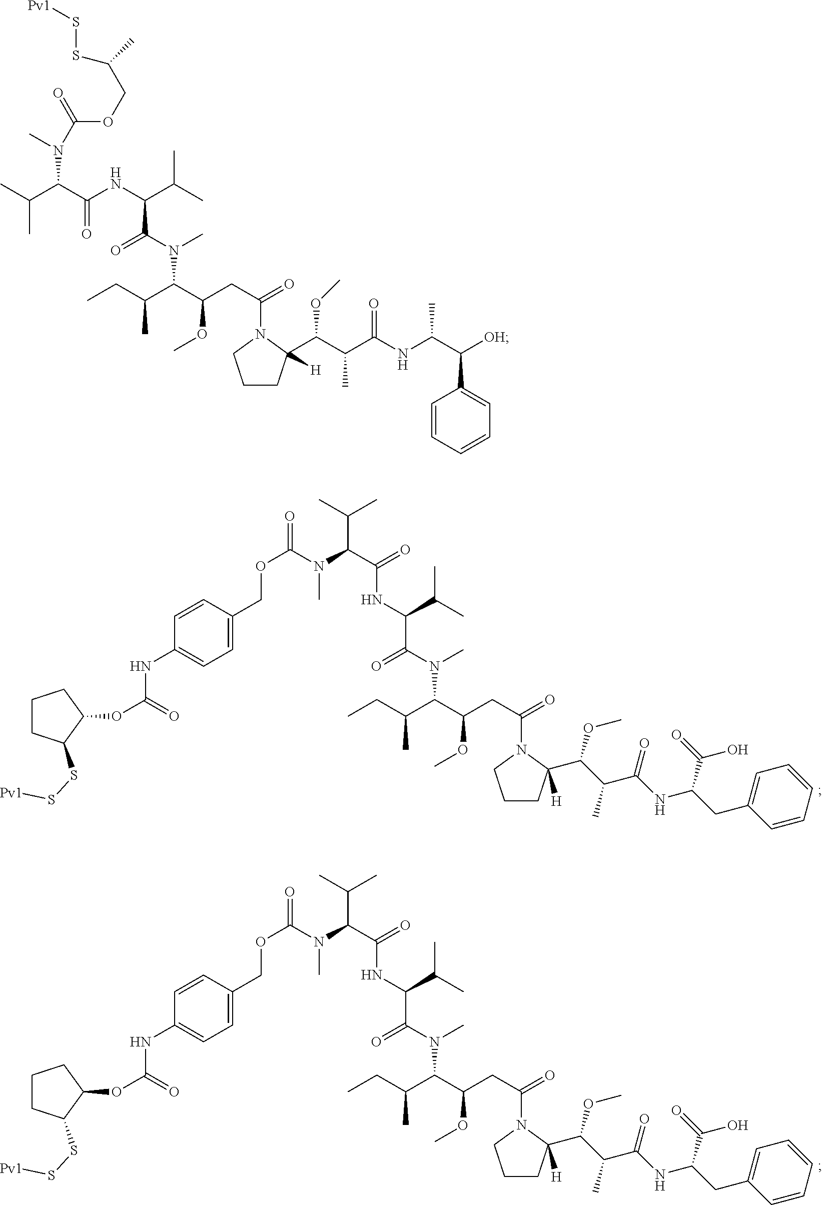

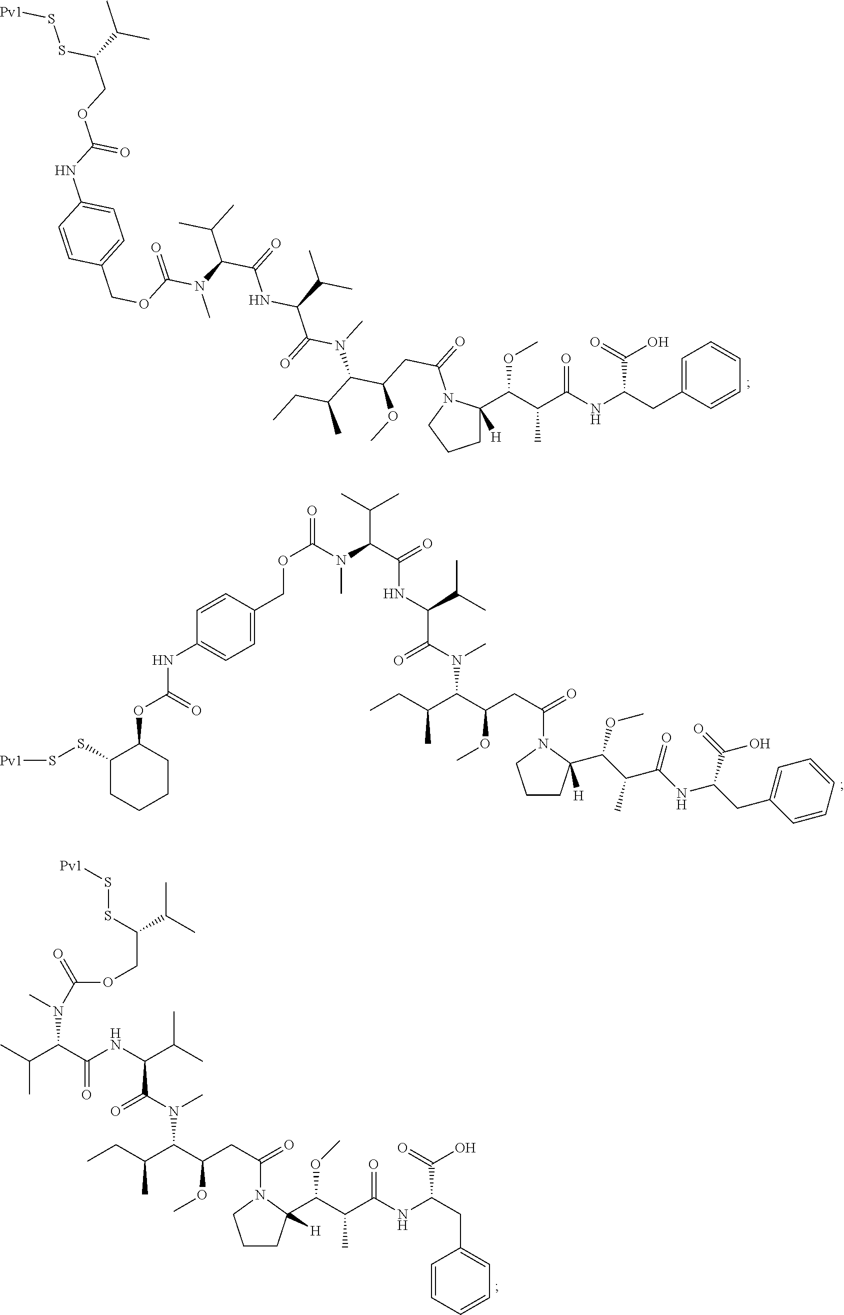

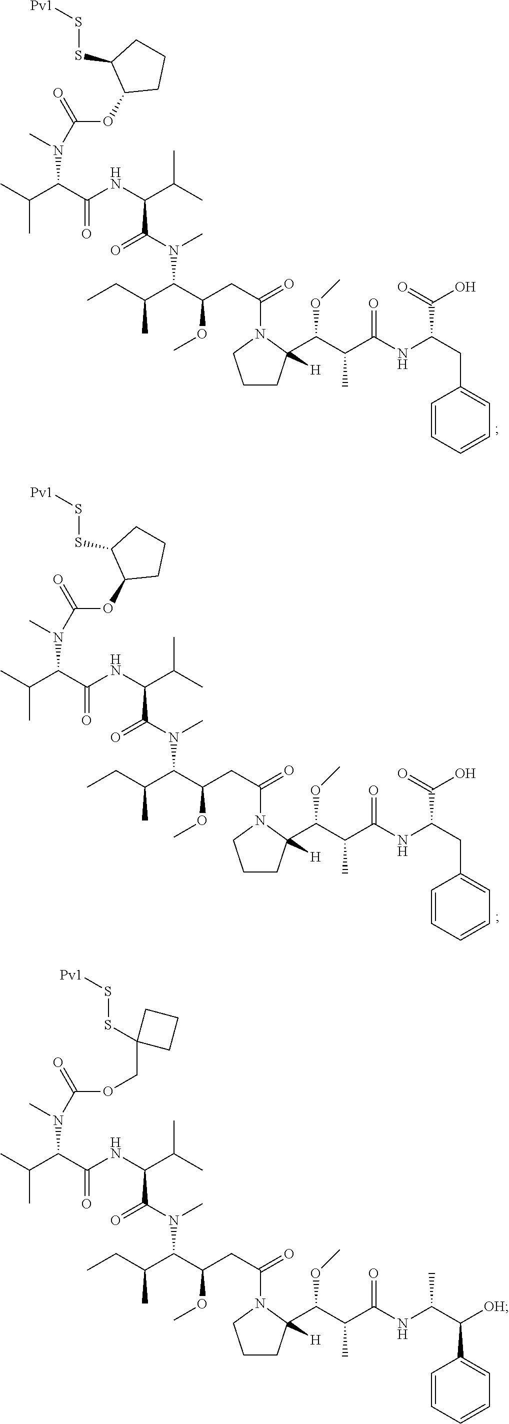

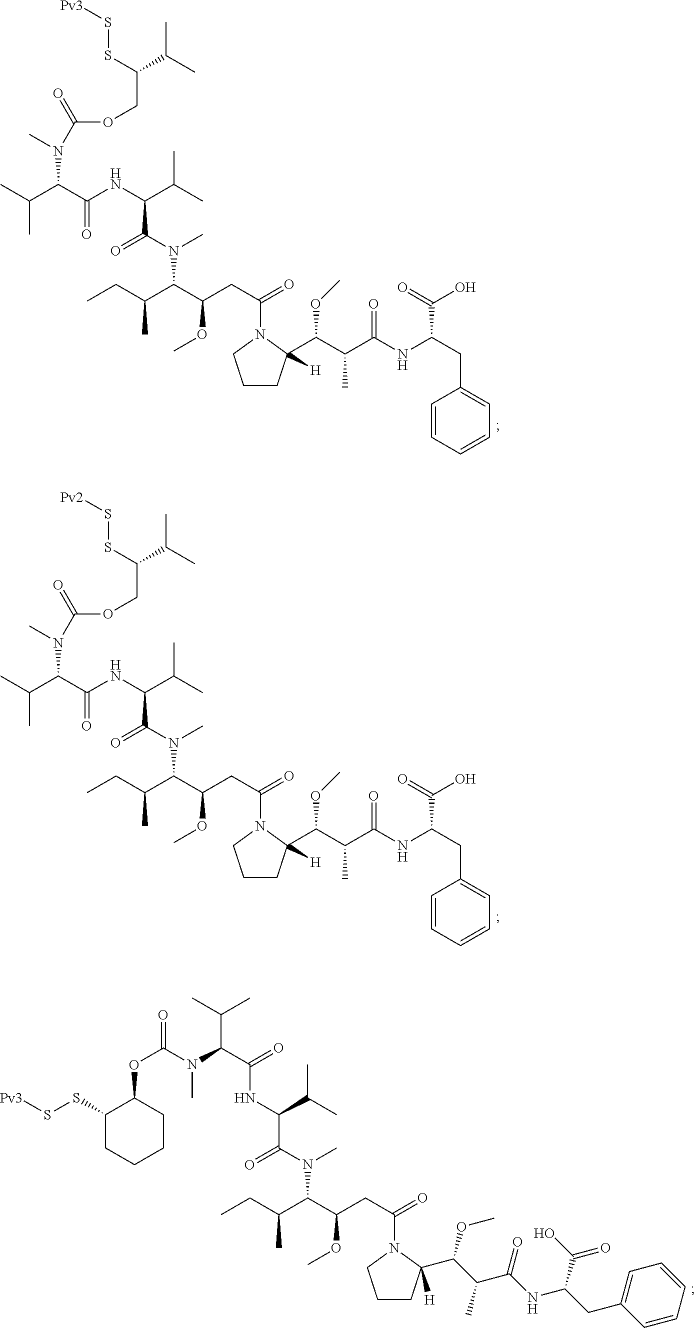

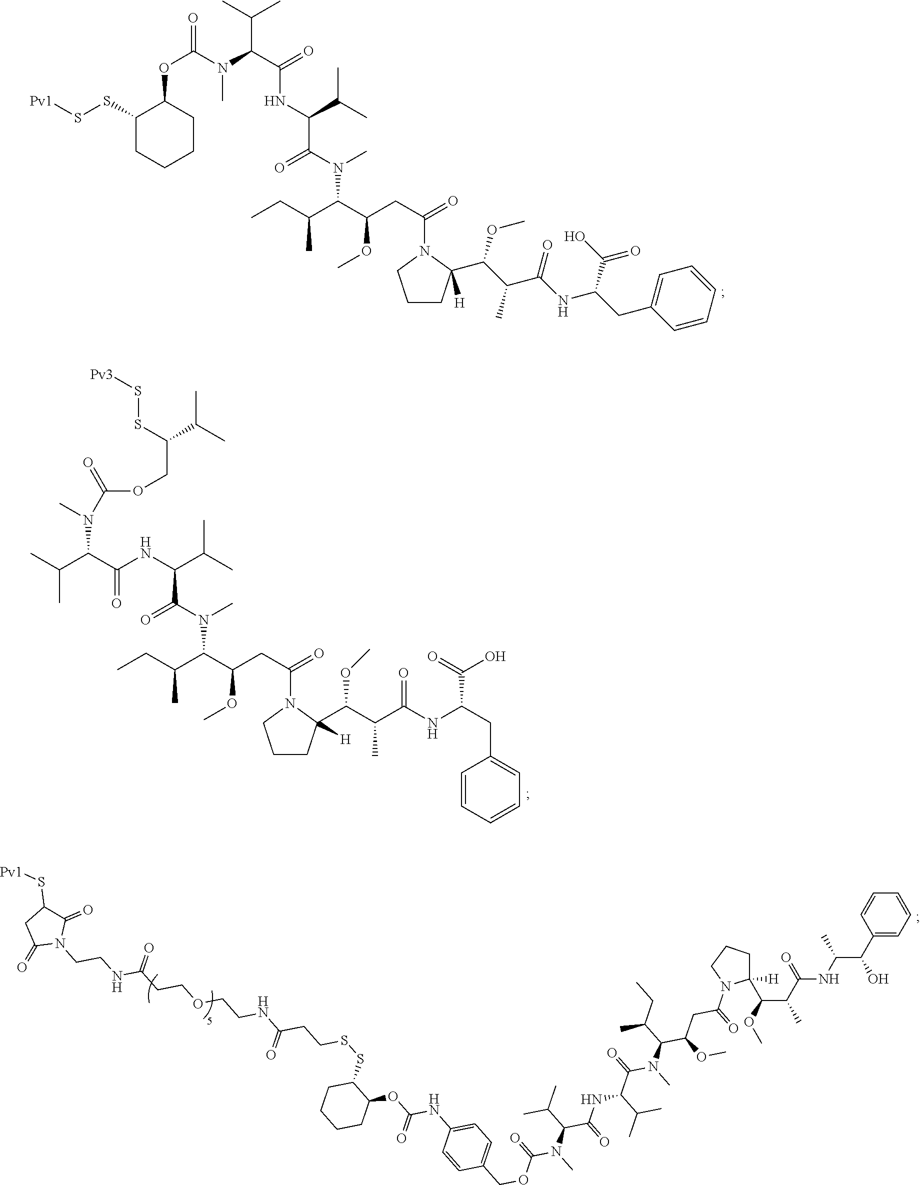

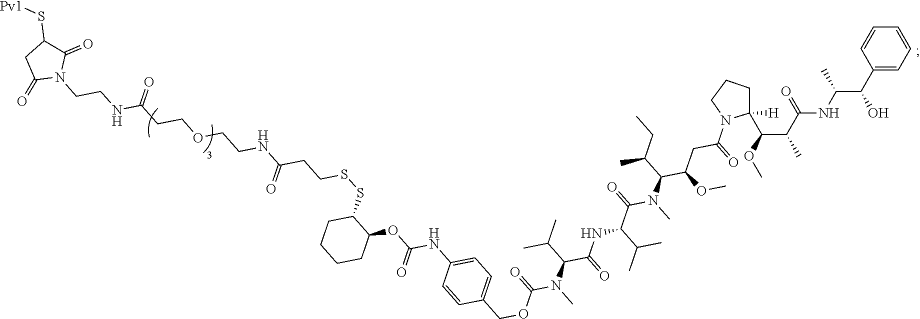

or a pharmaceutically acceptable salt of any of the aforementioned, wherein:
Pv1 is a peptide comprising the sequence:

(SEQ ID NO: 1)
ADDQNPWRAYLDLLFPTDTLLLDLLWCG;

Pv2 is a peptide comprising the sequence:

(SEQ ID NO: 2)
AEQNPIYWARYADWLFTTPLLLLDLALLVDADECG;

and
Pv3 is a peptide comprising the sequence:

(SEQ ID NO: 3)
ADDQNPWRAYLDLLFPTDTLLLDLLWDADECG.

The molecules of the invention can be tagged, for example, with a probe such as a fluorophore, radioisotope, and the like. In some embodiments, the probe is a fluorescent probe, such as LICOR. A fluorescent probe can include any moiety that can re-emit light upon light excitation (e.g., a fluorophore).

The Amino acids are represented by the IUPAC abbreviations, as follows: Alanine (Ala; A), Arginine (Arg; R), Asparagine (Asn; N), Aspartic acid (Asp; D), Cysteine (Cys; C), Glutamine (Gln; Q), Glutamic acid (Glu; E), Glycine (Gly; G), Histidine (His; H), Isoleucine (Ile; I), Leucine (Leu; L), Lysine (Lys; K), Methionine (Met; M), Phenylalanine (Phe; F), Proline (Pro; P), Serine (Ser; S), Threonine (Thr; T), Tryptophan (Trp; W), Tyrosine (Tyr; Y), Valine (Val; V).

The term “Pv1” means ADDQNPWRAYLDLLFPTDTLLLDLLWCG, which is the peptide of SEQ ID No. 1.

The term “Pv2” means AEQNPIYWARYADWLFTTPLLLLDLALLVDADECG, which is the peptide of SEQ ID No. 2.

The term “Pv3” means ADDQNPWRAYLDLLFPTDTLLLDLLWDADECG, which is the peptide of SEQ ID No. 3.

The term “Pv4” means Ac-AAEQNPIYWARYADWLFTTPLLLLDLALLVDADEGTKCG, which is the peptide of SEQ ID NO. 4.

The term “Pv5” means AAEQNPIYWARYADWLFTTPLLLLDLALLVDADEGTC, which is the peptide of SEQ ID NO. 5. The term “Pv6” means AAEQNPIYWWARYADWLFTTPLLLLDLALLVDADEGTCG, which is the peptide of SEQ ID NO. 6. In the compounds of the invention, the peptides R¹ are attached to the disulfide linker by a cysteine moiety.

The term “acidic and/or hypoxic mantle” refers to the environment of the cell in the diseased tissue in question having a pH lower than 7.0 and preferably lower than 6.5. An acidic or hypoxic mantle more preferably has a pH of about 5.5 and most preferably has a pH of about 5.0. The compounds of formula (I) insert across a cell membrane having an acidic and/or hypoxic mantle in a pH dependent fashion to insert R²L into the cell, whereupon the disulfide bond of the linker is cleaved to deliver free R²L (or R²L*, wherein L* is a product of degradation). Since the compounds of formula (I) are pH-dependent, they preferentially insert across a cell membrane only in the presence of an acidic or hypoxic mantle surrounding the cell and not across the cell membrane of “normal” cells, which do not have an acidic or hypoxic mantle.

The terms “pH-sensitive” or “pH-dependent” as used herein to refer to the peptide R¹ or to the mode of insertion of the peptide R¹ or of the compounds of the invention across a cell membrane, means that the peptide has a higher affinity to a cell membrane lipid bilayer having an acidic or hypoxic mantle than a membrane lipid bilayer at neutral pH. Thus, the compounds of the invention preferentially insert through the cell membrane to insert R²L to the interior of the cell (and thus deliver R²H as described above) when the cell membrane lipid bilayer has an acidic or hypoxic mantle (a “diseased” cell) but does not insert through a cell membrane when the mantle (the environment of the cell membrane lipid bilayer) is not acidic or hypoxic (a “normal” cell). It is believed that this preferential insertion is achieved as a result of the peptide R¹ forming a helical configuration, which facilitates membrane insertion.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. For example, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, C₁, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized □ (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring.

A five-membered heteroaryl ring is a heteroaryl group having five ring atoms wherein one or more (e.g., 1, 2 or 3) ring atoms are independently selected from N, O and S.

A six-membered heteroaryl ring is a heteroaryl group having six ring atoms wherein one or more (e.g., 1, 2 or 3) ring atoms are independently selected from N, O and S.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃-7). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2-pyrrolidinyl, morpholinyl, azetidinyl, tetrahydrofuranyl, tetrahydropyranyl, and piperazinyl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as α-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med. Chem. 2011, 54, 201-210; R. Xu et. al. J. Label Compd. Radiopharm. 2015, 58, 308-312).

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

Compounds of Formula (I) can be prepared, e.g., using a process as described below.

The peptides R¹ may be prepared using the solid-phase synthetic method first described by Merrifield in J.A.C.S., Vol. 85, pgs. 2149-2154 (1963), although other art-known methods may also be employed. The Merrifield technique is well understood and is a common method for preparation of peptides. Useful techniques for solid-phase peptide synthesis are described in several books such as the text “Principles of Peptide Synthesis” by Bodanszky, Springer Verlag 1984. This method of synthesis involves the stepwise addition of protected amino acids to a growing peptide chain which was bound by covalent bonds to a solid resin particle. By this procedure, reagents and by-products are removed by filtration, thus eliminating the necessity of purifying intermediates. The general concept of this method depends on attachment of the first amino acid of the chain to a solid polymer by a covalent bond, followed by the addition of the succeeding protected amino acids, one at a time, in a stepwise manner until the desired sequence is assembled. Finally, the protected peptide is removed from the solid resin support and the protecting groups are cleaved off.

The amino acids may be attached to any suitable polymer. The polymer must be insoluble in the solvents used, must have a stable physical form permitting ready filtration, and must contain a functional group to which the first protected amino acid can be firmly linked by a covalent bond. Various polymers are suitable for this purpose, such as cellulose, polyvinyl alcohol, polymethylmethacrylate, and polystyrene.

The preparation of various linkers provided herein is described in U.S. Pat. No. 10,933,069, and U.S. Application Publication Nos. 2021/0009536 and 2021/0009719.

Compounds of the invention can be prepared according to the following general scheme:

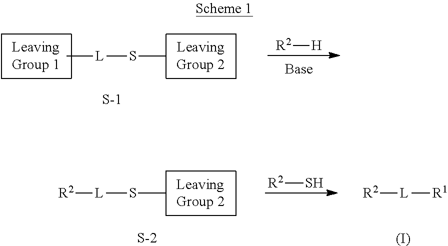

L is a thiol-containing moiety wherein the S atom of compound S-1 forms a disulfide bond with L. Compound S-1, which is flanked by orthogonal leaving groups, can be reacted with nucleophilic R²H compound to give compound S-2. Compound S-2 can then be reacted with a thiol containing peptide (R¹—SH) that participates in a disulfide exchange reaction to give a compound of Formula (I).

### Methods of Use

Provided herein is the use of the compounds of formula (I) in the treatment of diseases, such as cancer or neurodegenerative disease. Another aspect of the present invention is the use of the compounds of formula (I) in the treatment of diseases involving acidic or hypoxic diseased tissue, such as cancer. Hypoxia and acidosis are physiological markers of many disease processes, including cancer. In cancer, hypoxia is one mechanism responsible for development of an acid environment within solid tumors. As a result, hydrogen ions must be removed from the cell (e.g., by a proton pump) to maintain a normal pH within the cell. As a consequence of this export of hydrogen ions, cancer cells have an increased pH gradient across the cell membrane lipid bilayer and a lower pH in the extracellular milieu when compared to normal cells. One approach to improving the efficacy and therapeutic index of cytotoxic agents is to leverage this physiological characteristic to afford selective delivery of compound to hypoxic cells over healthy tissue.

In these methods of treatment, a therapeutically-effective amount of a compound of formula (I) or a pharmaceutically-acceptable salt thereof may be administered as a single agent or in combination with other forms of therapy, such as ionizing radiation or cytotoxic agents in the case of cancer. In combination therapy, the compound of formula (I) may be administered before, at the same time as, or after the other therapeutic modality, as will be appreciated by those of skill in the art. Either method of treatment (single agent or combination with other forms of therapy) may be administered as a course of treatment involving multiple doses or treatments over a period of time.

Examples of cancers that are treatable using the compounds of the present disclosure include, but are not limited to, bladder cancer, bone cancer, glioma, breast cancer, cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer, lung cancer, melanoma, prostate cancer, rectal cancer, renal clear cell carcinoma, skin cancer, stomach cancer, testicular cancer, thyroid cancer, and uterine cancer. In some embodiments, the cancer is selected from lung cancer, colorectal cancer, and prostate cancer. In some embodiments, the lung cancer is non-small cell lung cancer.

Examples of cancers that are treatuble using the compounds of the present disclosure further include Hodgkin lymphoma, anaplastic large cell lymphoma (ALCL), diffuse large B-cell lymphoma (DLBCL), ovarian cancer, urothelial cancer, non-small cell lung cancer (NSCLC), triple-negative breast cancer, squamous non-small cell lung cancer (sqNSCLC), squamous head and neck cancer, Non-Hodgkin lymphoma, pancreatic cancer, chronic myeloid leukemia (CMIL), acute myeloid leukemia (AML), fallopian tube cancer, and peritoneal cancer.

Examples of cancers that are treatable using the compounds of the present disclosure further include, but are not limited to, colorectal cancer, gastric cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, cancers treatable with compounds of the present disclosure include bladder cancer, bone cancer, glioma, breast cancer (e.g., triple-negative breast cancer), cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer (upper aerodigestive cancer), intestinal cancers, Kaposi's sarcoma, kidney cancer, laryngeal cancer, liver cancer (e.g., hepatocellular carcinoma), lung cancer (e.g., non-small cell lung cancer, adenocarcinoma), melanoma, prostate cancer, rectal cancer, renal clear cell carcinoma, skin cancer, stomach cancer, testicular cancer, thyroid cancer, and uterine cancer.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CMIL), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, a compound of formula (I) or a pharmaceutically-acceptable salt thereof may be used in combination with a chemotherapeutic agent, a targeted cancer therapy, an immunotherapy or radiation therapy. The agents can be combined with the present compounds in a single dosage form, or the agents can be administered simultaneously or sequentially as separate dosage forms. In some embodiments, the chemotherapeutic agent, targeted cancer therapy, immunotherapy or radiation therapy is less toxic to the patient, such as by showing reduced bone marrow toxicity, when administered together with a compound of formula (I), or a pharmaceutically acceptable salt thereof, as compared with when administered in combination with the corresponding microtubule targeting agent (e.g., R²—H).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with the compounds of the present invention include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds according to the invention may also be combined with immunotherapy drugs, including cytokines such as interferon alpha, interleukin 2, and tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-a), etoposide, and teniposide.

Other cytotoxic agents that can be administered in combination with the compounds of the invention include, for example, navelbene, CPT-11, anastrazole, letrazole, capecitabine, reloxafine, cyclophosphamide, ifosamide, and droloxafine.

Also suitable are cytotoxic agents such as, for example, epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB and PD-1, or antibodies to cytokines (IL-10, TGF-α, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines that can be administered in combination with the compounds of the invention include, for example, dendritic cells, synthetic peptides, DNA vaccines and recombinant viruses.

Other suitable agents for use in combination with the compounds of the present invention include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Compounds of the present invention may be combined with or administered in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with the compounds of the invention. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with compounds of the invention. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds of the present invention include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds of the invention. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds of the present invention. A further example of a PARP inhibitor that can be combined with a compound of the invention is talazoparib.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

The phrase “therapeutically effective amount” of a compound (therapeutic agent, active ingredient, drug, etc.) refers to an amount of the compound to be administered to a subject in need of therapy or treatment which alleviates a symptom, ameliorates a condition, or slows the onset of disease conditions, according to clinically acceptable standards for the disorder or condition to be treated. For instance, a therapeutically effective amount can be an amount which has been demonstrated to have a desired therapeutic effect in an in vitro assay, an in vivo animal assay, or a clinical trial. The therapeutically effective amount can vary based on the particular dosage form, method of administration, treatment protocol, specific disease or condition to be treated, the benefit/risk ratio, etc., among numerous other factors.

Said therapeutically effective amount can be obtained from a clinical trial, an animal model, or an in vitro cell culture assay. It is known in the art that the effective amount suitable for human use can be calculated from the effective amount determined from an animal model or an in vitro cell culture assay. For instance, as reported by Reagan-Shaw et al., FASEB J. 2008: 22(3) 659-61, “g/ml” (effective amount based on in vitro cell culture assays)=“mg/kg body weight/day” (effective amount for a mouse). Furthermore, the effective amount for a human can be calculated from the effective amount for a mouse based on the fact that the metabolism rate of mice is 6 times faster than that of humans.

As an example of treatment using a compound of formula (I) in combination with a cytotoxic agent, a therapeutically-effective amount of a compound of formula (I) may be administered to a patient suffering from cancer as part of a treatment regimen also involving a therapeutically-effective amount of ionizing radiation or a cytotoxic agent. In the context of this treatment regimen, the term “therapeutically-effective” amount should be understood to mean effective in the combination therapy. It will be understood by those of skill in the cancer-treatment field how to adjust the dosages to achieve the optimum therapeutic outcome.

Similarly, the appropriate dosages of the compounds of the invention for treatment of non-cancerous diseases or conditions (such as cardiovascular diseases) may readily be determined by those of skill in the medical arts.

The term “treating” as used herein includes the administration of a compound or composition which reduces the frequency of, delays the onset of, or reduces the progression of symptoms of a disease involving acidic or hypoxic diseased tissue, such as cancer, stroke, myocardial infarction, or long-term neurodegenerative disease, in a subject relative to a subject not receiving the compound or composition. This can include reversing, reducing, or arresting the symptoms, clinical signs, or underlying pathology of a condition in a manner to improve or stabilize a subject's condition (e.g., regression of tumor growth, for cancer or decreasing or ameliorating myocardial ischemia reperfusion injury in myocardial infarction, stroke, or the like cardiovascular disease). The terms “inhibiting” or “reducing” are used for cancer in reference to methods to inhibit or to reduce tumor growth (e.g., decrease the size of a tumor) in a population as compared to an untreated control population.

All publications (including patents) mentioned herein are incorporated herein by reference for the purpose of describing and disclosing, for example, the constructs and methodologies that are described in the publications, which might be used in connection with the disclosure herein described. The publications discussed throughout the text are provided solely for their disclosure prior to the filing date of the present application.

Disclosed herein are several types of ranges. When a range of any type is disclosed or claimed, the intent is to disclose or claim individually each possible number that such a range could reasonably encompass, including end points of the range as well as any sub-ranges and combinations of sub-ranges encompassed therein. When a range of therapeutically effective amounts of an active ingredient is disclosed or claimed, for instance, the intent is to disclose or claim individually every possible number that such a range could encompass, consistent with the disclosure herein. For example, by a disclosure that the therapeutically effective amount of a compound can be in a range from about 1 mg/kg to about 50 mg/kg (of body weight of the subject).

### Formulation, Dosage Forms and Administration

To prepare the pharmaceutical compositions of the present invention, a compound of Formula (I) or a pharmaceutically-acceptable salt thereof is combined as the active ingredient in intimate admixture with a pharmaceutical carrier according to conventional pharmaceutical compounding techniques, which carrier may take a wide variety of forms depending on the form of preparation desired for administration, e.g., oral or parenteral. In preparing the compositions in oral dosage form, any of the usual pharmaceutical media may be employed, such as for example, water, glycols, oils, alcohols, flavoring agents, preservatives, coloring agents, and the like in the case of oral liquid preparations such as for example, suspensions, elixirs, and solutions; or carriers such as starches, sugars, diluents, granulating agents, lubricants, binders, disintegrating agents, and the like in a case of oral solid preparations, such as for example, powders, capsules, and tablets. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, although other ingredients, for example, to aid solubility or for preservative purposes, may be included. Injectable suspensions may also be prepared, in which case appropriate liquid carriers, suspending agents, and the like may be employed. One of skill in the pharmaceutical and medical arts will be able to readily determine a suitable dosage of the pharmaceutical compositions of the invention for the particular disease or condition to be treated.

### EXAMPLES

### Mass Spectrometry Methods

Mass spectrometry was measured on an Agilent 1260 Infinity II with 6130B Quadrupole MS and Agilent 1290 Infinity II with 6125B Quadrupole MS.

Alternatively, Maldi-TOF (Matrix-assisted laser desorption/ionization-Time of Flight) mass spectrometry was measured on an Applied Biosystems Voyager System 6268. The sample was prepared as a matrix of α-cyano hydroxy cinnamic acid on an AB Science plate (Part #V700666).

ESI (Electrospray Ionization) mass spectrometry was measured on either an Agilent 1100 series LC-MS with a 1946 MSD or a Waters Xevo Qtof high-resolution MS, both providing a mass/charge species (m/z=3).

### HPLC Methods

HPLCs were recorded from an Agilent 1260 Infinity II machine. The HPLC methods are described in more detail as needed in each example below.

### Preparation of Linkers

The preparation of various linkers provided herein is described in U.S. Pat. No. 10,933,069, and U.S. Application Publication Nos. 2021/0009536 and 2021/0009719. For example, the sysnthesis of the following linkers is described in U.S. Application Publication No. 2021/0009719:

| Linker Name | Structure |
|---|---|
| L31 | 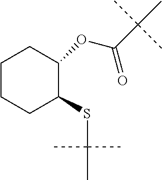 |
| L55* | 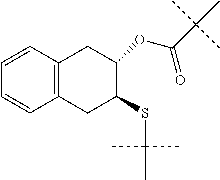 |
| L56* | 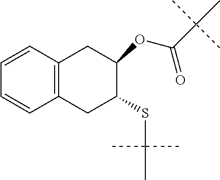 |
| L61* | 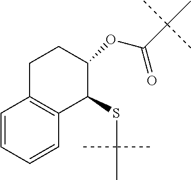 |
| L62* | 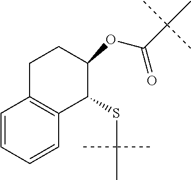 |
| L65 | 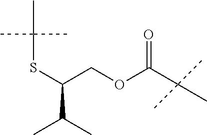 |
| L64 | 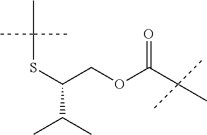 |

*Relative stereochemistry is shown. See U.S. Application Publication No. 2021/0009719 for details.

### Synthesis of Linker Compounds L50 and L51

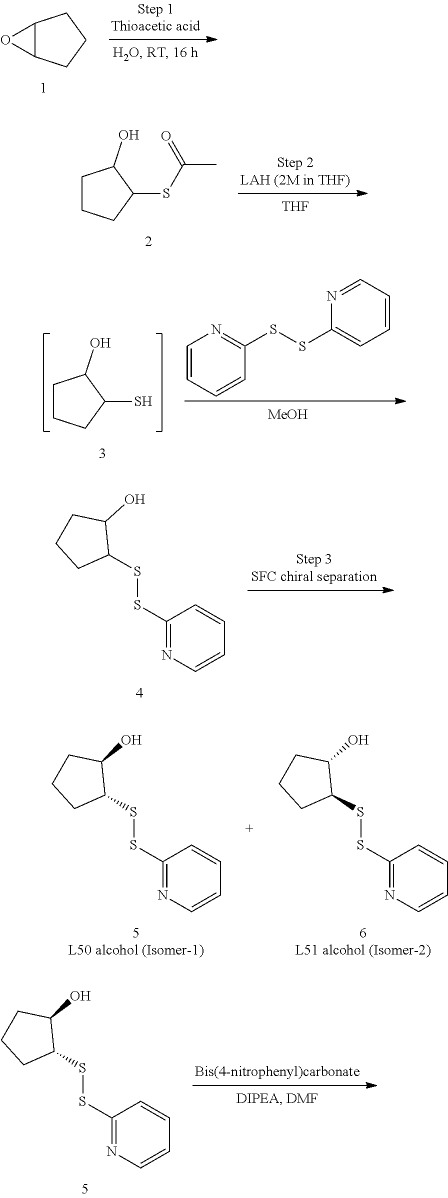

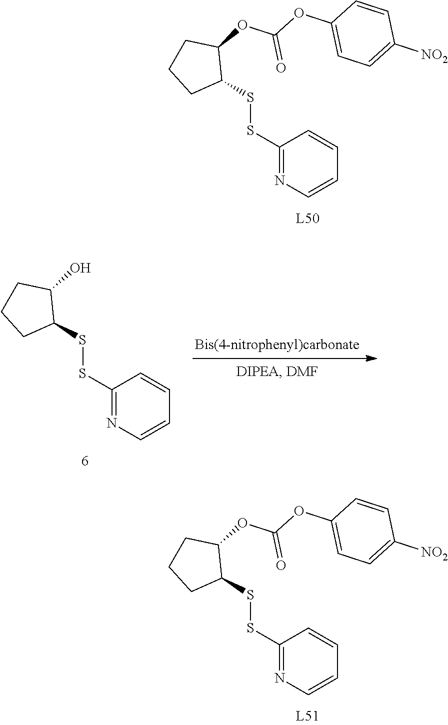 [SMILES: O=C(Oc1ccc([N+](=O)[O-])cc1)O[C@@H]1CCC[C@H]1SSc1ccccn1 ; O[C@H]1CCC[C@@H]1SSc1ccccn1 ; O=C(Oc1ccc([N+](=O)[O-])cc1)O[C@H]1CCC[C@@H]1SSc1ccccn1]

### Step 1: Synthesis of S-(2-hydroxycyclopentyl) ethanethioate

To a stirred solution of 6-oxabicyclo [3.1.0] hexane (5 g, 59.4 mmol) in Water (50 ml) was added thioacetic acid (4.98 g, 65.4 mmol) at RT. The reaction mixture was stirred at RT for 18 h. The reaction mixture was quenched with an aqueous solution of saturated NaHCO₃ and extracted with ethyl acetate. The organic layer was dried over Na₂SO₄ and evaporated to afford S-(2-hydroxy cyclopentyl) ethanethioate (6.1 g, 38.1 mmol, 64.0% yield) as a colorless liquid. The crude product was taken for next step without purification.

### Step 2: Synthesis of 2-mercaptocyclopentan-1-ol

To a stirred solution of S-(2-hydroxycyclopentyl) ethanethioate (6.1 g, 38.1 mmol) in THE (60 ml) at 0° C., a 2.0 M solution of LAH in THE (28.6 ml, 57.1 mmol) was added dropwise. The reaction mixture was stirred at RT for 3 h. The reaction mixture was cooled to 0° C. and quenched with an aqueous solution of 1.5 N HCl and extracted with DCM. The organic layer was dried over Na₂SO₄ and evaporated to afford 2-mercaptocyclopentan-1-ol (5 g, 42.3 mmol, 111% yield) as a colorless liquid. The crude product was taken for next step without purification. ¹H NMR (400 MHz, DMSO-d₆): δ 4.90 (s, 1H), 3.78 (s, 1H), 2.93-2.87 (m, 1H), 2.44-2.42 (m, 1H), 2.19-2.05 (m, 1H), 1.96-1.88 (m, 1H), 1.69-1.67 (m, 2H), 1.50-1.35 (m, 2H).

### Step 3: 2-(pyridin-2-yldisulfaneyl) cyclopentan-1-ol

To a stirred solution of 2-mercaptocyclopentan-1-ol (5 g, 42.3 mmol) in Methanol (60 ml) was added 1,2-di(pyridin-2-yl) disulfane (13.98 g, 63.5 mmol) at 0° C. The reaction mixture was stirred at RT for 18 h. The reaction mixture was evaporated to dryness. Ice cold water was added, extracted with ethyl acetate. The organic layer was separated, washed with brine, dried over Na₂SO₄ and evaporated to get a crude residue. The crude residue was purified twice by flash column chromatography using 10% ethyl acetate in petroleum ether to get 2-(pyridin-2-yldisulfaneyl) cyclopentan-1-ol as a racemic mixture. LCMS: [M+H]⁺ calcd for C₁₀H₁₃NOS₂, 227.04; found 228.1 (M+H). SFC chiral purity: Column: Lux A1; Co-solvent: 40% MeOH; Flow rate: 4 mL/min; RT (min): 2.98; Area %: 49.92; RT (min): 4.26; Area %: 48.79. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 5.76; Purity (Max): 99.41%

### SFC Separation of isomers (1R,2R)-2-(pyridin-2-yldisulfaneyl) cyclopentan-1-ol (L-50 alcohol) & (1S,2S)-2-(pyridin-2-yldisulfaneyl) cyclopentan-1-ol (L-51 alcohol)

The isomers were separated by SFC purification of racemic 2-(pyridin-2-yldisulfaneyl) cyclopentan-1-ol. The obtained SFC fraction isomer-1 (first eluted peak) was concentrated under reduced pressure at 30° C. to afford (1R,2R)-2-(pyridin-2-yldisulfaneyl)cyclopentan-1-ol (L-50 alcohol) (1.2 g, 5.18 mmol, 12.25% yield) as a colorless oil. Absolute stereochemistry was assigned as set forth in Yamshita H., Bull. Chem. Soc. Jpn., 61, 1213-1220 (1988). LCMS: [M+H]⁺ calcd for C₁₀H₁₃NOS₂, 227.04; found 228.1 (M+H). HPLC: Column: X-Bridge C8 (50×4.6) mm, 3.5 μm; Mobile phase: A: 0.1% TFA in H₂O; Mobile phase: B: 0.1% TFA in ACN; Flow: 2.0 mL/min; RT (min): 2.71; Purity (Max): 98.19%. SFC chiral purity: Column: Lux A1; Co-solvent: 40% MeOH; Flow rate: 40 mL/min; RT (min): 2.94; Area %: 100.0. ¹H NMR (400 MHz, CDCl₃): δ 8.55 (s, 1H), 7.65-7.61 (m, 1H), 7.54-7.51 (m, 1H), 7.21-7.17 (m, 1H), 4.05-4.04 (m, 1H), 3.03 (t, J=8.00 Hz, 1H), 2.12-2.05 (m, 2H), 1.72-1.64 (m, 5H).

### Synthesis of the Precursor to Linker L50

To a stirred solution of (1R,2R)-2-(pyridin-2-yldisulfaneyl)cyclopentan-1-ol (1.1 g, 4.84 mmol) in DMF (10 ml) were added bis(4-nitrophenyl) carbonate (2.94 g, 9.68 mmol) and DIPEA (2.51 ml, 14.52 mmol) at RT. The reaction mixture was stirred at RT for 18 h. The reaction mixture was diluted with ice cold water and extracted with ethyl acetate. The organic layer was washed with brine, dried over Na₂SO₄ to get the crude product. The crude product was purified by reverse phase chromatography using 0.1% HCOOH in H₂O and ACN. The product fraction was concentrated under reduced pressure to get the pure product which was lyophilized to afford 4-nitrophenyl ((1R,2R)-2-(pyridin-2-yl disulfaneyl)cyclopentyl) carbonate (1.7 g, 4.32 mmol, 89% yield) as a pale yellow gum. LCMS: [M+H]⁺ calcd for C₁₇H₁₆N₂O₅S₂, 392.05; found 392.9 (M+H). HPLC: Column: X-Bridge C8 (50×4.6) mm, 3.5 μm; Mobile phase: A: 0.1% TFA in H₂O; Mobile phase: B: 0.1% TFA in ACN; Flow: 2.0 mL/min; RT (min): 5.01; Purity (Max): 99.73%. SFC chiral purity: Column: YMC Amylose-SA; Co-solvent: 30% IPA; Flow rate: 3 mL/min; RT (min): 4.26; Area %: 99.95. ¹H NMR (400 MHz, CDCl₃): δ 400 MHz, CDCl₃: δ 8.50 (s, 1H), 8.29-8.27 (m, 2H), 7.71-7.65 (m, 2H), 7.39-7.36 (m, 2H), 7.14-7.11 (m, 1H), 5.25 (t, J=3.20 Hz, 1H), 3.60-3.55 (m, 1H), 2.30-2.27 (m, 2H), 2.03-1.79 (m, 3H), 1.70-1.69 (m, 1H).

### Synthesis of the Precursor to Linker L51

To a stirred solution of (1S,2S)-2-(pyridin-2-yldisulfaneyl)cyclopentan-1-ol (1.1 g, 4.84 mmol) in DMF (10 ml) were added bis(4-nitrophenyl) carbonate (2.94 g, 9.68 mmol) and DIPEA (2.51 ml, 14.52 mmol) at RT. The reaction mixture was stirred at RT for 18 h. The reaction mixture was diluted with ice cold water and extracted with ethyl acetate. The organic layer was washed with brine, dried over Na₂SO₄ to get the crude product. The crude product was purified by reverse phase chromatography using 0.1% HCOOH in H₂O and ACN. The product fraction was concentrated under reduced pressure to get the pure product which was lyophilized to afford 4-nitrophenyl ((1S,2S)-2-(pyridin-2-yl disulfaneyl)cyclopentyl) carbonate (1.7 g, 4.24 mmol, 88% yield) as a pale yellow gum compound. LCMS: [M+H]⁺ calcd for C₁₇H₁₆N₂O₅S₂, 392.05; found 392.8 (M+H). HPLC: Column: X-Bridge C8 (50×4.6) mm, 3.5 μm; Mobile phase: A: 0.1% TFA in H₂O; Mobile phase: B: 0.1% TFA in ACN; Flow rate: 2.0 mL/min; RT (min): 5.01; Purity (Max): 97.86%. SFC chiral purity: Column: YMC Amylose-SA; Co-solvent: 30% IPA; Flow rate: 3 mL/min; RT (min): 3.56; Area %: 99.74. ¹H NMR (400 MHz, CDCl₃): δ 400 MHz, CDCl₃: δ 8.50 (s, 1H), 8.29-8.27 (m, 2H), 7.71-7.65 (m, 2H), 7.39-7.36 (m, 2H), 7.14-7.11 (m, 1H), 5.25 (t, J=3.20 Hz, 1H), 3.60-3.55 (m, 1H), 2.30-2.27 (m, 2H), 2.03-1.79 (m, 3H), 1.70-1.69 (m, 1H).

### Alternative Synthesis of Linker L51

Linker L51 can be prepared according to the enzymatic chiral resolution process disclosed in International Application WO 2022/150596, which is incorporated herein in its entirety (see, for example, Example 11 of WO 2022/150596).

### Synthesis of Compounds of the Disclosure

### Example 1: Synthesis of Compound 1

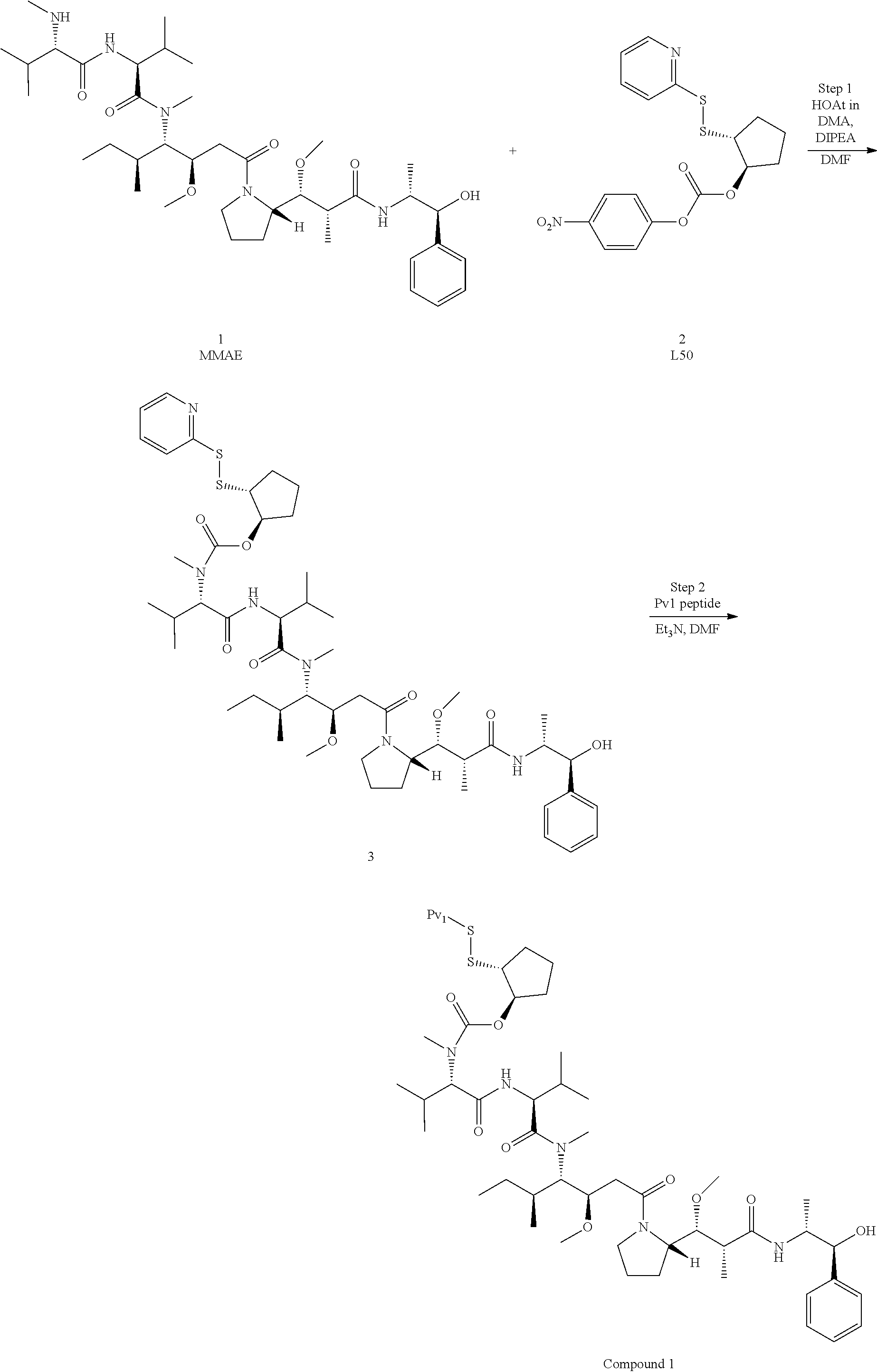

### Step 1: Synthesis of (1R,2R)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (3)

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (150 mg, 0.209 mmol) in DMF (1 mL) was added 4-nitrophenyl ((1R,2R)-2-(pyridin-2-yldisulfaneyl)cyclopentyl) carbonate (L50) (98 mg, 0.251 mmol) at 0° C. Then, a 1 M solution of 1-hydroxy-7-azabenzotriazole in DMA (0.104 ml, 0.104 mmol) and DIPEA (0.054 ml, 0.313 mmol) were added and the reaction mixture was stirred at RT for 18 h. The reaction mixture was purified by preparative using 0.1% HCOOH in H₂O and ACN. The product fraction was lyophilized to afford (1R,2R)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (138 mg, 0.140 mmol, 67.1% yield) as a white solid. LCMS: [M+H]⁺ calcd for C₅₀H₇₈N₆O₉S₂, 970.53; found 971.3 (M+H). HPLC: Column: X-Bridge C8 (50×4.6) mm, 3.5 μm; Mobile phase: A: 0.1% TFA in H₂O; Mobile phase: B: 0.1% TFA in ACN; Flow: 2.0 mL/min; RT (min): 5.49; Purity (Max): 98.69%.

### Step 2: Synthesis of Compound 1

To a stirred solution of (1R,2R)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (115 mg, 0.118 mmol) in DMF (1.5 ml) was added Pv1 peptide (425.6 mg, 0.130 mmol) and triethylamine (0.02 ml, 0.141 mmol) at 0° C. The reaction mixture was stirred at RT for 3 h. The reaction mixture was purified by preparative HPLC using 0.1% TFA in H₂O and ACN. The product fraction was lyophilized to afford Compound 1 (205 mg, 0.049 mmol, 41.5% yield) as a white solid. The product obtained is a di-TFA salt. LCMS: [M+H]⁺ calcd for C₁₉₇H₂₉₉F₆N₄₁O₅₂S₂, 4135.15; found 1380.3 (M+3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 12.29; Purity (Max): 99.69%

### Example 2: Synthesis of Compound 2

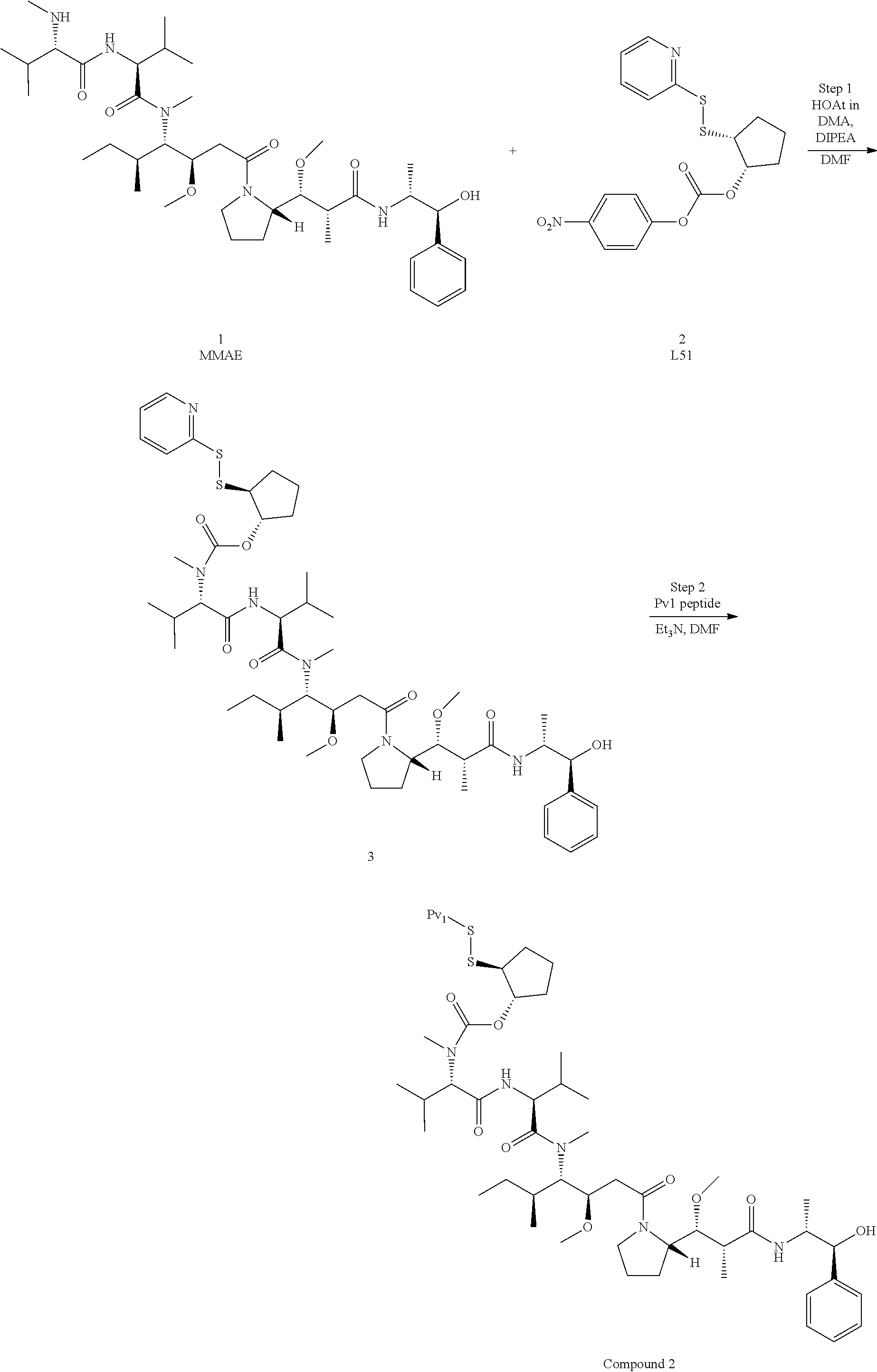

### Step 1: (1S,2S)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (180 mg, 0.251 mmol) in DMF (1 mL) was added 4-nitrophenyl ((1S,2S)-2-(pyridin-2-yldisulfaneyl)cyclopentyl) carbonate (L51) (118 mg, 0.301 mmol) at 0° C. Then, a 1 M solution of 1-hydroxy-7-azabenzotriazole in DMA (0.125 ml, 0.125 mmol) and DIPEA (0.066 ml, 0.376 mmol) were added and the reaction mixture was stirred at RT for 16 h. The reaction mixture was purified by preparative using 0.1% HCOOH in H₂O and ACN. The product fraction was lyophilized to afford (1S,2S)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (200 mg, 0.251 mmol, 79% yield) as a white solid. LCMS: [M+H]⁺ calcd for C₅₀H₇₈N₆O₉S₂, 970.53; found 971.4 (M+H). Column: X-Bridge C8 (50×4.6) mm, 3.5 μm; Mobile phase: A: 0.1% TFA in H₂O; Mobile phase: B: 0.1% TFA in ACN; Flow: 2.0 mL/min; RT (min): 5.48; Purity (Max): 95.59%.

### Step 2: Synthesis of Compound 2

To a stirred solution of (1S,2S)-2-(pyridin-2-yldisulfaneyl)cyclopentyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (200 mg, 0.206 mmol) in DMF (2 ml) were added Pv1 peptide (742.9 mg, 0.226 mmol) and triethylamine (0.035 ml, 0.247 mmol) at 0° C. The reaction mixture was stirred at RT for 1 h 30 min. The reaction mixture was purified by preparative HPLC using 0.1% TFA in H₂O and ACN. The product fraction was lyophilized to afford Compound 2 (410 mg, 0.099 mmol, 48% yield) as a white solid. The product obtained is a di-TFA salt. LCMS: [M+H]⁺ calcd for C₁₉₇H₂₉₉F₆N₄₁O₅₂S₂, 4135.15; found 1380.0 (M+3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 11.94; Purity (Max): 99.66%

### Example 3: Synthesis of Compound 3

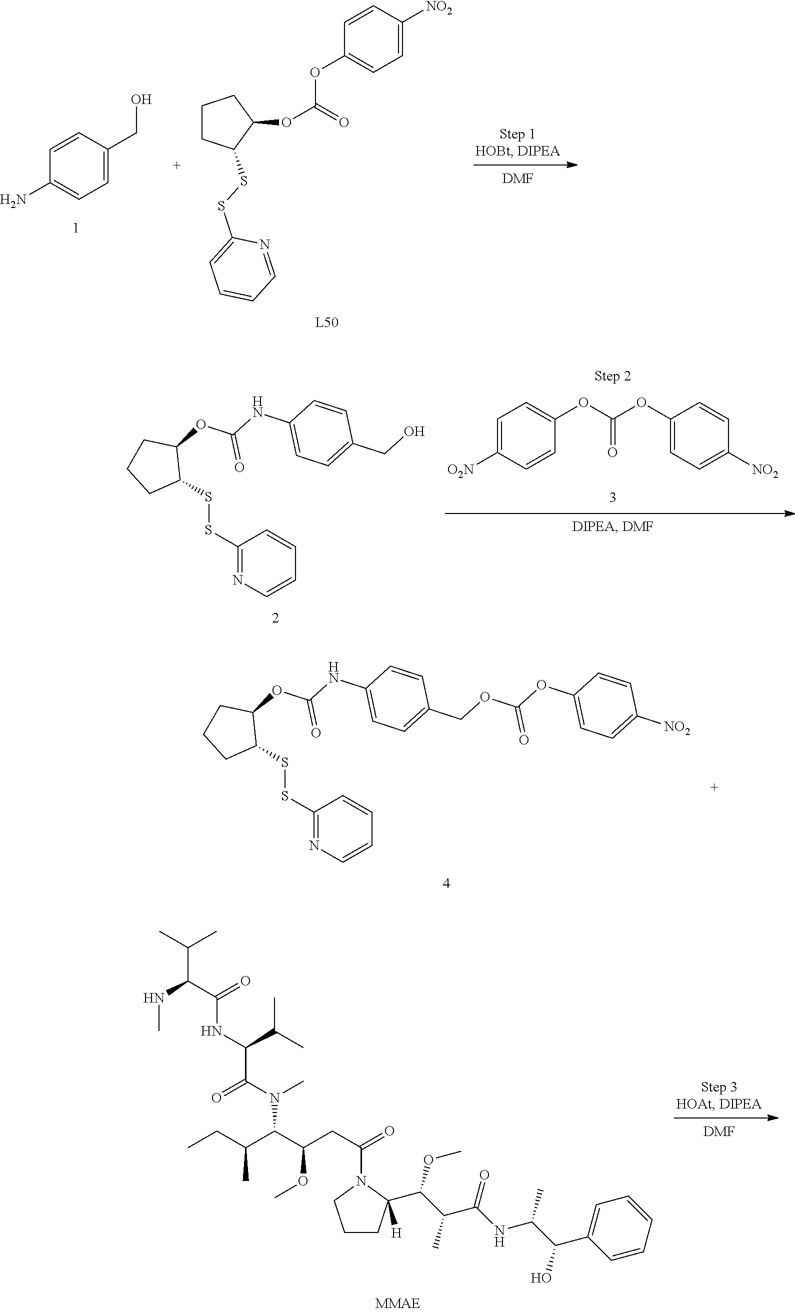

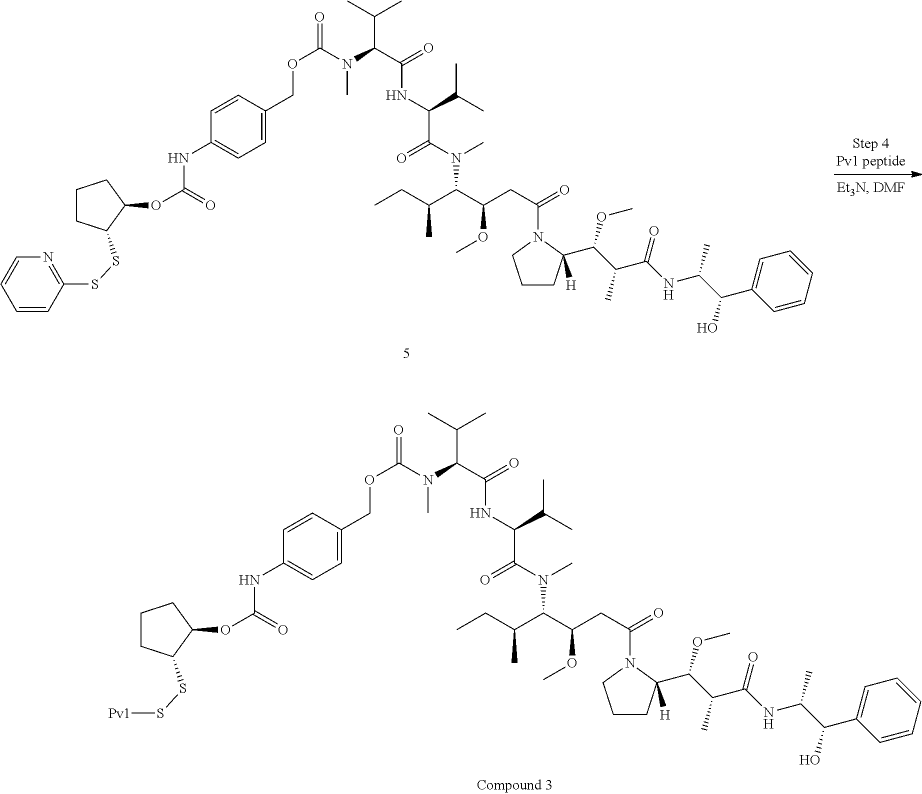

### Step 1: Synthesis of (1R,2R)-2-(pyridin-2-yldisulfaneyl) cyclopentyl (4-hydroxymethyl) phenyl) carbamate

A stirred solution of (4-aminophenyl) methanol (120 mg, 0.974 mmol) and 4-nitrophenyl ((1R,2R)-2-(pyridin-2-yldisulfaneyl) cyclopentyl) carbonate (L50) (382 mg, 0.974 mmol) in DMF (1 ml) was cooled with ice. To the above solution HOBt (65.8 mg, 0.487 mmol) and DIPEA (0.338 ml, 1.949 mmol) were added. The reaction mixture was stirred at RT for 18 h. The reaction mixture was diluted with ice cold water, extracted with ethyl acetate and washed with brine. The organic layer was concentrated to obtain a crude residue. The crude residue was purified by flash column chromatography using 40% ethyl acetate in petroleum ether to afford (1R,2R)-2-(pyridin-2-yldisulfaneyl)cyclopentyl (4-(hydroxymethyl)phenyl)carbamate (350 mg, 0.876 mmol, 90% yield) as a brown gum. LCMS: [M+H]⁺ calcd for C₁₅H₂₀F₆N₂O₃S₂, 376.09; found 377.6 (M+H). ¹H NMR (400 MHz, DMSO-d₆): δ 9.61 (s, 1H), 8.45 (d, J=4.80 Hz, 1H), 7.79-7.77 (m, 2H), 7.39-7.22 (m, 2H), 7.19-7.14 (m, 3H), 5.08 (t, J=5.60 Hz, 1H), 5.00 (s, 1H), 4.41 (d, J=5.60 Hz, 2H), 3.51-3.34 (m, 1H), 2.17-2.00 (m, 2H), 1.78-1.67 (m, 4H).

### Step 2: Synthesis of (1R,2R)-2-(pyridin-2-yldisulfaneyl) cyclopentyl (4-((((4-nitrophenoxy) carbonyl) oxy) methyl) phenyl) carbamate

To a stirred solution of (1R,2R)-2-(pyridin-2-yldisulfaneyl)cyclopentyl (4-(hydroxymethyl)phenyl)carbamate (0.35 g, 0.930 mmol) in DMF (5 ml) were added bis(4-nitrophenyl) carbonate (1.131 g, 3.72 mmol) and DIPEA (0.242 ml, 1.394 mmol) at RT. The reaction mixture was stirred at RT for 18 h. The reaction mixture was diluted with ice cold water and extracted with ethyl acetate. The organic layer was washed with brine, dried over Na₂SO₄ and concentrated under reduced pressure to obtain a crude residue. The crude residue was purified by flash column chromatography using 20% ethyl acetate in petroleum ether to afford (1R,2R)-2-(pyridin-2-yldisulfaneyl)cyclopentyl (4-((((4-nitrophenoxy)carbonyl)oxy)methyl)phenyl)carbamate (420 mg, 0.740 mmol, 80% yield) as a brown gum. LCMS: [M+H]⁺ calcd for C₂₅H₂₃N₃O₇S₂, 541.10; found 542.1 (M+H). ¹H NMR (400 MHz, DMSO-d₆): δ 9.78 (s, 1H), 8.45 (d, J=5.20 Hz, 1H), 8.33-8.31 (m, 2H), 7.79-7.77 (m, 2H), 7.59-7.56 (m, 2H), 7.49-7.47 (m, 2H), 7.39-7.37 (m, 2H), 7.24-7.21 (m, 1H), 5.23 (s, 2H), 5.03 (t, J=2.40 Hz, 1H), 3.52-3.51 (m, 1H), 2.20-2.10 (m, 2H), 1.78-1.68 (m, 4H).

### Step 3: Synthesis of 4-(((((1R,2R)-2-(pyridin-2-yldisulfaneyl)cyclopentyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (MMAE) (150 mg, 0.209 mmol) in DMF (1 ml) were added (1R,2R)-2-(pyridin-2-yldisulfaneyl)cyclopentyl (4-((((4-nitrophenoxy)carbonyl)oxy)methyl)phenyl)carbamate (113 mg, 0.209 mmol), 1 M solution of 1-hydroxy-7-azabenzotriazole in DMA (0.104 ml, 0.104 mmol) and DIPEA (0.054 ml, 0.313 mmol) at 0° C. The reaction mixture was stirred at RT for 18 h. The reaction mixture was purified by preparative HPLC using 0.1% HCOOH in H₂O and ACN. The product fraction was lyophilized to afford 4-(((((1R,2R)-2-(pyridin-2-yldisulfaneyl)cyclopentyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (150 mg, 0.133 mmol, 63.6% yield) as a white solid. LCMS: [M+H]⁺ calcd for C₅₈H₈₅N₇O₁S₂, 1119.57; found 1121.4 (M+H). HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 13.61; Purity (Max): 99.26%.

### Step 4: Synthesis of Compound 3

To an ice cooled solution of 4-(((((1R,2R)-2-(pyridin-2-yldisulfaneyl)cyclopentyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (60 mg, 0.054 mmol) in DMF (0.5 ml) were added Pv1 peptide (176 mg, 0.054 mmol) and triethylamine (8.96 μl, 0.064 mmol). The reaction mixture was stirred at RT for 18 h. The reaction mixture was purified by preparative HPLC using 0.1% TFA in H₂O and ACN. The product fraction was lyophilized to afford Compound 3 (65 mg, 0.015 mmol, 27.8% yield) as a white solid. The product obtained is di-TFA salt. LCMS: [M+H]⁺ calcd for C₂₀₅H₃₀₆N₄₂O₅₄S₂, 4284.19; found 1430.2 (M+3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 12.20; Purity (Max): 99.84%.

### Example 4: Synthesis of Compound 4

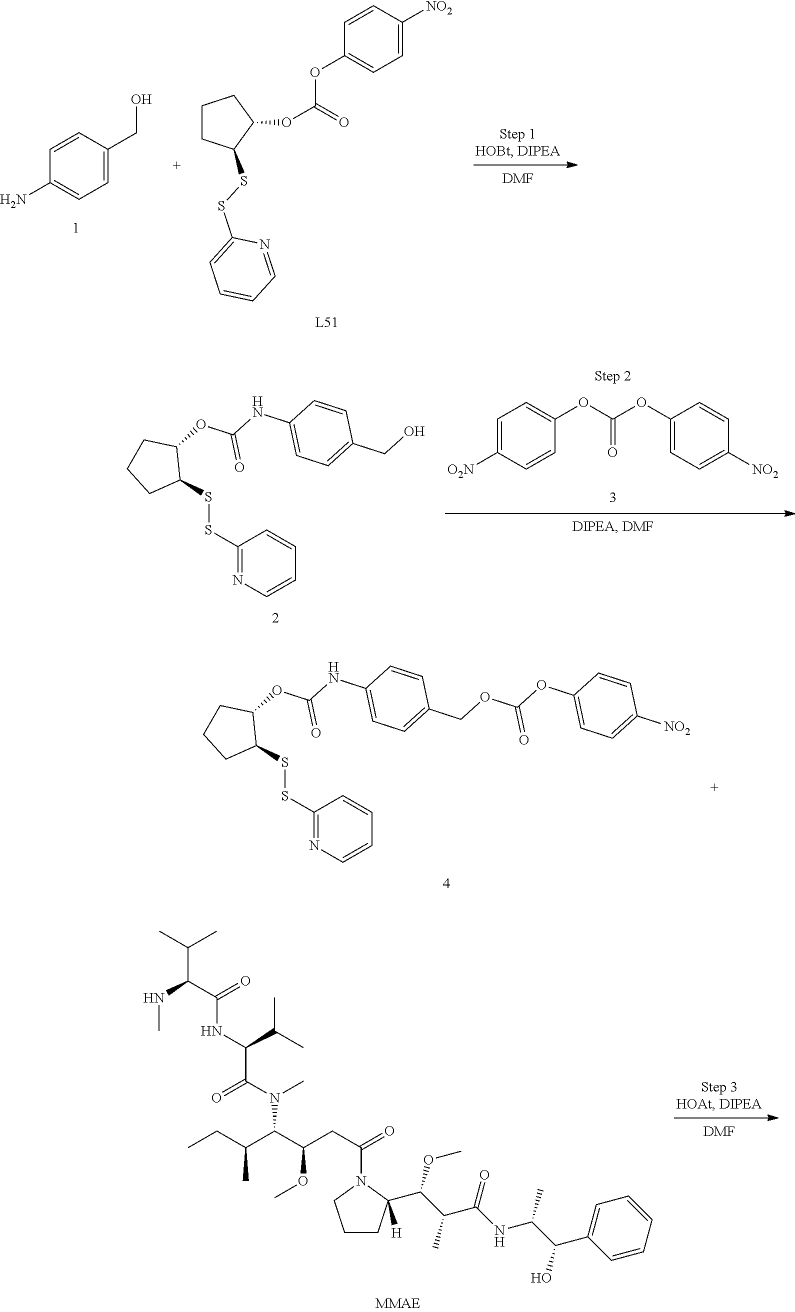

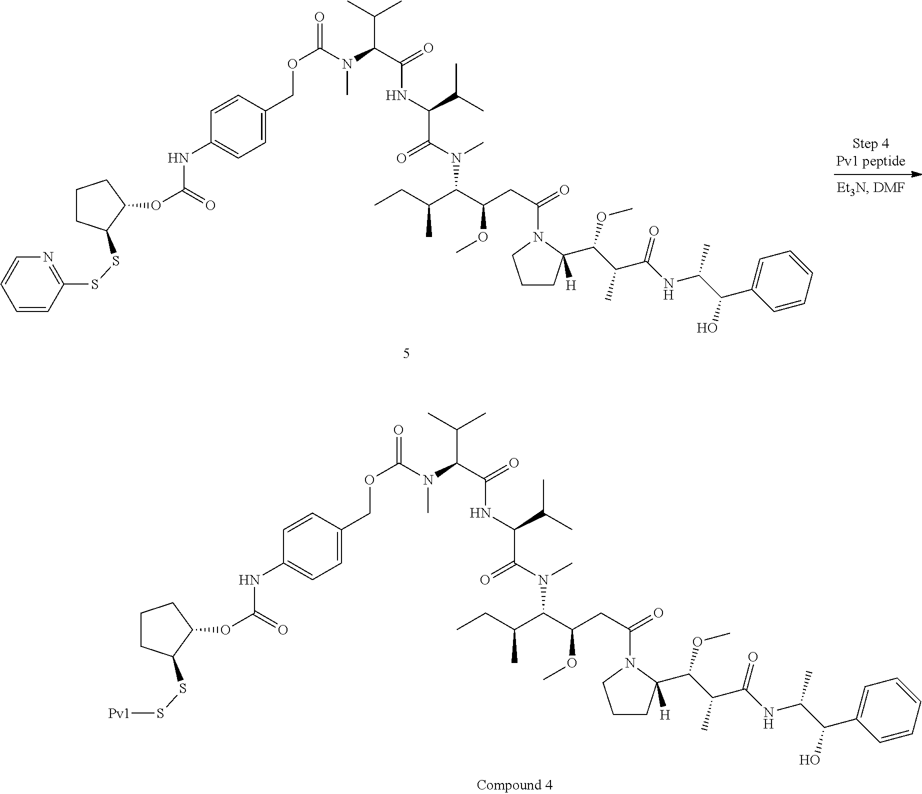

### Step 1: Synthesis of (1S,2S)-2-(pyridin-2-yldisulfaneyl) cyclopentyl (4-(hydroxymethyl)phenyl) carbamate

A stirred solution of (1S,2S)-2-(pyridin-2-yldisulfaneyl)cyclopentyl 2-(4-nitrophenyl)acetate (L-51) (380 mg, 0.974 mmol) and (4-aminophenyl)methanol (120 mg, 0.974 mmol) in DMF (2.5 ml) was cooled to 0° C. Then, DIPEA (0.339 ml, 1.949 mmol) was added followed by HOBt (74.6 mg, 0.487 mmol) at 0° C. The reaction mixture was stirred at RT for 18 h. Ice-cold water was added to the reaction mixture and extracted with ethyl acetate. The ethyl acetate layer was dried over Na₂SO₄ and concentrated under reduced pressure to get the crude product. The crude product was purified by flash column chromatography using 50% EtOAc in petroleum ether as eluent. The product fractions were evaporated under reduced pressure to afford (1S,2S)-2-(pyridin-2-yldisulfaneyl) cyclopentyl (4-(hydroxymethyl) phenyl) carbamate (304 mg, 0.798 mmol, 82% yield) as brown gum. LCMS: [M+H]⁺ calcd for C₁₅H₂₀F₆N₂O₃S₂, 376.09; found 377.1 (M+H). ¹H NMR (400 MHz, DMSO-d₆): δ 9.61 (s, 1H), 8.45 (d, J=4.80 Hz, 1H), 7.79-7.77 (m, 2H), 7.39-7.22 (m, 2H), 7.19-7.14 (m, 3H), 5.08 (t, J=5.60 Hz, 1H), 5.00 (s, 1H), 4.41 (d, J=5.60 Hz, 2H), 3.51-3.34 (m, 1H), 2.17-2.00 (m, 2H), 1.78-1.67 (m, 4H).

### Step 2: Synthesis of (1S,2S)-2-(pyridin-2-yldisulfaneyl) cyclopentyl (4-((((4-nitrophenoxy) carbonyl) oxy) methyl) phenyl) carbamate

To a solution of (1S,2S)-2-(pyridin-2-yldisulfaneyl)cyclopentyl (4-(hydroxymethyl) phenyl) carbamate (300 mg, 0.797 mmol) and bis(4-nitrophenyl) carbonate (970 mg, 3.19 mmol) in DMF (5 ml) was added DIPEA (0.208 ml, 1.195 mmol) at 0° C. and the reaction mixture was stirred at RT for 18 h. Ice-cold water was added to the reaction mixture and extracted with ethyl acetate. The ethyl acetate layer was washed with cold water, brine, dried over Na₂SO₄ and concentrated under reduced pressure to obtain the crude product. The crude product was purified by flash column chromatography using 25% ethyl acetate in petroleum ether as eluent. The product fractions were concentrated under reduced pressure to afford (1S,2S)-2-(pyridin-2-yldisulfaneyl)cyclopentyl (4-((((4-nitrophenoxy) carbonyl)oxy)methyl)phenyl)carbamate (330 mg, 0.599 mmol, 75% yield) as yellow gummy solid. LCMS: [M+H]⁺ calcd for C₂₅H₂₃N₃O₇S₂, 541.10; found 542.0 (M+H). ¹H NMR (400 MHz, DMSO-d₆): δ 9.78 (s, 1H), 8.45 (d, J=5.20 Hz, 1H), 8.31-8.33 (m, 2H), 7.81-7.77 (m, 1H), 7.57 (d, J=8.80 Hz, 2H), 7.48 (d, J=8.40 Hz, 2H), 7.38 (d, J=8.40 Hz, 2H), 7.24-7.21 (m, 1H), 5.23 (s, 2H), 5.03 (t, J=2.40 Hz, 1H), 3.54-3.49 (m, 1H), 2.20-2.10 (m, 2H), 1.80-1.67 (m, 4H).

### Step 3: Synthesis of 4-(((((1S,2S)-2-(pyridin-2-yldisulfaneyl)cyclopentyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (150 mg, 0.209 mmol) and (1S,2S)-2-(pyridin-2-yldisulfaneyl)cyclopentyl (4-((((4-nitrophenoxy)carbonyl) oxy)methyl)phenyl)carbamate (113 mg, 0.209 mmol) in DMF (1 ml) were added a 1 M solution of 1-hydroxy-7-azabenzotriazole in DMA (0.104 ml, 0.104 mmol) and DIPEA (0.055 ml, 0.313 mmol) at 0° C. The reaction mixture was stirred at RT for 18 h. The reaction mixture was purified by preparative HPLC using 0.1% HCOOH in H₂O and ACN. The product fraction was lyophilized to afford 4-(((((1S,2S)-2-(pyridin-2-yldisulfaneyl)cyclopentyl) oxy)carbonyl)amino) benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (150 mg, 0.129 mmol, 61.9% yield) as white solid. LCMS: [M+H]⁺ calcd for C₅₈H₈₅N₇O₁₁S₂, 1119.57; found 1120.6 (M+H). HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 13.60; Purity (Max): 96.55%.

### Step 4: Synthesis of Compound 4

To a stirred solution of 4-(((((1S,2S)-2-(pyridin-2-yldisulfaneyl)cyclopentyl) oxy)carbonyl)amino) benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (70 mg, 0.062 mmol) and Pv1 peptide (203.2 mg, 0.062 mmol) in DMF (0.75 ml) was added triethylamine (10.45 μl, 0.074 mmol) at 0° C. The reaction mixture was stirred at RT for 26 h. The reaction mixture was purified by preparative HPLC using 0.1% TFA in H₂O and ACN. The product fraction was lyophilized to afford Compound 4 (41 mg, 9.56 μmol, 15.4% yield) as white solid. The product obtained is a di-TFA salt. LCMS: [M+H]⁺ calcd for C₂₀₅H₃₀₆N₄₂O₅₄S₂, 4284.19; found 1430.1 (M+3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 12.33; Purity (Max): 99.61%.

### Example 5: Synthesis of Compound 5

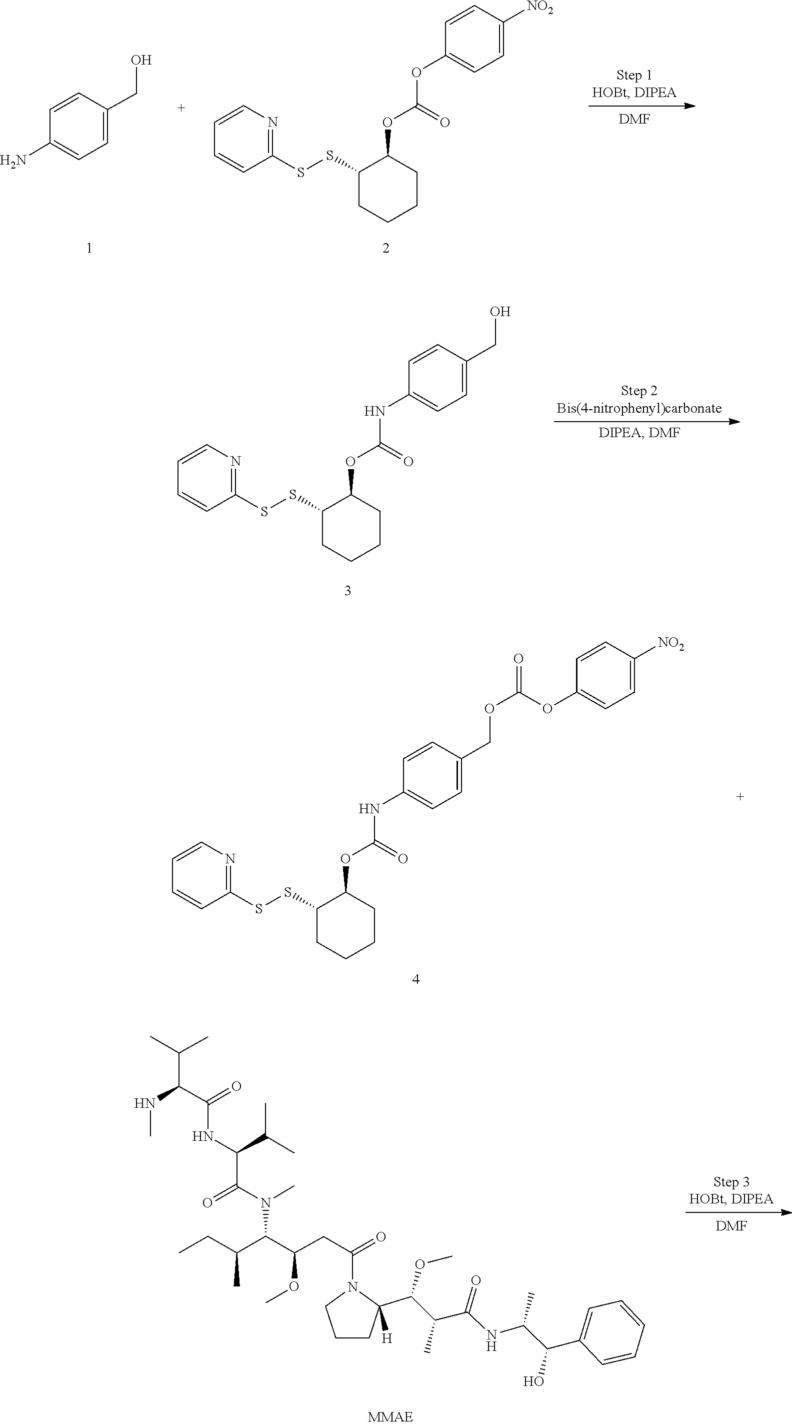

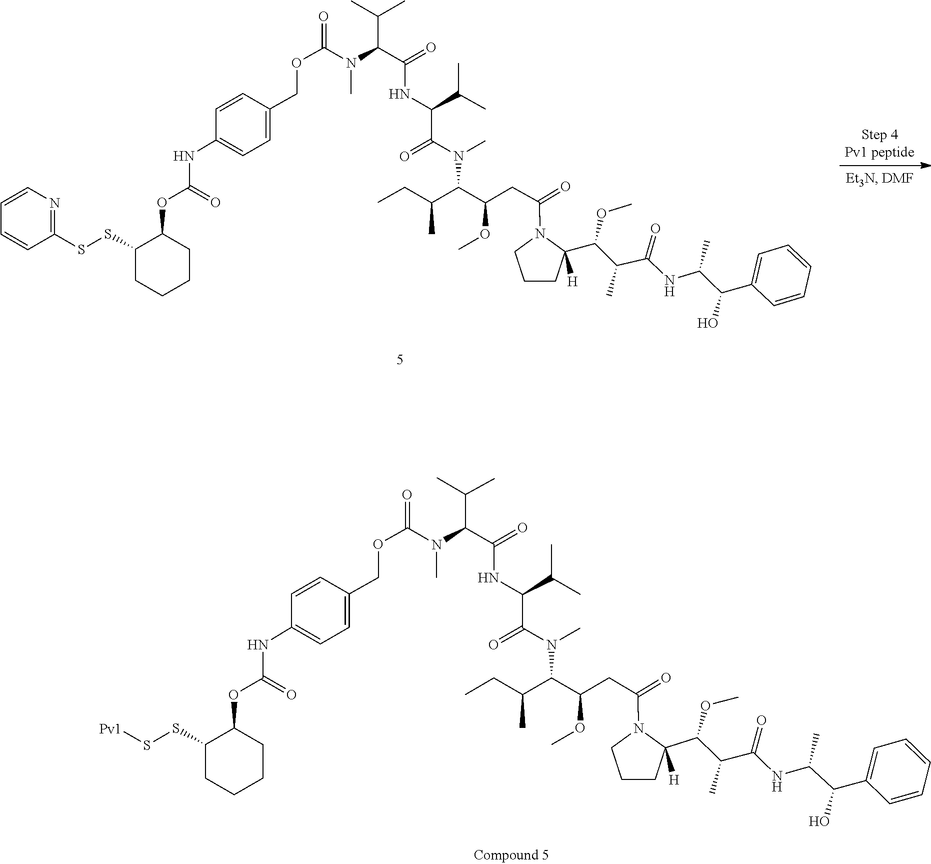

### Step 1: Synthesis of (1S,2S)-2-(pyridin-2-yldisulfaneyl) cyclohexyl (4-(hydroxymethyl)phenyl) carbamate

A stirred solution of (4-aminophenyl) methanol (20 mg, 0.162 mmol) and 4-nitrophenyl ((1S,2S)-2-(pyridin-2-yldisulfaneyl) cyclohexyl) carbonate (79 mg, 0.195 mmol) in DMF (1 ml) was cooled with ice. DIPEA (0.057 ml, 0.325 mmol) and 1H-benzo[d] [1,2,3] triazol-1-ol (10.97 mg, 0.081 mmol) were added and the reaction mixture was stirred at RT for 36 h. The reaction mixture was diluted with water and extracted with ethyl acetate. The organic layer was washed with brine, dried over Na₂SO₄ and concentrated to get a crude residue. The crude residue was purified by flash column chromatography using 75-80% ethyl acetate in petroleum ether as eluent. The product fraction was evaporated to afford (1S,2S)-2-(pyridin-2-yldisulfaneyl) cyclohexyl (4-(hydroxymethyl) phenyl) carbamate (40 mg, 0.090 mmol, 55.4% yield) was obtained as a gummy solid. LCMS: [M+H]⁺ calcd for C₁₉H₂₂N₂O₃S₂, 390.11; found 391.1 (M+H). HPLC: Column: X-Bridge C8 (50×4.6) mm, 3.5 μm; Mobile phase: A: 0.1% TFA in H₂O; Mobile phase: B: 0.1% TFA in ACN; Flow: 2.0 mL/min; RT (min): 3.80; Purity (Max): 87.78%.

### Step 2: Synthesis of (1S,2S)-2-(pyridin-2-yldisulfaneyl) cyclohexyl (4-((((4-nitrophenoxy) carbonyl) oxy) methyl) phenyl) carbamate

To a stirred solution of (1S,2S)-2-(pyridin-2-yldisulfaneyl)cyclohexyl (4-(hydroxymethyl)phenyl)carbamate (20 mg, 0.051 mmol) in DMF (1 ml) were added bis(4-nitrophenyl) carbonate (62.3 mg, 0.205 mmol) and DIPEA (0.013 ml, 0.077 mmol) at 0° C. The reaction mixture was stirred at RT for 18 h. To the reaction mixture, ice cold water was added and extracted with ethyl acetate. The ethyl acetate layer was dried over Na₂SO₄ concentrated under reduced pressure to obtain the crude product. The crude product was purified by flash column chromatography using 15% ethyl acetate and petroleum ether as eluent. The product fraction was concentrated under reduced pressure to afford (1S,2S)-2-(pyridin-2-yldisulfaneyl)cyclohexyl (4-((((4-nitrophenoxy)carbonyl) oxy)methyl)phenyl)carbamate (12 mg, 0.021 mmol, 40.3% yield) as a gummy solid. LCMS: [M+H]⁺ calcd for C₂₆H₂₅N₃O₇S₂, 555.11; found 555.9 (M+H).

### Step 3: Synthesis of (4-(((((1S,2S)-2-(pyridin-2-yldisulfaneyl)cyclohexyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

A solution of (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (15.51 mg, 0.022 mmol) and (1S,2S)-2-(pyridin-2-yldisulfaneyl)cyclohexyl (4-((((4-nitrophenoxy)carbonyl) oxy)methyl)phenyl) carbamate (12 mg, 0.022 mmol) in DMF (0.8 ml) was cooled with ice. To this DIPEA (5.66 μl, 0.032 mmol) and a 1 M solution of 1-hydroxy-7-azabenzotriazole in DMA (10.80 μl, 10.80 μmol) was added and the reaction mixture stirred at RT for 16 h. The reaction mixture was purified by preparative HPLC using 0.1% HCOOH in H₂O and ACN. The product fraction was lyophilized to afford 4-(((((1S,2S)-2-(pyridin-2-yldisulfaneyl)cyclohexyl)oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (15 mg, 0.013 mmol, 60.5% yield) as a white solid. LCMS: [M+H]⁺ calcd for C₅₉H₈₇N₇O₁₁S₂, 1133.59; found 1135.1 (M+H). HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 15.62; Purity (Max): 98.83%.

### Step 4: Synthesis of Compound 5

A solution of 4-(((((1R,2R)-2-(pyridin-2-yldisulfaneyl)cyclohexyl) oxy)carbonyl)amino) benzyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (15 mg, 0.013 mmol) and Pv1 peptide (47.7 mg, 0.015 mmol) in DMF (0.5 ml) was cooled with ice. To this triethylamine (2.211 μl, 0.016 mmol) was added and the reaction mixture was stirred at RT for 4 h. The reaction mixture was purified by preparative HPLC using 0.1% TFA in H₂O and ACN. The product fraction was lyophilized to afford Compound 5 (42 mg, 9.58 μmol, 72.5% yield) as a white solid. The product obtained is a di-TFA salt. LCMS: [M+H]⁺ calcd for C₂₀₆H₃₀₈N₄₂O₅₄S₂, 4298.21; found 1435.0 (M+3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 12.93; Purity (Max): 98.12%.

### Example 6: Synthesis of Compound 6

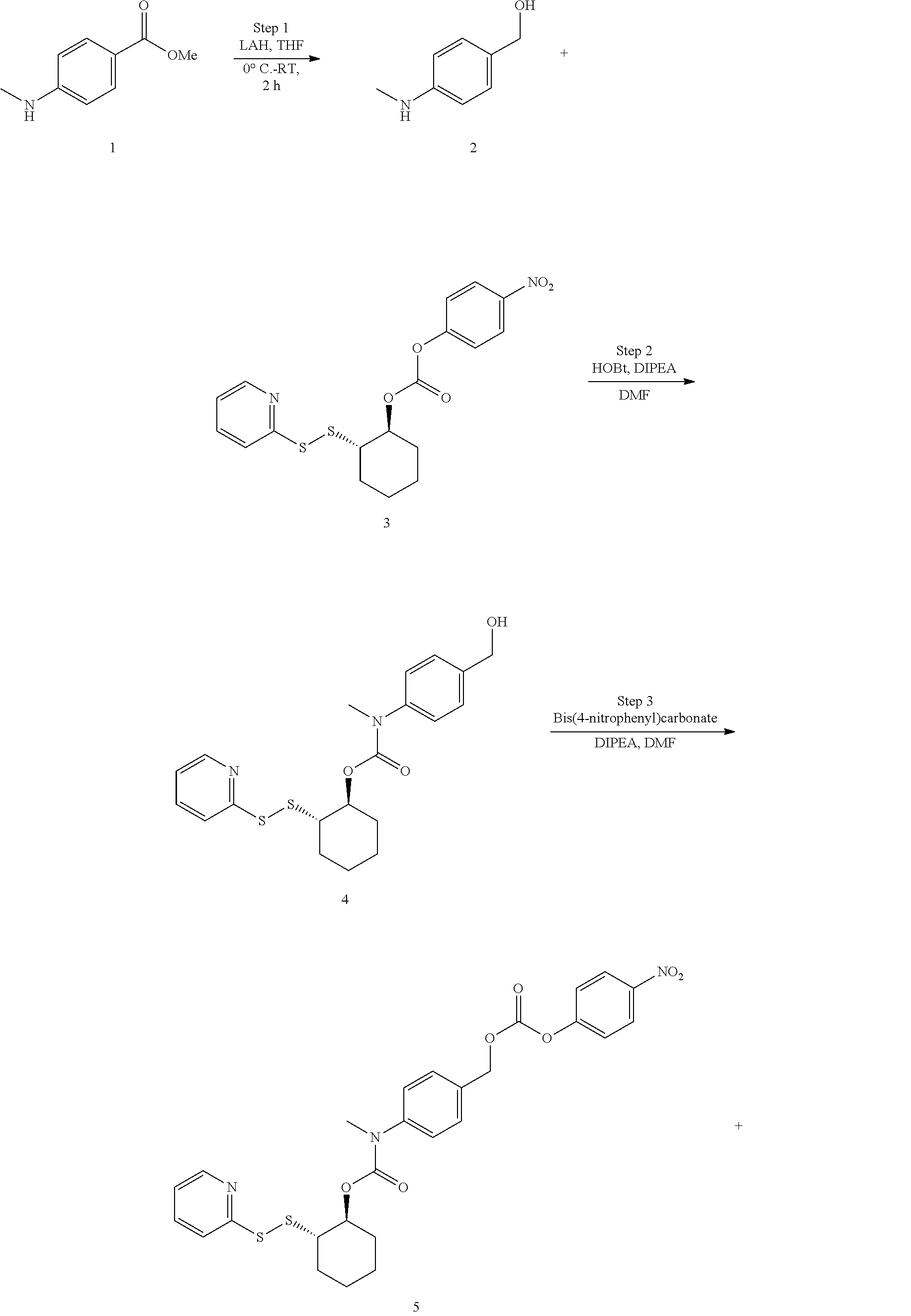

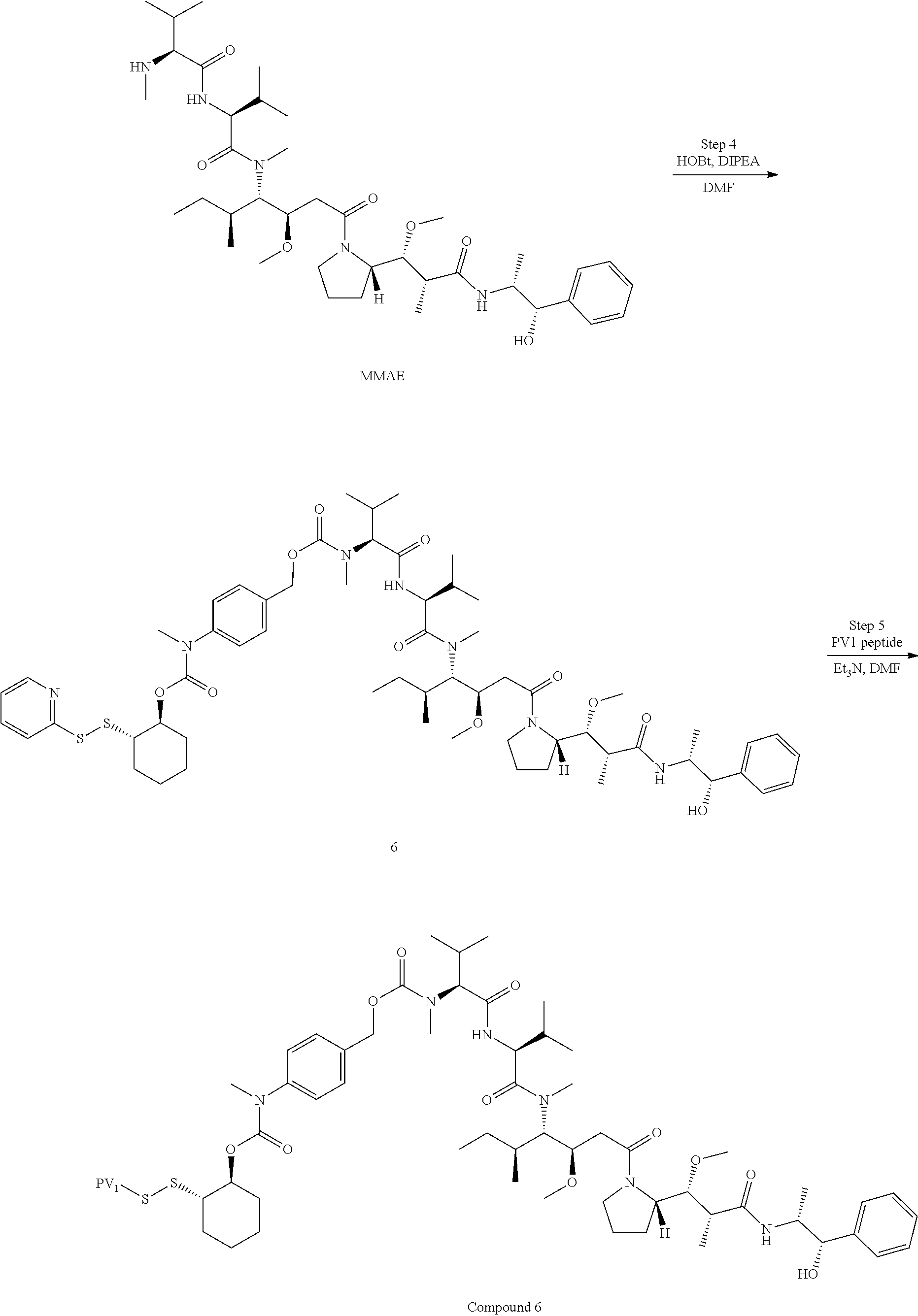

### Step 1: Synthesis of (4-(methylamino)phenyl) methanol

To a stirred solution of methyl 4-(methylamino) benzoate (0.2 g, 1.211 mmol) in THE (2 ml) was added LAH 2M solution in THE (0.726 ml, 1.453 mmol) at 0° C. The reaction mixture was stirred at RT for 2 h. The reaction mixture was quenched with saturated NH₄Cl solution. The ethyl acetate layer was separated, concentrated and purified by flash column chromatography using 20% ethyl acetate in petroleum ether. The product fraction was evaporated to afford (4-(methylamino) phenyl) methanol (150 mg, 0.847 mmol, 70.0% yield) as a yellow liquid. LCMS: [M+H]⁺ calcd for C₈H₁₁NO, 137.08; found 138.2 (M+H). ¹H NMR (400 MHz, DMSO-d₆): δ 7.03 (d, J=8.40 Hz, 2H), 6.50-6.50 (m, 2H), 5.49-5.48 (m, 1H), 4.33 (t, J=5.60 Hz, 1H), 4.04 (d, J=6.80 Hz, 2H), 2.66 (s, 3H).

### Step 2: Synthesis of (1S,2S)-2-(pyridin-2-yldisulfaneyl) cyclohexyl (4-(hydroxymethyl)phenyl) (methyl)carbamate

To a stirred solution of (4-(methylamino)phenyl)methanol (30 mg, 0.219 mmol) in DMF (2 ml) were added 4-nitrophenyl ((1S,2S)-2-(pyridin-2-yldisulfaneyl)cyclohexyl) carbonate (107 mg, 0.262 mmol), DIPEA (0.076 ml, 0.437 mmol) and 1H-benzo[d][1,2,3]triazol-1-ol (14.78 mg, 0.109 mmol) at 0° C. The reaction mixture was stirred at 80° C. for 18 h. The reaction mixture was diluted with water and extracted with ethyl acetate. The organic layer was dried over Na₂SO₄ and concentrated to get a crude residue. The crude residue was purified by flash column chromatography. The product was eluted with 35% ethyl acetate in petroleum ether. The product fraction was evaporated to afford (1S,2S)-2-(pyridin-2-yldisulfaneyl) cyclohexyl (4-(hydroxymethyl) phenyl) (methyl)carbamate (40 mg, 0.057 mmol, 26.1% yield) as a yellow liquid. LCMS: [M+H]⁺ calcd for C₂₀H₂₄N₂O₃S₂, 404.12; found 405.1 (M+H).

### Step 3: Synthesis of (1S,2S)-2-(pyridin-2-yldisulfaneyl) cyclohexyl methyl(4-((((4-nitrophenoxy) carbonyl) oxy) methyl) phenyl) carbamate

To a stirred solution of (1S,2S)-2-(pyridin-2-yldisulfaneyl)cyclohexyl (4-(hydroxymethyl) phenyl)(methyl)carbamate (40 mg, 0.099 mmol) in DMF (1 ml) were added bis(4-nitrophenyl) carbonate (120 mg, 0.396 mmol) and DIPEA (0.035 ml, 0.198 mmol) at 0° C. The reaction mixture was stirred at RT for 6 h. The reaction mixture was diluted with ice cold water and extracted with ethyl acetate. The organic layer was washed with brine, dried over Na₂SO₄ and concentrated under reduced pressure to get a crude residue. The crude residue was purified by flash column chromatography. The product was eluted with 20% ethyl acetate in petroleum ether. The product fraction was evaporated to afford (1S,2S)-2-(pyridin-2-yldisulfaneyl)cyclohexyl methyl(4-((((4-nitrophenoxy)carbonyl)oxy)methyl)phenyl)carbamate (25 mg, 0.044 mmol, 44.3% yield) as colorless gummy solid. LCMS: [M+H]⁺ calcd for C₂₇H₂₇N₃O₇S₂, 569.13; found 570.1 (M+H).

### Step 4: Synthesis of 4-(methyl((((1S,2S)-2-(pyridin-2-yldisulfaneyl) cyclohexyl) oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (25 mg, 0.035 mmol) and (1S,2S)-2-(pyridin-2-yldisulfaneyl)cyclohexyl methyl(4-((((4-nitrophenoxy)carbonyl) oxy)methyl)phenyl)carbamate (19.83 mg, 0.035 mmol) in DMF (1 ml) were added DIPEA (9.12 μl, 0.052 mmol) and a 1 M solution of 1-hydroxy-7-azabenzotriazole in DMA (0.017 ml, 0.017 mmol) at 0° C. The reaction mixture was stirred at RT for 18 h. The crude reaction mixture was purified by preparative HPLC using 0.1% HCOOH in H₂O and ACN. The product fraction was lyophilized to afford 4-(methyl((((1S,2S)-2-(pyridin-2-yldisulfaneyl) cyclohexyl) oxy)carbonyl)amino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (33 mg, 0.026 mmol, 74.8% yield) as a white solid. LCMS: [M+H]⁺ calcd for C₆₀H₈₉N₇O₁₁S₂, 1147.61; found 1149.6 (M+H).

### Step 5: Synthesis of Compound 6

A solution of (1R,2R)-2-(pyridin-2-yldisulfaneyl)cyclohexyl (4-((5S,8S,11S,12R)-11-((S)-sec-butyl)-12-(2-((R)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)(methyl)carbamate (23 mg, 0.020 mmol) and PV1 peptide (72.2 mg, 0.022 mmol) in DMF (1 ml) was cooled with ice. To this triethylamine (2.432 mg, 0.024 mmol) was added. The reaction mixture was stirred at RT for 4 h. The reaction mixture was purified by preparative HPLC using 0.1% TFA in H₂O and ACN. The product fraction lyophilized to afford Compound 6 (45 mg, 10.03 μmol, 50.1% yield) as a white solid. The product obtained is a di-TFA salt. LCMS: [M+H]⁺ calcd for C₂₀₇H₃₁₀N₄₂O₅₄S₂, 4312.22; found 1437.7 (M−3)/3. HPLC: Column: Atlantis dC18 (250×4.6) mm, 5 μm; Mobile phase: A: 0.1% TFA in H₂O; Mobile phase: B: 0.1% TFA in ACN; Flow: 1.0 mL/min; RT (min): 12.66; Purity (Max): 96.18%.

The following compounds of Table 2 were prepared using the procedures described in the examples above.

TABLE 2

| Example | Structure |
|---|---|
| 7 | 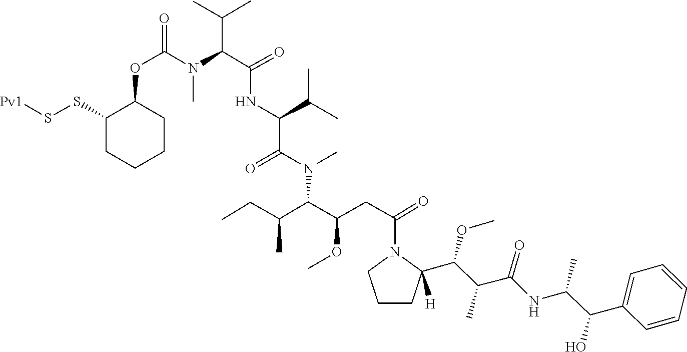 |
| 8 | 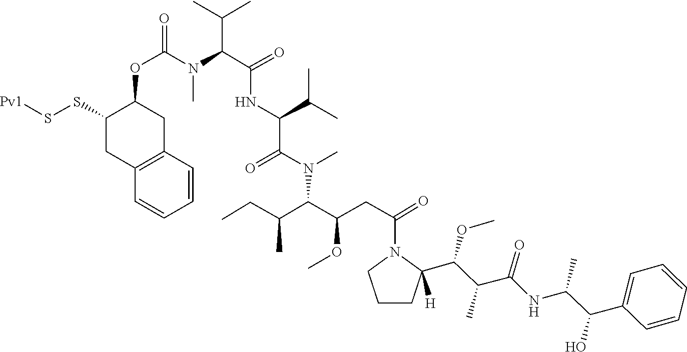 |
| 9 | 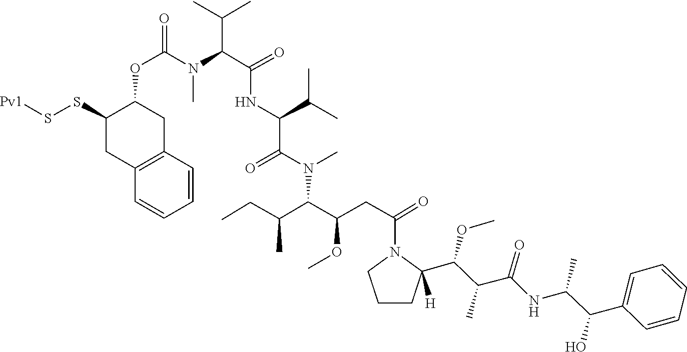 |
| 10 | 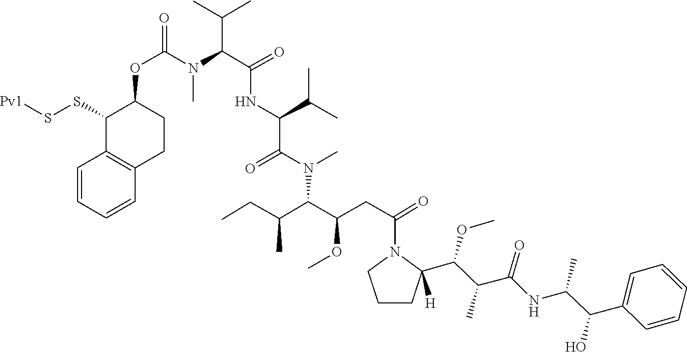 |
| 11 | 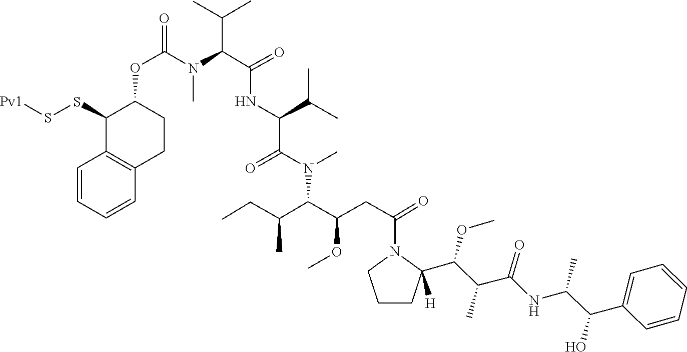 |
| 12 | 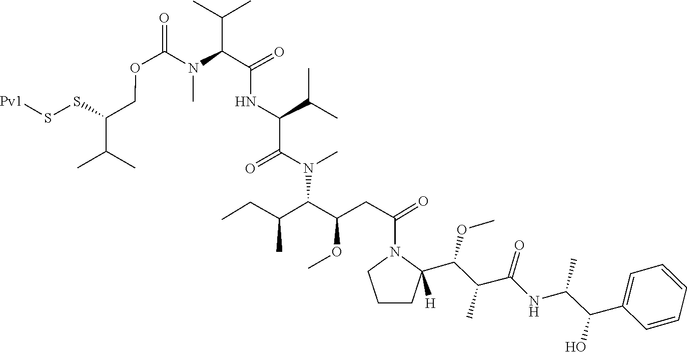 |
| 13 | 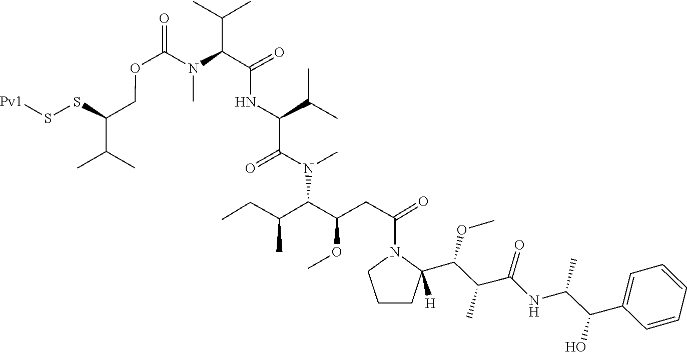 |
| 14 | 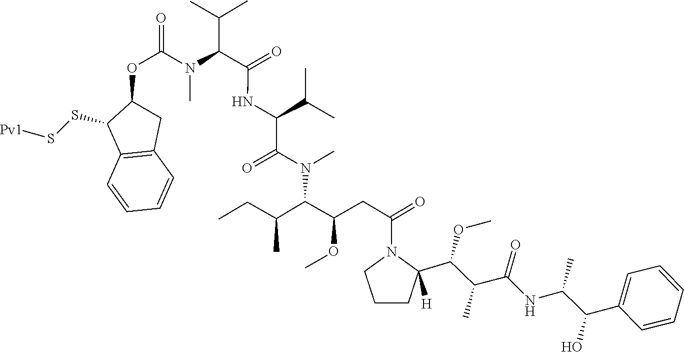 |
| 15 | 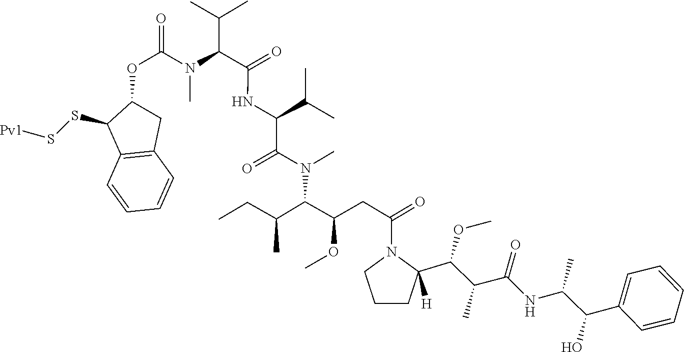 |
| 16 | 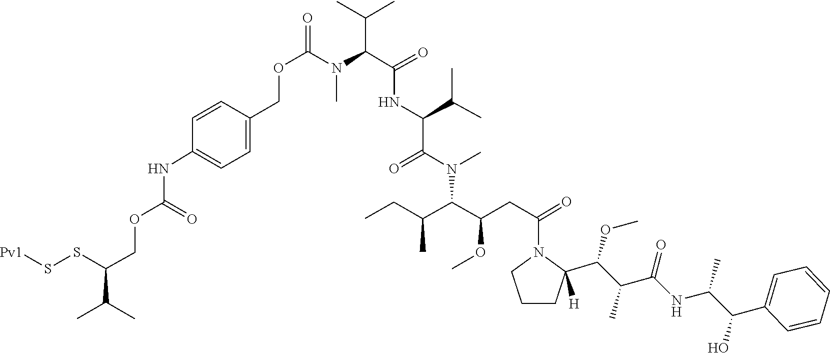 |
| 17 | 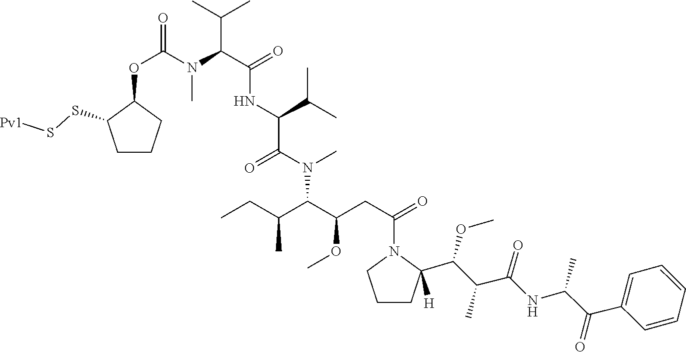 |
| 18 | 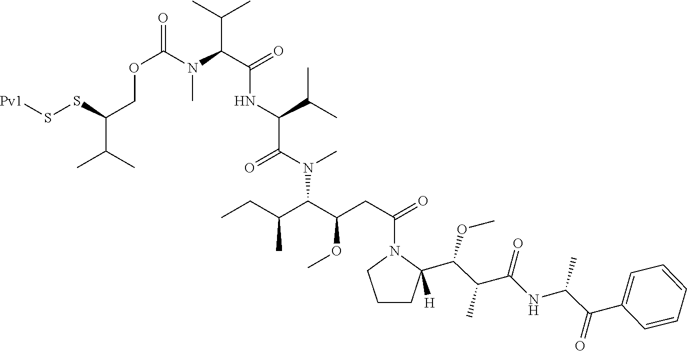 |
| 19 | 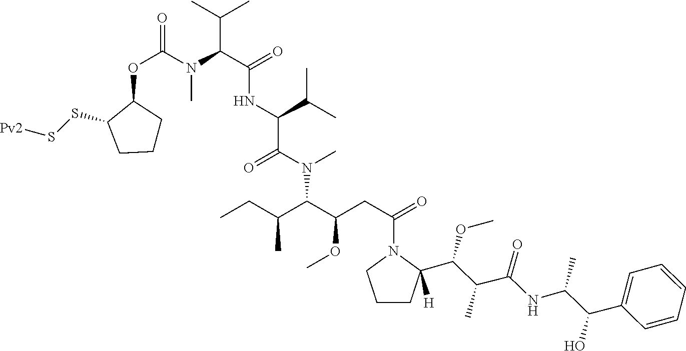 |
| 20 | 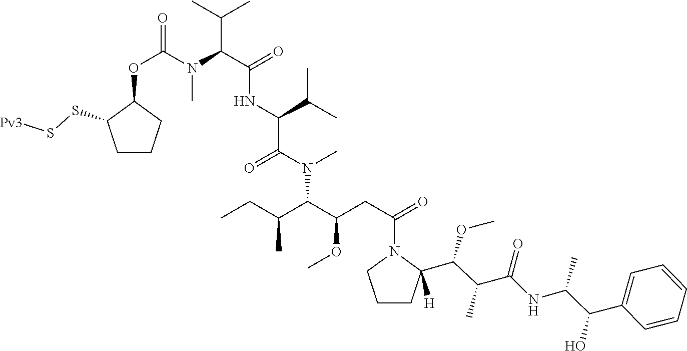 |

### Example 21: Synthesis of Compound 21

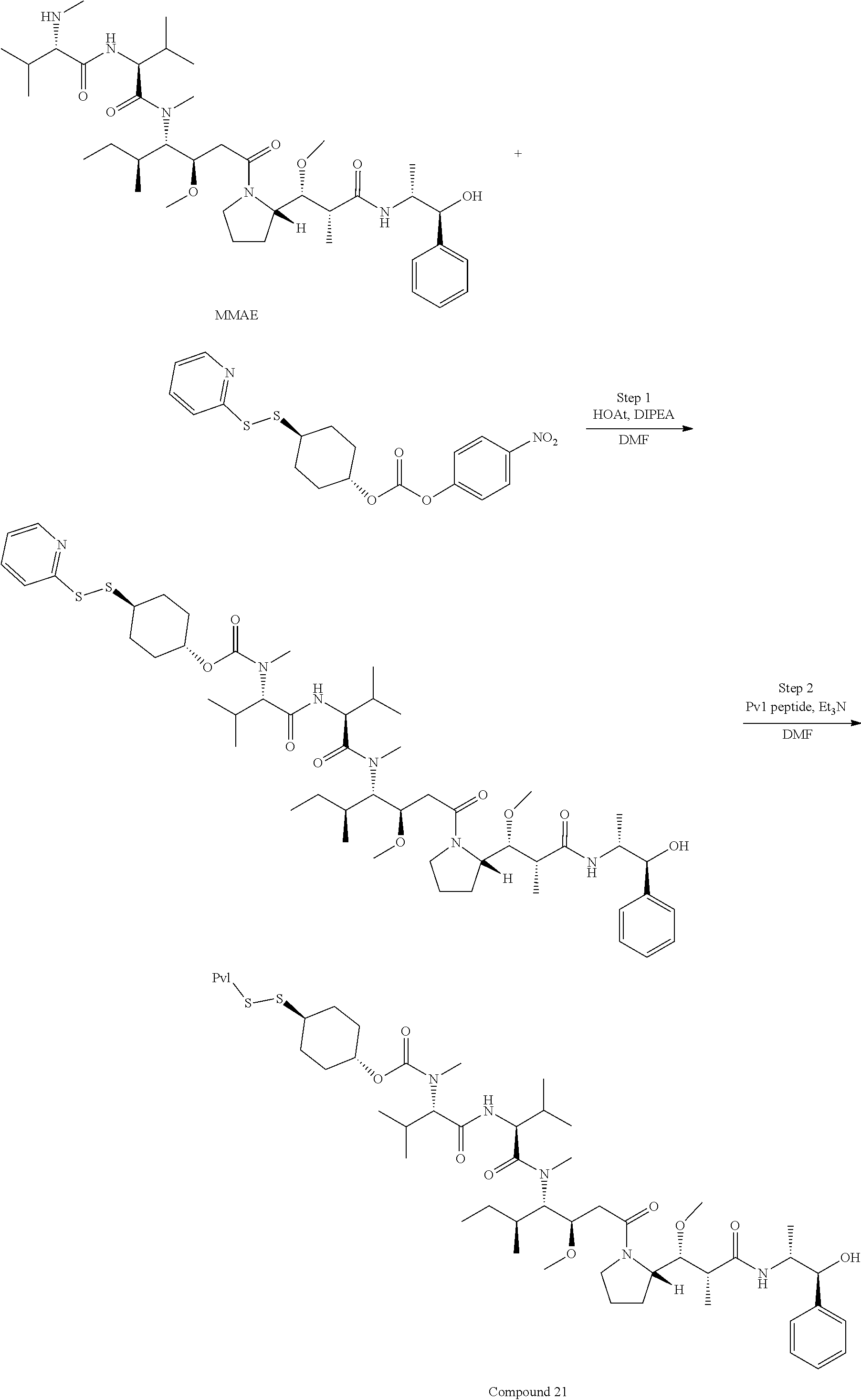

### Step 1: Synthesis of trans-4-(pyridin-2-yldisulfaneyl)cyclohexyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (20 mg, 0.028 mmol) and 4-nitrophenyl (trans-4-(pyridin-2-yldisulfaneyl)cyclohexyl) carbonate (11.32 mg, 0.028 mmol) in DMF (0.5 ml) was added DIPEA (7.3 μL, 0.042 mmol) followed by 1-hydroxy-7-azabenzotriazole in DMA (13.92 μl, 13.92 μmol) at 0° C. The reaction mixture was stirred at RT for 18 h. The reaction mixture was purified by preparative HPLC using 0.1% HCOOH in H₂O and ACN. The preparative fraction was lyophilized to afford: trans-4-(pyridin-2-yldisulfaneyl)cyclohexyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (19 mg, 0.019 mmol, 69.2% yield) as a white solid. LCMS: [M+H]⁺ calcd for C₅₁H₈₀N₆O₉S₂, 985.34; found 985.3.

### Step 2: Synthesis of Compound 21

A solution of trans-4-(pyridin-2-yldisulfaneyl)cyclohexyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (19 mg, 0.019 mmol) in DMF (0.5 ml) was cooled to 0° C. Pv1 peptide (69.54 mg, 0.021 mmol) and triethylamine (3.22 μl, 0.023 mmol) were added and the reaction mixture was stirred at RT for 1 h. The reaction mixture was purified by preparative HPLC using 0.1% TFA in H₂O and ACN. The preparative fraction was lyophilized to afford Compound 21 (80 mg, 0.019 mmol, 99.9% yield) as a white solid. The product obtained was a di-TFA salt. LCMS: [M+H]⁺ calcd for C₁₉₈H₃₀₁N₄₁O₅₂S₂, 4151.941; found 1384.8 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 11.826; Purity (Max): 99.71%.

### Example 22: Synthesis of Compound 22

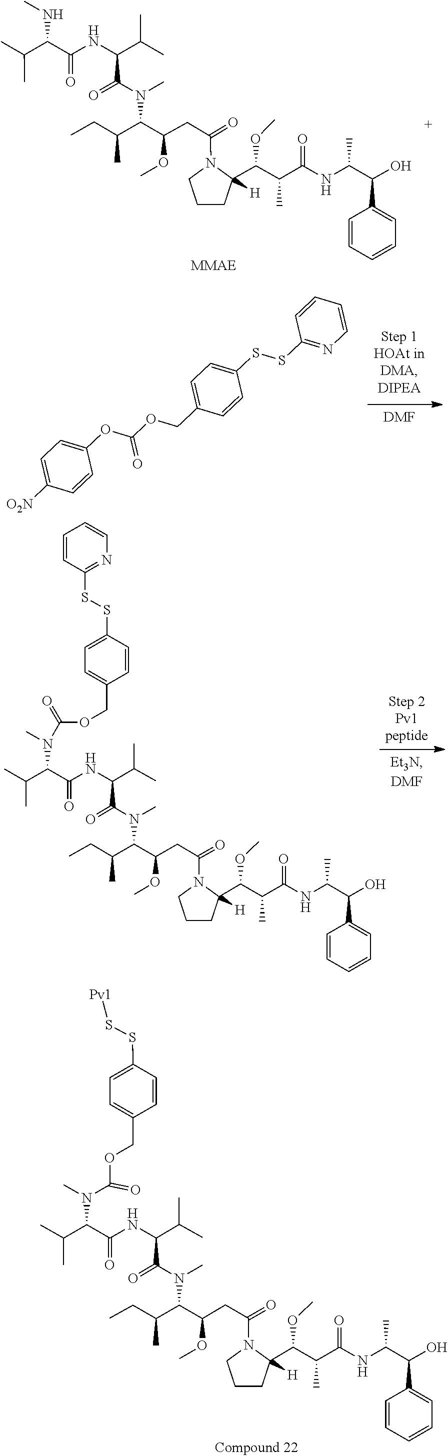

### Step 1: 4-(pyridin-2-yldisulfaneyl)benzyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (50 mg, 0.069 mmol) and 4-nitrophenyl (4-(pyridin-2-yldisulfaneyl)benzyl) carbonate (37.52 mg, 0.090 mmol) in DMF (1 ml) was added DIPEA (24.13 μL, 0.014 mmol) followed by 1-hydroxy-7-azabenzotriazole in DMA (0.47 ml, 0.035 mmol) at 0° C. The reaction mixture was stirred at RT for 18 h. The reaction mixture was purified by preparative HPLC using 0.1% HCOOH in H₂O and ACN. The preparative fraction was lyophilized to afford: 4-(pyridin-2-yldisulfaneyl)benzyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (22 mg, 0.022 mmol, 31.80% yield) as a white solid. LCMS: [M+H]⁺ calcd for C₅₂H₇₆N₆O₉S₂, 993.333; found 994.5.

### Step 2: Synthesis of Compound 22

A solution of 4-(pyridin-2-yldisulfaneyl)benzyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (22 mg, 0.022 mmol) in DMF (1 ml) was cooled to 0° C. Pv1 peptide (80 mg, 0.024 mmol) and triethylamine (12.34 μl, 0.088 mmol) were added and the reaction mixture was stirred at RT for 4 h. The reaction mixture was purified by preparative HPLC using 0.1% TFA in H₂O and ACN. The preparative fraction was lyophilized to afford Compound 22 (40 mg, 0.022 mmol, 43.41% yield) as a white solid. The product obtained is a di-TFA salt. LCMS: [M+H]⁺ calcd for C₁₉₉H₂₉₇N₄₁O₅₂S₂, 4159.920; found 1385.1 [(M−3)/3]; HPLC: Column X-Bridge C8(50×4.6) mm, 3.5μm, Mobile phase: A: 0.1% TFA in water, Mobile phase: B: 0.1% TFA in ACN, Flow: 2.0 mL/min; RT (min): 5.52; Purity (Max): 99.147%.

### Example 23: Synthesis of Compound 23

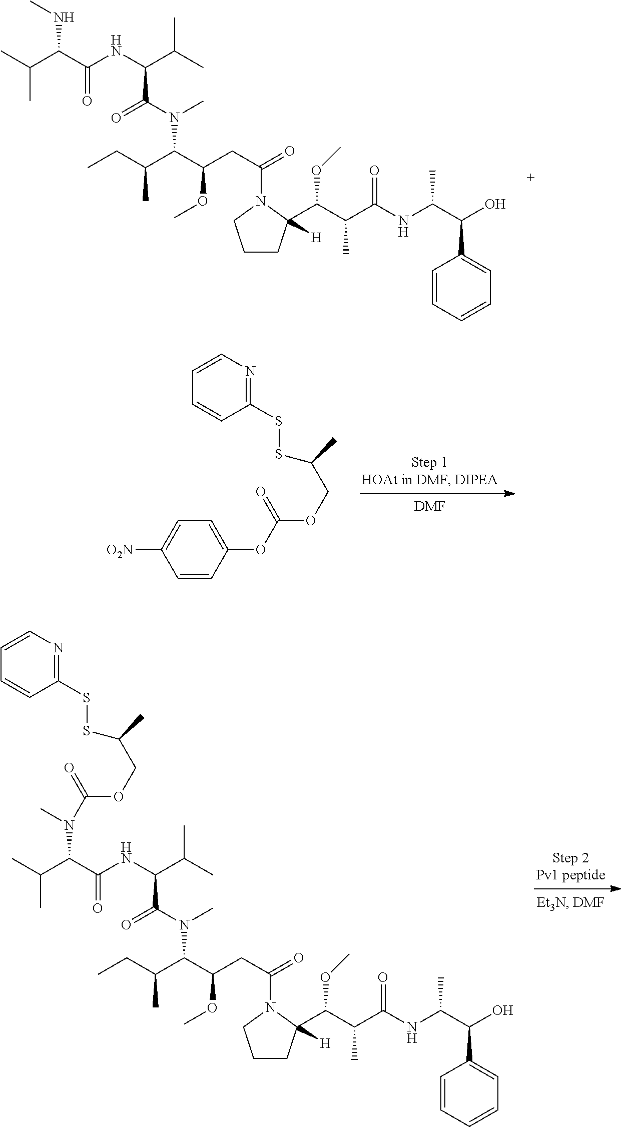

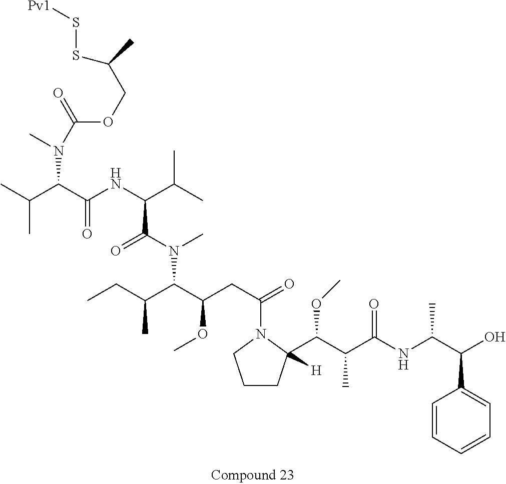

### Step 1: (S)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (50 mg, 0.069 mmol) and (S)-4-nitrophenyl (2-(pyridin-2-yldisulfaneyl)propyl) carbonate (30.61 mg, 0.090 mmol) in DMF (1 ml) was added DIPEA (24.13 μL, 0.014 mmol) followed by 1-hydroxy-7-azabenzotriazole in DMA (0.47 ml, 0.035 mmol) at 0° C. The reaction mixture was stirred at RT for 18 h. The reaction mixture was purified by preparative HPLC using 0.1% HCOOH in H₂O and ACN. The preparative fraction was lyophilized to afford: (S)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (40 mg, 0.041 mmol, 60.77% yield) as a white solid. LCMS: [M+H]⁺ calcd for C₄₈H₇₆N₆O₉S₂, 945.289; found 945.5.

### Step 2: Synthesis of Compound 23

A solution of (S)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (40 mg, 0.042 mmol) in DMF (1 ml) was cooled to 0° C. Pv1 peptide (152 mg, 0.046 mmol) and triethylamine (23.59 μl, 0.169 mmol) were added and the reaction mixture was stirred at RT for 4 h. The reaction mixture was purified by preparative HPLC using 0.1% TFA in H₂O and ACN. The preparative fraction was lyophilized to afford Compound 23 (126.3 mg, 0.030 mmol, 72.59% yield) as a white solid. The product obtained is a di-TFA salt. LCMS: [M+H]⁺ calcd for C₁₉₅H₂₉₇N₄₁O₅₂S₂, 4111.876; found 1371.1 [(M+3)/3]; HPLC: Column X-Bridge C8(50×4.6) mm, 3.5μm, Mobile phase: A: 0.1% TFA in water, Mobile phase: B: 0.1% TFA in ACN, Flow: 2.0 mL/min; RT (min): 5.43; Purity (Max): 98.857%.

### Example 24: Synthesis of Compound 24

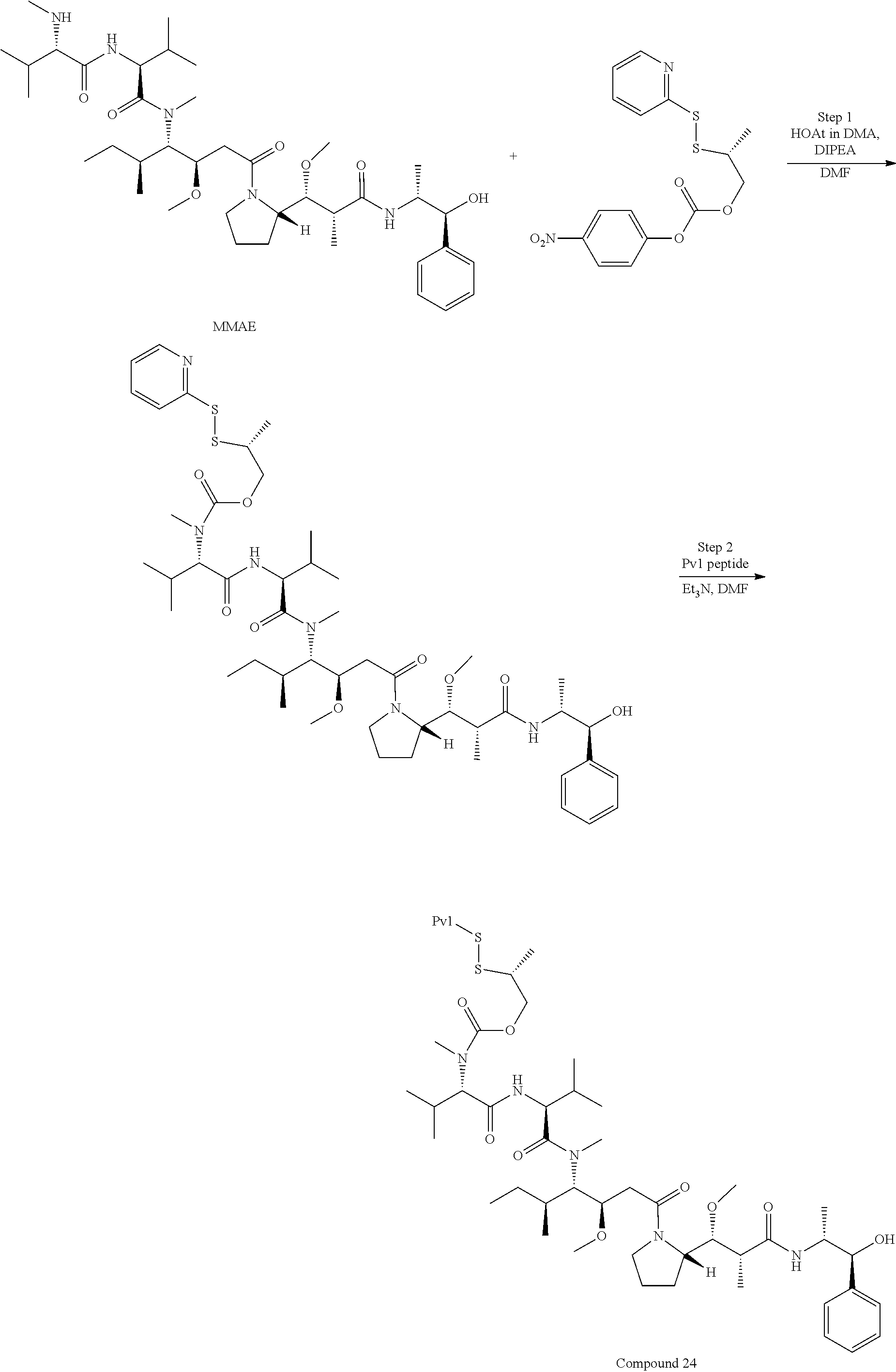

### Step 1: (R)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (50 mg, 0.069 mmol) and (R)-4-nitrophenyl (2-(pyridin-2-yldisulfaneyl)propyl) carbonate (30.619 mg, 0.083 mmol) in DMF (1 ml) was added DIPEA (24.13 μL, 0.014 mmol) followed by 1-hydroxy-7-azabenzotriazole in DMA (0.47 ml, 0.035 mmol) at 0° C. The reaction mixture was stirred at RT for 18 h. The reaction mixture was purified by preparative HPLC using 0.1% HCOOH in H₂O and ACN. The preparative fraction was lyophilized to afford: (R)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (40 mg, 0.042 mmol, 60.77% yield) as a white solid. LCMS: [M+H]⁺ calcd for C₄₈H₇₆N₆O₉S₂, 945.289; found 946.4.

### Step 2: Synthesis of Compound 24

A solution of (R)-2-(pyridin-2-yldisulfaneyl)propyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (40 mg, 0.042 mmol) in DMF (1 ml) was cooled to 0° C. Pv1 peptide (138.73 mg, 0.042 mmol) and triethylamine (11.79 μl, 0.084 mmol) were added and the reaction mixture was stirred at RT for 2 h. The reaction mixture was purified by preparative HPLC using 0.1% TFA in H₂O and ACN. The preparative fraction was lyophilized to afford Compound 24 (40 mg, 0.01 mmol, 22.98% yield) as a white solid. The product obtained is a di-TFA salt. LCMS: [M+H]⁺ calcd for C₁₉₅H₂₉₇N₄₁O₅₂S₂, 4111.876; found 1369.1 [(M−3)/3]; HPLC: Column X-Bridge C8(50×4.6) mm, 3.5 μm, Mobile phase: A: 0.1% TFA in water, Mobile phase: B: 0.1% TFA in ACN, Flow: 2.0 mL/min; RT (min): 5.43; Purity (Max): 98.413%.

### Example 25: Synthesis of Compound 25

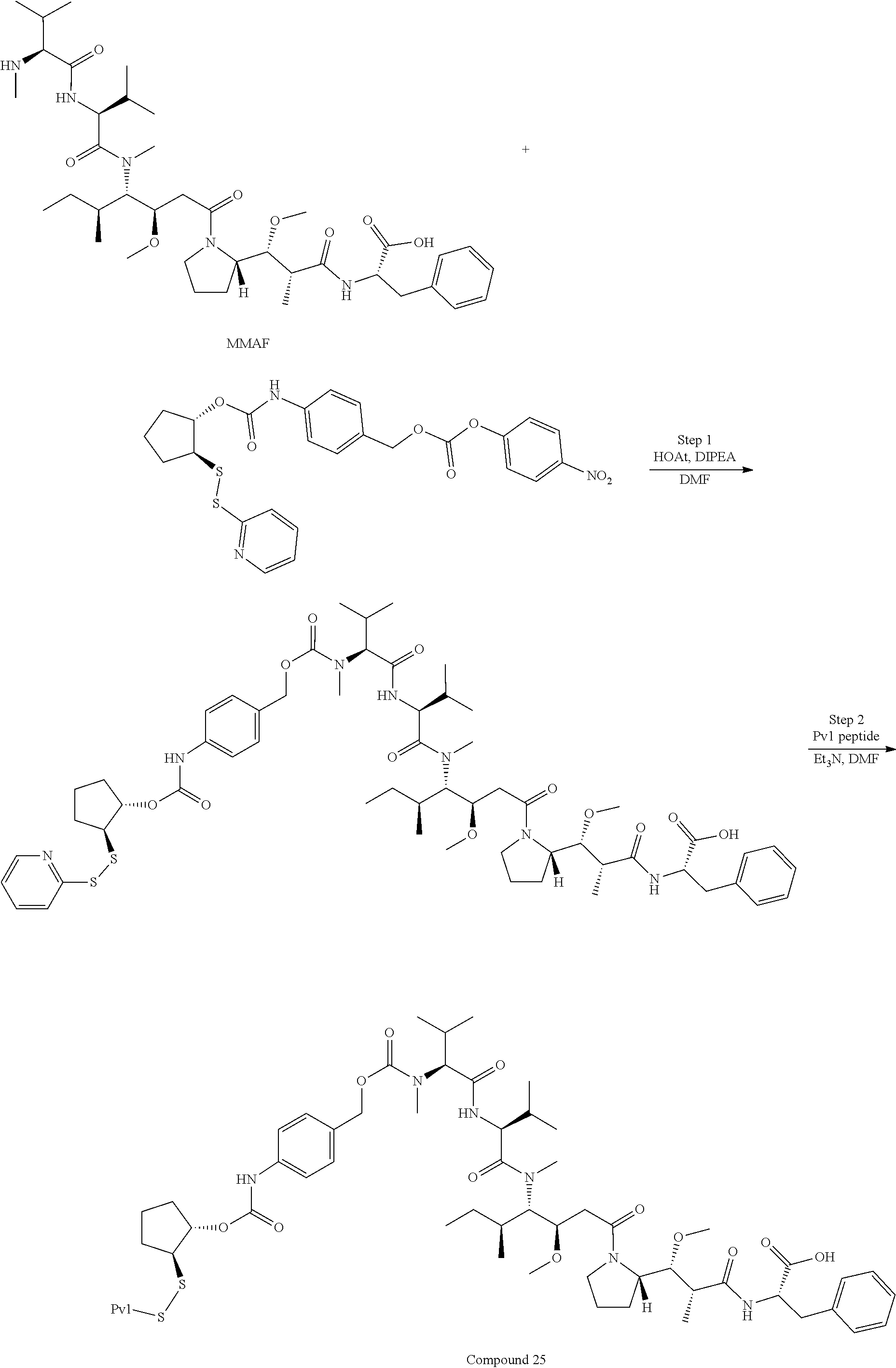

### Step 1: ((2R,3R)-3-((S)-1-((3R,4S,5S)-4-((S)—N, 3-dimethyl-2-((S)-3-methyl-2-(methyl(((4-(((((1S,2S)-2-(pyridin-2-yldisulfaneyl) cyclopentyl) oxy) carbonyl) amino) benzyl) oxy) carbonyl) amino) butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methy lamino) butanamido)butanamido)-3-methoxy-5-methylheptanoyl)pyrrolidin-2-yl)-3-methoxy-2-methylpropanoyl)-L-phenylalanine (100 mg, 0.137 mmol) and (1S,2S)-2-(pyridin-2-yldisulfaneyl)cyclopentyl (4-((((4-nitrophenoxy)carbonyl)oxy)methyl)phenyl)carbamate (74 mg, 0.137 mmol) in DMF (1 ml) was added DIPEA (0.036 ml, 0.205 mmol) followed by 1-hydroxy-7-azabenzotriazole in DMA (0.068 ml, 0.068 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((S)-1-((3R,4S,5S)-4-((S)—N,3-dimethyl-2-((S)-3-methyl-2-(methyl(((4-(((((1S,2S)-2-(pyridin-2-yldisulfaneyl)cyclopentyl)oxy)carbonyl)amino)benzyl)oxy)carbonyl)amino)butanamido)butana mido)-3-methoxy-5-methylheptanoyl)pyrrolidin-2-yl)-3-methoxy-2-methylpropanoyl)-L-phenylalanine (80 mg, 0.066 mmol, 51.61% yield) as a white solid. LCMS: [M+H]⁺ calcd for C₅₈H₈₃N₇O₁₂S₂, 1134.459; found 1134.5.

### Step 2: Synthesis of Compound 25

A solution of ((2R,3R)-3-((S)-1-((3R,4S,5S)-4-((S)—N,3-dimethyl-2-((S)-3-methyl-2-(methyl(((4-(((((1S,2S)-2-(pyridin-2-yldisulfaneyl) cyclopentyl) oxy) carbonyl) amino) benzyl) oxy) carbonyl) amino) butanamido) butanamido)-3-methoxy-5-methylheptanoyl)pyrrolidin-2-yl)-3-methoxy-2-methylpropanoyl)-L-phenylalanine (50 mg, 0.044 mmol) in DMF (1 ml) was cooled to 0° C. PV1 peptide (145 mg, 0.044 mmol) and triethylamine (7.37 μl, 0.053 mmol) were added and the reaction mixture was stirred at RT for 18 h. The reaction mixture was purified by preparative HPLC using 0.1% TFA in H₂O and ACN. The preparative fraction was lyophilized to afford Compound 25 (40 mg, 0.01 mmol, 21.10% yield) as a white solid. The product obtained is a di-TFA salt. LCMS: [M+H]⁺ calcd for C₂₀₅H₃₀₄N₄₂O₅₅S₂, 4301.046; found 1434.8 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 12.056; Purity (Max): 99.47%.

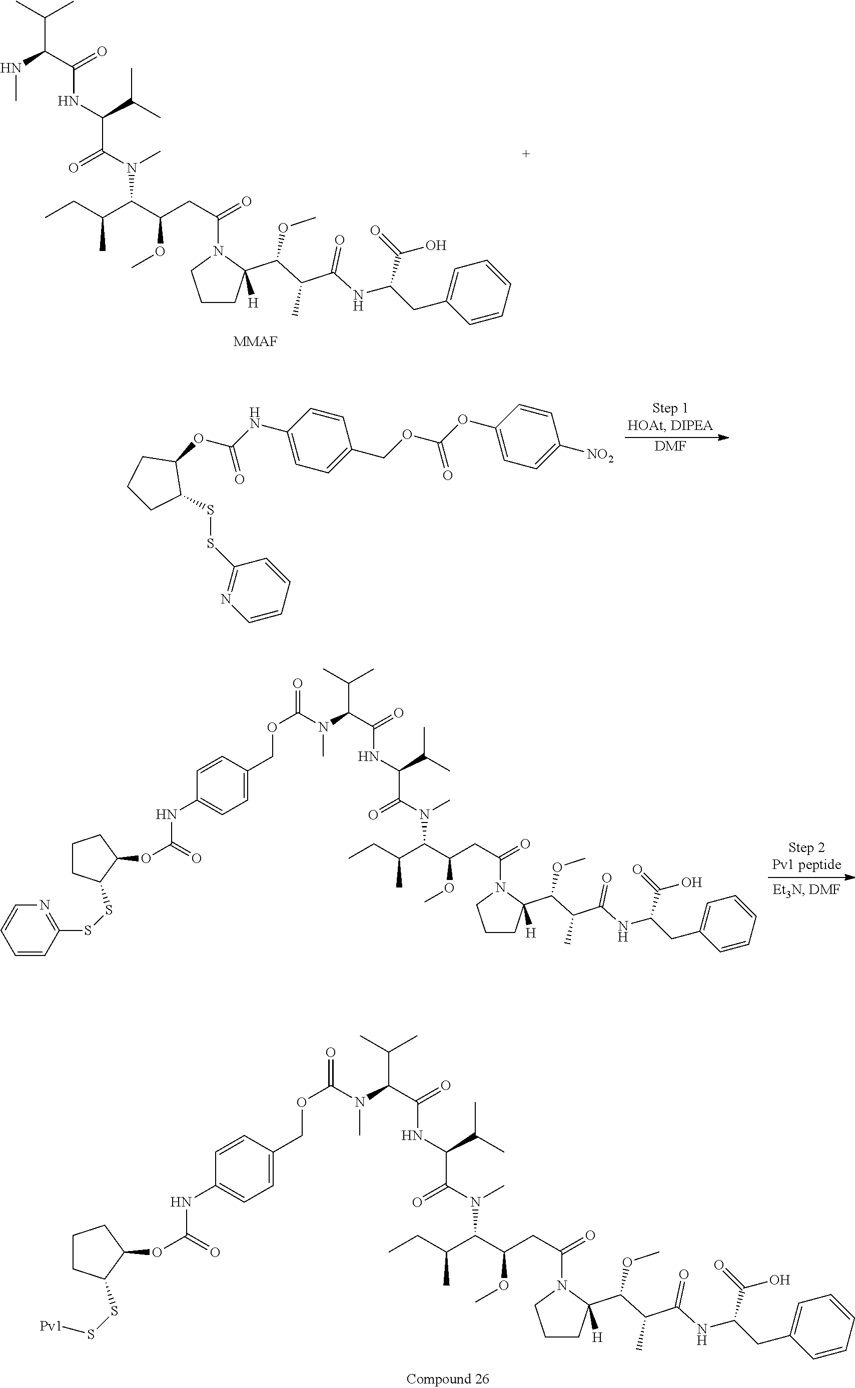

### Example 26: Synthesis of Compound 26

### Step 1: ((2R,3R)-3-((S)-1-((3R,4S,5S)-4-((S)—N, 3-dimethyl-2-((S)-3-methyl-2-(methyl(((4-(((((1R,2R)-2-(pyridin-2-yldisulfaneyl) cyclopentyl) oxy) carbonyl) amino) benzyl) oxy) carbonyl) amino) butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methylamino) butanamido)butanamido)-3-methoxy-5-methylheptanoyl)pyrrolidin-2-yl)-3-methoxy-2-methylpropanoyl)-L-phenylalanine (40 mg, 0.054 mmol) and (1R,2R)-2-(pyridin-2-yl disulfaneyl) cyclopentyl (4-((((4-nitro phenoxy)carbonyl)oxy)methyl)phenyl)carbamate (29.59 mg, 0.054 mmol) in DMF (1 ml) was added DIPEA (14.20 μl, 0.082 mmol) followed by 1-hydroxy-7-azabenzotriazole in DMA (2.73 μl, 0.027 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((S)-1-((3R,4S,5S)-4-((S)—N,3-dimethyl-2-((S)-3-methyl-2-(methyl(((4-(((((1R,2R)-2-(pyridin-2-yldisulfaneyl)cyclopentyl)oxy)carbonyl)amino)benzyl)oxy)carbonyl)amino)butanamido)butanamido)-3-methoxy-5-methylheptanoyl)pyrrolidin-2-yl)-3-methoxy-2-methylpropanoyl)-L-phenylalanine (35 mg, 0.031 mmol, 56.46% yield) as a white solid. LCMS: [M+H]⁺ calcd for C₅₈H₈₃N₇O₁₂S₂, 1134.459; found 1133.8.

### Step 2: Synthesis of Compound 26

A solution of ((2R,3R)-3-((S)-1-((3R,4S,5S)-4-((S)—N,3-dimethyl-2-((S)-3-methyl-2-(methyl(((4-(((((1R,2R)-2-(pyridin-2-yldisulfaneyl) cyclopentyl) oxy)c arbonyl) amino) benzyl) oxy) carbonyl) amino) butanamido) butanamido)-3-methoxy-5-methylheptanoyl)pyrrolidin-2-yl)-3-methoxy-2-methylpropanoyl)-L-phenylalanine (35 mg, 0.031 mmol) in DMF (1 ml) was cooled to 0° C. PV1 peptide (101.15 mg, 0.031 mmol) and triethylamine (5.16 μl, 0.037 mmol) were added and the reaction mixture was stirred at RT for 18 h. The reaction mixture was purified by preparative HPLC using 0.1% TFA in H₂O and ACN. The preparative fraction was lyophilized to afford Compound 26 (15 mg, 0.003 mmol, 11.30% yield) as a white solid. The product obtained is a di-TFA salt. LCMS: [M+H]⁺ calcd for C₂₀₅H₃₀₄N₄₂O₅₅S₂, 4301.046; found 1434.5 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 12.243; Purity (Max): 99.10%.

### Example 27: Synthesis of Compound 27

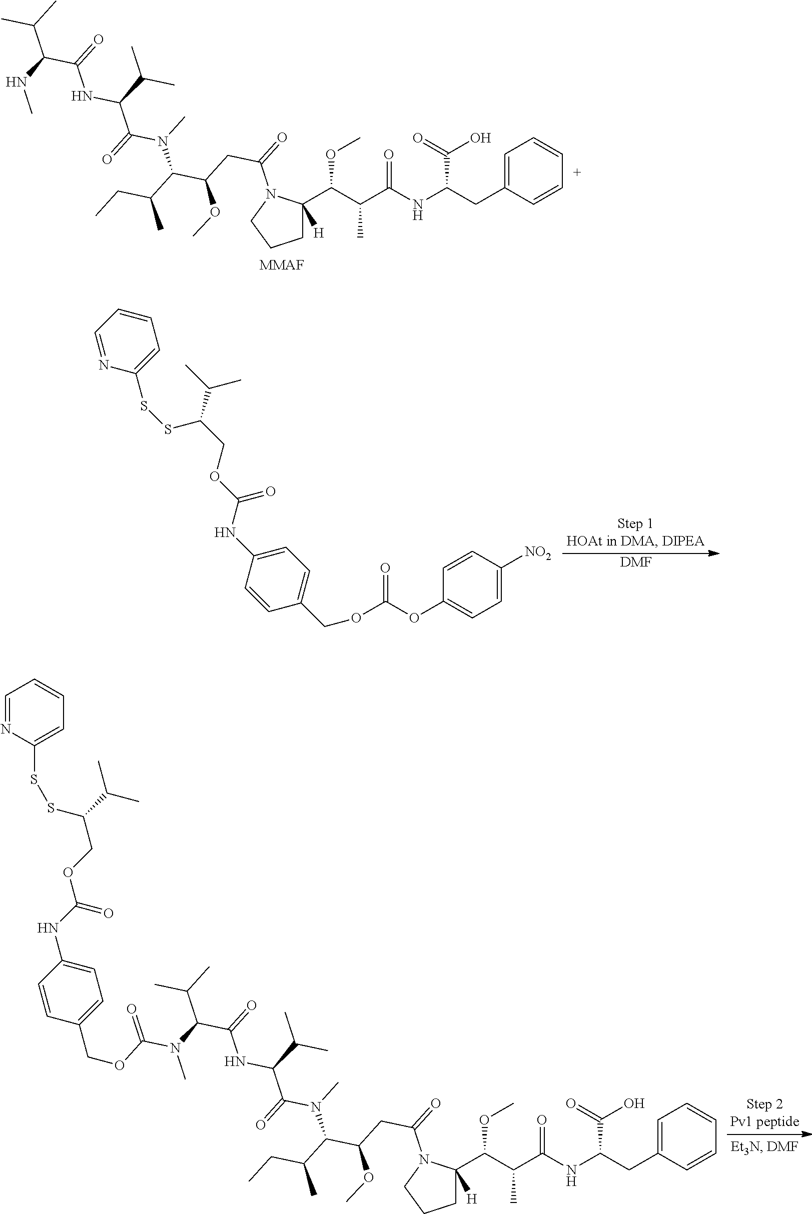

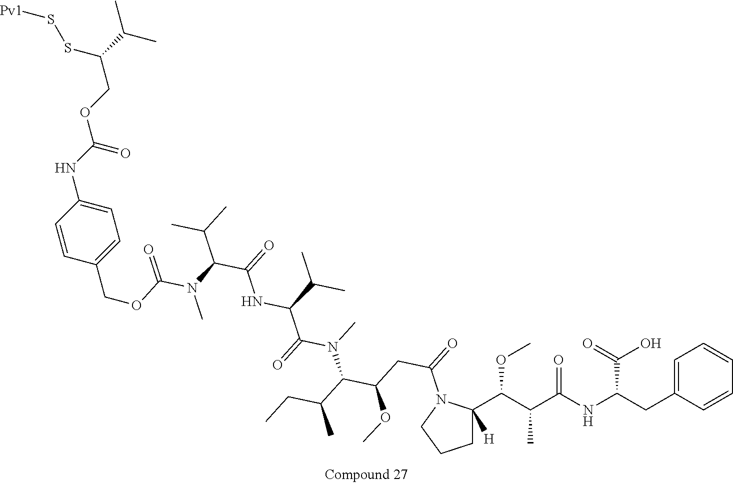

### Step 1: ((2R,3R)-3-((S)-1-((3R,4S,5S)-4-((S)—N,3-dimethyl-2-((S)-3-methyl-2-(methyl(((4-((((R)-3-methyl-2-(pyridin-2-yldisulfaneyl)butoxy) carbonyl)amino) benzyl) oxy) carbonyl) amino) butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methylamino) butanamido)butanamido)-3-methoxy-5-methylheptanoyl)pyrrolidin-2-yl)-3-methoxy-2-methylpropanoyl)-L-phenylalanine (40 mg, 0.054 mmol) and (R)-3-methyl-2-(pyridin-2-yl disulfaneyl) butyl (4-((((4-nitro phenoxy)carbonyl)oxy)methyl)phenyl)carbamate (1) (29.70 mg, 0.054 mmol) in DMF (1 ml) was added DIPEA (10.59 μl, 0.082 mmol) followed by 1-hydroxy-7-azabenzotriazole in DMA (3.71 μl, 0.027 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((S)-1-((3R,4S,5S)-4-((S)—N,3-dimethyl-2-((S)-3-methyl-2-(methyl(((4-((((R)-3-methyl-2-(pyridin-2-yldisulfaneyl)butoxy)carbonyl)amino)benzyl)oxy)carbonyl)amino)butanamido)butanamido)-3-methoxy-5-methylheptanoyl)pyrrolidin-2-yl)-3-methoxy-2-methylpropanoyl)-L-phenylalanine (35 mg, 0.029 mmol, 56.36% yield) as a white solid. LCMS: [M+H]⁺ calcd for C₅₈H₈₅N₇O₁₂S₂, 1136.475; found 1136.5.

### Step 2: Synthesis of Compound 27

A solution of ((2R,3R)-3-((S)-1-((3R,4S,5S)-4-((S)—N,3-dimethyl-2-((S)-3-methyl-2-(methyl(((4-((((R)-3-methyl-2-(pyridin-2-yldisulfaneyl)butoxy)carbonyl)amino) benzyl)oxy) carbonyl)amino) butanamido) butanamido)-3-methoxy-5-methylheptanoyl)pyrrolidin-2-yl)-3-methoxy-2-methylpropanoyl)-L-phenylalanine (35 mg, 0.031 mmol) in DMF (1 ml) was cooled to 0° C. Pv1 peptide (100.9 mg, 0.031 mmol) and triethylamine (5.15 μl, 0.037 mmol) were added and the reaction mixture was stirred at RT for 18 h. The reaction mixture was purified by preparative HPLC using 0.1% TFA in H₂O and ACN. The preparative fraction was lyophilized to afford Compound 27 (64 mg, 0.014 mmol, 47.22% yield) as a white solid. The product obtained is a di-TFA salt. LCMS: [M+H]⁺ calcd for C₂₀₅H₃₀₆N₄₂O₅₅S₂, 4303.062; found 1435.4 [(M+3)/3]; HPLC: Column X-Bridge C8(50×4.6) mm, 3.5 μm, Mobile phase: A: 0.1% TFA in water, Mobile phase: B: 0.1% TFA in ACN, Flow: 2.0 mL/min; RT (min): 5.83; Purity (Max): 96.842%.

### Example 28: Synthesis of Compound 28

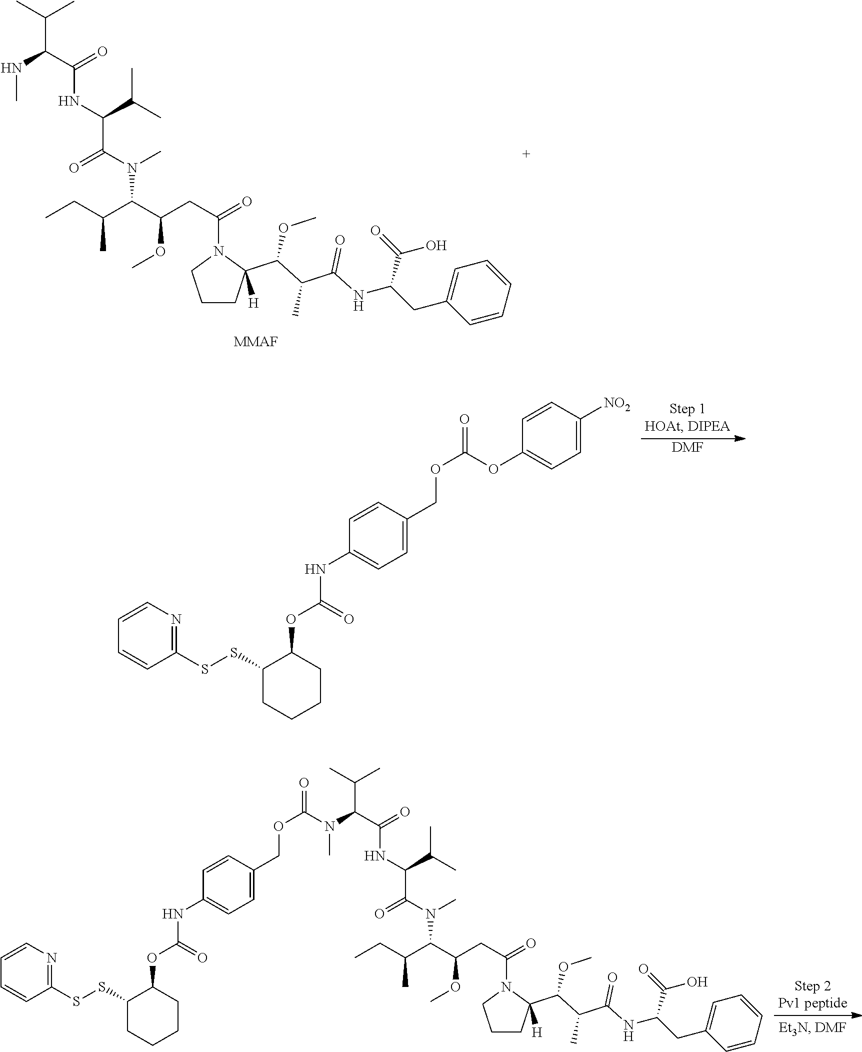

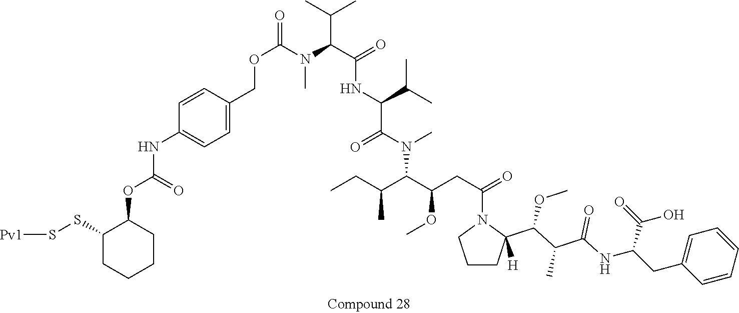

### Step 1: ((2R,3R)-3-((S)-1-((3R,4S,5S)-4-((S)—N, 3-dimethyl-2-((S)-3-methyl-2-(methyl(((4-(((((1S,2S)-2-(pyridin-2-yldisulfaneyl) cyclohexyl) oxy) carbonyl) amino) benzyl) oxy) carbonyl) amino) butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methyl amino) butanamido)butanamido)-3-methoxy-5-methylheptanoyl)pyrrolidin-2-yl)-3-methoxy-2-methylpropanoyl)-L-phenylalanine (51 mg, 0.069 mmol) and (1S,2S)-2-(pyridin-2-yldisulfaneyl)cyclohexyl (4-((((4-nitrophenoxy)carbonyl)oxy)methyl)phenyl)carbamate (38.71 mg, 0.069 mmol) in DMF (1 ml) was added DIPEA (18.10 μl, 0.104 mmol) followed by 1-hydroxy-7-azabenzotriazole in DMA (3.48 μl, 0.034 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((S)-1-((3R,4S,5S)-4-((S)—N,3-dimethyl-2-((S)-3-methyl-2-(methyl(((4-(((((1S,2S)-2-(pyridin-2-yldisulfaneyl)cyclohexyl)oxy)carbonyl)amino)benzyl)oxy)carbonyl)amino)butanamido)butanamido)-3-methoxy-5-methylheptanoyl)pyrrolidin-2-yl)-3-methoxy-2-methylpropanoyl)-L-phenylalanine (33 mg, 0.029 mmol, 41.23% yield) as a white solid. LCMS: [M+H]⁺ calcd for C₅₉H₈₅N₇O₁₂S₂, 1148.486; found 1148.5.

### Step 2: Synthesis of Compound 28

A solution of ((2R,3R)-3-((S)-1-((3R,4S,5S)-4-((S)—N,3-dimethyl-2-((S)-3-methyl-2-(methyl(((4-(((((1S,2S)-2-(pyridin-2-yldisulfaneyl) cyclohexyl) oxy) carbonyl) amino) benzyl) oxy) carbonyl) amino) butanamido) butanamido)-3-methoxy-5-methylheptanoyl)pyrrolidin-2-yl)-3-methoxy-2-methylpropanoyl)-L-phenylalanine (33 mg, 0.029 mmol) in DMF (1 ml) was cooled to 0° C. Pv1 peptide (103.63 mg, 0.031 mmol) and triethylamine (4.80 μl, 0.034 mmol) were added and the reaction mixture was stirred at RT for 18 h. The reaction mixture was purified by preparative HPLC using 0.1% TFA in H₂O and ACN. The preparative fraction was lyophilized to afford Compound 28 (75 mg, 0.017 mmol, 60.49% yield) as a white solid. The product obtained is a di-TFA salt. LCMS: [M+H]⁺ calcd for C₂₀₆H₃₀₆N₄₂O₅₅S₂, 4315.073; found 1439.3 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 12.516; Purity (Max): 99.59%.

### Example 29: Synthesis of Compound 29

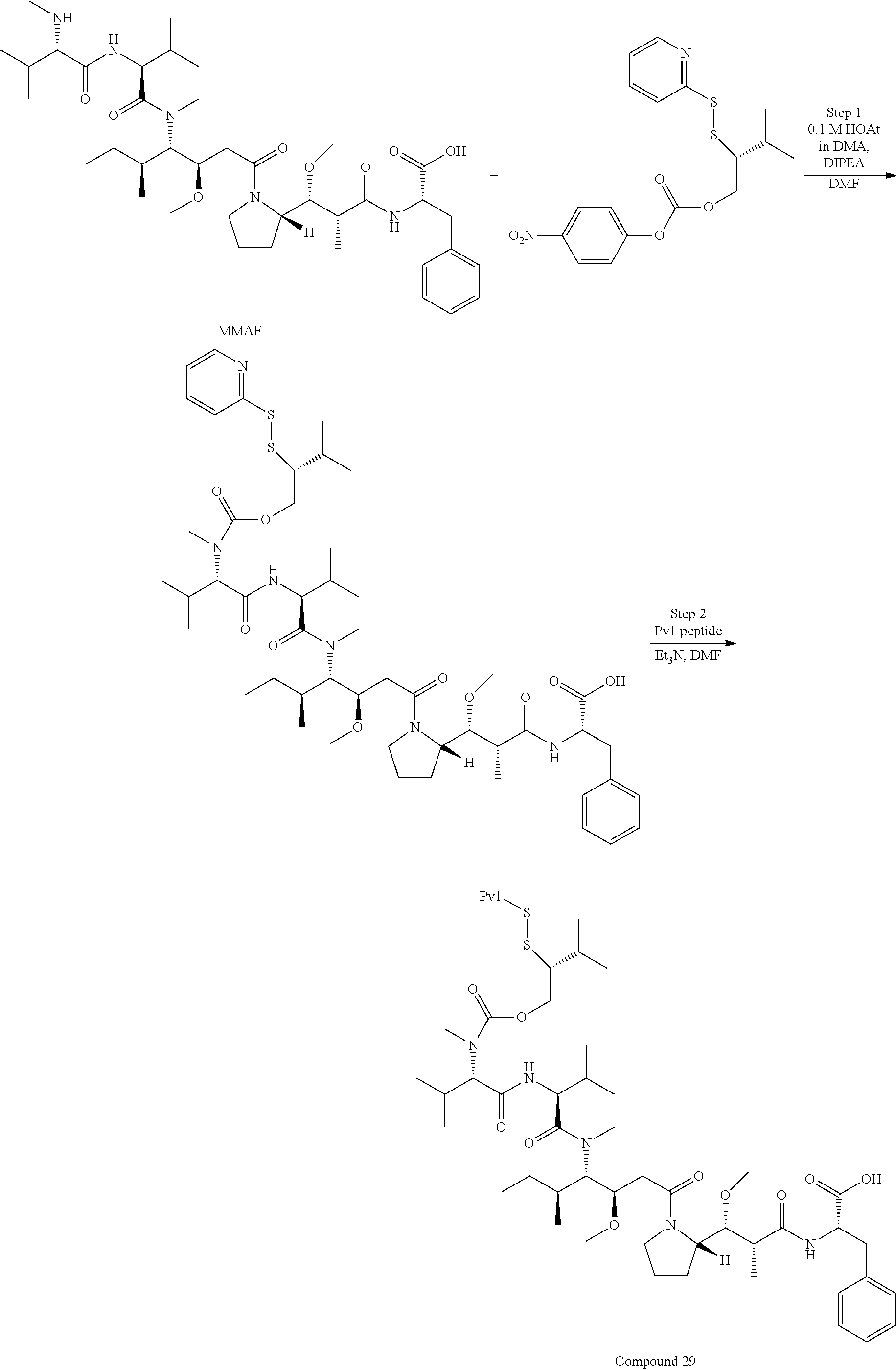

### Step 1: ((2R,3R)-3-((R)-1-((3R,4S,5S)-4-((S)—N,3-dimethyl-2-((S)-3-methyl-2-(methyl(((R)-3-methyl-2-(pyridin-2-yldisulfaneyl)butoxy)carbonyl)amino)butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methylamino) butanamido)butanamido)-3-methoxy-5-methylheptanoyl)pyrrolidin-2-yl)-3-methoxy-2-methylpropanoyl)-L-phenylalanine (230 mg, 0.314 mmol) and (R)-3-methyl-2-(pyridin-2-yldisulfaneyl)butyl (4-nitrophenyl) carbonate (124 mg, 0.314 mmol) in DMF (1 ml) was added DIPEA (0.11 ml, 0.628 mmol) followed by 1-hydroxy-7-azabenzotriazole in DMA (0.157 ml, 0.157 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((R)-1-((3R,4S,5S)-4-((S)—N,3-dimethyl-2-((S)-3-methyl-2-(methyl(((R)-3-methyl-2-(pyridin-2-yl disulfaneyl) butoxy) carbonyl) amino) butanamido) butanamido)-3-methoxy-5-methylheptanoyl)pyrrolidin-2-yl)-3-methoxy-2-methylpropanoyl)-L-phenylalanine (150 mg, 0.148 mmol, 48.35% yield) as a white solid. LCMS: [M+H]⁺ calcd for C₅₀H₇₈N₆O₁₀S₂, 987.326; found 986.4 (M−H)

### Step 2: Synthesis of Compound 29

A solution of ((2R,3R)-3-((R)-1-((3R,4S,5S)-4-((S)—N,3-dimethyl-2-((S)-3-methyl-2-(methyl(((R)-3-methyl-2-(pyridin-2-yldisulfaneyl)butoxy)carbonyl)amino)butanamido)butanamido)-3-methoxy-5-methylheptanoyl)pyrrolidin-2-yl)-3-methoxy-2-methylpropanoyl)-L-phenylalanine (150 mg, 0.152 mmol) in DMF (1 ml) was cooled to 0° C. Pv1 peptide (498 mg, 0.152 mmol) and triethylamine (41.8 μl, 0.304 mmol) were added and the reaction mixture was stirred at RT for 3 h. The reaction mixture was purified by preparative HPLC using 0.1% TFA in H₂O and ACN. The preparative fraction was lyophilized to afford Compound 29 (425 mg, 0.101 mmol, 67.34% yield) as a white solid. The product obtained is a di-TFA salt. LCMS: [M+H]⁺ calcd for C₁₉₇H₂₉₉N₄₁O₅₃S₂, 4153.913; found 1385.7 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 12.257; Purity (Max): 98.793%.

### Example 30: Synthesis of Compound 30

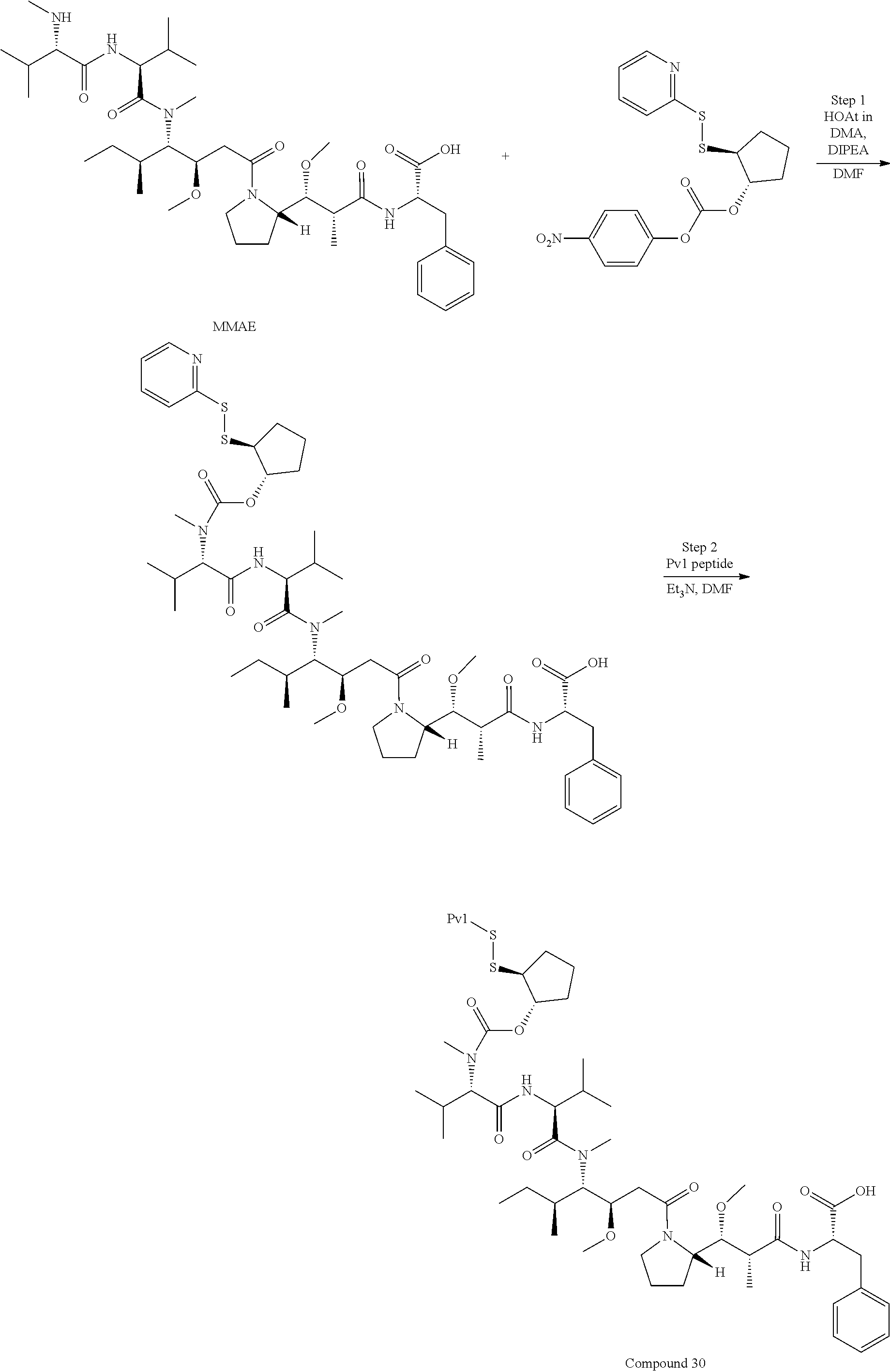

### Step 1: ((2R,3R)-3-((R)-1-((3R,4S,5S)-4-((S)—N, 3-dimethyl-2-((S)-3-methyl-2-(methyl((((1S,2S)-2-(pyridin-2-yldisulfaneyl)cyclopentyl)oxy)carbonyl)amino)butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methylamino) butanamido)butanamido)-3-methoxy-5-methylheptanoyl)pyrrolidin-2-yl)-3-methoxy-2-methylpropanoyl)-L-phenylalanine (56 mg, 0.076 mmol) and 4-nitrophenyl ((1S,2S)-2-(pyridin-2-yldisulfaneyl)cyclopentyl) carbonate (35.84 mg, 0.092 mmol) in DMF (1 ml) was added DIPEA (20.42 μl, 0.115 mmol) followed by 1-hydroxy-7-azabenzotriazole in DMA (5.20 μl, 0.038 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((R)-1-((3R,4S,5S)-4-((S)—N,3-dimethyl-2-((S)-3-methyl-2-(methyl((((1S,2S)-2-(pyridin-2-yldisulfaneyl) cyclopentyl) oxy) carbonyl) amino) butanamido)butanamido)-3-methoxy-5-methylheptanoyl)pyrrolidin-2-yl)-3-methoxy-2-methylpropanoyl)-L-phenylalanine (2) (45 mg, 0.041 mmol, 59.69% yield) as a white solid. LCMS: [M+H]⁺ calcd for C₅₀H₇₆N₆O₁₀S₂, 985.310; found 983.4 (M−H)

### Step 2: Synthesis of Compound 30

A solution of ((2R,3R)-3-((R)-1-((3R,4S,5S)-4-((S)—N,3-dimethyl-2-((S)-3-methyl-2-(methyl((((1S,2S)-2-(pyridin-2-yldisulfaneyl)cyclopentyl)oxy)carbonyl)amino)butanamido)butanamido)-3-methoxy-5-methylheptanoyl)pyrrolidin-2-yl)-3-methoxy-2-methylpropanoyl)-L-phenylalanine (43 mg, 0.044 mmol) in DMF (1 ml) was cooled to 0° C. Pv1 peptide (143 mg, 0.044 mmol) and triethylamine (12.16 μl, 0.087 mmol) were added and the reaction mixture was stirred at RT for 3 h. The reaction mixture was purified by preparative HPLC using 0.1% TFA in H₂O and ACN. The preparative fraction was lyophilized to afford Compound 30 (102 mg, 0.024 mmol, 56.29% yield) as a white solid. The product obtained is a di-TFA salt. LCMS: [M+H]⁺ calcd for C₁₉₇H₂₉₇N₄₁O₅₃S₂, 4151.897; found 1383.1 [(M−3)/3]; HPLC: Column X-Bridge C₈(50×4.6) mm, 3.5μm, Mobile phase: A: 0.1% TFA in water, Mobile phase: B: 0.1% TFA in ACN, Flow: 2.0 mL/min; RT (min): 5.596; Purity (Max): 98.55%

### Example 31: Synthesis of Compound 31

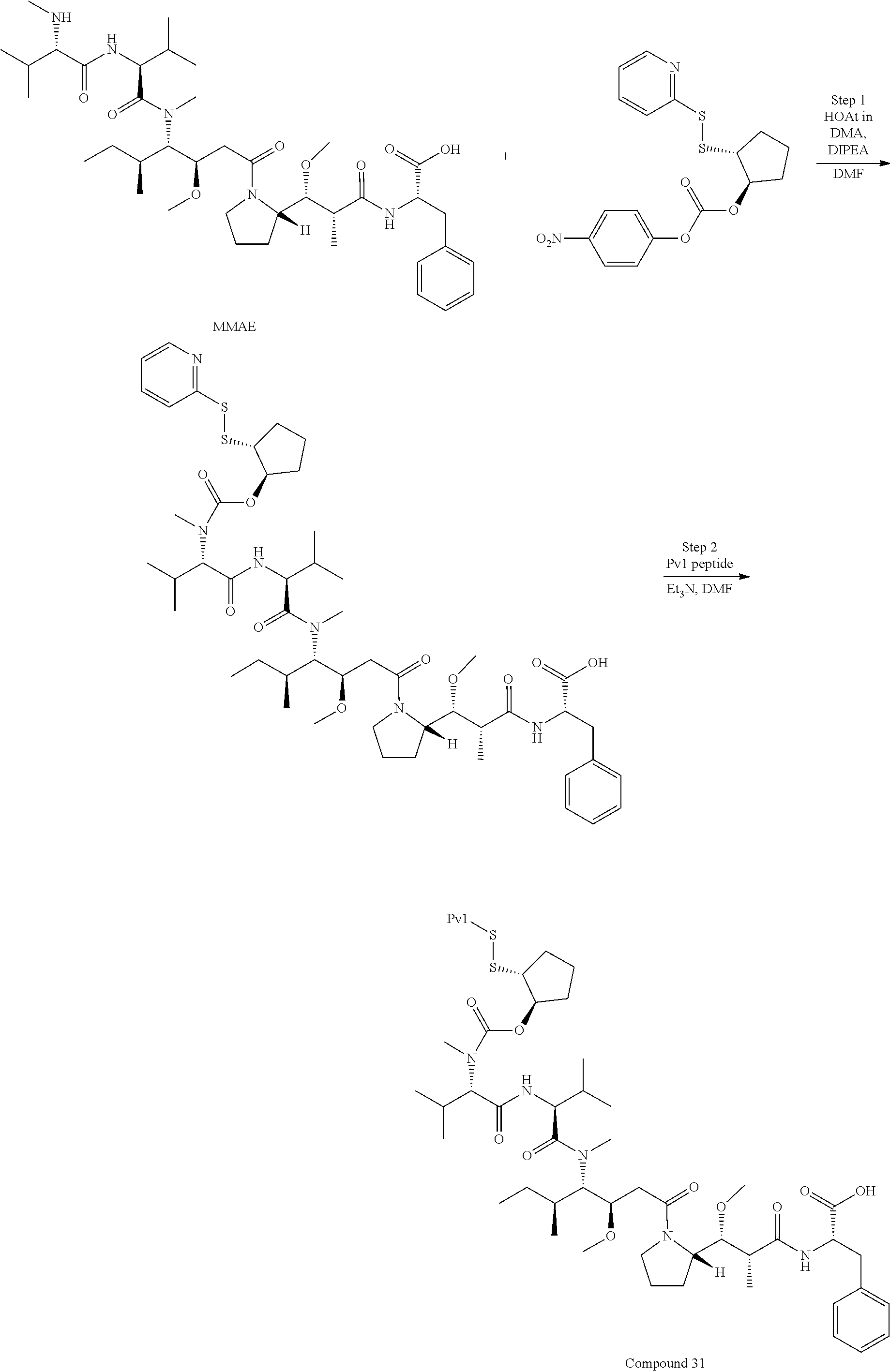

### Step 1: ((2R,3R)-3-((R)-1-((3R,4S,5S)-4-((S)—N, 3-dimethyl-2-((S)-3-methyl-2-(methyl((((1R,2R)-2-(pyridin-2-yldisulfaneyl)cyclopentyl)oxy)carbonyl)amino)butanamido)butanamido)-3-methoxy-5-methylheptanoyl)pyrrolidin-2-yl)-3-methoxy-2-methylpropanoyl)-L-phenylalanine

To a stirred solution of ((2R,3R)-3-((S)-1-((3R,4S,5S)-4-((S)—N,3-dimethyl-2-((S)-3-methyl-2-(methylamino) butanamido)butanamido)-3-methoxy-5-methylheptanoyl)pyrrolidin-2-yl)-3-methoxy-2-methylpropanoyl)-L-phenylalanine (56 mg, 0.076 mmol) and (4-nitrophenyl ((1R,2R)-2-(pyridin-2-yldisulfaneyl)cyclopentyl) carbonate (35.84 mg, 0.092 mmol) in DMF (1 ml) was added DIPEA (20.42 μl, 0.115 mmol) followed by 1-hydroxy-7-azabenzotriazole in DMA (5.20 μl, 0.038 mmol) at 0° C. The reaction mixture was stirred at RT for 18 h. The reaction mixture was purified by preparative HPLC using 0.1% HCOOH in H₂O and ACN. The preparative fraction was lyophilized to afford: ((2R,3R)-3-((R)-1-((3R,4S,5S)-4-((S)—N,3-dimethyl-2-((S)-3-methyl-2-(methyl((((1R,2R)-2-(pyridin-2-yldisulfaneyl) cyclopentyl) oxy) carbonyl) amino) butanamido)butanamido)-3-methoxy-5-methylheptanoyl)pyrrolidin-2-yl)-3-methoxy-2-methylpropanoyl)-L-phenylalanine (28 mg, 0.028 mmol, 37.14% yield) as a white solid. LCMS: [M+H]⁺ calcd for C₅₀H₇₆N₆O₁₀S₂, 985.310; found 984.4 (M−H)

### Step 2: Synthesis of Compound 31

A solution of ((2R,3R)-3-((R)-1-((3R,4S,5S)-4-((S)—N,3-dimethyl-2-((S)-3-methyl-2-(methyl((((1R,2R)-2-(pyridin-2-yldisulfaneyl)cyclopentyl)oxy)carbonyl)amino)butanamido)butanamido)-3-methoxy-5-methylheptanoyl)pyrrolidin-2-yl)-3-methoxy-2-methylpropanoyl)-L-phenylalanine (25 mg, 0.025 mmol) in DMF (1 ml) was cooled to 0° C. Pv1 peptide (83 mg, 0.025 mmol) and triethylamine (2.56 μl, 0.025 mmol) were added and the reaction mixture was stirred at RT for 3 h. The reaction mixture was purified by preparative HPLC using 0.1% TFA in H₂O and ACN. The preparative fraction was lyophilized to afford Compound 31 (70 mg, 0.017 mmol, 66.44% yield) as a white solid. The product obtained is a di-TFA salt. LCMS: [M+H]⁺ calcd for C₁₉₇H₂₉₇N₄₁O₅₃S₂, 4151.897; found 1384.9 [(M+3)/3]; HPLC: Column Atlantis dC18 (250×4.6) mm, 5 m, Mobile Phase A: 0.1% TFA in MilliQ water, Mobile Phase B: ACN; Flow: 1.0 mL/min; RT (min): 12.134; Purity (Max): 98.998%

### Example 32: Synthesis of Compound 32

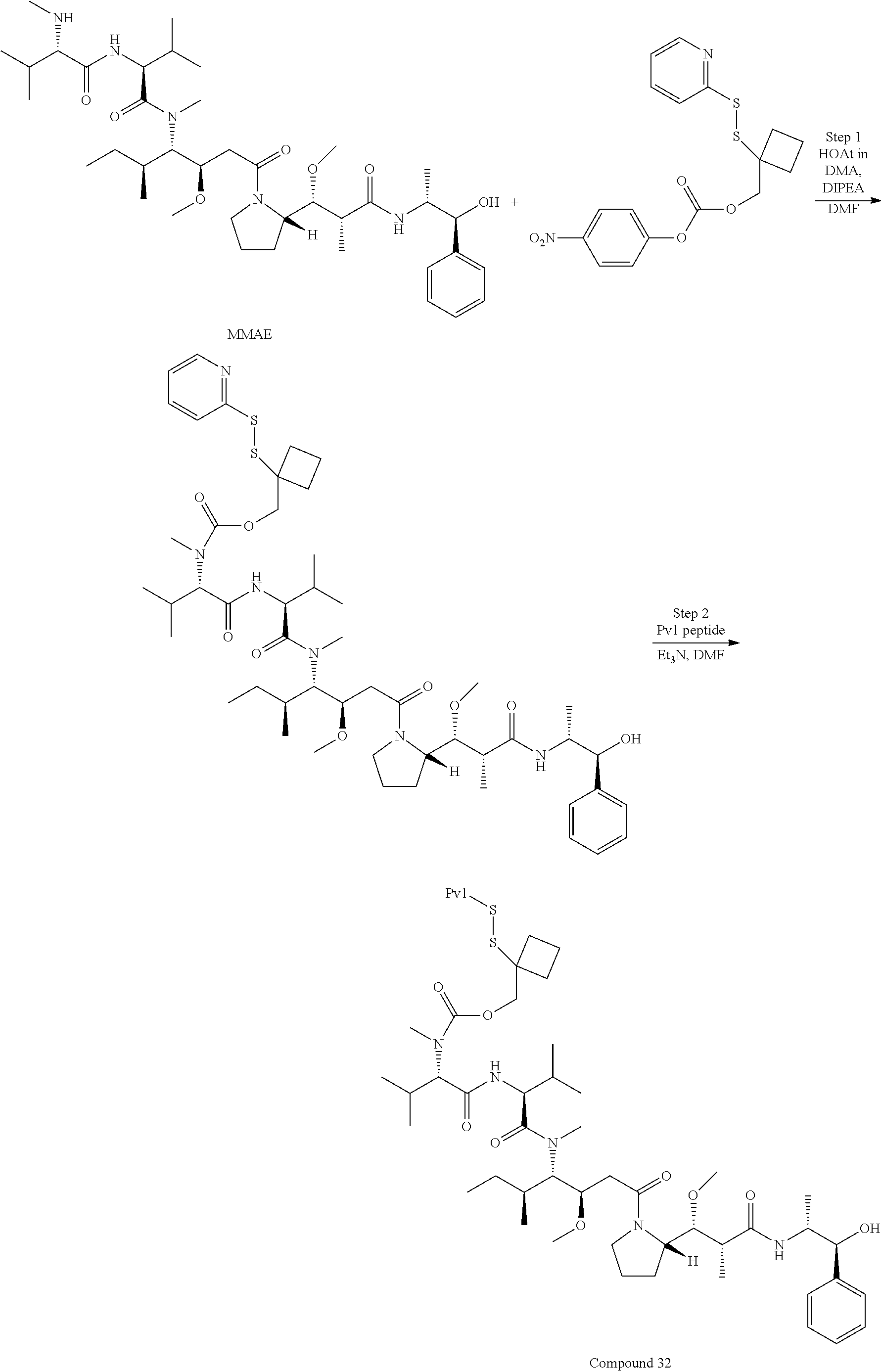

### Step 1: (1-(pyridin-2-yldisulfaneyl)cyclobutyl)methyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate

To a stirred solution of (S)—N-((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (50 mg, 0.069 mmol) and 4-nitrophenyl ((1-(pyridin-2-yldisulfaneyl)cyclobutyl)methyl) carbonate (2) (36.55 mg, 0.083 mmol) in DMF (1 ml) was added DIPEA (24.13 μL, 0.014 mmol) followed by 1-hydroxy-7-azabenzotriazole in DMA (0.47 ml, 0.035 mmol) at 0° C. The reaction mixture was stirred at RT for 18 h. The reaction mixture was purified by preparative HPLC using 0.1% HCOOH in H₂O and ACN. The preparative fraction was lyophilized to afford: (1-(pyridin-2-yldisulfaneyl)cyclobutyl)methyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (35 mg, 0.034 mmol, 49.45% yield) as a white solid. LCMS: [M+H]⁺ calcd for C₅₀H₇₇N₇O₁₁S₂, 1016.324; found 1015.0 (M−H)

### Step 2: Synthesis of Compound 32

A solution of (1-(pyridin-2-yldisulfaneyl)cyclobutyl)methyl ((S)-1-(((S)-1-(((3R,4S,5S)-1-((R)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (35 mg, 0.034 mmol) in DMF (1 ml) was cooled to 0° C. Pv1 peptide (112.9 mg, 0.034 mmol) and triethylamine (9.6 μl, 0.068 mmol) were added and the reaction mixture was stirred at RT for 4 h. The reaction mixture was purified by preparative HPLC using 0.1% TFA in H₂O and ACN. The preparative fraction was lyophilized to afford Compound 32 (82 mg, 0.020 mmol, 57.45% yield) as a white solid. The product obtained is a di-TFA salt. LCMS: [M+H]⁺ calcd for C₁₉₇H₂₉₉N₄₁O₅₂S₂, 4137.914; found 1378.1 [(M−3)/3]; HPLC: Column X-Bridge C8(50×4.6) mm, 3.5 μm, Mobile phase: A: 0.1% TFA in water, Mobile phase: B: 0.1% TFA in ACN, Flow: 2.0 mL/min; RT (min): 5.53; Purity (Max): 98.659%

The following compounds of Table 3 were prepared using the procedures described in the examples above.

TABLE 3

| Example | Structure |
|---|---|
| 33 | 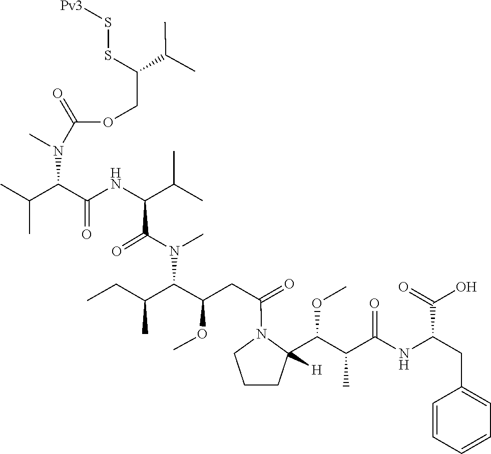 |
| 34 | 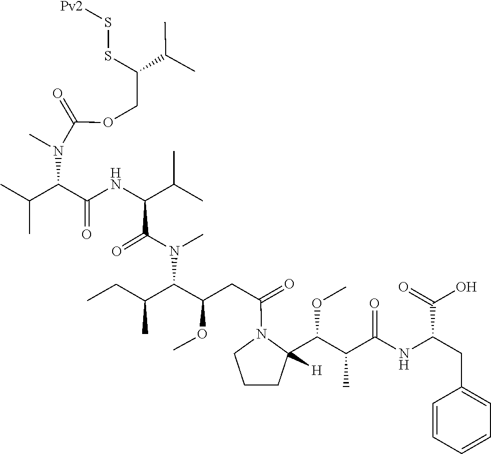 |
| 35 | 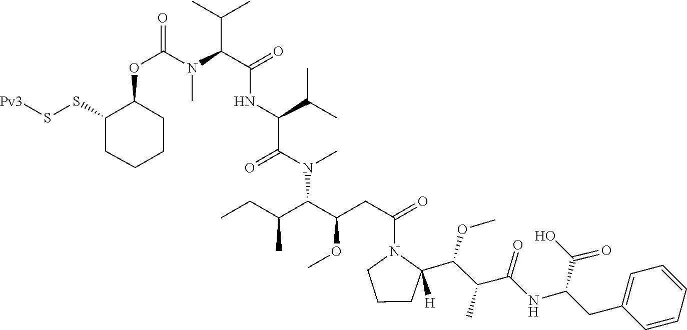 |
| 36 | 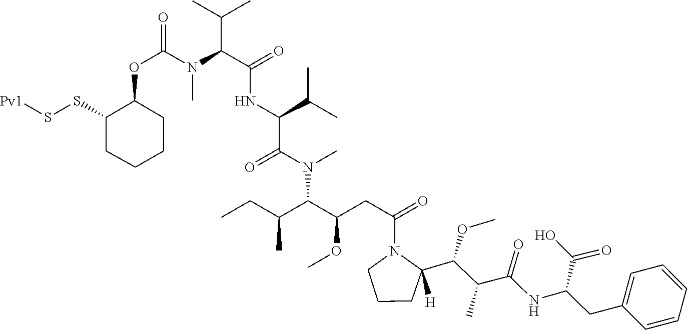 |
| 37 | 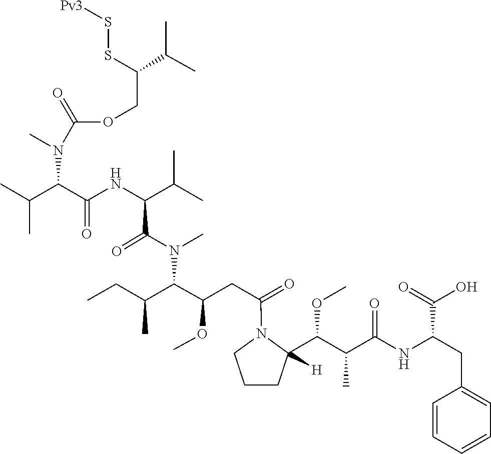 |

### Example 38: Synthesis of Compound 38

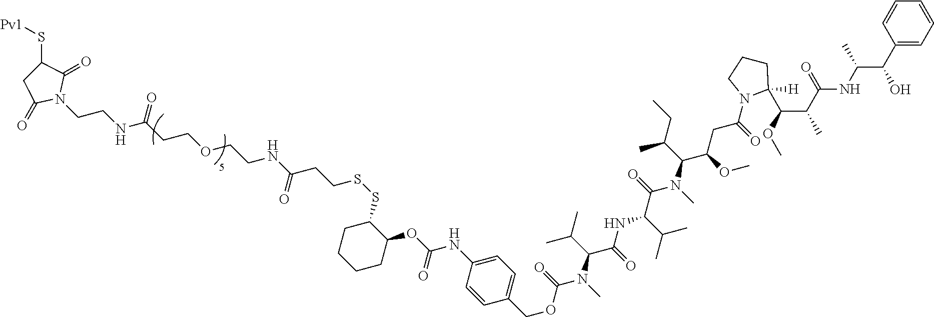

### Step 1: Synthesis of allyl 2,2-dimethyl-4-oxo-3,8,11,14,17,20-hexaoxa-5-azatricosan-23-oate (38-2)

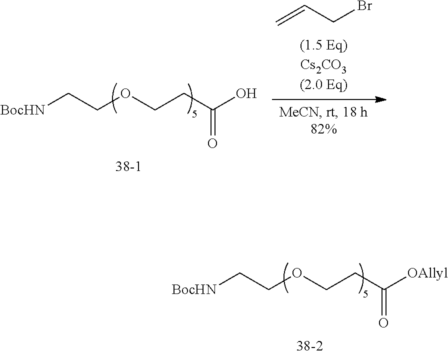

To a solution of 38-1 (2.00 g, 1.0 Eq, 4.88 mmol) in acetonitrile (50 mL) were added cesium carbonate (3.18 g, 2.0 Eq, 9.77 mmol) and allyl bromide (630 μL, 1.50 Eq, 7.33 mmol). The reaction mixture was stirred at room temperature for 18 h. The remaining cesium carbonate was filtered off and the solvent removed in vacuo. Purification by flash chromatography (EtOAc/cyclohexane, 0% for 2 CV, 0% to 100% in 10 CV) gave the title compound (1.80 g, 82%) as a white solid. ¹H NMR (400 MHz, DMSO-d₆) 6.75 (t, J=5.7 Hz, 1H), 5.95-5.85 (m, 1H), 5.34-5.24 (m, 1H), 5.22-5.16 (m, 1H), 4.55 (dt, J=5.3, 1.6 Hz, 2H), 3.64 (t, J=6.2 Hz, 2H), 3.54-3.50 (m, 16H) 3.4 (t, J=6.1 Hz, 2H), 3.05 (q, J=6.0 Hz, 2H), 2.57 (t, J=6.2 Hz, 2H), 1.37 (s, 9H).

### Step 2: Synthesis of allyl 1-amino-3,6,9,12,15-pentaoxaoctadecan-18-oate hydrochloride (38-3)

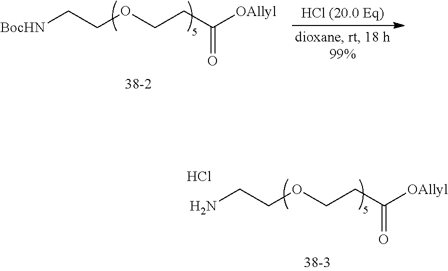

To a solution of 38-2 (1.80 g, 1.0 Eq, 4.00 mmol) in dioxane (20 mL) was added 4N HCl in dioxane (20.0 mL, 20.0 Eq, 80.0 mmol) and the reaction was stirred at room temperature for 18 h. The reaction was concentrated in vacuo and the residue was triturated with diethyl ether to afford the title compound (1.55 g, 99%) as a colorless oil. ¹H NMR (400 MHz, MeOD-d₄) δ 5.95 (ddt, J=17.2, 10.5, 5.6 Hz, 1H), 5.37-5.27 (m, 1H), 5.24-5.20 (m, 1H), 4.61 (dt, J=5.6, 1.5 Hz, 2H), 3.68-3.62 (m, 20H), 3.17-3.11 (m, 2H), 2.64 (t, J=6.0 Hz, 2H).

### Step 3: Synthesis of allyl 1-(((1S,2S)-2-(((4-(hydroxymethyl)phenyl)carbamoyl)oxy)cyclohexyl)disulfaneyl)-3-oxo-7,10,13,16,19-pentaoxa-4-azadocosan-22-oate (38-4)

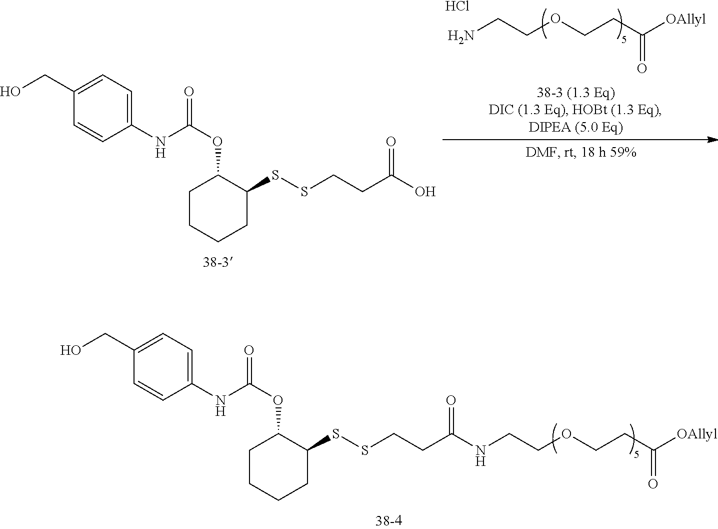

To a solution of 38-3′ (500 mg, 1.0 Eq, 1.30 mmol) in anhydrous DMF (10 mL) were added 1H-benzo[d][1,2,3]triazol-1-ol (228 mg, 1.3 Eq, 1.69 mmol), N,N′-diisopropylcarbodiimide (262 μL, 1.3 Eq, 1.69 mmol) and N-ethyl-N-isopropylpropan-2-amine (838 mg, 5.0 Eq, 6.49 mmol). The mixture was stirred for 10 min. Next, allyl 1-amino-3,6,9,12,15-pentaoxaoctadecan-18-oate hydrochloride 38-3 (651 mg, 1.3 Eq, 1.69 mmol) in DMF (10 mL) was added and the solution continued to stir at room temperature for 18 h. The mixture was purified by reverse phase chromatography (methanol/water(0.1% formic acid), 5% for 2 CV, 5% to 95% in 12 CV, 95% for 2 CV) to afford the title compound (545 mg, 59%) as a white solid. ¹H NMR (400 MHz, DMSO-d₆) δ 9.58 (s, 1H), 7.97 (t, J=5.7 Hz, 1H), 7.41 (d, J=8.3 Hz, 2H), 7.24-7.16 (m, 2H), 5.90 (ddt, J=17.3, 10.6, 5.4 Hz, 1H), 5.38-5.12 (m, 2H), 4.67-4.58 (m, 1H), 4.55 (dt, J=5.4, 1.5 Hz, 2H), 4.43-4.37 (m, 2H), 3.64 (t, J=6.2 Hz, 2H), 3.52-3.44 (m, 16H), 3.38 (t, J=5.9 Hz, 2H), 3.21-3.13 (m, 2H), 2.92-2.81 (m, 3H), 2.59-2.53 (m, 2H), 2.44 (t, J=7.2 Hz, 2H), 2.16-2.00 (m, 2H), 1.77-1.26 (m, 6H). LC-MS (ESI+) Exact mass calculated for [C₃₃H₅₃N₂O₁₁S₂]⁺ [M+H]⁺: 717, found: 717.

### Step 4: Synthesis of allyl 1-(((1S,2S)-2-(((4-((((4-nitrophenoxy)carbonyl)oxy)methyl)phenyl)carbamoyl)oxy)cyclohexyl)disulfaneyl)-3-oxo-7,10,13,16,19-pentaoxa-4-azadocosan-22-oate (38-5)

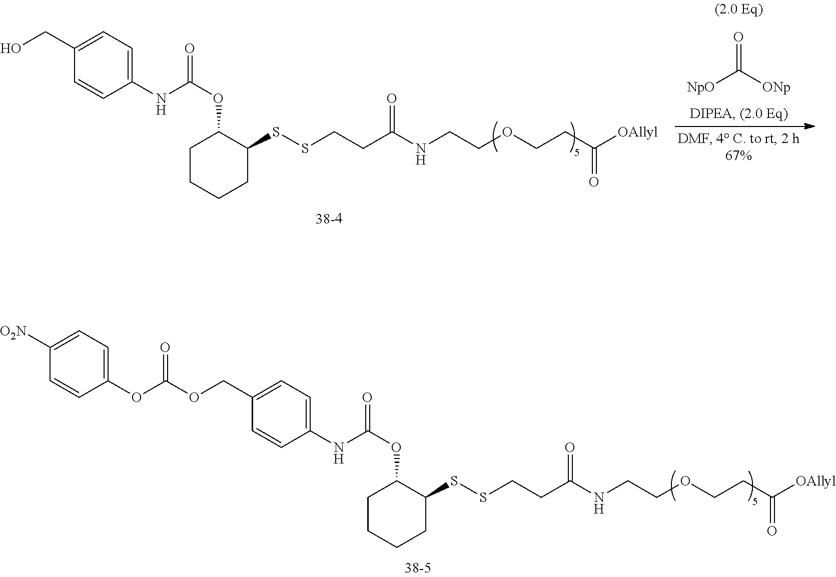

To a solution of 38-4 (540 mg, 1.0 Eq, 753 μmol) in anhydrous DMF (15 mL) at 4° C. was added bis(4-nitrophenyl) carbonate (458 mg, 2.0 Eq, 1.51 mmol) and diisopropylethylamine (388 μL, 3.0 Eq, 2.26 mmol). The mixture was allowed to warm to room temperature and stirred for 18 h. The mixture was purified by reverse phase chromatography (methanol/water(0.1% formic acid), 5% for 2 CV, 5% to 95% in 12 CV, 95% for 2 CV) to afford the title compound (444 mg, 67%) as a white solid. ¹H NMR (400 MHz, MeOD-d₄) δ 8.37-8.26 (m, 2H), 7.55-7.43 (m, 4H), 7.42-7.34 (m, 2H), 5.93 (ddt, J=17.2, 10.8, 5.5 Hz, 1H), 5.34-5.27 (m, 1H), 5.26-5.23 (m, 2H), 5.22-5.18 (m, 1H), 4.74-4.64 (m, 1H), 4.60-4.56 (m, 2H), 3.73 (t, J=6.2 Hz, 2H), 3.61-3.56 (m, 16H), 3.49 (t, J=5.3 Hz, 2H), 2.95 (td, J=7.2, 1.8 Hz, 2H), 2.88-2.79 (m, 1H), 2.61-2.55 (m, 4H), 2.23-2.12 (m, 2H), 1.82-1.38 (m, 6H), 3 protons are most probably covered by the methanol signal. LC-MS (ESI+) Exact mass calculated for [C₄₀H₅₆N₃O₁₅S₂]⁺ [M+H]⁺: 882, found: 882.

### Step 5: Synthesis of Compound 38-6

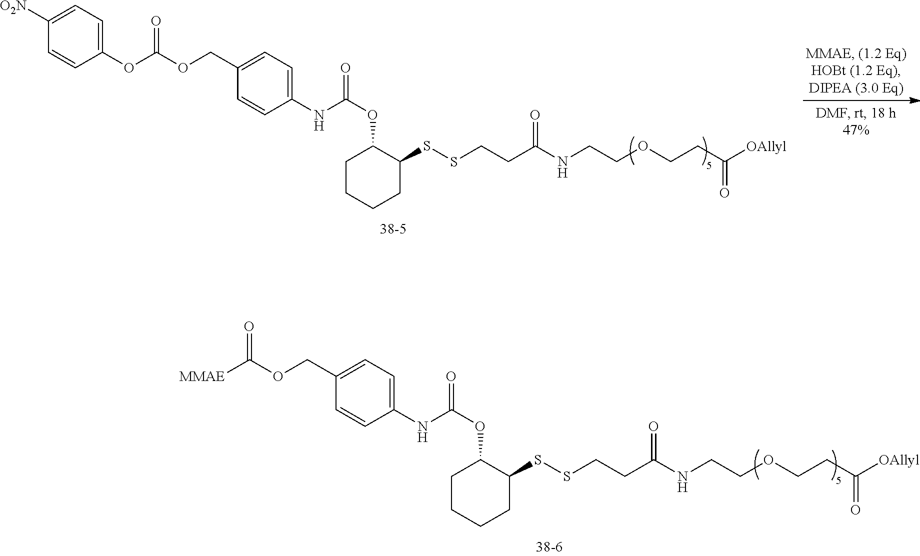

To a solution of 38-5 (400 mg, 1.0 Eq, 454 μmol) in DMF (4 mL) were added HOBt (93.1 mg, 1.2 Eq, 544 μmol), DIPEA (234 μL, 3.0 Eq, 1.36 mmol), MMAE (391 mg, 1.2 Eq, 544 μmol) and 3 Å molecular sieves. The reaction was stirred at room temperature for 18 h. The mixture was purified by reverse phase chromatography (methanol/water(0.1% formic acid), 5% for 2 CV, 5% to 95% in 12 CV, 95% for 2 CV) to afford the title compound (313 mg, 47%) as a fluffy white solid. LC-MS (ESI+) Exact mass calculated for [C₇₃H₁₁₈N₇O₁₉S₂]⁺ [M+H]⁺: 1461, found: 1461.

### Step 7: Synthesis of Compound 38-8

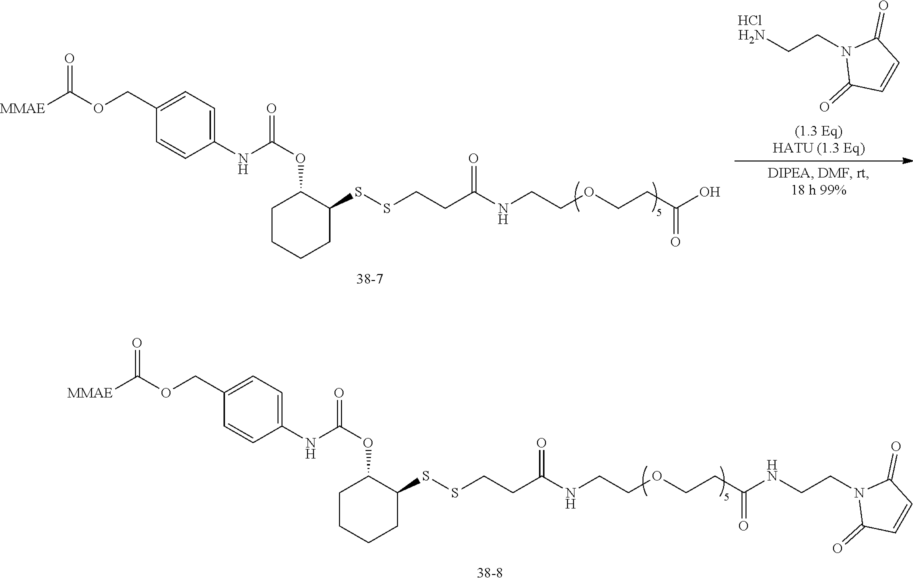

To a solution of 38-7 (60 mg, 1.0 Eq, 42 μmol) in DMF (5 mL) was added HATU (21 mg, 1.3 Eq, 55 μmol) and diisopropylethylamine (29 μL, 4.0 Eq, 0.17 mmol). After 15 min stirring at room temperature a solution of 1-(2-aminoethyl)-1H-pyrrole-2,5-dione hydrochloride (9.7 mg, 1.3 Eq, 55 μmol) in DMF (5 mL) was added and the mixture was stirred at room temperature for 18 h. The reaction was purified by reverse phase chromatography (acetonitrile/water(0.1% formic acid), 5% for 2 CV, 5% to 95% in 12 CV, 95% for 2 CV) to afford the title compound as a white solid (65 mg, 99%). LC-MS (ESI+) Exact mass calculated for [C₇₆H₁₂₀N₉O₂₀S₂]⁺ [M+H]+: 1542.8, found: 1543.3

### Step 8: Synthesis of Compound 38

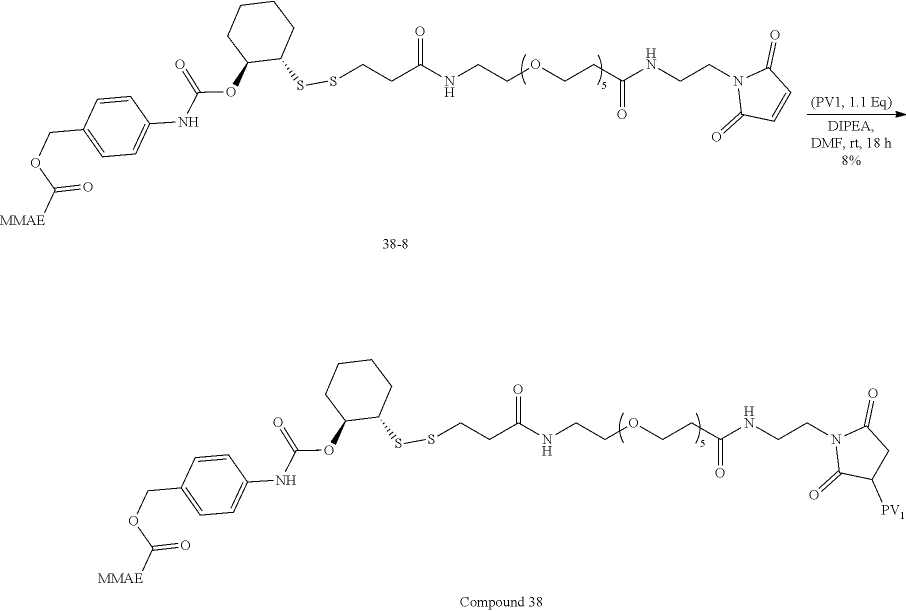

To a solution of 38-8 (65 mg, 1.0 Eq, 42 μmol) in DMF (3 mL) was added Pv1 (150 mg, 1.1 Eq, 46 μmol) and diisopropylethylamine (51 μL, 7.0 Eq, 0.29 mmol). The mixture was stirred at room temperature for 18 h and purified by reverse phase chromatography (acetonitrile/water(0.1% formic acid), 5% for 2 CV, 5% to 95% in 12 CV, 95% for 2 CV) to afford Compound 38 as a white solid (16 mg, 8%). HPLC: 96% @220 nm. LC-MS (ESI−) Exact mass calculated for [C₂₂₈H₃₄₁N₄₄O₆₄S₃]³⁻ [M-3H]³⁻: 1605.8, found: 1605.9. Exact mass calculated for [C₂₂₈H₃₄₀N₄₄O₆₄S₃]⁴⁻ [M-4H]⁴⁻: 1204.1, found: 1204.1

### Example 39: Synthesis of Compound 39

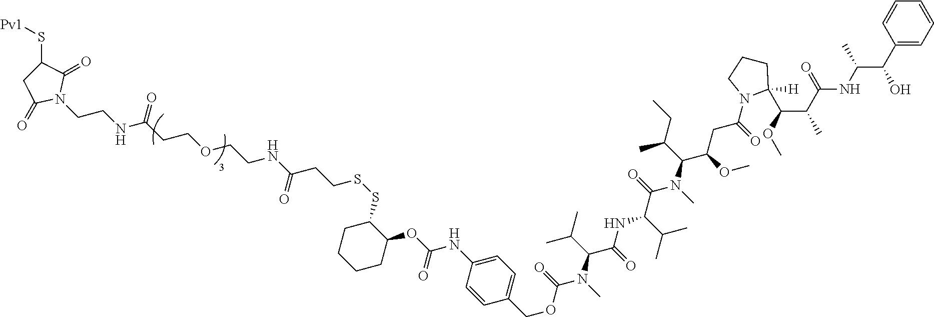

### Step 1: Synthesis of allyl 2,2-dimethyl-4-oxo-3,8,11,14-tetraoxa-5-azaheptadecan-17-oate (39-2)

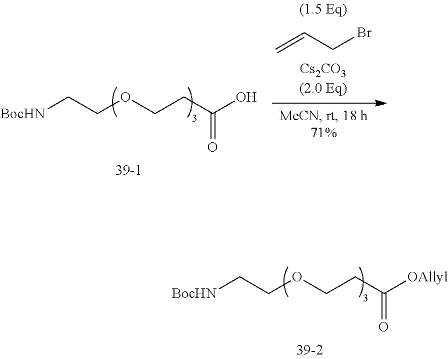

To a solution of 39-1 (2.00 g, 1.0 Eq. 6.23 mmol) in acetonitrile (50 mL) were added cesium carbonate (4.06 g, 2.0 Eq, 12.5 mmol) and allyl bromide (803 μL, 1.5 Eq, 9.35 mmol). The reaction mixture was stirred at room temperature for 18 h. The remaining cesium carbonate was filtered off and the solvent removed in vacuo. Purification by flash chromatography (EtOAc/cyclohexane, 0% for 2 CV, 0% to 100% in 10 CV) gave the title compound (1.60 g, 71%) as a white powder. ¹H NMR (400 MHz, CDCl₃) δ 5.91 (ddt, J=17.2, 10.4, 5.7 Hz, 1H), 5.37-5.18 (m, 2H), 5.11-4.73 (br s, 1H), 4.59 (dt, J=5.7, 1.4 Hz, 2H), 3.77 (t, J=6.5 Hz, 2H), 3.68-3.58 (m, 8H), 3.53 (dd, J=5.5, 4.7 Hz, 2H), 3.30 (t, J=5.1 Hz, 2H), 2.63 (t, J=6.5 Hz, 2H), 1.43 (s, 9H).

### Step 2: Synthesis of allyl 3-(2-(2-(2-aminoethoxy)ethoxy)ethoxy)propanoate hydrochloride (39-3)

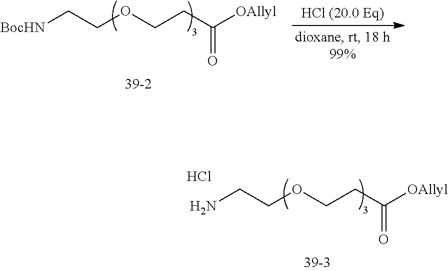

To a solution of 39-2 (1.60 g, 1.00 Eq, 4.23 mmol) in dioxane (20 mL) was added 4N HCl in dioxane (22.1 mL, 20.0 Eq, 88.5 mmol) and the reaction was stirred at room temperature for 18 h. The reaction was concentrated in vacuo and the residue was triturated with diethyl ether to afford the title compound (1.32 g, 99%) as a colorless oil. ¹H NMR (400 MHz, MeOD-d₄) 5.99-5.89 (m, 1H), 5.32 (dq, J=17.2, 1.6 Hz, 1H), 5.22 (dq, J=10.5, 1.4 Hz, 1H), 4.60 (dt, J=5.6, 1.5 Hz, 2H), 3.78-3.74 (m, 2H), 3.72-3.69 (m, 2H) 3.67-3.65 (m, 6H), 3.64-3.62 (m, 4H), 3.14-3.11 (m, 2H), 2.63 (t, J=6.0 Hz, 2H).

### Step 3: Synthesis of allyl 1-(((1S,2S)-2-(((4-(hydroxymethyl)phenyl)carbamoyl)oxy)cyclohexyl)disulfaneyl)-3-oxo-7,10,13-trioxa-4-azahexadecan-16-oate (39-4)

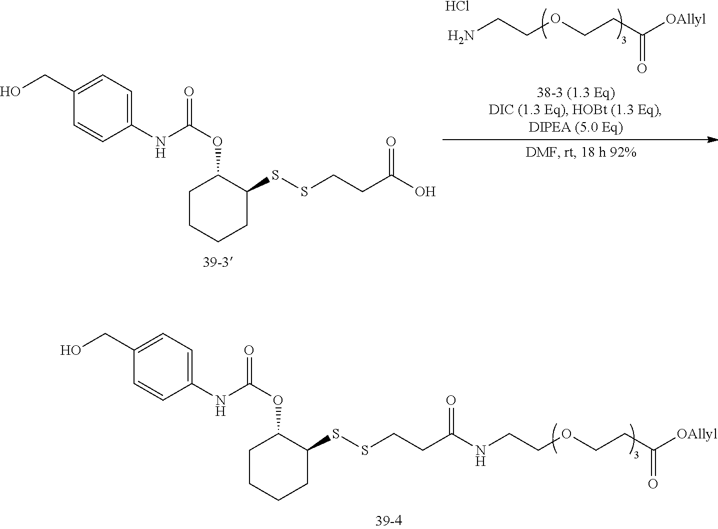

To a solution of 39-3′ (500 mg, 1.0 Eq, 1.30 mmol) in anhydrous DMF (10 mL) were added 1H-benzo[d][1,2,3]triazol-1-ol (228 mg, 1.3 Eq, 1.69 mmol), N,N′-diisopropylcarbodiimide (262 μL, 1.3 Eq, 1.69 mmol) and diisopropylethylamine (838 mg, 5.0 Eq, 6.49 mmol). The mixture was stirred for 10 min. Next, allyl 3-(2-(2-(2-aminoethoxy)ethoxy)ethoxy)propanoate hydrochloride (502 mg, 1.3 Eq, 1.69 mmol) in DMF (10 mL) was added and the solution continued to stir at room temperature for 18 h. The mixture was purified by reverse phase chromatography (methanol/water(0.1% formic acid), 5% for 2 CV, 5% to 95% in 12 CV, 95% for 2 CV) to afford the title compound (747 mg, 92%) as a white solid. ¹H NMR (400 MHz, DMSO-d₆) δ 7.97 (t, J=5.7 Hz, 1H), 7.41 (m, 2H), 7.21-7.19 (m, 2H), 5.90 (ddt, J=17.2, 10.6, 5.3 Hz, 1H), 5.34-5.17 (m, 2H), 5.07-5.02 (m, 1H), 4.65-4.57 (m, 1H), 4.55 (dt, J=5.3, 1.6 Hz, 2H), 4.43-4.48 (m, 2H), 3.63 (t, J=6.2 Hz, 2H), 3.53-3.43 (m, 8H), 3.38 (t, J=5.9 Hz, 2H), 3.22-3.13 (m, 3H), 2.92-2.83 (m, 3H), 2.57 (t, J=6.2 Hz, 2H), 2.44 (t, J=7.2 Hz, 2H), 2.17-1.99 (m, 2H), 1.77-1.28 (m, 6H). LC-MS (ESI+) Exact mass calculated for [C₂₉H₄₅N₂O₉S₂]⁺ [M+H]⁺: 629, found: 629.

### Step 4: Synthesis of allyl 1-(((1S,2S)-2-(((4-((((4-nitrophenoxy)carbonyl)oxy)methyl)phenyl)carbamoyl)oxy)cyclohexyl)disulfaneyl)-3-oxo-7,10,13-trioxa-4-azahexadecan-16-oate (5)

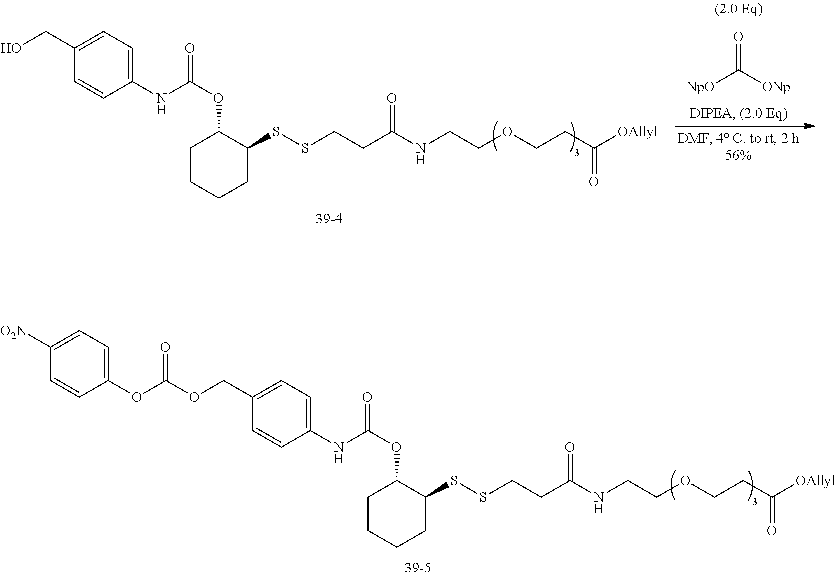

To a solution of 39-4 (740 mg, 1.0 Eq, 1.18 mmol) in anhydrous DMF (15 mL) at 4° C. was added bis(4-nitrophenyl) carbonate (716 mg, 2.0 Eq, 2.35 mmol) and diisopropylethylamine (607 μL, 3.0 Eq, 3.53 mmol). The mixture was allowed to warm to room temperature and stirred for 18 h. The mixture was purified by reverse phase chromatography (methanol/water(0.1% formic acid), 5% for 2 CV, 5% to 95% in 12 CV, 95% for 2 CV) to afford the title compound (520 mg, 56%) as a white solid. ¹H NMR (400 MHz, DMSO-d₆) δ 8.35-8.27 (m, 2H), 7.97 (t, J=5.6 Hz, 1H), 7.60-7.54 (m, 2H), 7.53-7.48 (m, 2H), 7.40-7.36 (m, 2H), 5.89 (ddt, J=17.3, 10.6, 5.3 Hz, 1H), 5.32-5.25 (m, 1H), 5.24-5.21 (m, 2H), 5.21-5.17 (m, 1H), 4.69-4.59 (m, 1H), 4.55 (dt, J=5.3, 1.6 Hz, 2H), 3.63 (t, J=6.2 Hz, 2H), 3.51-3.43 (m, 8H), 3.37 (t, J=5.9 Hz, 2H), 3.22-3.12 (m, 3H), 2.88 (t, J=6.9 Hz, 3H), 2.56 (t, J=6.2 Hz, 2H), 2.44 (t, J=7.2 Hz, 2H), 2.17-2.02 (m, 2H), 1.75-1.31 (m, 6H). LC-MS (ESI+) Exact mass calculated for [C₃₆H₄₈N₃O₁₃S₂]⁺ [M+H]⁺: 794, found: 794.

### Step 5: Synthesis of 39-6

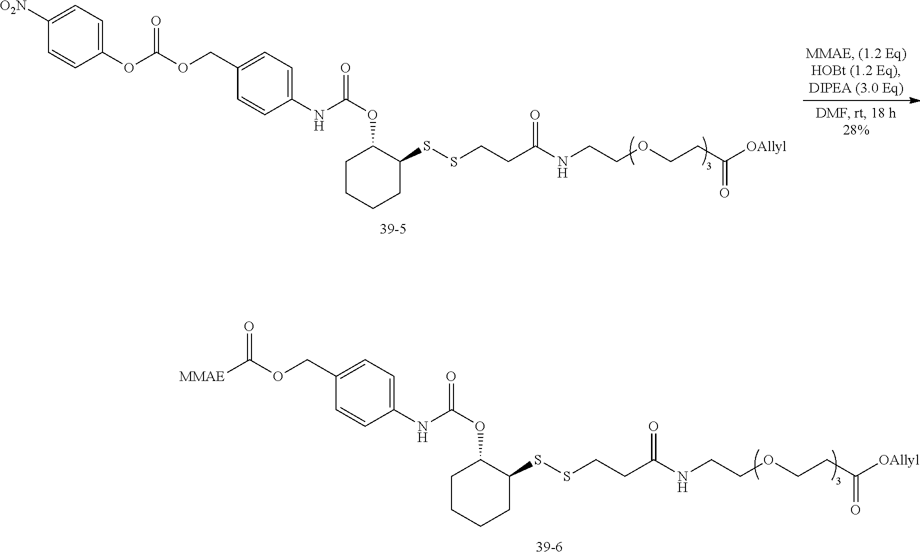

To a solution of 39-5 (420 mg, 1.0 Eq, 529 μmol) in DMF (4 mL) were added HOBt (109 mg, 1.2 Eq, 635 μmol), diisopropylethylamine (273 μL, 3.0 Eq, 1.59 mmol), MMAE (456 mg, 1.2 Eq, 635 μmol) and 3 Å molecular sieves. The reaction was stirred at room temperature for 18 h. The mixture was purified by reverse phase chromatography (methanol/water(0.1% formic acid), 5% for 2 CV, 5% to 95% in 12 CV, 95% for 2 CV) to afford the title compound (313 mg, 28%) as a fluffy white solid. LC-MS (ESI+) Exact mass calculated for [C₆₉H₁₁₀N₇O₁₇S₂]⁺ [M+H]*: 1372.7, found: 1372.9.

### Step 6: Synthesis of 39-7

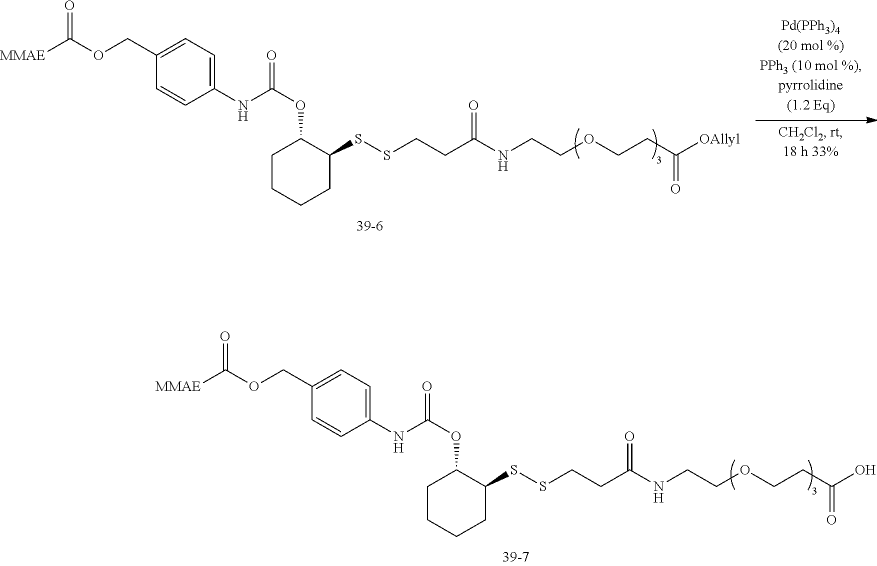

To a solution of 39-6 (200 mg, 1.0 Eq, 146 μmol) in dry CH₂C₁₂ (2 mL) was added triphenylphosphine (3.8 mg, 10 mol-%, 15 μmol). The solution was purged with nitrogen for 2 minutes then Pd(PPh₃)₄ (33.7 mg, 20 mol-%, 29.1 μmol) and pyrrolidine (14 μL, 1.2 Eq, 175 μmol) were added. The mixture was allowed to stir at room temperature for 18 h. The reaction was concentrated in vacuo and the residue was purified by reverse phase chromatography (methanol/water(0.1% formic acid), 5% for 2 CV, 5% to 95% in 12 CV, 95% for 2 CV) to afford the title compound (64 mg, 33%) as a fluffy yellow solid. The ¹H NMR spectrum is too complex for the interpretation. LC-MS (ESI+) Exact mass calculated for [C₆₆H₁₀₆N₇O₁₇S₂]⁺ [M+H]⁺: 1332.7, found: 1332.5.

### Step 7: Synthesis of 39-8

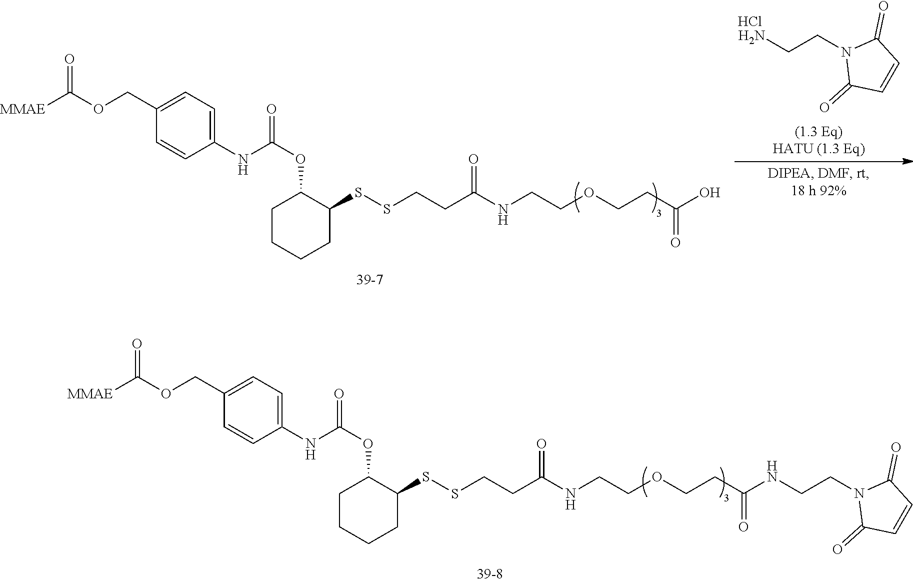

To a solution of 39-7 (60 mg, 1.0 Eq, 45 μmol) in DMF (4 mL) was added HATU (22 mg, 1.3 Eq, 59 μmol) and diisopropylethylamine (31 μL, 4.0 Eq, 0.18 mmol). After 15 min stirring at room temperature a solution of 1-(2-aminoethyl)-1H-pyrrole-2,5-dione hydrochloride (10 mg, 1.3 Eq, 59 μmol) in DMF (4 mL) was added and the mixture was stirred at room temperature for 18 h. The reaction was purified by reverse phase chromatography (acetonitrile/water(0.1% formic acid), 5% for 2 CV, 5% to 95% in 12 CV, 95% for 2 CV) to afford the title compound as a white solid (60 mg, 92%). LC-MS (ESI+) Exact mass calculated for [C₇₂H₁₁₂N₉O₁₈S₂]⁺ [M+H]+: 1454.7, found: 1454.8.

### Step 8: Synthesis of Compound 39

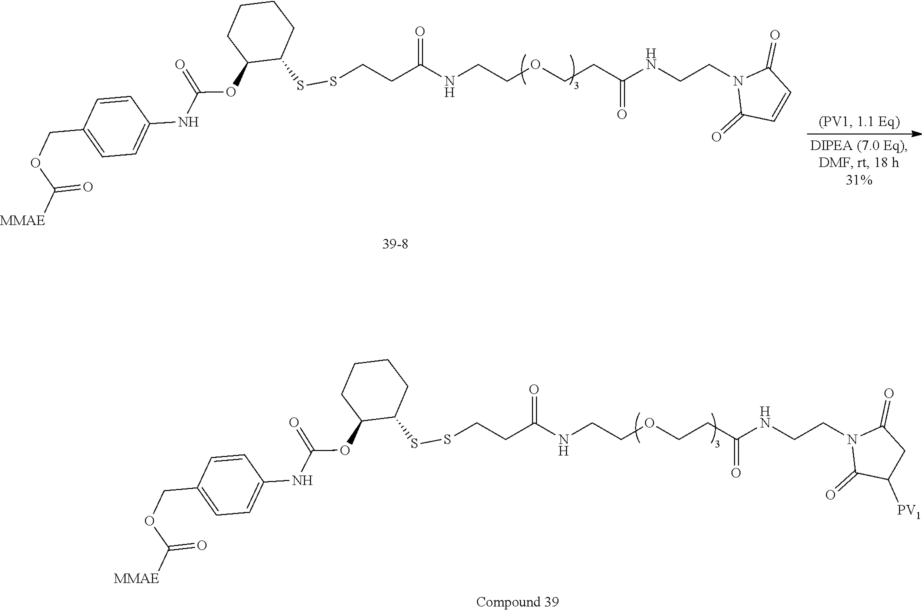

To a solution of 39-8 (60 mg, 1.0 Eq, 41 μmol) in DMF (3 mL) was added Pv1 (150 mg, 1.1 Eq, 45 μmol) and diisopropylethylamine (50 μL, 7.0 Eq, 0.29 mmol). The mixture was stirred at room temperature for 18 h and purified by reverse phase chromatography (acetonitrile/water(0.1% formic acid), 5% for 2 CV, 5% to 95% in 12 CV, 95% for 2 CV) to afford the title compound as a white solid (60 mg, 31%). HPLC: 99% @220 nm. LC-MS (ESI+) Exact mass calculated for [C₂₂₄H₃₃₃N₄₄O₆₂S₃]³⁻ [M−H]⁻: 1576.5, found: 1576.2. Exact mass calculated for [C₂₂₄H₃₃₂N₄₄O₆₂S₃]⁴⁻ [M−H]⁻: 1182.1, found: 1182.8

### Example A: In Vitro Growth Delay Assay in Cancer Cells

Cells (HCT116 colorectal cells, PC3 prostate cells, NCI-H1975 NSCLC cells, and NCI-H₂₉₂ NSCLC cells) were plated at 3000 cells per well in 96 well black walled-clear bottom plates (Griener) in growth media containing 10% FBS. Cells were allowed to adhere at room temperature for 60 minutes before returning to a 37° C., 5% CO₂ incubator. After 24 hours, media was removed and replaced with fresh growth media containing various drug concentrations. Each drug concentration was added in triplicate. Non-drug treated controls contained growth media only. Cells were returned to the incubator. Ninety-six hours after addition of drug, cells were fixed with 4% paraformaldehyde for 20 minutes and stained with Hoechst at 1 μg/mL. The plates were imaged on a Cytation 5 auto imager (BioTek) and cells were counted using CellProfiler (http://cellprofiler.org). The percent cell growth delay was calculated and data plotted using GraphPad Prism.

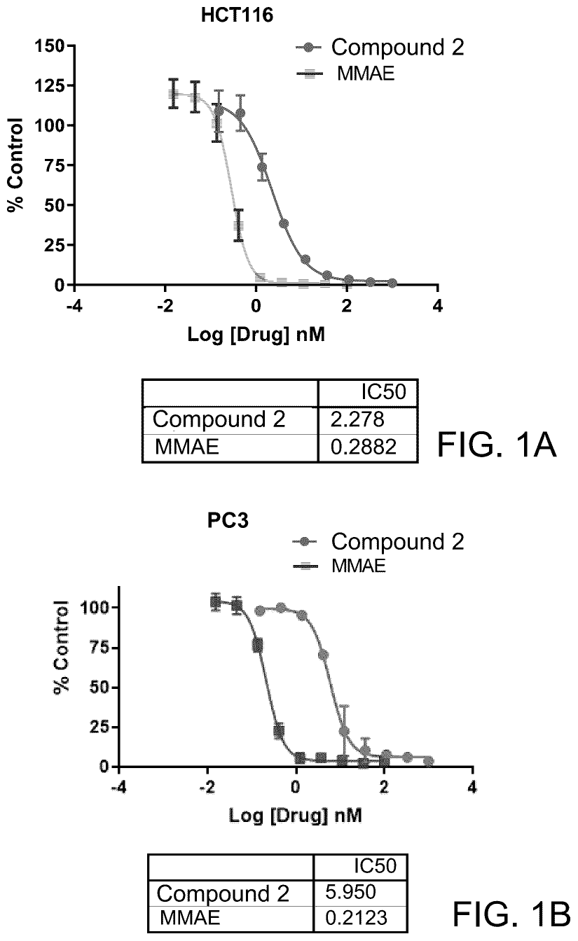

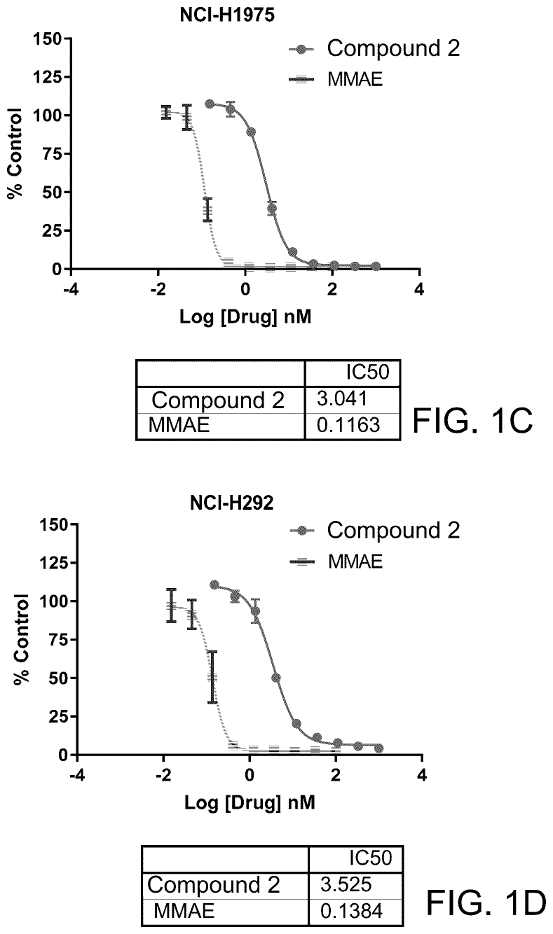

FIG. 1A shows a plot of the growth delay of HCT116 colorectal cells in vitro after four day incubation with the indicated concentrations of Compound 2 or unconjugated MMAE.

FIG. 1B shows a plot of the growth delay of PC3 prostate cells in vitro after four day incubation with the indicated concentrations of Compound 2 or unconjugated MMAE.

FIG. 1C shows a plot of the growth delay of NCI-H1975 NSCLC cells in vitro after four day incubation with the indicated concentrations of Compound 2 or unconjugated MMAE.

FIG. 1D shows a plot of the growth delay of NCI-H292 NSCLC cells in vitro after four day incubation with the indicated concentrations of Compound 2 or unconjugated MMAE.

The following table shows the HCT116 colorectal cell 4-day growth inhibition (IC₅₀) after treatment with the indicated example compound.

| Example No. | IC₅₀ (nM) |
|---|---|
| 1 | 2.0 |
| 2 | 1.9 |
| 3 | 2.8 |
| 4 | 3.0 |
| 5 | 10.4 |
| 6 | 7.8 |
| 7 | 12.1 |
| 8 | ND |
| 9 | ND |
| 10 | 1.1 |
| 11 | 6.3 |
| 12 | 1.4 |
| 13 | 1.6 |
| 14 | ND |
| 15 | ND |
| 16 | 1.7 |
| 17 | 4.3 |
| 18 | 5.6 |
| 19 | 4.2 |
| 20 | 0.7 |
| 21 | ND |
| 22 | ND |
| 23 | ND |
| 24 | ND |
| 25 | 165 |
| 26 | 155 |
| 27 | 158 |
| 28 | 297 |
| 29 | 322 |
| 30 | 750 |
| 31 | 733 |
| 32 | ND |
| 33 | 291 |
| 34 | 285 |
| 35 | 2825 |
| 36 | 1107 |
| 37 | 291 |
| 38 | ND |
| 39 | ND |

ND = not determined.

### Example B: In Vitro Cell Cycle Arrest Functional Assay in Cancer Cells

Cell Incubation with MMAE and Compound 2 and Staining with Propidium Iodide

HCT116 cells were seeded in 6-well tissue culture plates at 500,000 cells per well in 2 mL of DMEM and incubated overnight in a 37° C., 5% CO₂ incubator. 200 μL of dilutions of MMAE and Compound 2 which were made at 10× concentrations in DMEM+4% DMSO were added to appropriate wells of the 6-well plates and plates were incubated for 24 hours. After exposure of HCT116 cells to either MMAE or Compound 2, cells were harvested for propidium iodide staining and flow cytometry. Media was collected from each well and transferred into conical 15 mL centrifuge tubes to collect nonadherent cells. PBS (1 mL) was added to wash wells and was then transferred to the 15 mL tubes. Tryp-LE (1 mL) was added to each well and plates were incubated for 5 minutes in a 37° C., 5% CO₂ incubator until the cells lifted off the well surface. A solution of DMEM+10% fetal bovine serum (1 mL) was added to each well. Wells were triturated and cells transferred to tubes. A solution of DMEM+10% Fetal bovine serum (1 mL) was added to wells to ensure collection of cells. These were again transferred to the 15 mL tubes. Cell counts and viability for each sample was assessed by trypan blue exclusion on a Bio-Rad TC20 cell counter. Cells were centrifuged at 1200 rpm for 5 minutes. Supernatant was decanted and cells were resuspended in PBS at 1×10⁶ cells/mL for staining with propidium iodide.

### Analysis of Propidium Iodide-Stained Cells by Flow Cytometry

### Cell Staining Conditions

| | |
|---|---|
| Propidium iodide | Final concentration - 300 μg/mL |
| RNase | Final concentration - 50 μg/mL |

An aliquot (1 mL) of each cell suspension was transferred into a deep-well polypropylene plate. The plate was centrifuged at 1200 rpm for 5 minutes. The supernatant was decanted, and cells were resuspended in 330 μL of cold PBS. A volume of 670 μL of cold ethanol was slowly added to the sides of each well. Cells were gently triturated achieve uniform 67% ethanol for cell fixation on ice for 3 hours prior to staining. After fixation, the plate was centrifuged for 5 minutes at 1200 rpm and the ethanol:PBS was decanted. Cell were resuspended in 200 μL of a solution of RNase at 300 μg/mL and propidium iodide at 50 μg/mL in PBS. The plate was sealed and incubated in the dark for 30 minutes at 37° C. or overnight at room temperature, after which cells were resuspended and transferred to a small volume polypropylene plate for flow cytometry. Propidium iodide-stained cells were analyzed using a BD Acuri flow cytometer. For each sample, three plots were created:

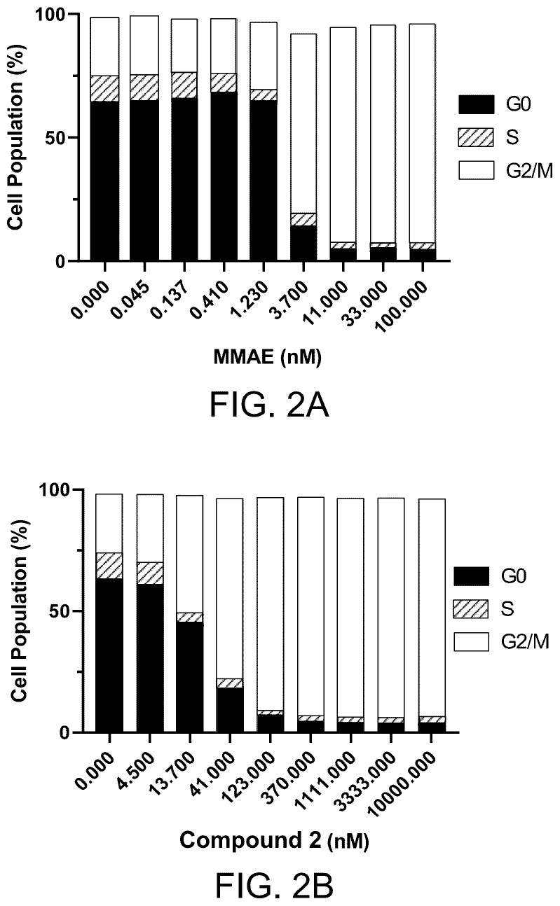

FIG. 2A shows a cell cycle analysis of HCT116 colorectal cells in vitro after 24 h incubation with the indicated doses of unconjugated MMAE.

FIG. 2B shows cell cycle analysis of HCT116 colorectal cells in vitro after 24 h incubation with the indicated doses of Compound 2.

Cells display dose responsive accumulation in G2/M, with an IC₅₀ of 2.6 nM for MMAE and an IC₅₀ of 19.6 nM for Compound 2.

### Example C: Plasma Pharmacokinetics of Compound 2 in a Rat Model

### Animal Dosing

Female Sprague Dawley rats underwent jugular vein cannulation and insertion of a vascular access button (VAB, Instech Labs Cat #VABR1B/22) at Envigo Labs prior to shipment. Magnetic, aluminum caps (Instech Labs Cat #Cat #VABRC) were used to protect the access port for the jugular catheters allowing the animals to be housed 2 per cage on corn cob bedding for 4-5 days prior to the study. Rats were administered a single intravenous dose of 10 mg/kg Compound 2 prepared in a vehicle of 5% mannitol in citrate buffer. At 2 min, 30 min, 1, 2 hours, 4 hours, 7 hours and 24 hours following compound administration, blood (250 μL) was collected into K2EDTA filled microtainers from fed rats. Plasma was isolated by centrifugation and 100 μL aliquots were transferred to 96-well polypropylene plates on dry ice. Samples were stored at −80° C. until processed for quantification by LC-MS/MS.

### LC-MS/MS Determination of Plasma Conjugate Concentration

A 20 μL volume of each sample (double blanks (D-BLK), blanks (BLK), standards (STDs), quality controls (QCs) or matrix sample) was added to a clean, 1 mL 96-well protein precipitation plate containing 20 μL of 4% phosphoric acid in water. Fortified samples were vortexed at 700 rpm for 2 minutes and subsequently centrifuged for 1 minute at 1500 rpm to consolidate all liquid to the bottom of the plate. A 20 μL volume of working internal standard (WIS) was added to each matrix sample followed 180 μL of acetonitrile:methanol:formic acid, (500:500:1, v:v:v). Samples were vortexed at 700 rpm for 2 minutes and centrifuged at 3000 rpm for 10 minutes at 4° C. A 50 μL volume of supernatant was transferred to a clean LoBind 0.700-mL 96-well polypropylene collection plate followed by the addition of 100 μL of water:acetonitrile:formic acid (900:100:1, v:v:v). Final samples were vortexed at 700 rpm for 2 minutes and 5 μL was injected onto the LC-MS/MS system for analysis.

### LC-MS/MS Determination of Plasma MMAE Concentration

A 25 μL volume of each matrix sample was added to the wells of a 96-well polypropylene plate followed by 150 μL of ammonium formate buffer (pH 6.9) and 25 μL of working internal standard (WIS). For double blank controls, the WIS was substituted by 25 μL of water:acetonitrile:formic acid (500:500:1, v:v:v). Fortified samples were vortexed at 700 rpm for 2 minutes. Working on a negative pressure manifold, a 200 μL volume of fortified matrix sample was added to a supported liquid extraction plate and samples were allowed to percolate through the plate frit under a negative pressure of 650-700 torr for up to 1 minute. Samples were allowed to completely absorb into the plate for 5 minutes. Prior to elution, a 2-mL 96-well TrueTaper plate was placed within the vacuum manifold for use as a collection plate. A 1000 μL volume of MTBE was added to the original sample plate and the solvent was allowed to flow under gravity for 5 minutes. A negative pressure of ˜650 torr was applied for 10-30 sections or until the sample was completely evacuated from the wells. Collected elutions were evaporated under a heated stream of nitrogen at 40° C. Samples were reconstituted in 100 μL of acetonitrile:water:200 mM ammonium formate (90:5:5, v:v:v) and covered with a silicone cap mat. Final samples were vortexed at 900 rpm for 2 minutes and subsequently centrifuged at 3000 rpm for 5 minutes at 4° C. Analysis was accomplished by injecting a 10 μL sample onto an LC-MS/MS system.

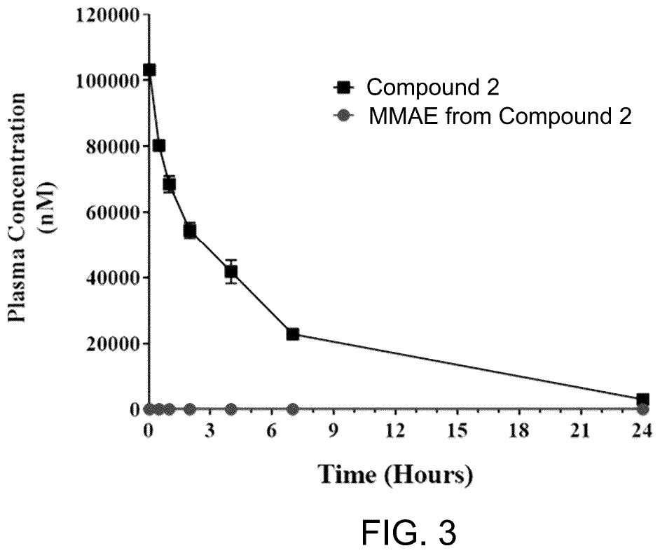

FIG. 3 shows a plot of the plasma concentration of Compound 2 and released MMAE after a single IV dose of 10 mg/kg of Compound 2 in the rat (data are expressed as means±SD). As shown in FIG. 3, 0.02% of the MMAE warhead was released after 24 h in circulation. FIG. 3 demonstrates that Compound 2 is stable in plasma for at least 24 h.

### Example D: Tissue Pharmacokinetics of Compound 2 in a Mouse Model

### Animal Dosing

Six-week-old female athymic nude Foxn^{nu} mice were obtained from Taconic Labs (Cat #NCRNU-F) and were housed 5 per cage on Alpha-Dri bedding in a disposable caging system (Innovive). Human HCT 116 cancer cells derived from colorectal carcinoma were diluted 1:1 in Phenol Red-free Matrigel and subcutaneously implanted into the left flank of each mouse at a density of 2.5×10⁶ cells in 100 μL. When xenografts reached a minimal volume of 300 mm³, mice were administered a single intraperitoneal injection of 0.5 mg/kg MMAE or 3 mg/kg Compound 2 prepared in a vehicle of 5% mannitol in citrate. Tumor, quadriceps muscle and bone marrow samples were collected from fed, anesthetized mice at 4, 24 and 48 hours after compound administration. MMAE concentrations in tissues were determined via LCMS.

### LC-MS/MS Determination of Plasma and Tissue MMAE Concentration

Plasma MMAE

A 75 μL volume of acetonitrile:formic acid (1000:1, v:v) containing internal standard (MMAE-D8) to the wells of a 96-well protein precipitation plate resting on top of a 0.700 mL 96-well LoBind polypropylene plate. Double blank and carryover sample wells received 75 μL of acetonitrile:formic acid (1000:1, v:v) without internal standard. A 25 μL volume of each matrix sample was added to the plate wells containing internal standard. Fortified samples were vortexed at 700 rpm for 1 minute and centrifuged at 3000 rpm for 2 minutes at 4° C. The protein precipitation plate was discarded. A 50 μL volume of mobile phase A (acetonitrile:water:200 mM ammonium formate (90:5:5, v:v:v)) was added to the 96-well polypropylene collection plate which was covered with a silicone cap mat. Final samples were vortexed at 700 rpm for 2 minutes and analysis was accomplished by injecting a 2 μL sample onto an LC-MS/MS system.

Tumor and Muscle MMAE

Thawed tissue samples kept on wet ice were adjusted to 100 mg/mL with PBS based on tissue weight. Samples were homogenized on a Precellys Evolution machine at 7200 rpm for 2×30 second cycles with a 10 second pause in between each cycle. Homogenates were centrifuged at 14,000 rpm for 5 minutes at 4° C. and the supernatants were transferred to clean 2 mL LoBind Eppendorf tubes. A 100 μL volume of homogenate was added to a clean 2 mL 96-well polypropylene plate followed by 75 μL of ammonium formate buffer, pH 6.9, and 25 μL of working internal standard (WIS). Double blank controls received 75 μL of water:acetonitrile:formic acid (1:1:0.001, v:v:v) without internal standard. Fortified samples were covered with a silicone cap mat and vortexed at 700 rpm for 2 minutes. Working on a negative pressure manifold, 200 μL of fortified matrix samples were added to a supported liquid extraction (SLE) plate and samples were allowed to percolate through the plate frit with a negative pressure of ˜650-700 torr for up to 1-minute. Samples were allowed to completely absorb into the SLE plate for 5 minutes. Prior to sample elution, a 2-mL 96-well TrueTaper collection plate was placed within the vacuum manifold as the collection vessel. Samples were evaporated under a heated stream of nitrogen at 40° C. and reconstituted in 150 μL of acetonitrile:water:200 mM ammonium formate (90:5:5, v:v:v). The collection plate was covered with a silicone cap mat and vortexed at 900 rpm for 2 minutes. Final samples were centrifuged at 3000 rpm for 5 minutes at 4° C. and analysis was accomplished by injecting a 2 μL sample onto an LC-MS/MS system.

Bone Marrow MMAE

Thawed bone marrow sample pellets kept on wet ice were adjusted to a final concentration of 1.0×10⁷ with ice-cold RIPA buffer. Samples were homogenized on a Precellys Evolution machine at 7200 rpm for 2×30 second cycles with a 10 second pause in between each cycle. Bone marrow cell homogenates were centrifuged at 14,000 rpm for 5 minutes at 4° C. and the supernatants were transferred to clean 2 mL LoBind Eppendorf tubes. A 200 μL volume of each bone marrow cell homogenate was added to the wells of a clean 2 mL 96-well polypropylene plate followed by 175 μL of ammonium formate buffer, pH 6.9, and 25 μL of working internal standard (WIS). Double blank controls received 175 μL of water:acetonitrile (1:1, v:v:v) only without internal standard. Fortified samples were covered with a silicone cap mat and vortexed at 700 rpm for 2 minutes. Working on a negative pressure manifold, 400 μL of fortified matrix samples were added to a supported liquid extraction (SLE) plate and samples were allowed to percolate through the plate frit with a negative pressure of ˜650-700 torr for up to 1-minute. Samples were allowed to completely absorb into the SLE plate for 5 minutes. Prior to sample elution, a 2-mL 96-well TrueTaper collection plate was placed within the vacuum manifold as the collection vessel. Elution was accomplished by applying 900 μL of MTBE:ethyl acetate (1:1, v:v) to the system and allowing the solvent to flow under gravity for 5 minutes. Negative pressure of −650 torr was applied for 10-30 seconds or until the wells were completely evacuated. The elution process was repeated. Samples were evaporated under a heated stream of nitrogen at 40° C. and reconstituted in 25 μL of water:acetonitrile:formic acid (900:100:1, v:v:v). The collection plate was covered with a silicone cap mat and vortexed at 900 rpm for 2 minutes. Final samples were centrifuged at 3000 rpm for 5 minutes at 4° C. and analysis was accomplished by injecting 2 μL was injected onto an LC-MS/MS system.

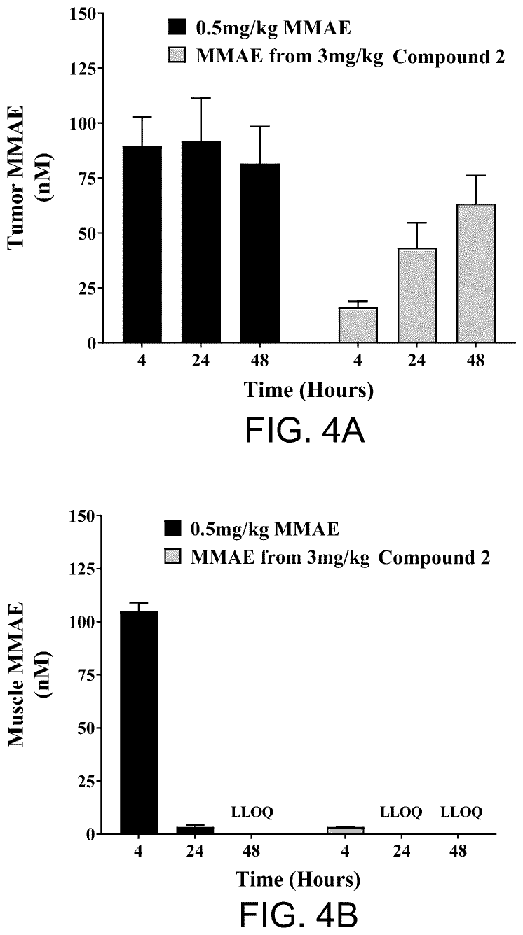

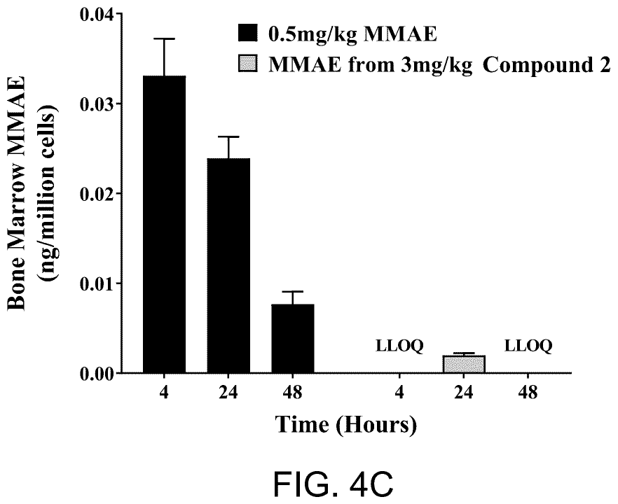

FIG. 4A shows a plot of the levels of unconjugated MMAE in mouse tumor determined by LCMS after a single intraperitoneal injection of either 0.5 mg/kg MMAE or 3 mg/kg Compound 2 in HCT116 colorectal tumor bearing female nude mice.

FIG. 4B shows a plot of the levels of unconjugated MMAE in mouse muscle determined by LCMS after a single intraperitoneal injection of either 0.5 mg/kg MMAE or 3 mg/kg Compound 2 in HCT116 colorectal tumor bearing female nude mice.

FIG. 4C shows a plot of the levels of unconjugated MMAE in mouse bone marrow determined by LCMS after a single intraperitoneal injection of either 0.5 mg/kg MMAE or 3 mg/kg Compound 2 in HCT116 colorectal tumor bearing female nude mice.

Dosing the unconjugated MMAE warhead results in indiscriminate distribution of MMAE across all tissues. In contrast, dosing Compound 2 results in tumor selective delivery of MMAE warhead, with efficient delivery of MMAE to tumor, but not to healthy tissues.

### Example E: Efficacy of Compound 1 in a HCT116 Colorectal Xenograft Model

Six-week-old female athymic nude Foxn^{nu} mice were obtained from Taconic Labs (Cat #NCRNU-F) and were housed 5 per cage on Alpha-Dri bedding in a disposable caging system. Human HCT 116 cells derived from colorectal carcinoma were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle, 0.25 mg/kg MMAE or 40 mg/kg Compound 1 (equivalent 7 mg/kg unconjugated MMAE). Doses were prepared by diluting 0.1 mg/μL DMSO stocks in 5% mannitol in citrate buffer and were administered for two doses at a volume of 12 mL/kg (300 μL per 25 g mouse). Xenograft tumors were measured by calipers and volume was calculated using the equation for ellipsoid volume: Volume=π/6×(length)×(width)². Animals were removed from the study due to death, tumor size exceeding 2000 mm³ or loss of >20% body weight. The below table shows the dosing schedule of various treatment groups.

| | | | | | Adminis- | |
|---|---|---|---|---|---|---|
| | | | | Dosing | tration | Number |
| Group | Treatment | Dose | | Schedule | Route | of Mice |
| 1 | Vehicle | NA | | QDx2 | i.p. | 8 |
| | (5% | | | | | |
| | mannitol | | | | | |
| | in citrate | | | | | |
| | buffer) | | | | | |
| 2 | MMAE | 0.25 | mg/kg | QDx2 | i.p. | 8 |
| 3 | Compound 1 | 40 | mg/kg | QDx2 | i.p. | 8 |

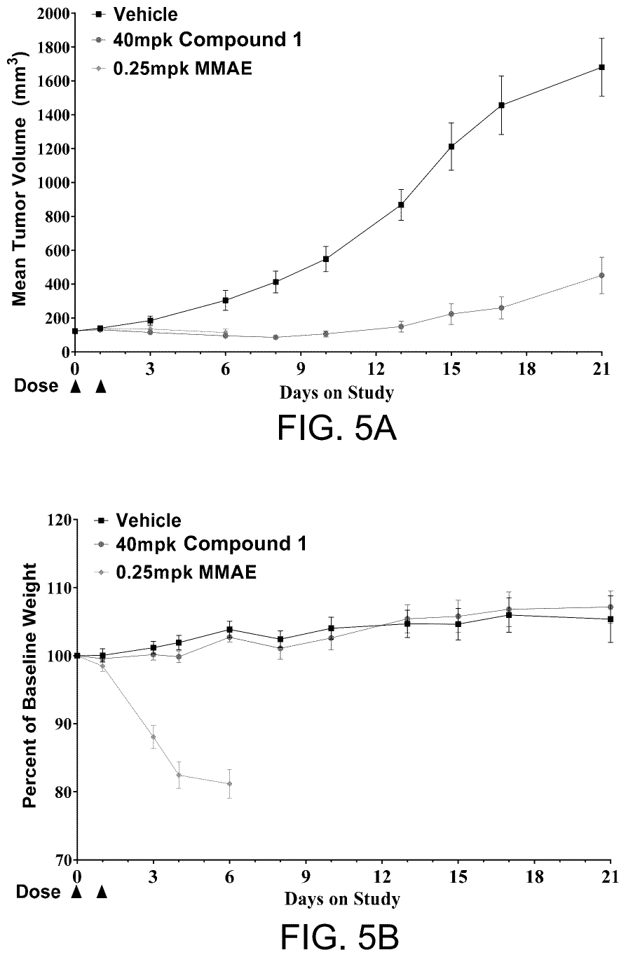

FIG. 5A shows a plot of the mean tumor volume resulting from dosing either 0.25 mg/kg MMAE or 40 mg/kg Compound 1 (7 mg/kg MMAE equivalent) in nude mice bearing HCT116 HER2 negative colorectal flank tumors. Animals were dosed once daily intraperitoneally for a total of two days.

FIG. 5B shows a plot of the percent change in body weight of nude mice bearing HCT116 HER2 negative colorectal flank tumors, dosed with either 0.25 mg/kg MMAE or 40 mg/kg Compound 1 (7 mg/kg MMAE equivalent).

Animals dosed with unconjugated MMAE experienced rapid decline in body weight and were removed from the study by day 6. In contrast, animals dosed with Compound 1 experienced no change in body weight. These data demonstrate that Compound 1 demonstrates potent anti-tumor activity and safety in a pre-clinical colorectal cancer model.

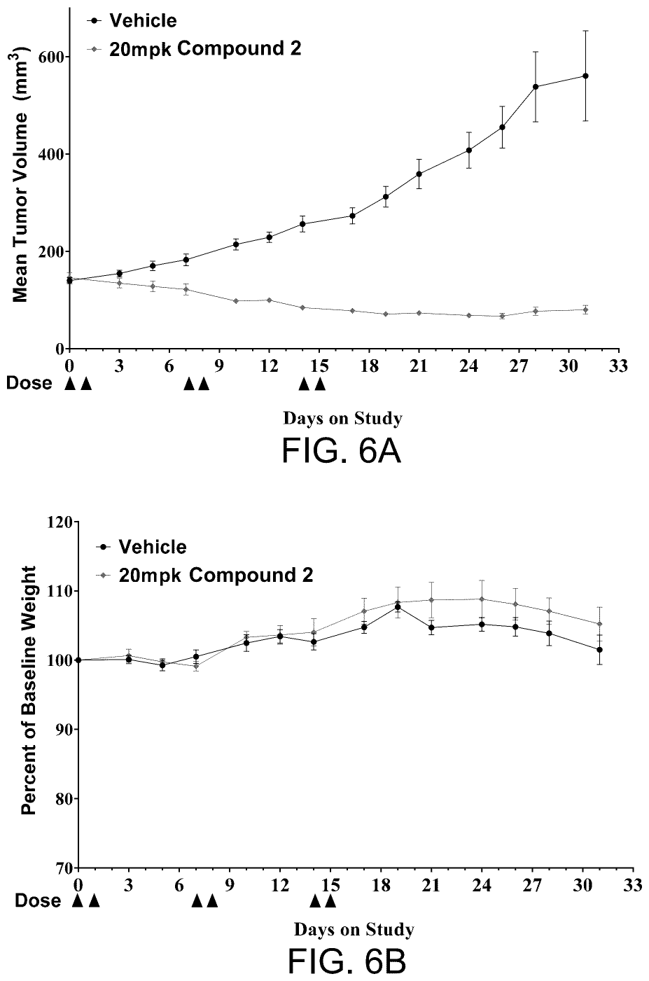

### Example F: Efficacy of Compound 2 in a PC3 Prostate Xenograft Model (Goes with FIG. 6)

Six-week-old female athymic nude Foxn^{nu} mice were obtained from Taconic Labs (Cat #NCRNU-F) and were housed 5 per cage on Alpha-Dri bedding in a disposable caging system. Human PC3 cells derived from prostate carcinoma were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle or 20 mg/kg Compound 2. Doses were prepared by diluting 0.1 mg/μL DMSO stocks in 5% mannitol in citrate buffer and were administered QD×2/week for 3 weeks at a volume of 12 mL/kg (300 μL per 25 g mouse). Xenograft tumors were measured by calipers and volume was calculated using the equation for ellipsoid volume. Volume=π/6×(length)×(width)². Animals were removed from the study due to death, tumor size exceeding 2000 mm³ or loss of >20% body weight. The below table shows the dosing schedule of various treatment groups.

| | | | | Adminis- | |
|---|---|---|---|---|---|
| | | | Dosing | tration | Number |
| Group | Treatment | Dose | Schedule | Route | of Mice |
| 1 | Vehicle | NA | QDx2/wk × | i.p. | 5 |
| | (5% mannitol | | 3 wks | | |
| | in citrate | | | | |
| | buffer) | | | | |
| 2 | Compound 2 | 20 mg/kg | QDx2/wk × | i.p. | 5 |
| | | | 3 wks | | |

FIG. 6A shows a plot of the mean tumor volume resulting from dosing 20 mg/kg Compound 2 in nude mice bearing PC3 prostate adenocarcinoma flank tumors. Animals were dosed once daily two times per week intraperitoneally for three weeks.

FIG. 6B displays percent change in body weight of animals in this study. Data are expressed as means±SEM.

These data demonstrate that Compound 2 demonstrates potent anti-tumor activity and safety in a pre-clinical prostate cancer model. Animals dosed with Compound 2 experienced no change in body weight.

### Example G: Efficacy of Compound 2 in a NCI-H1975 Non-Small Cell Lung Xenograft Model

Six-week-old female athymic nude Foxn^{nu} mice were obtained from Taconic Labs (Cat #NCRNU-F) and were housed 5 per cage on Alpha-Dri bedding in a disposable caging system. Human NCI-H1975 cells derived from non-small cell lung cancer were diluted 1:1 in Phenol Red-free Matrigel and subcutaneously implanted into the left flank of each mouse at a density of 5×10⁶ cells in 100 μL. When xenografts reached a mean volume of 100-200 mm³, mice were randomized into groups and treated as detailed in the table below. Mice were administered intraperitoneal (IP) doses of vehicle, 10 or 20 mg/kg Compound 2. Doses were prepared by diluting 0.1 mg/μL DMSO stocks in 5% mannitol in citrate buffer and were administered QD×2/week for 3 weeks at a volume of 12 mL/kg (300 μL per 25 g mouse). Xenograft tumors were measured by calipers and volume was calculated using the equation for ellipsoid volume: Volume=π/6×(length)×(width)². Animals were removed from the study due to death, tumor size exceeding 2000 mm³ or loss of >20% body weight. The below table shows the dosing schedule of various treatment groups.

| | | | | Adminis- | |
|---|---|---|---|---|---|
| | | | Dosing | tration | Number |
| Group | Treatment | Dose | Schedule | Route | of Mice |
| 1 | Vehicle | NA | QDx2/wk × | i.p. | 5 |
| | (5% mannitol | | 3 wks | | |
| | in citrate | | | | |
| | buffer) | | | | |
| 2 | Compound 2 | 10 mg/kg | QDx2/wk × | i.p. | 5 |
| | | | 3 wks | | |
| 2 | Compound 2 | 20 mg/kg | QDx2/wk × | i.p. | 5 |
| | | | 3 wks | | |

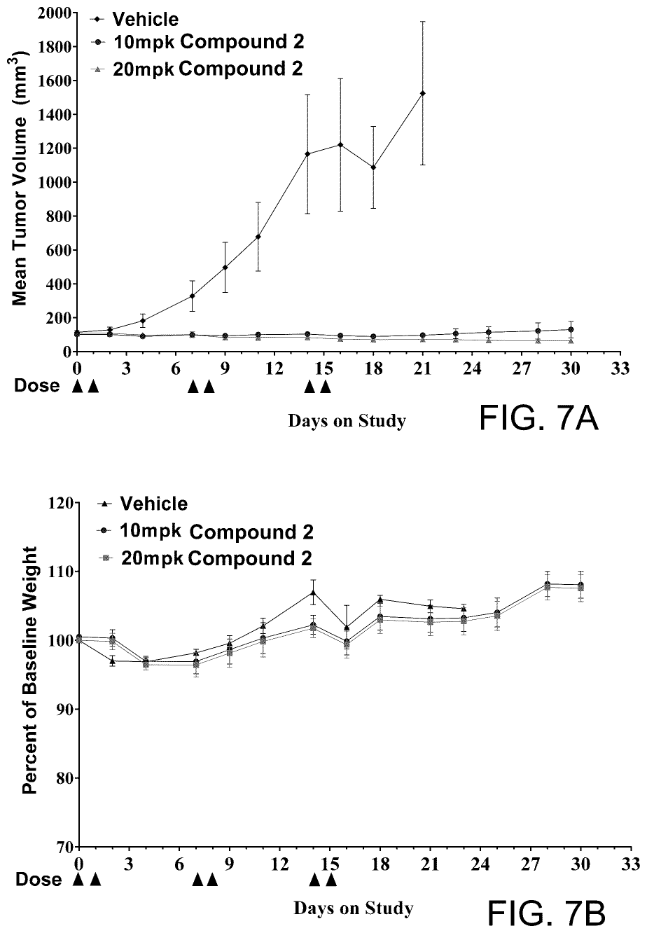

FIG. 7A shows a plot of the mean tumor volume resulting from dosing 10 or 20 mg/kg Compound 2 in nude mice bearing NCI-H1975 non-small cell lung cancer flank tumors. Animals were dosed once daily two times per week intraperitoneally for three weeks.

FIG. 7B displays percent change in body weight of animals in this study. Data are expressed as means±SEM.

These data demonstrate that Compound 2 demonstrates potent anti-tumor activity and safety in a pre-clinical non-small cell lung cancer model. Animals dosed with Compound 2 experienced no change in body weight.

### Example H: Safety of Compound 2 in Nude Mice

Six-week-old female athymic nude Foxn^{nu} mice were obtained from Taconic Labs (Cat #NCRNU-F) and were housed 3 per cage on Alpha-Dri bedding in a disposable caging system. Mice were administered intraperitoneal (IP) doses of vehicle, 10 or 20 mg/kg Compound 2. Doses were prepared by diluting 0.1 mg/μL DMSO stocks in 5% mannitol in citrate buffer and were administered daily for four consecutive days at a volume of 12 mL/kg (300 μL per 25 g mouse). The below table shows the dosing schedule of various treatment groups.

| | | | | Adminis- | |
|---|---|---|---|---|---|
| | | | Dosing | tration | Number |
| Group | Treatment | Dose | Schedule | Route | of Mice |
| 1 | Vehicle | NA | QDx4 | i.p. | 3 |
| | (5% mannitol | | | | |
| | in citrate | | | | |
| | buffer) | | | | |
| 2 | Compound 1 | 10 mg/kg | QDx4 | i.p. | 3 |
| 3 | Compound 2 | 10 mg/kg | QDx4 | i.p. | 3 |

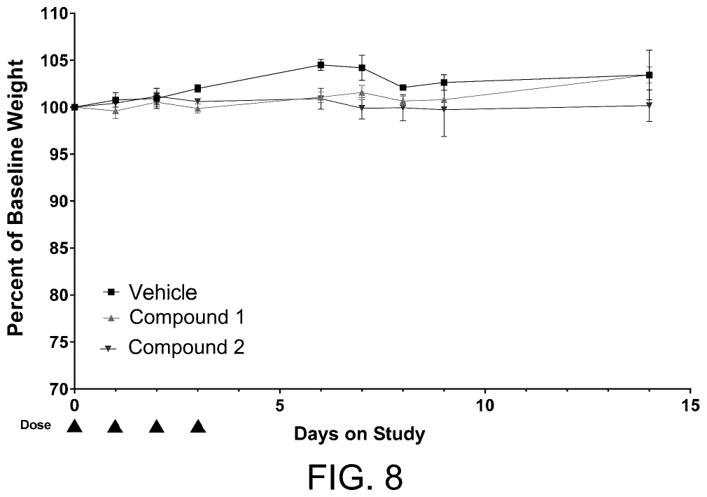

FIG. 8 shows a plot of body weights of nude mice dosed with 10 mg/kg Compound 1 and Compound 2 once daily for four consecutive days.

Animals dosed with Compound 1 and Compound 2 show no change in body weight, demonstrating the safety of these conjugates in the mouse.

### Example I: Tissue Pharmacokinetics of Compound 13 and Compound 7 in a Mouse Model

### Animal Dosing

Six-week-old female athymic nude Foxn^{nu} mice were obtained from Taconic Labs (Cat #NCRNU-F) and were housed 5 per cage on Alpha-Dri bedding in a disposable caging system (Innovive). Human HCT 116 cancer cells derived from colorectal carcinoma were diluted 1:1 in Phenol Red-free Matrigel and subcutaneously implanted into the left flank of each mouse at a density of 2.5×10⁶ cells in 100 μL. When xenografts reached a minimal volume of 300 mm³, mice were administered a single intraperitoneal injection of 10 mg/kg Compound 13 or Compound 7 prepared in a vehicle of 5% mannitol in citrate. Tumor was collected from fed, anesthetized mice at 2, 4, 8 and 24 hours after compound administration. MMAE concentrations in tumor was determined by LCMS and peptide concentrations determined by ELISA.

### LC-MS/MS Determination of Tissue MMAE Concentration

Thawed tissue samples kept on wet ice were adjusted to 100 mg/mL with PBS based on tissue weight. Samples were homogenized on a Precellys Evolution machine at 7200 rpm for 2×30 second cycles with a 10 second pause in between each cycle. Homogenates were centrifuged at 14,000 rpm for 5 minutes at 4° C. and the supernatants were transferred to clean 2 mL LoBind Eppendorf tubes. A 100 μL volume of homogenate was added to a clean 2 mL 96-well polypropylene plate followed by 75 μL of ammonium formate buffer, pH 6.9, and 25 μL of working internal standard (WIS). Double blank controls received 75 μL of water:acetonitrile:formic acid (1:1:0.001, v:v:v) without internal standard. Fortified samples were covered with a silicone cap mat and vortexed at 700 rpm for 2 minutes. Working on a negative pressure manifold, 200 μL of fortified matrix samples were added to a supported liquid extraction (SLE) plate and samples were allowed to percolate through the plate frit with a negative pressure of ˜650-700 torr for up to 1-minute. Samples were allowed to completely absorb into the SLE plate for 5 minutes. Prior to sample elution, a 2-mL 96-well TrueTaper collection plate was placed within the vacuum manifold as the collection vessel. Samples were evaporated under a heated stream of nitrogen at 40° C. and reconstituted in 150 μL of acetonitrile:water:200 mM ammonium formate (90:5:5, v:v:v). The collection plate was covered with a silicone cap mat and vortexed at 900 rpm for 2 minutes. Final samples were centrifuged at 3000 rpm for 5 minutes at 4° C. and analysis was accomplished by injecting 2 μL was injected onto an LC-MS/MS system.

### ELISA Measurement of Total Peptide Tissue Concentrations

96-well plates were coated with 100 μL/well of 0.1 μM BSA-labelled peptide prepared in 0.2 M Carbonate-Bicarbonate Buffer, pH 9.4 and incubated overnight at 4° C. Plates were washed 4× with an ELISA wash buffer (PBS+0.05% Tween 20), incubated for 2 hours at room temperature with Blocking Buffer (PBS+5% dry milk+0.05% Tween 20) (300 μL/well) and washed again 4× with ELISA wash buffer. Concurrently, 2× Compound 7/Compound 13 standards (in respective tissue matrix) or sample tumor homogenates diluted with antibody diluent (PBS+2% dry milk+0.05% Tween 20), were pre-incubated with 1-10 ng/mL of a primary antibody specific for the Pv1 peptide for 30 minutes at room temperature. Pre-incubated samples were added to pre-coated, pre-blocked assay plates at 100 μL well and incubated for 1 hour at room temperature. Plates were washed 4× with ELISA wash buffer and incubated with 100 μL/well of a secondary goat anti-mouse IgG HRP antibody (1:5,000 in antibody diluent) for 1 hour at room temperature. Plates were washed 4× with ELISA wash buffer and incubated with 100 μL well of SuperSignal substrate at room temperature with gentle shaking for 1 minute. Luminescence was read from the plate on a BioTek Cytation 5 plate reader.

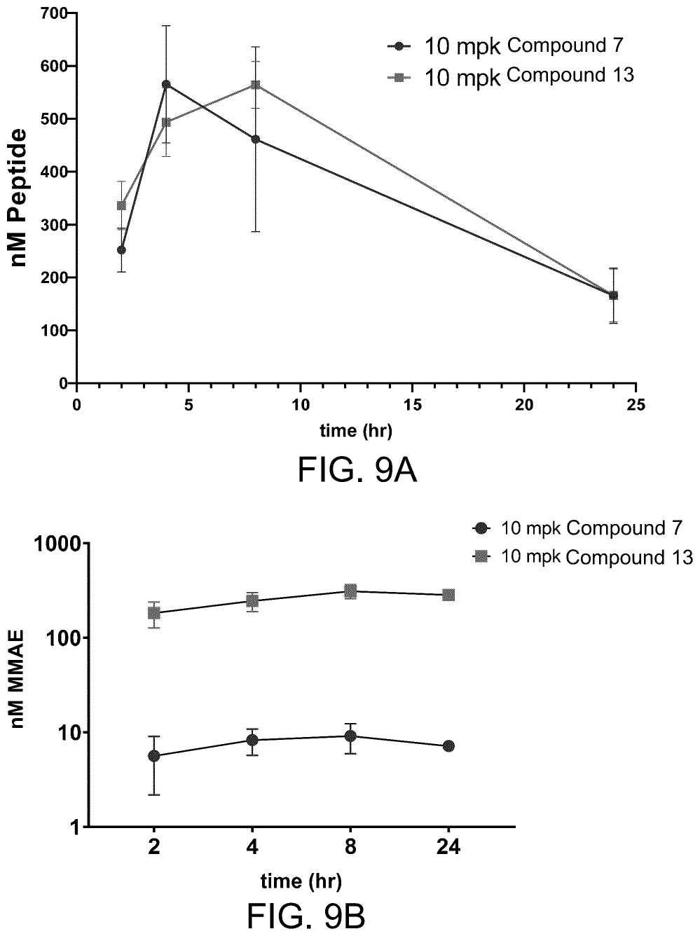

FIG. 9A shows a plot of the peptide concentrations in tumor after a single 10 mg/kg IP dose of either Compound 7 or Compound 13 in HCT116 colorectal tumor bearing female nude mice (data are expressed as means±SD).

FIG. 9B shows a plot of the MMAE concentrations in tumor after a single 10 mg/kg IP dose of either Compound 7 or Compound 13 in HCT116 colorectal tumor bearing female nude mice (data are expressed as means±SD).

The data demonstrate that while both conjugates insert similarly into tumor, Compound 13 is more labile, releasing 30-40× more warhead in tumor relative to Compound 7.

### Example J: Efficacy of Compound 13 in a HT-29 Colorectal Xenograft Model

Six-week-old female athymic nude Foxn^{nu} mice were obtained from Taconic Labs (Cat #NCRNU-F) and were housed 5 per cage on Alpha-Dri bedding in a disposable caging system. Human HT-29 cells derived from colorectal cancer were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle or 5 mg/kg Compound 13. Doses were prepared by diluting 0.1 mg/μL DMSO stocks in 5% mannitol in citrate buffer and were administered on days 0-3, 5 and 16-19 at a volume of 12 mL/kg (300 μL per 25 g mouse). Xenograft tumors were measured by calipers and volume was calculated using the equation for ellipsoid volume: Volume=π/6×(length)×(width)². Animals were removed from the study due to death, tumor size exceeding 2000 mm³ or loss of >20% body weight. The below table shows the dosing schedule of various treatment groups.

| | | | | Adminis- | |
|---|---|---|---|---|---|
| | | | Dosing | tration | Number |
| Group | Treatment | Dose | Schedule | Route | of Mice |
| 1 | Vehicle | NA | Days 0-3, 5, | i.p. | 8 |
| | (5% mannitol | | 16-19 | | |
| | in citrate | | | | |
| | buffer) | | | | |
| 2 | Compound 13 | 5 mg/kg | Days 0-3, 5, | i.p. | 8 |
| | | | 16-19 | | |

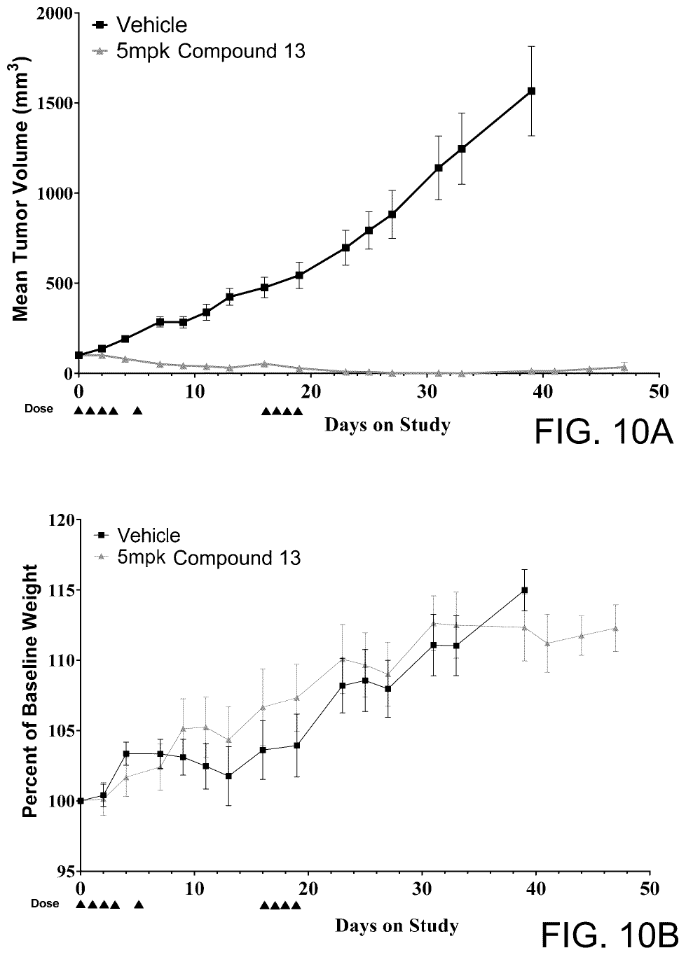

FIG. 10A shows a plot of the mean tumor volume resulting from dosing 5 mg/kg Compound 13 in nude mice bearing HT-29 colorectal cancer flank tumors. Animals were dosed once daily intraperitoneally on days 0-3, 5 and 16-19.

FIG. 10B displays percent change in body weight of animals in this study. Data are expressed as means±SEM.

Animals dosed with Compound 13 experienced no change in body weight. These data demonstrate that Compound 13 demonstrates potent anti-tumor activity and safety in a pre-clinical colorectal cancer model.

### Example K: Efficacy Compound 7 in a HT-29 Colorectal Xenograft Model

Six-week-old female athymic nude Foxn^{nu} mice were obtained from Taconic Labs (Cat #NCRNU-F) and were housed 5 per cage on Alpha-Dri bedding in a disposable caging system. Human HT-29 cells derived from colorectal cancer were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle, 40 or 80 mg/kg Compound 7. Doses were prepared by diluting 0.1 mg/μL DMSO stocks in 5% mannitol in citrate buffer and were administered QD×4/week for 2 weeks at a volume of 12 mL/kg (300 μL per 25 g mouse). Xenograft tumors were measured by calipers and volume was calculated using the equation for ellipsoid volume: Volume=π/6×(length)×(width)². Animals were removed from the study due to death, tumor size exceeding 2000 mm³ or loss of >20% body weight. The below table shows the dosing schedule of various treatment groups.

| | | | | Adminis- | |
|---|---|---|---|---|---|
| | | | Dosing | tration | Number |
| Group | Treatment | Dose | Schedule | Route | of Mice |
| 1 | Vehicle | NA | QDx4/wk × | i.p. | 8 |
| | (5% mannitol | | 2 wks | | |
| | in citrate | | | | |
| | buffer) | | | | |
| 2 | Compound 7 | 40 mg/kg | QDx4/wk × | i.p. | 8 |
| | | | 2 wks | | |
| 3 | Compound 7 | 80 mg/kg | QDx4/wk × | i.p. | 8 |
| | | | 2 wks | | |

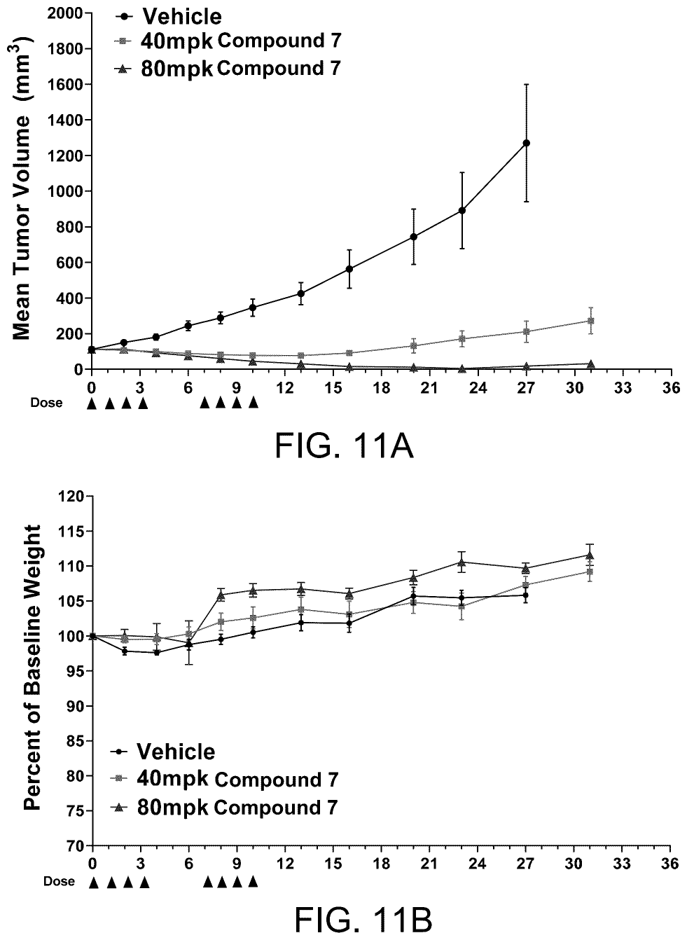

FIG. 11A shows a plot of the mean tumor volume resulting from dosing 40 and 80 mg/kg Compound 7 in nude mice bearing HT-29 colorectal cancer flank tumors. Animals were dosed once daily intraparenterally for four consecutive days a week for two weeks.

FIG. 11B displays percent change in body weight of animals in this study. Data are expressed as means±SEM.

These data demonstrate that Compound 7 demonstrates efficacy and safety in the HT-29 model at higher doses relative to Compound 13, concordant with the differing release profiles of the two conjugates.

### Example L: Tissue Pharmacokinetics of Compound 13, Compound 1, and Compound 2 in a Mouse Model

### Animal Dosing

Six-week-old female athymic nude Foxn^{nu} mice were obtained from Taconic Labs (Cat #NCRNU-F) and were housed 5 per cage on Alpha-Dri bedding in a disposable caging system (Innovive). Human HCT 116 cancer cells derived from colorectal carcinoma were diluted 1:1 in Phenol Red-free Matrigel and subcutaneously implanted into the left flank of each mouse at a density of 2.5×10⁶ cells in 100 μL. When xenografts reached a minimal volume of 300 mm³, mice were administered a single intraperitoneal injection of 10 mg/kg Compound 13, Compound 1, or Compound 2 prepared in a vehicle of 5% mannitol in citrate. Tumor was collected at 4 and 24 hours after compound administration. MMAE concentrations in tumor was determined by LCMS and peptide concentrations determined by ELISA.

### LC-MS/MS Determination of Tissue MMAE Concentration

Thawed tissue samples kept on wet ice were adjusted to 100 mg/mL with PBS based on tissue weight. Samples were homogenized on a Precellys Evolution machine at 7200 rpm for 2×30 second cycles with a 10 second pause in between each cycle. Homogenates were centrifuged at 14,000 rpm for 5 minutes at 4° C. and the supernatants were transferred to clean 2 mL LoBind Eppendorf tubes. A 100 μL volume of homogenate was added to a clean 2 mL 96-well polypropylene plate followed by 75 μL of ammonium formate buffer, pH 6.9, and 25 μL of working internal standard (WIS). Double blank controls received 75 μL of water:acetonitrile:formic acid (1:1:0.001, v:v:v) without internal standard. Fortified samples were covered with a silicone cap mat and vortexed at 700 rpm for 2 minutes. Working on a negative pressure manifold, 200 μL of fortified matrix samples were added to a supported liquid extraction (SLE) plate and samples were allowed to percolate through the plate frit with a negative pressure of ˜650-700 torr for up to 1-minute. Samples were allowed to completely absorb into the SLE plate for 5 minutes. Prior to sample elution, a 2-mL 96-well TrueTaper collection plate was placed within the vacuum manifold as the collection vessel. Samples were evaporated under a heated stream of nitrogen at 40° C. and reconstituted in 150 μL of acetonitrile:water:200 mM ammonium formate (90:5:5, v:v:v). The collection plate was covered with a silicone cap mat and vortexed at 900 rpm for 2 minutes. Final samples were centrifuged at 3000 rpm for 5 minutes at 4° C. and analysis was accomplished by injecting 2 μL onto an LC-MS/MS system.

### ELISA Measurement of Total Peptide Tissue Concentrations

96-well plates were coated with 100 μL/well of 0.1 μM BSA-labelled peptide prepared in 0.2 M Carbonate-Bicarbonate Buffer, pH 9.4 and incubated overnight at 4° C. Plates were washed 4× with an ELISA wash buffer (PBS+0.05% Tween 20), incubated for 2 hours at room temperature with Blocking Buffer (PBS+5% dry milk+0.05% Tween 20) (300 μL/well) and washed again 4× with ELISA wash buffer. Concurrently, 2× auristatin-conjugate standards (in respective tissue matrix) or sample tumor homogenates diluted with antibody diluent (PBS+2% dry milk+0.05% Tween 20), were pre-incubated with 1-10 ng/mL of a primary antibody specific for the Pv1 peptide for 30 minutes at room temperature. Pre-incubated samples were added to pre-coated, pre-blocked assay plates at 100 μL/well and incubated for 1 hour at room temperature. Plates were washed 4× with ELISA wash buffer and incubated with 100 μL/well of a secondary goat anti-mouse IgG HRP antibody (1:5,000 in antibody diluent) for 1 hour at room temperature. Plates were washed 4× with ELISA wash buffer and incubated with 100 μL/well of SuperSignal substrate at room temperature with gentle shaking for 1 minute. Luminescence was read from the plate on a BioTek Cytation 5 plate reader.

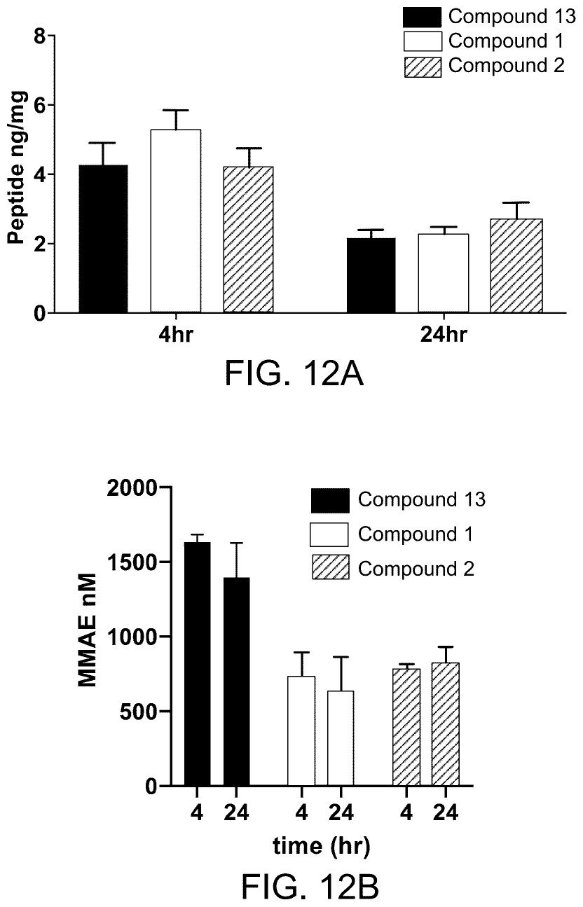

FIG. 12A shows a plot of the peptide concentrations in tumor after a single 10 mg/kg intraperitoneal dose of Compound 13, Compound 1, or Compound 2 in HCT116 colorectal tumor bearing female nude mice (data are expressed as means±SD).

FIG. 12B shows a plot of the MMAE concentrations in tumor after a single 10 mg/kg intraperitoneal dose of Compound 13, Compound 1, or Compound 2 in HCT 116 colorectal tumor bearing female nude mice (data are expressed as means±SD).

The data demonstrate that while conjugates insert similarly into tumor, Compound 1 and Compound 2 release intermediate levels of MMAE relative to Compound 13.

### Example M: Tissue Pharmacokinetics of Compound 13, Compound 7, Compound 5 and Compound 6 in a Mouse Model

### Animal Dosing

Six-week-old female athymic nude Foxn^{nu} mice were obtained from Taconic Labs (Cat #NCRNU-F) and were housed 5 per cage on Alpha-Dri bedding in a disposable caging system (Innovive). Human HCT 116 cancer cells derived from colorectal carcinoma were diluted 1:1 in Phenol Red-free Matrigel and subcutaneously implanted into the left flank of each mouse at a density of 2.5×10⁶ cells in 100 μL. When xenografts reached a minimal volume of 300 mm³, mice were administered a single intraperitoneal injection of 10 mg/kg Compound 13, Compound 7, Compound 5 or Compound 6 prepared in a vehicle of 5% mannitol in citrate. Tumor was collected at 4 and 24 hours after compound administration. MMAE concentrations in tumor was determined by LCMS and peptide concentrations determined by ELISA.

### LC-MS/MS Determination of Tissue MMAE Concentration

Thawed tissue samples kept on wet ice were adjusted to 100 mg/mL with PBS based on tissue weight. Samples were homogenized on a Precellys Evolution machine at 7200 rpm for 2×30 second cycles with a 10 second pause in between each cycle. Homogenates were centrifuged at 14,000 rpm for 5 minutes at 4° C. and the supernatants were transferred to clean 2 mL LoBind Eppendorf tubes. A 100 μL volume of homogenate was added to a clean 2 mL 96-well polypropylene plate followed by 75 μL of ammonium formate buffer, pH 6.9, and 25 μL of working internal standard (WIS). Double blank controls received 75 μL of water:acetonitrile:formic acid (1:1:0.001, v:v:v) without internal standard. Fortified samples were covered with a silicone cap mat and vortexed at 700 rpm for 2 minutes. Working on a negative pressure manifold, 200 μL of fortified matrix samples were added to a supported liquid extraction (SLE) plate and samples were allowed to percolate through the plate frit with a negative pressure of ˜650-700 torr for up to 1-minute. Samples were allowed to completely absorb into the SLE plate for 5 minutes. Prior to sample elution, a 2-mL 96-well TrueTaper collection plate was placed within the vacuum manifold as the collection vessel. Samples were evaporated under a heated stream of nitrogen at 40° C. and reconstituted in 150 μL of acetonitrile:water:200 mM ammonium formate (90:5:5, v:v:v). The collection plate was covered with a silicone cap mat and vortexed at 900 rpm for 2 minutes. Final samples were centrifuged at 3000 rpm for 5 minutes at 4° C. and analysis was accomplished by injecting a 2 μL sample onto an LC-MS/MS system.

### ELISA Measurement of Total Peptide Tissue Concentrations

96-well plates were coated with 100 μL/well of 0.1 μM BSA-labelled peptide prepared in 0.2 M Carbonate-Bicarbonate Buffer, pH 9.4 and incubated overnight at 4° C. Plates were washed 4× with an ELISA wash buffer (PBS+0.05% Tween 20), incubated for 2 hours at room temperature with Blocking Buffer (PBS+5% dry milk+0.05% Tween 20) (300 μL/well) and washed again 4× with ELISA wash buffer. Concurrently, 2× auristatin-conjugate standards (in respective tissue matrix) or sample tumor homogenates diluted with antibody diluent (PBS+2% dry milk+0.05% Tween 20), were pre-incubated with 1-10 ng/mL of a primary antibody specific for the Pv1 peptide for 30 minutes at room temperature. Pre-incubated samples were added to pre-coated, pre-blocked assay plates at 100 μL/well and incubated for 1 hour at room temperature. Plates were washed 4× with ELISA wash buffer and incubated with 100 μL/well of a secondary goat anti-mouse IgG HRP antibody (1:5,000 in antibody diluent) for 1 hour at room temperature. Plates were washed 4× with ELISA wash buffer and incubated with 100 μL/well of SuperSignal substrate at room temperature with gentle shaking for 1 minute. Luminescence was read from the plate on a BioTek Cytation 5 plate reader.

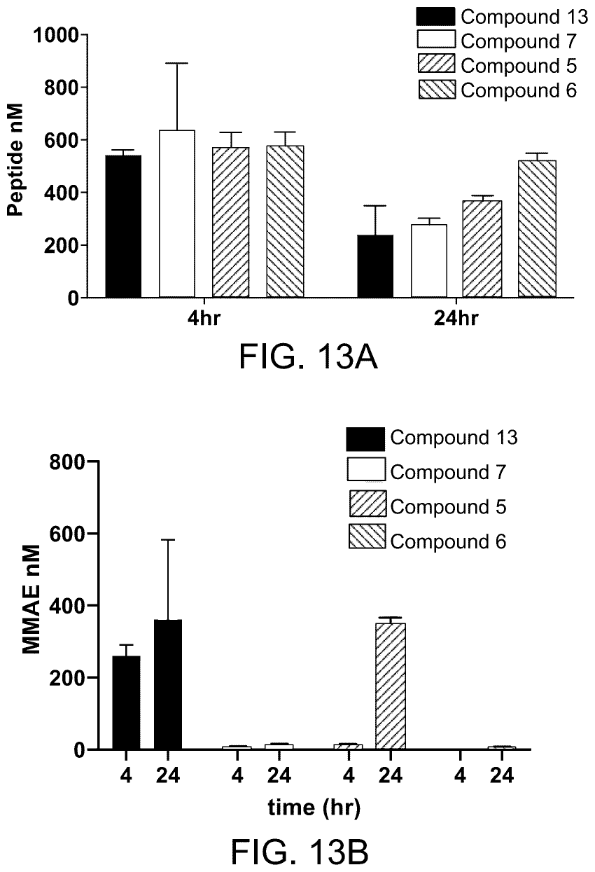

FIG. 13A shows a plot of the levels of peptide in mouse tumor determined by ELISA and LCMS after a single 10 mg/kg intraperitoneal injection of Compound 13, Compound 7, Compound 5 or Compound 6 in HCT116 colorectal tumor bearing female nude mice (data are expressed as means±SD).

FIG. 13B shows a plot of the levels of unconjugated MMAE in mouse tumor determined by ELISA and LCMS after a single 10 mg/kg intraperitoneal injection of Compound 13, Compound 7, Compound 5 or Compound 6 in HCT116 colorectal tumor bearing female nude mice (data are expressed as means±SD).

The data demonstrate that while conjugates insert similarly into tumor, they release warhead into tumor with a wide range of kinetics.

### Example N: Efficacy of Compound 5 in a HCT116 Colorectal Xenograft Model

Six-week-old female athymic nude Foxn^{nu} mice were obtained from Taconic Labs (Cat #NCRNU-F) and were housed 5 per cage on Alpha-Dri bedding in a disposable caging system. Human HCT116 cells derived from colorectal cancer were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle, 1, 5, or 10 mg/kg Compound 5. Doses were prepared by diluting 0.1 mg/μL DMSO stocks in 5% mannitol in citrate buffer and were administered QD×2/week for 3 weeks at a volume of 12 mL/kg (300 μL per 25 g mouse). Xenograft tumors were measured by calipers and volume was calculated using the equation for ellipsoid volume: Volume=π/6×(length)×(width)². Animals were removed from the study due to death, tumor size exceeding 2000 mm³ or loss of >20% body weight. The below table shows the dosing schedule of various treatment groups.

| | | | | | Adminis- | |
|---|---|---|---|---|---|---|
| | | | | Dosing | tration | Number |
| Group | Treatment | Dose | | Schedule | Route | of Mice |
| 1 | Vehicle | NA | | QDx4 | i.p. | 8 |
| | (5% mannitol | | | | | |
| | in citrate | | | | | |
| | buffer) | | | | | |
| 2 | Compound 5 | 1 | mg/kg | QDx4 | i.p. | 8 |
| 3 | Compound 5 | 5 | mg/kg | QDx4 | i.p. | 8 |
| 4 | Compound 5 | 10 | mg/kg | QDx4 | i.p. | 8 |

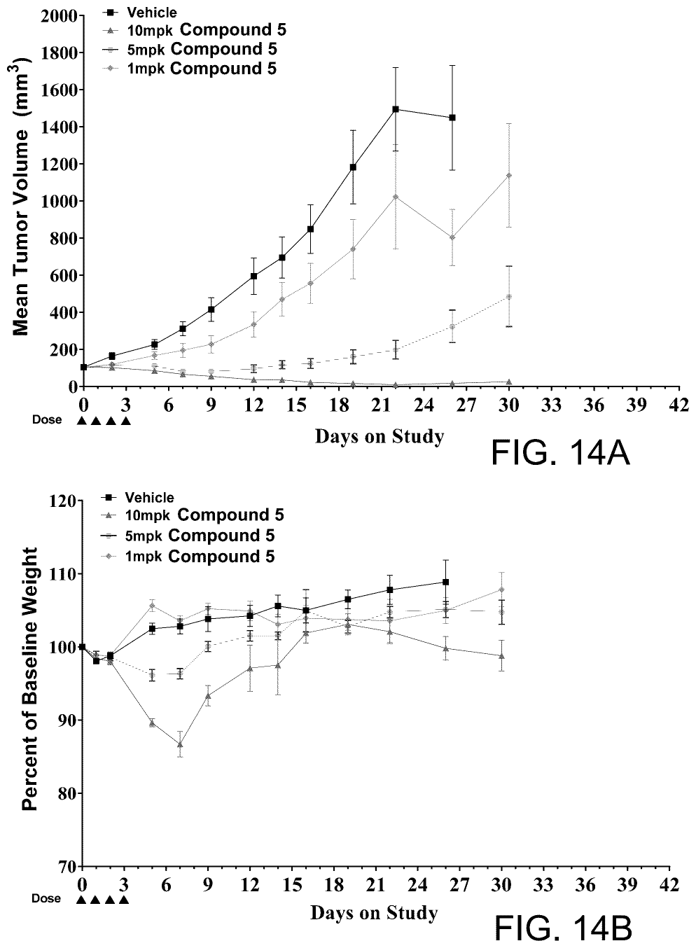

FIG. 14A shows a plot of the mean tumor volume resulting from dosing 1, 5 and 10 mg/kg Compound 5 in nude mice bearing HCT116 colorectal cancer flank tumors. Animals were dosed once daily intraparenterally for four consecutive days.

FIG. 14B displays percent change in body weight of animals in this study. Data are expressed as means±SEM.

These data demonstrate that Compound 5 demonstrates dose responsive efficacy in the HCT116 model.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A compound of Formula (I):
R²-L- R¹ (I)
or a pharmaceutically acceptable salt thereof, wherein:
R¹ is a peptide;
R² is a radical of an auristatin compound; and
L is a linker having a structure selected from: 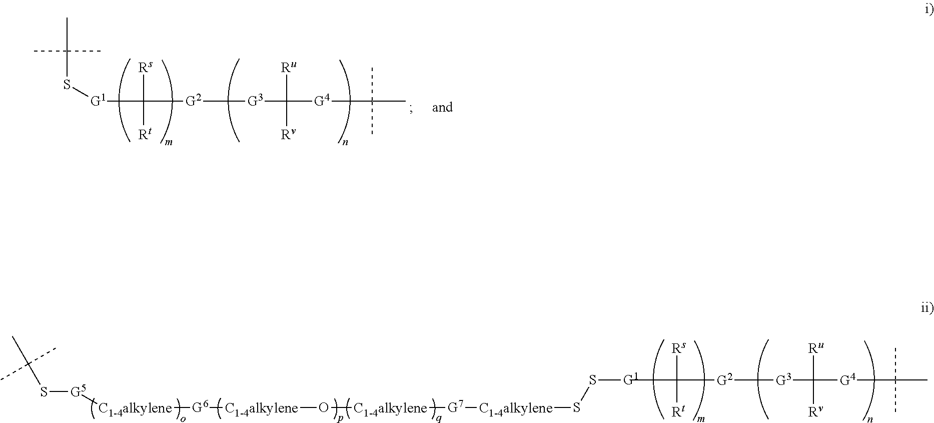
wherein the terminal S atom of the linker is bonded with a cysteine residue of the peptide to form a disulfide bond; and wherein:
G¹ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G¹ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR)NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
G² is selected from —NR^{G}C(O)—, —NR^{G}—O—, —S—, —C(O)—, —OC(O)—, —NR^{G}C(O)—, —OC(O)NR^{G}—, and —S(O₂)—;
G³ is selected from C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, and C₂₋₆ alkynyl substituent of G³ are optionally substituted with 1, 2, or 3 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
G⁴ is selected from —C(O)—, —NR^{G}C(O)—, —NR^{G}—, —O—, —OC(O)—, —NR^{G}C(O)—, and —S(O₂)—;
G⁵ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G⁵ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NRS(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G⁵ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR)NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NRS(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
G⁶ is selected from —NR^{G}C(O)—, —NR^{G}—, —O—, —C(O)—, —OC(O)—, —NR^{G}C(O)—, —OC(O)NR^{G}—, and —S(O₂)—;
G⁷ is selected from —NR^{G}C(O)—, —NR^{G}—O—, —S—, —C(O)O—, —OC(O)—, —NR^{G}C(O)—, —OC(O)NR^{G}—, and —S(O₂)—;
each R^{s} and R^{t} are independently selected from H, halo, C₁₋₆ alkyl, and C₁₋₆ haloalkyl;
or each R^{s} and R^{t}, together with the C atom to which they are attached, form a C₃₋₆ cycloalkyl ring;
R^{u} and R^{v} are independently selected from H, halo, C₁₋₆ alkyl, and C₁₋₆ haloalkyl;
each R^{G} is independently selected from H and C₁₋₄ alkyl;
each R^{a}, R^{b}, R^{c}, R^{d}, R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a2}, SR^{a2}, C(O)R^{b2}, C(O)NR^{c2}R^{d2}, C(O)OR^{a}, OC(O)R^{b2}, OC(O)NR^{c2}R^{d2}, NR^{c2}R^{d2}, NR^{c2}C(O)R^{b2} NR^{c2}C(O)NR^{c2}R^{d2}, NR^{c2}C(O)OR^{a2}, C(═NR^{e2})NR^{c2}R^{d2}, NR^{c2}C(═NR^{e2})NR^{c2}R^{d2}, S(O)R^{b2}, S(O)NR^{c2}R^{d2}, S(O)₂R^{b2}, NR^{c2}S(O)₂R^{b2}, NR²S(O)₂NR^{c2}R^{d2}, and S(O)₂NR^{c2}R^{d2};
each R^{a1}, R^{b2}, R^{c2}, and R^{d2} is independently selected from H, C₁₋₆ alkyl, C₁₋₆ haloalkyl, C₂₋₆ alkenyl, and C₂₋₆ alkynyl, wherein said C₁₋₆ alkyl, C₁₋₆ haloalkyl, C₂₋₆ alkenyl, and C₂₋₆ alkynyl of R^{a2}, R^{b2}, R^{c2}, and R^{d2} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, and R^{e2} is independently selected from H and C₁₋₄ alkyl;
m is 0, 1, 2, 3, or 4;
n is 0 or 1;
o is 0 or 1;
p is 1, 2, 3, 4, 5, or 6; and
q is 0 or 1.

2. A compound of Formula (I):
R²-L-R¹ (I)
or a pharmaceutically acceptable salt thereof, wherein:
R¹ is a peptide;
R² is a radical of an auristatin compound; and
L is a linker having the structure: 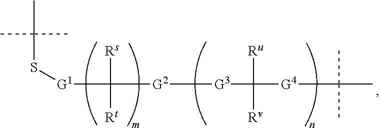
wherein the S atom of the linker is bonded with a cysteine residue of the peptide to form a disulfide bond; and wherein:
G¹ is selected from a bond, C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said C₁₋₆ alkyl, C₂₋₆ alkenyl, and C₂₋₆ alkynyl substituent of G¹ are optionally substituted with 1, 2, or 3 substituents independently selected from CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{d}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each R^{s} and R^{t} are independently selected from H, halo, C₁₋₆ alkyl, and C₁₋₆ haloalkyl;
G² is selected from —NR^{G}C(O)—, —NR^{G}—O—, —S—, —C(O)—, —OC(O)—, —NR^{G}C(O)—, —OC(O)NR^{G}—, and —S(O₂)—;
G³ is selected from C₆₋₁₀ aryl, C₃₋₁₄ cycloalkyl, 5-14 membered heteroaryl, and 4-14 membered heterocycloalkyl, wherein said C₆₋₁₀ aryl, C₃₋₁₄ cycloalkyl, 5-14 membered heteroaryl, and 4-14 membered heterocycloalkyl of G³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, and C₂₋₆ alkynyl substituent of G³ are optionally substituted with 1, 2, or 3 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{u} and R^{v} are independently selected from H, halo, C₁₋₆ alkyl, and C₁₋₆ haloalkyl;
G⁴ is selected from —C(O)—, —NR^{G}C(O)—, —NR^{G}—O—, —S—, —C(O)O—, —OC(O)—, —NR^{G}C(O)—, and —S(O₂)—;
each R^{G} is independently selected from H and C₁₋₄ alkyl;
each R^{a}, R^{b}, R^{c}, R^{d}, R^{a1}, R^{b1}, R^{c1}, and R^{d1} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a2}, SR^{a2}, C(O)R^{b2}, C(O)NR^{c2}R^{d2}, C(O)OR^{a}, OC(O)R^{b2}, OC(O)NR^{c2}R^{d2}, NR^{e2}R^{d2}, NR^{c2}C(O)R^{b2}, NR^{c2}C(O)NR^{c2}R^{d2}, NR^{c2}C(O)OR^{a2}, C(═NR^{e2})NR^{c2}R^{d2}, NR^{c2}C(═NR^{e2})NR^{c2}R^{d2}, S(O)R^{b2}, S(O)NR^{c2}R^{d2}, S(O)₂R^{b2}, NR^{e}2S(O)₂R^{b2}, NR^{e2}S(O)₂NR^{c2}R^{d2}, and S(O)₂NR^{c2}R^{d2};
each R^{a1}, R^{b2}, R^{c2}, and R^{d2} is independently selected from H, C₁₋₆ alkyl, C₁₋₆ haloalkyl, C₂₋₆ alkenyl, and C₂₋₆ alkynyl, wherein said C₁₋₆ alkyl, C₁₋₆ haloalkyl, C₂₋₆ alkenyl, and C₂₋₆ alkynyl of R^{a2}, R^{b2}, R^{c2}, and R^{d2} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, and R^{e2} is independently selected from H and C₁₋₄ alkyl;
m is 0, 1, 2, 3, or 4; and
n is 0 or 1.

3. The compound of claim 1, or a pharmaceutically acceptable salt thereof, wherein R¹ is a peptide having 5 to 50 amino acids.

4. The compound of claim 1, or a pharmaceutically acceptable salt thereof, wherein R¹ is a peptide capable of selectively delivering R²L- across a cell membrane having an acidic or hypoxic mantle.

5. The compound of claim 1, or a pharmaceutically acceptable salt thereof, wherein R¹ is a peptide capable of selectively delivering R²L- across a cell membrane having an acidic or hypoxic mantle having a pH less than about 6.0.

6. The compound of claim 1, or a pharmaceutically acceptable salt thereof, wherein R¹ is a peptide comprising at least one of the following sequences:
(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG.

7. The compound of claim 1, or a pharmaceutically acceptable salt thereof, wherein R¹ is a peptide comprising at least the following sequence:
(SEQ ID NO. 1; Pv1)
ADDQNPWRAYLDLLFPTDTLLLDLLWCG.

8. The compound of claim 1, or a pharmaceutically acceptable salt thereof, wherein R¹ is a peptide comprising at least the following sequence:
(SEQ ID NO. 2; Pv2)
AEQNPIYWARYADWLFTTPLLLLDLALLVDADECG.

9. The compound of claim 1, or a pharmaceutically acceptable salt thereof, wherein R¹ is a peptide comprising at least the following sequence:
(SEQ ID NO. 3; Pv3)
ADDQNPWRAYLDLLFPTDTLLLDLLWDADECG.

10. The compound of claim 1, or a pharmaceutically acceptable salt thereof, wherein R² is a radical of a monomethyl auristatin compound.

11. The compound of claim 1, or a pharmaceutically acceptable salt thereof, wherein R² is a radical of monomethyl auristatin E.

12. The compound of claim 1, or a pharmaceutically acceptable salt thereof, wherein R² is a radical of monomethyl auristatin F.

13. The compound of claim 1, or a pharmaceutically acceptable salt thereof, wherein R² has the structure:

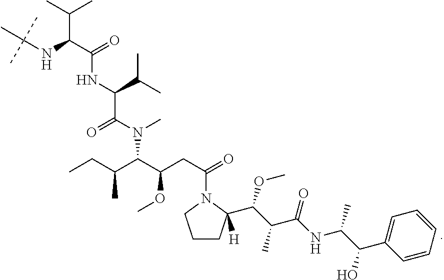

14. The compound of claim 1, or a pharmaceutically acceptable salt thereof, wherein R² has the structure:

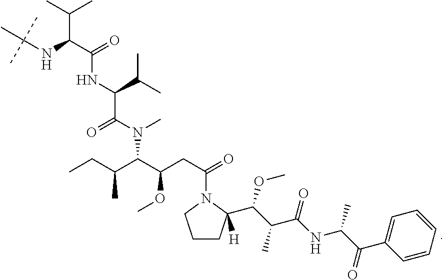

15. The compound of claim 1, or a pharmaceutically acceptable salt thereof, wherein R² has the structure:

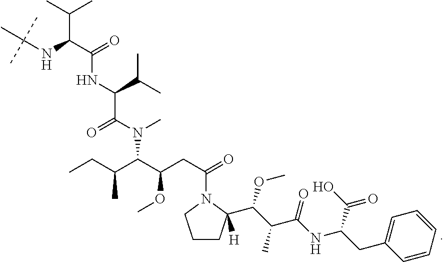

16. The compound of claim 1, or a pharmaceutically acceptable salt thereof, wherein L is a linker having the structure:

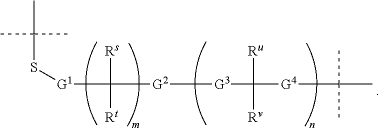

17. The compound of claim 1, or a pharmaceutically acceptable salt thereof, wherein L is a linker having the structure:

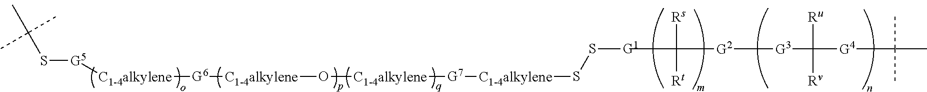

18. The compound of claim 1, or a pharmaceutically acceptable salt thereof, wherein G¹ is selected from a bond, C₆₋₁₀ aryl, C₃₋₁₄ cycloalkyl, 5-14 membered heteroaryl, and 4-14 membered heterocycloalkyl.

19. The compound of claim 1, or a pharmaceutically acceptable salt thereof, wherein G¹ is selected from a bond, phenyl, and C₄₋₆ cycloalkyl.

20. The compound of claim 1, or a pharmaceutically acceptable salt thereof, wherein G¹ is selected from a bond and C₃₋₁₄ cycloalkyl.

21. The compound of claim 1, or a pharmaceutically acceptable salt thereof, wherein G¹ is a bond.

22. The compound of claim 1, or a pharmaceutically acceptable salt thereof, wherein G¹ is C₃₋₁₄ cycloalkyl.

23. The compound of claim 1, or a pharmaceutically acceptable salt thereof, wherein G¹ is cyclopentyl or cyclohexyl, wherein said cyclopentyl and cyclohexyl are each optionally fused with a phenyl group.

24. The compound of claim 1, or a pharmaceutically acceptable salt thereof, wherein G¹ is phenyl.

25. The compound of claim 1, or a pharmaceutically acceptable salt thereof, wherein each R^{s} and R^{t} are independently selected from H and C₁₋₆ alkyl.

26. The compound of claim 1, or a pharmaceutically acceptable salt thereof, wherein each R^{s} and R^{t} are independently selected from H and isopropyl.

27. The compound of claim 1, or a pharmaceutically acceptable salt thereof, wherein each R^{s} and R^{t} are independently selected from H, methyl, and isopropyl.

28. The compound of claim 1, or a pharmaceutically acceptable salt thereof, wherein R^{s} and R^{t} together with the C atom to which they are attached form a cyclobutyl ring.

29. The compound of claim 1, or a pharmaceutically acceptable salt thereof, wherein m is 0, 1, or 2.

30. The compound of claim 1, or a pharmaceutically acceptable salt thereof, wherein m is 0.

31. The compound of claim 1, or a pharmaceutically acceptable salt thereof, wherein m is 2.

32. The compound of claim 1, or a pharmaceutically acceptable salt thereof, wherein G² is selected from —OC(O)— and —OC(O)NR^{G}—.

33. The compound of claim 1, or a pharmaceutically acceptable salt thereof, wherein G² is —OC(O)—.

34. The compound of claim 1, or a pharmaceutically acceptable salt thereof, wherein G³ is selected from C₆₋₁₀ aryl and 5-14 membered heteroaryl.

35. The compound of claim 1, or a pharmaceutically acceptable salt thereof, wherein G³ is C₆₋₁₀ aryl.

36. The compound of claim 1, or a pharmaceutically acceptable salt thereof, wherein G³ is phenyl.

37. The compound of claim 1, or a pharmaceutically acceptable salt thereof, wherein R^{u} and R are each H.

38. The compound of claim 1, or a pharmaceutically acceptable salt thereof, wherein G⁴ is —OC(O)—.

39. The compound of claim 1, or a pharmaceutically acceptable salt thereof, wherein n is 0.

40. The compound of claim 1, or a pharmaceutically acceptable salt thereof, wherein n is 1.

41. The compound of claim 1, or a pharmaceutically acceptable salt thereof, wherein G⁵ is the following group:

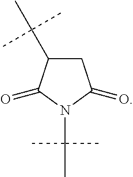

42. The compound of claim 1, or a pharmaceutically acceptable salt thereof, wherein G⁶ is —NR^{G}C(O)—.

43. The compound of claim 1, or a pharmaceutically acceptable salt thereof, wherein G⁷ is —NR^{G}C(O)—.

44. The compound of claim 1, or a pharmaceutically acceptable salt thereof, wherein o is 1.

45. The compound of claim 1, or a pharmaceutically acceptable salt thereof, wherein p is 3.

46. The compound of claim 1, or a pharmaceutically acceptable salt thereof, wherein p is 5.

47. The compound of claim 1, or a pharmaceutically acceptable salt thereof, wherein q is 1.

48. The compound of claim 1, or a pharmaceutically acceptable salt thereof, wherein each R^{G} is independently selected from H and methyl.

49. The compound of claim 1, or a pharmaceutically acceptable salt thereof, wherein each R^{G} is H.

50. The compound of claim 1, or a pharmaceutically acceptable salt thereof, wherein L has one of the following structures:

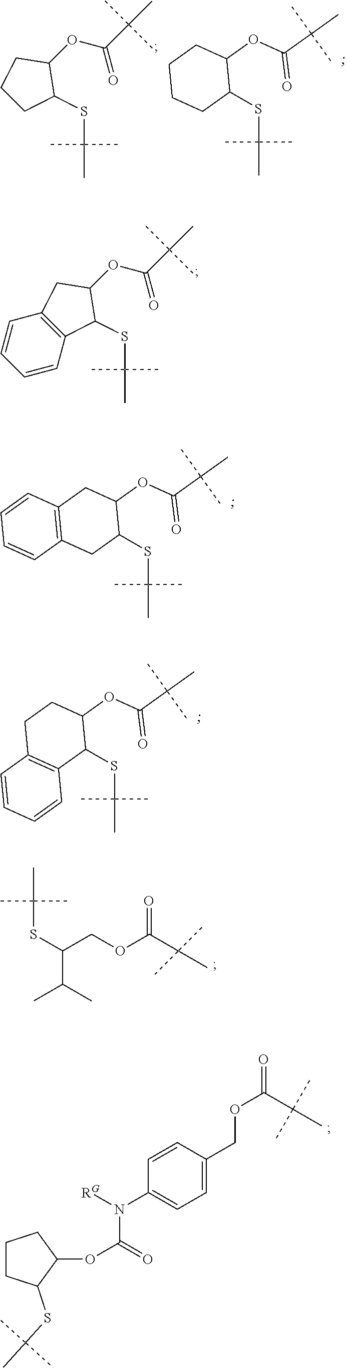

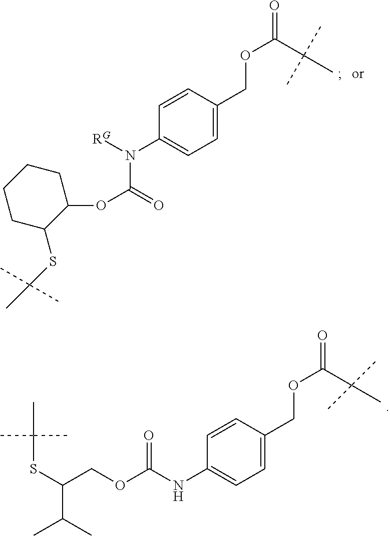

51. The compound of claim 1, or a pharmaceutically acceptable salt thereof, wherein L has one of the following structures:

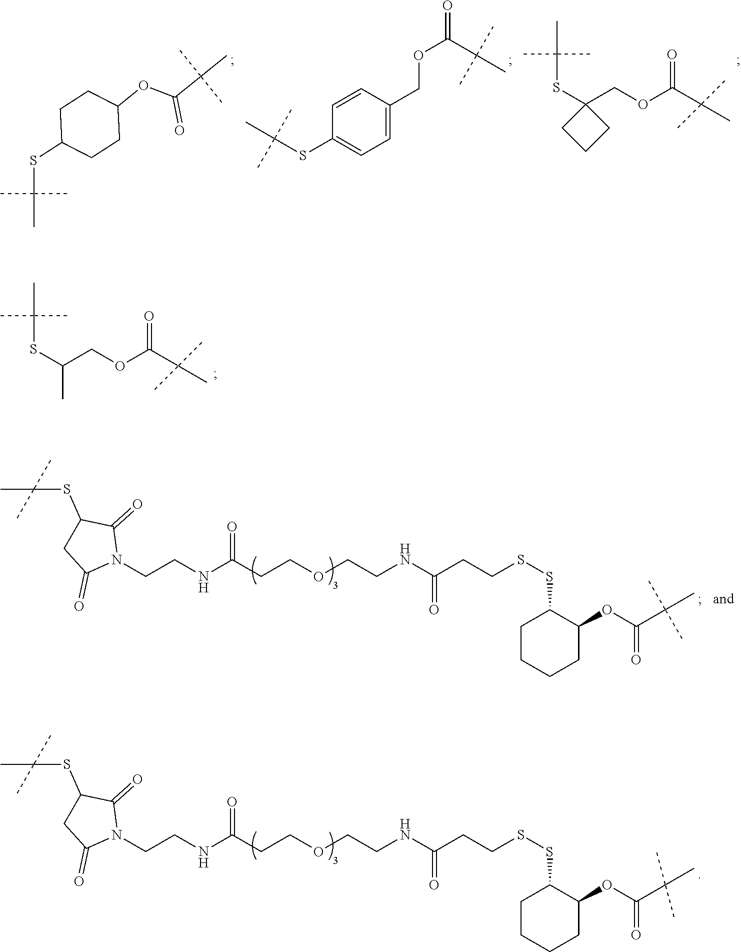

52. The compound of claim 1, having Formula (II): 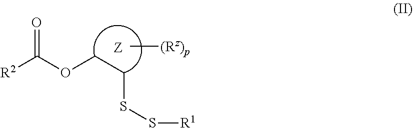
or a pharmaceutically acceptable salt thereof, wherein:
R¹ is a peptide;
R² is a radical of an auristatin compound;
Ring Z is a monocyclic C₅₋₇ cycloalkyl ring or a monocyclic 5-7 membered heterocycloalkyl ring;
each R^{Z} is independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d} NR^{c}R^{d}NR^{c}C(O)R^{b}, NR^{c}C(O)OR^{d}, and NR^{c}C(O)NR^{c}R^{d};
or two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₆ alkyl, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{d}, and NR^{c}C(O)NR^{c}R^{d};
R^{a}, R^{b}, R^{c}, and R^{d} are each independently selected from H, C₁₋₄ alkyl, C₂₋₄ alkenyl, C₂₋₄ alkynyl, each optionally substituted with 1, 2, or 3 substituents independently selected from halo, OH, CN, and NO₂; and
p is 0, 1, 2, or 3.

53. The compound of claim 52, or a pharmaceutically acceptable salt thereof, wherein R¹ is a peptide comprising the sequence of SEQ ID NO: 1, SEQ ID NO:2, or SEQ ID NO:3.

54. The compound of claim 52, or a pharmaceutically acceptable salt thereof, wherein R¹ is Pv1, Pv2, or Pv3.

55. The compound of claim 52, or a pharmaceutically acceptable salt thereof, wherein R¹ is attached to the core via a cysteine residue of R¹ wherein one of the sulfur atoms of the disulfide moiety in Formula II is derived from the cysteine residue.

56. The compound of claim 52, or a pharmaceutically acceptable salt thereof, wherein R² has the structure:

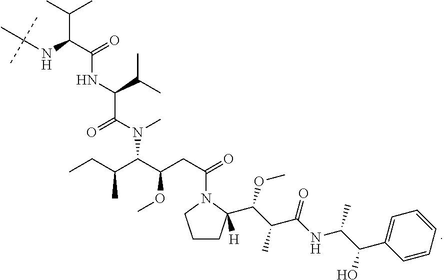

57. The compound of claim 52, or a pharmaceutically acceptable salt thereof, wherein R² has the structure:

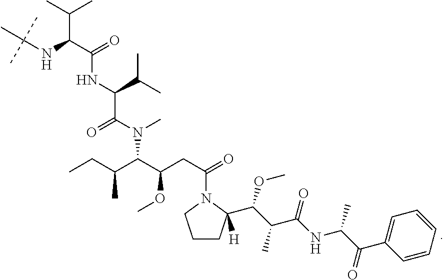

58. The compound of claim 52, or a pharmaceutically acceptable salt thereof, wherein R² is attached to the core through an N atom.

59. The compound of claim 52, or a pharmaceutically acceptable salt thereof, wherein Ring Z is a monocyclic C₅₋₇ cycloalkyl ring.

60. The compound of claim 52, or a pharmaceutically acceptable salt thereof, wherein Ring Z is a cyclopentyl ring.

61. The compound of claim 52, or a pharmaceutically acceptable salt thereof, wherein Ring Z is a cyclohexyl ring.

62. The compound of claim 52, or a pharmaceutically acceptable salt thereof, wherein two adjacent R^{Z} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)OR^{d}, and NR^{c}C(O)NR^{c}R^{d}.

63. The compound of claim 52, or a pharmaceutically acceptable salt thereof, wherein p is 0.

64. The compound of claim 52, or a pharmaceutically acceptable salt thereof, wherein p is 1.

65. The compound of claim 52, or a pharmaceutically acceptable salt thereof, wherein p is 2.

66. The compound of claim 52, or a pharmaceutically acceptable salt thereof, wherein p is 3.

67. The compound of claim 52, wherein the compound has Formula (III) or Formula (IV): 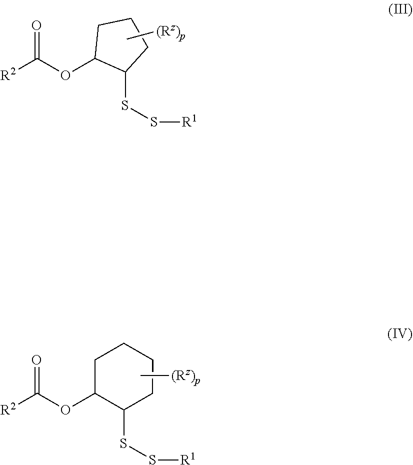
or a pharmaceutically acceptable salt thereof.

68. The compound of claim 1, wherein the compound is selected from one of the following: 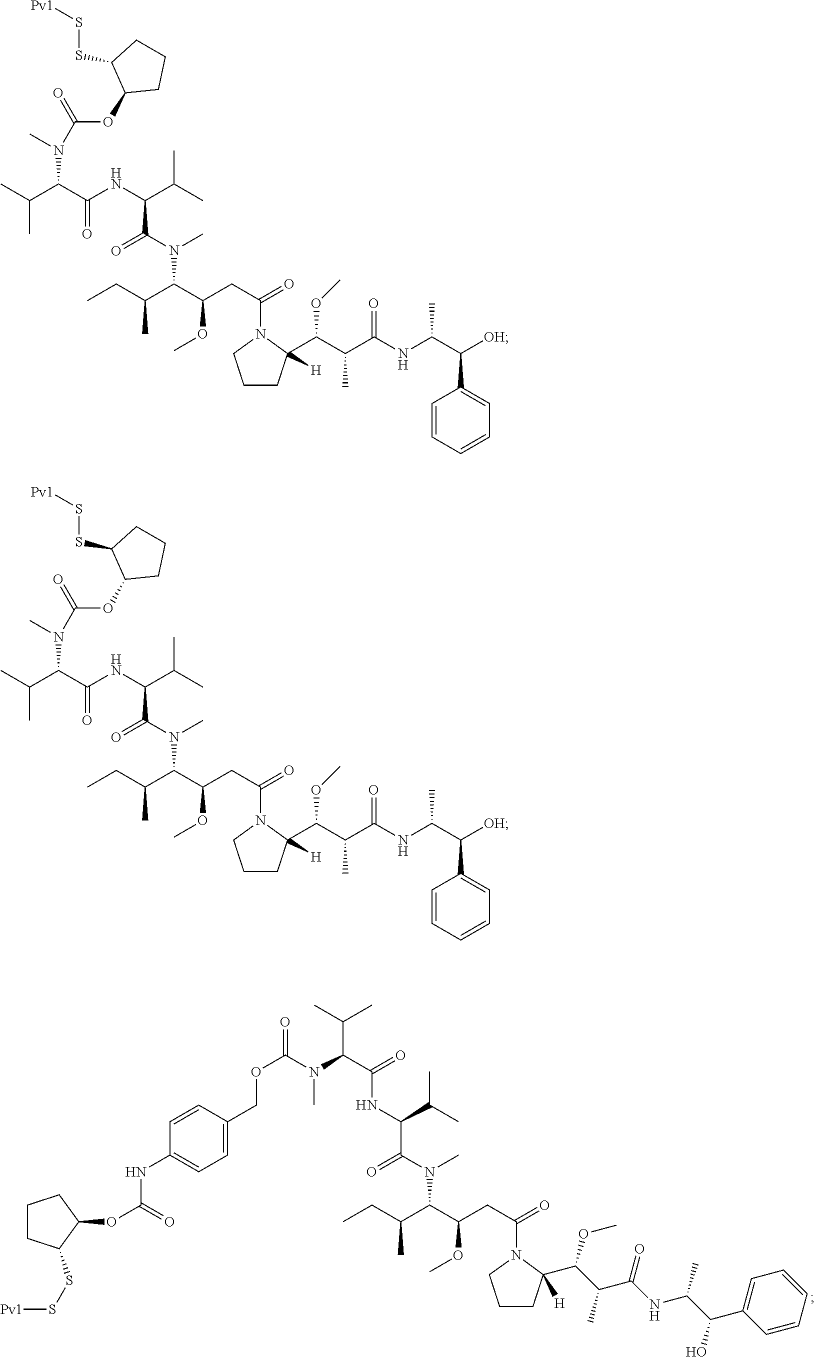 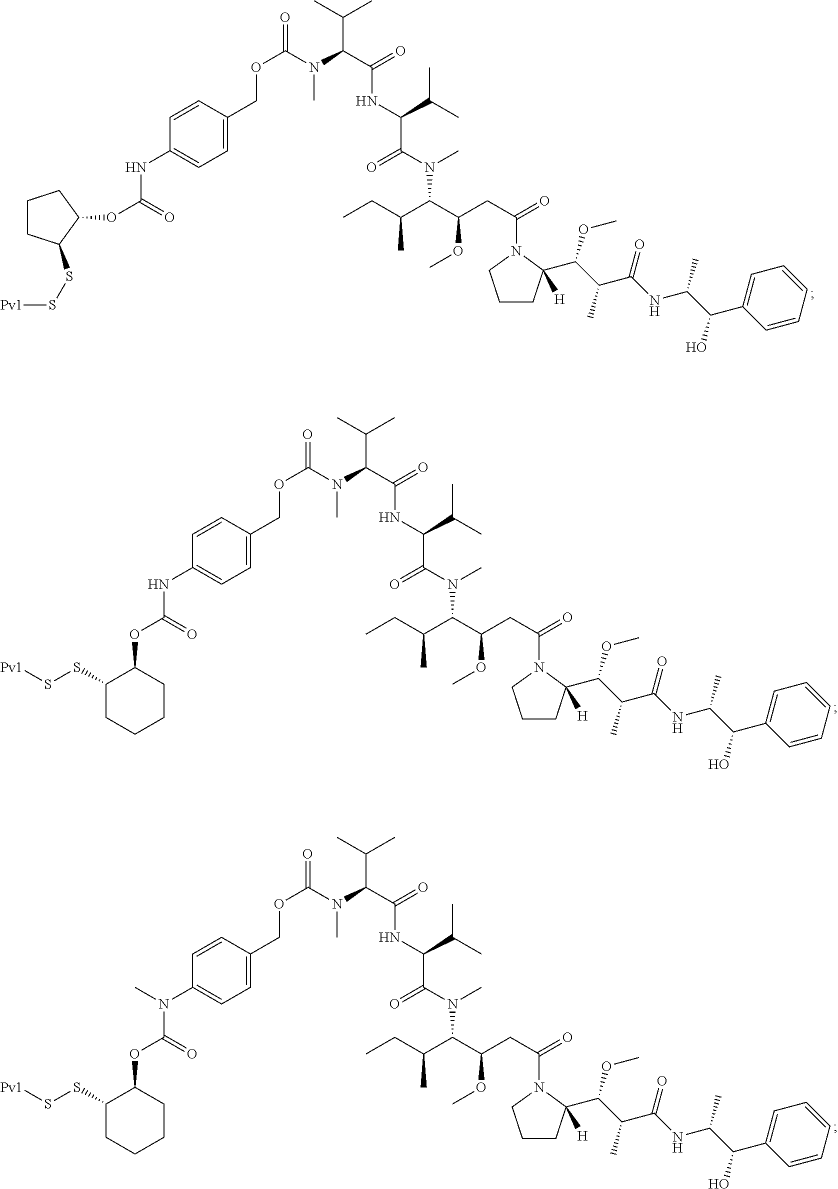 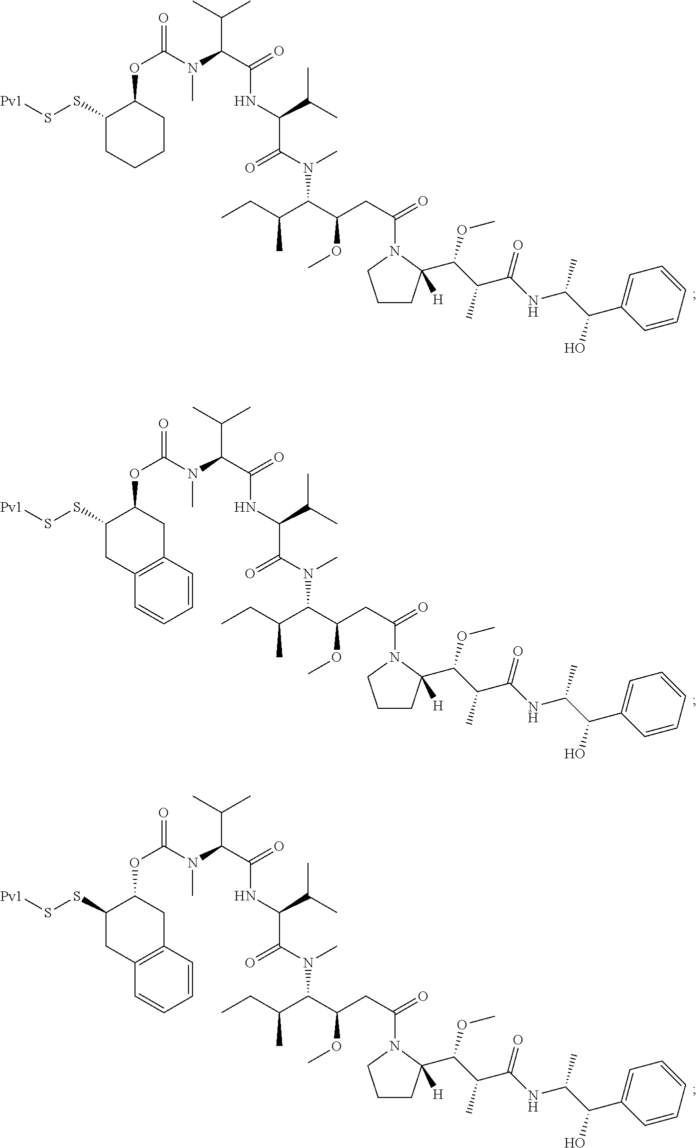 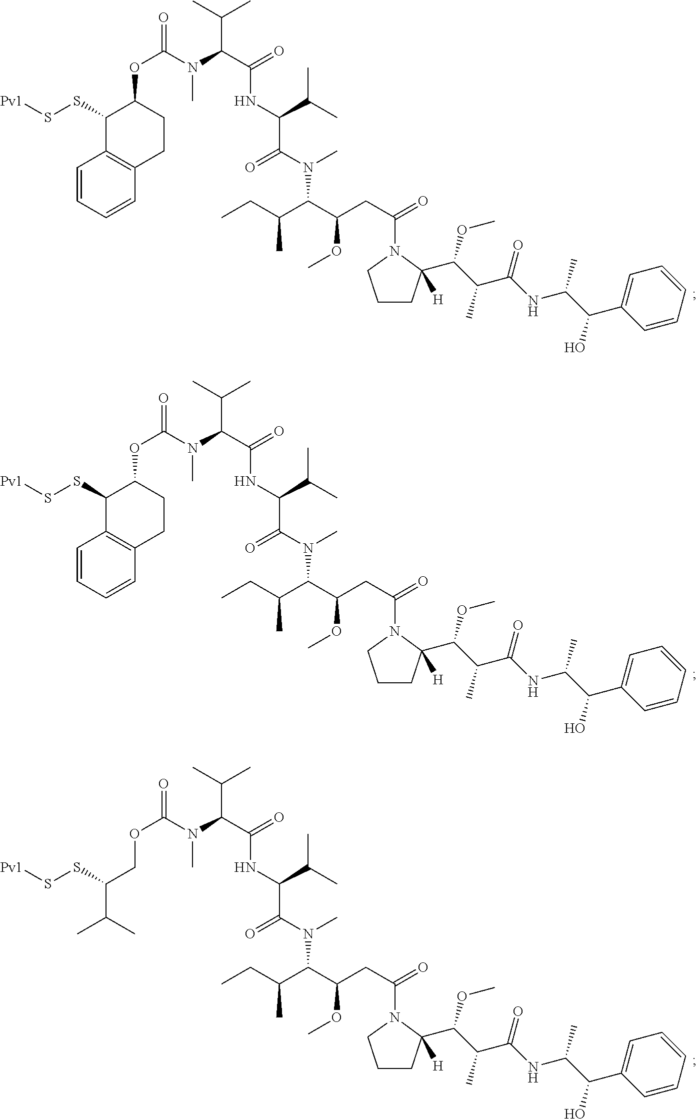 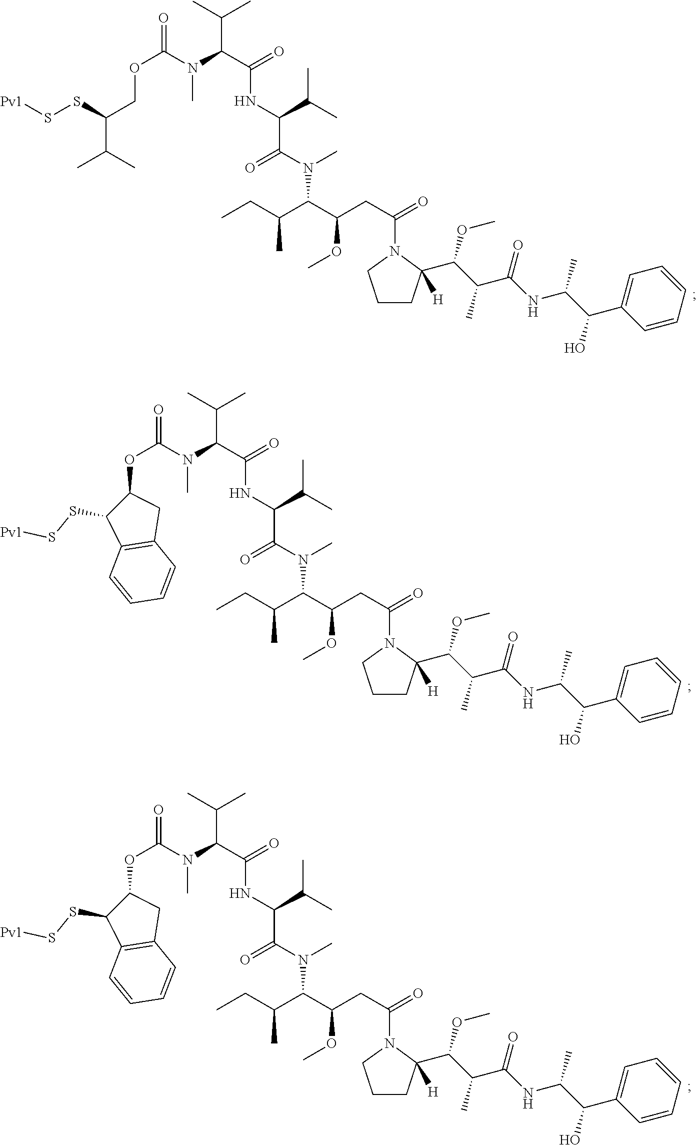 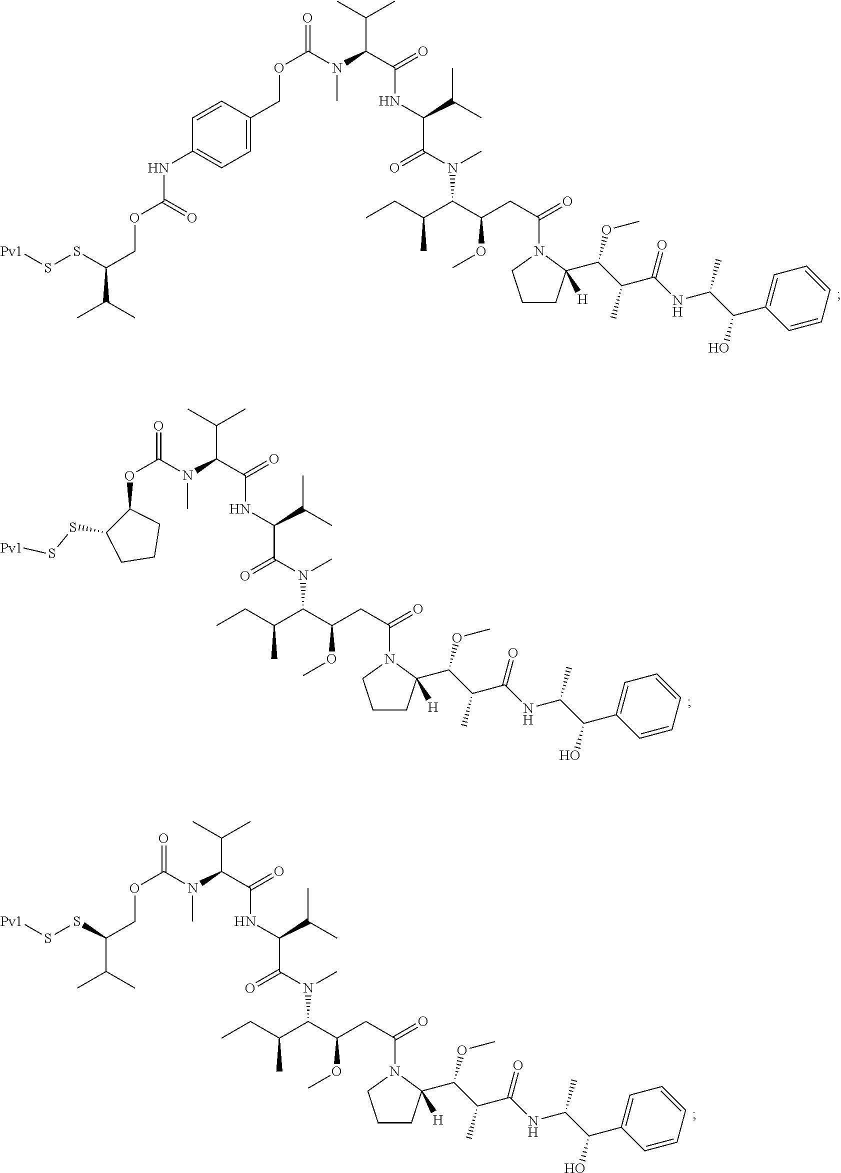 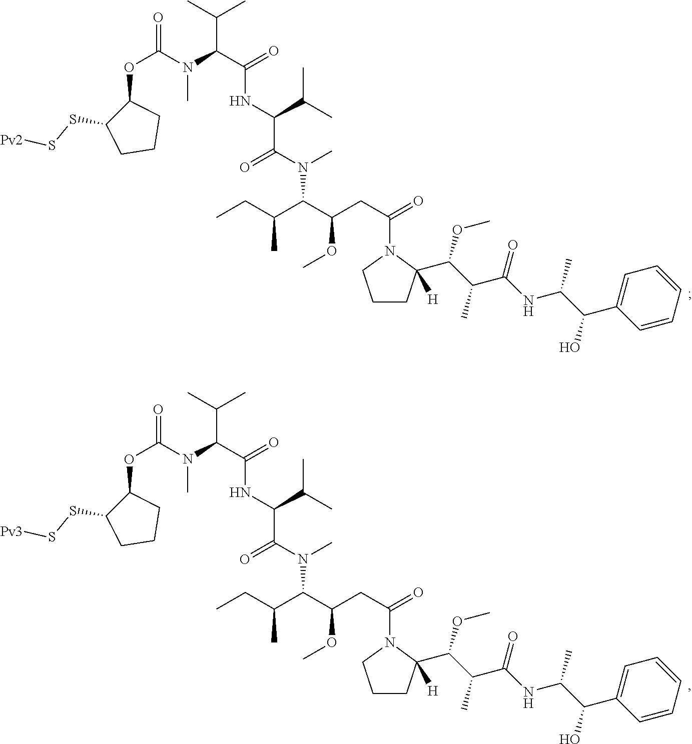
or a pharmaceutically acceptable salt of any of the aforementioned, wherein:
Pv1 is a peptide comprising the sequence:
(SEQ ID NO: 1)
ADDQNPWRAYLDLLFPTDTLLLDLLWCG;
Pv2 is a peptide comprising the sequence:
(SEQ ID NO: 2)
AEQNPIYWARYADWLFTTPLLLLDLALLVDADECG;
and
Pv3 is a peptide comprising the sequence:
(SEQ ID NO: 3)
ADDQNPWRAYLDLLFPTDTLLLDLLWDADECG.

69. The compound of claim 1, wherein the compound is selected from: 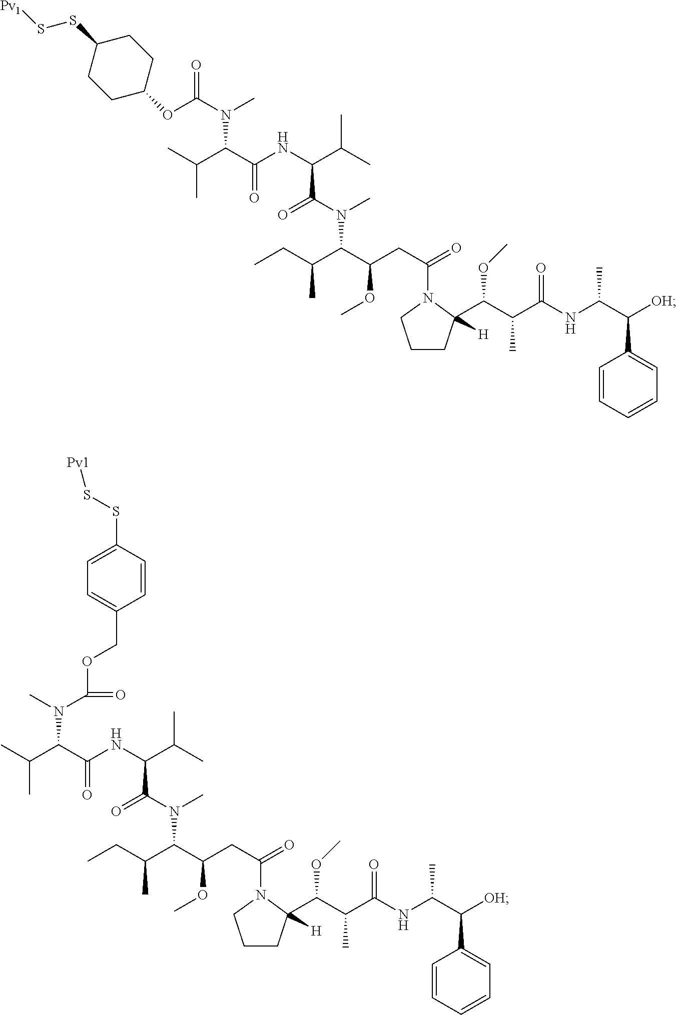 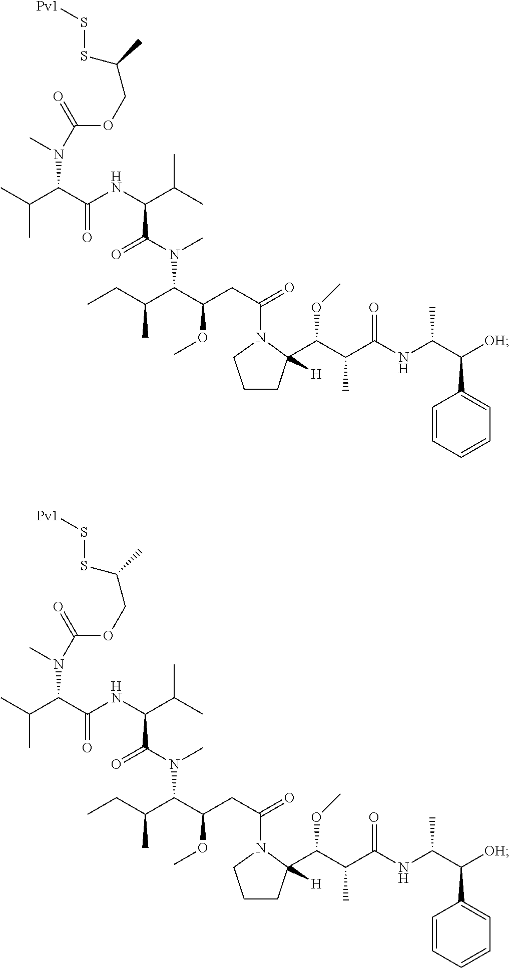 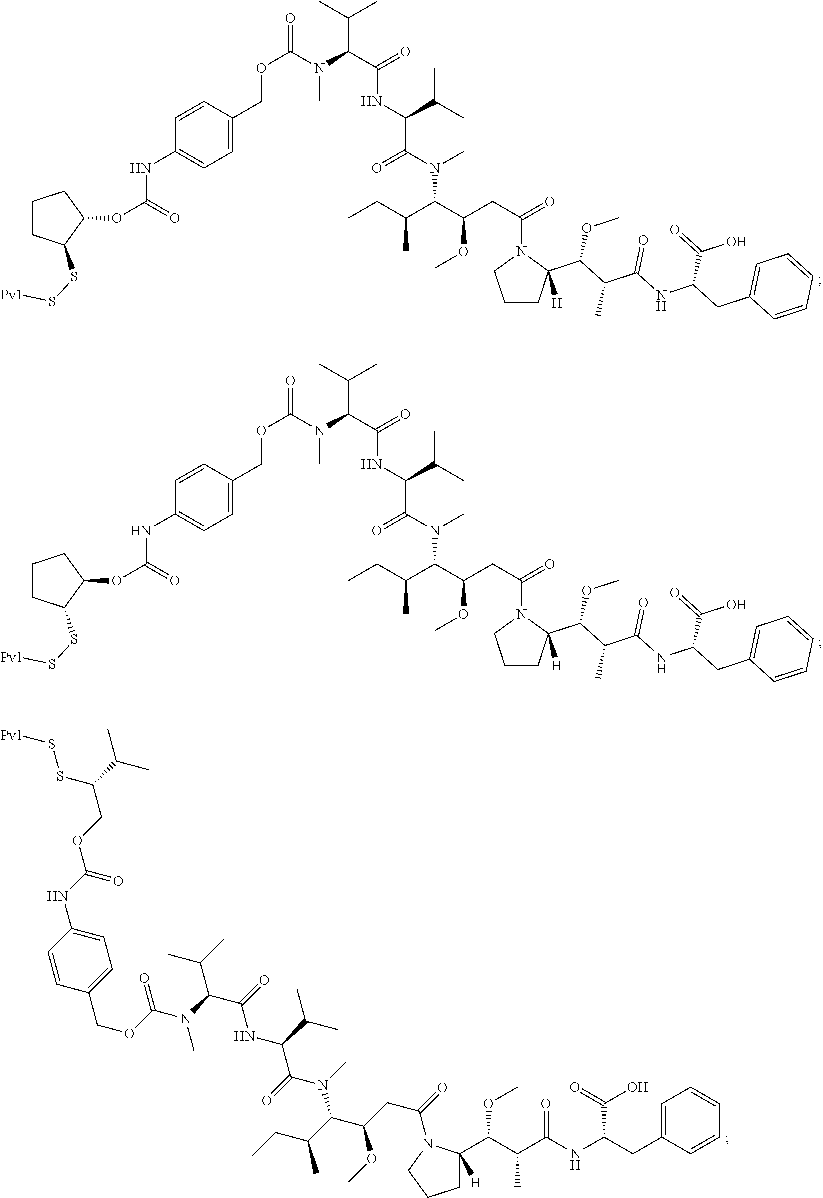 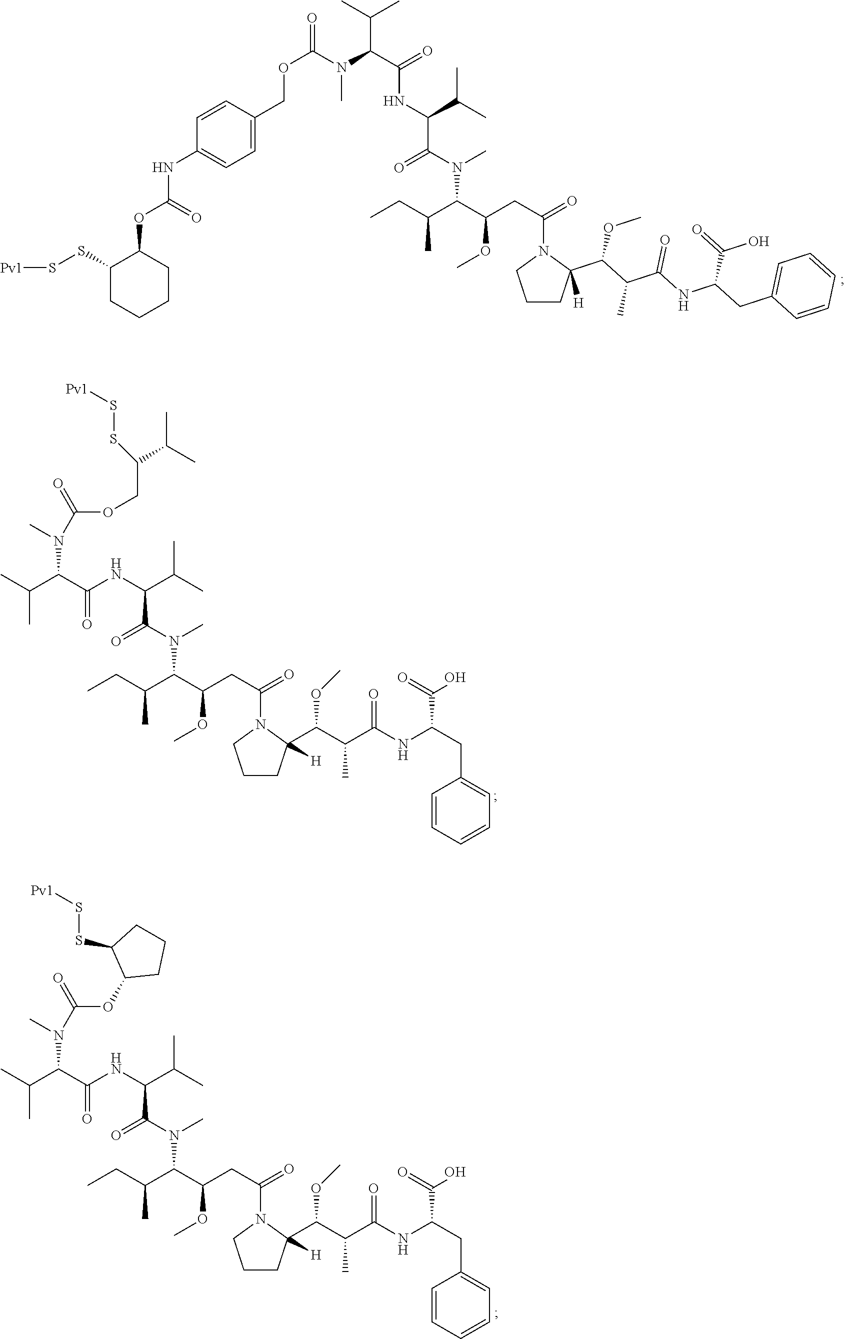 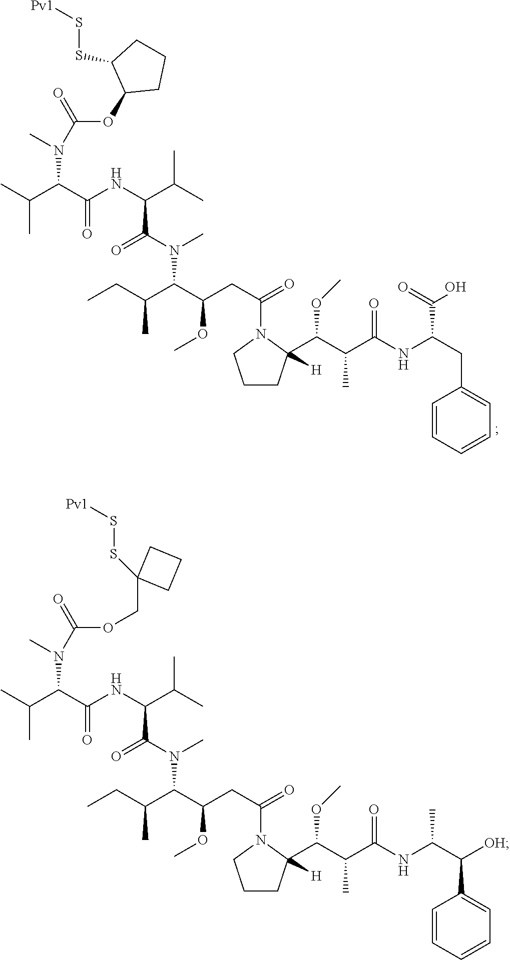 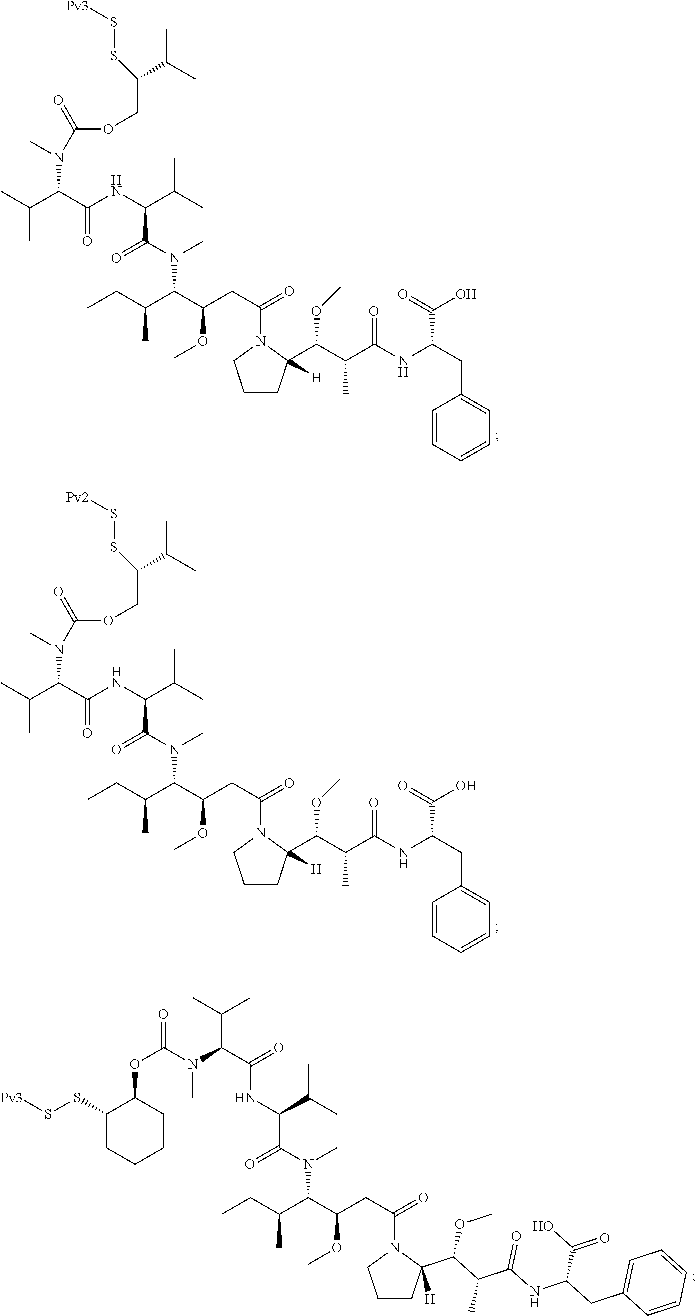 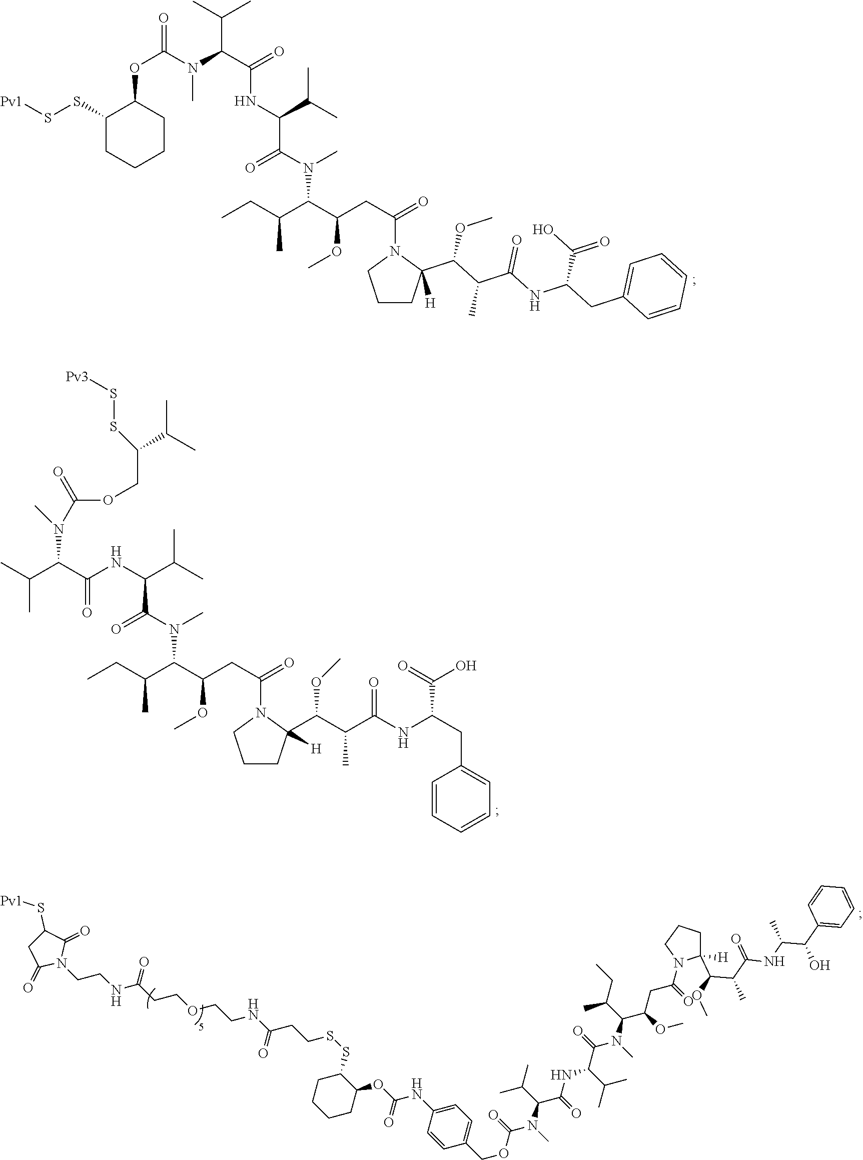 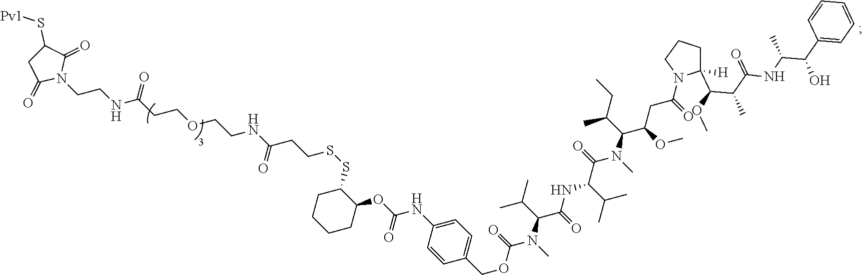
or a pharmaceutically acceptable salt of any of the aforementioned, wherein:
Pv1 is a peptide comprising the sequence:
(SEQ ID NO: 1)
ADDQNPWRAYLDLLFPTDTLLLDLLWCG;
Pv2 is a peptide comprising the sequence:
(SEQ ID NO: 2)
AEQNPIYWARYADWLFTTPLLLLDLALLVDADECG;
and
Pv3 is a peptide comprising the sequence:
(SEQ ID NO: 3)
ADDQNPWRAYLDLLFPTDTLLLDLLWDADECG.

70. A pharmaceutical composition that comprises a compound of claim 1, or a pharmaceutically acceptable salt thereof.

71. A method of treating cancer in a patient in need thereof comprising administering to the patient a therapeutically effective amount of a compound of claim 1, or a pharmaceutically acceptable salt thereof.

72. The method of claim 71, wherein the cancer is selected from bladder cancer, bone cancer, glioma, breast cancer, cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer, lung cancer, melanoma, prostate cancer, rectal cancer, renal clear cell carcinoma, skin cancer, stomach cancer, testicular cancer, thyroid cancer, and uterine cancer.

73. The method of claim 71, wherein the cancer is selected from lung cancer, colorectal cancer, and prostate cancer.

74. The method of claim 73, wherein the lung cancer is non-small cell lung cancer.

75. The method of claim 71, wherein the cancer is selected from Hodgkin lymphoma, anaplastic large cell lymphoma (ALCL), diffuse large B-cell lymphoma (DLBCL), ovarian cancer, urothelial cancer, non-small cell lung cancer (NSCLC), triple-negative breast cancer, squamous non-small cell lung cancer (sqNSCLC), squamous head and neck cancer, Non-Hodgkin lymphoma, pancreatic cancer, chronic myeloid leukemia (CML), acute myeloid leukemia (AML), fallopian tube cancer, and peritoneal cancer.
